(19) United States
(12) Patent Application Publication
(10) Pub. No.: US 2019/0134174 A1
(43) Pub. Date: May 9, 2019

(54) COMPOSITIONS AND METHODS FOR TUMOR VACCINATION AND IMMUNOTHERAPY INVOLVING HER2/NEU

(71) Applicant: ETUBICS CORPORATION, Seattle, WA (US)

(72) Inventors: Frank R. JONES, Seattle, WA (US); Joseph BALINT, Seattle, WA (US); Wayne GODFREY, Seattle, WA (US); Elizabeth GABITZSCH, Seattle, WA (US)

(21) Appl. No.: 16/306,088

(22) PCT Filed: Jun. 2, 2017
(86) PCT No.: PCT/US17/35718

§ 371 Date: Nov. 30, 2018

Related U.S. Application Data
(60) Provisional application No. 62/345,575, filed on Jun. 3, 2016.
(60) Provisional application No. 62/361,292, filed on Jul. 12, 2016.

(51) Int. Cl. A61K 39/00; C12N 15/85; A61P 35/04

(52) U.S. Cl. CPC A61K 39/001106; C12N 15/85; A61K 2039/5256; A61K 2039/5254; A61P 35/04

(57) ABSTRACT

In certain embodiments, methods and compositions are provided for generating immune responses against tumor antigens such as a HER2/neu antigen or epitope. In particular embodiments there may be provided methods for constructing and producing recombinant adenovirus-based vector vaccines containing nucleic acid sequences encoding tumor antigens such as a HER2/neu antigen or epitope that allow for vaccinations in individuals with preexisting immunity to adenovirus.

### CROSS REFERENCE

This application claims the benefit of U.S. Provisional Patent Application No. 62/361,292 filed Jul. 12, 2016, and U.S. Provisional Patent Application No. 62/345,575 filed Jun. 3, 2016, the disclosures of which are herein incorporated by reference in their entireties.

### STATEMENT OF GOVERNMENT INTEREST

The invention was made with government support under SBIR Grant No. 1R43CA139663-01, SBIR Contract No. HHSN261201100090C, SBIR Contract No. HHSN261201300066C awarded by the National Cancer Institute (NCI), and Award W81XWH-12-1-0574; BC113107 from the Department of Defense. The government has certain rights in the invention.

### BACKGROUND

Vaccines help the body fight disease by training the immune system to recognize and destroy harmful substances and diseased cells. Vaccines can be largely grouped into two types, preventive and treatment vaccines. Prevention vaccines are given to healthy people to prevent the development of specific diseases, while treatment vaccines, also referred to as immunotherapies, are given to a person who has been diagnosed with disease to help stop the disease from growing and spreading or as a preventive measure.

Viral vaccines are currently being developed to help fight infectious diseases and cancers. These viral vaccines work by inducing expression of a small fraction of genes associated with a disease within the host's cells, which in turn, enhance the host's immune system to identify and destroy diseased cells. As such, clinical response of a viral vaccine can depend on the ability of the vaccine to obtain a high-level immunogenicity and have sustained long-term expression.

Therefore, there remains a need to discover novel compositions and methods for enhanced therapeutic response to complex diseases such as cancer.

### SUMMARY

In various aspects, the present disclosure provides a composition comprising a replication-defective virus vector comprising a nucleic acid sequence encoding a HER2/neu antigen that is a fragment of a native HER2/neu protein. In some aspects, the HER2/neu antigen does not have an intracellular domain of a native HER2/neu protein. In some aspects, the HER2/neu antigen has a transmembrane domain and an extracellular domain of a native HER2/neu protein. In some aspects, the HER2/neu antigen has a sequence at least 80%, at least 85%, at least 90%, at least 92%, at least 95%, at least 97%, or at least 99% identical to SEQ ID NO: 1 or SEQ ID NO: 2, the nucleic acid sequence has a sequence at least 80%, at least 85%, at least 90%, at least 92%, at least 95%, at least 97%, or at least 99% identical to SEQ ID NO: 1 or positions 1033-3107 of SEQ ID NO: 3, and/or the replication-defective virus vector has a sequence at least 80%, at least 85%, at least 90%, at least 92%, at least 95%, at least 97%, or at least 99% identical to SEQ ID NO: 3.

In some aspects, the replication-defective virus vector is an adenovirus vector. In some aspects, the adenovirus vector comprises a deletion in an E1 region, an E2b region, an E3 region, an E4 region, or a combination thereof. In some aspects, the adenovirus vector comprises a deletion in an E2b region. In some aspects, the adenovirus vector comprises a deletion in an E1 region, an E2b region, and an E3 region.

In some aspects, the compositon comprises from at least 1×10⁹ to at least 5×10¹² virus particles. In some aspects, the composition comprises at least 5×10⁹ virus particles. In some aspects, the composition comprises at least 5×10¹⁰ virus particles. In some aspects, the composition comprises at least 5×10¹¹ virus particles. In some aspects, the composition comprises at least 5×10¹² virus particles.

In some aspects, the replication-defective virus vector further comprises a nucleic acid sequences encoding a costimulatory molecule. In some aspects, the replication-defective virus vector further comprises a nucleic acid sequence encoding an immunological fusion partner. In further aspects, the costimulatory molecule comprises B7, ICAM-1, LFA-3, or a combination thereof. In some aspects, the costimulatory molecule comprises a combination of B7, ICAM-1, and LFA-3.

In some aspects, the composition further comprises a plurality of nucleic acid sequences encoding a plurality of costimulatory molecules positioned in the same replication-defective virus vector.

In some aspects, the composition further comprises a plurality of nucleic acid sequences encoding a plurality of costimulatory molecules positioned in separate replication-defective virus vectors.

In some aspects, the composition further comprises a nucleic acid sequence encoding one or more target antigens or immunological epitopes thereof. In some aspects, the replication-defective virus vector further comprises a nucleic acid sequence encoding one or more target antigens or immunological epitopes thereof. In some aspects, the one or more target antigens is a tumor neo-antigen, tumor neo-epitope, tumor-specific antigen, tumor-associated antigen, tissue-specific antigen, bacterial antigen, viral antigen, yeast antigen, fungal antigen, protozoan antigen, parasite antigen, mitogen, or a combination thereof. In some aspects, the one or more target antigens is folate receptor alpha, WT1, p53, MAGE-A1, MAGE-A2, MAGE-A3, MAGE-A4, MAGE-A6, MAGE-A10, MAGE-A12, BAGE, DAM-6, -10, GAGE-1, -2, -8, GAGE-3, -4, -5, -6, -7B, NA88-A, NY-ESO-1, MART-1, MC1R, Gp100, Tyrosinase, TRP-1, TRP-2, ART-4, CAMEL, CEA, Cyp-B, HER2/neu, BRCA1, BRACHYURY, BRACHYURY(TIVS7-2, polymorphism), BRACHYURY (IVS7 T/C polymorphism), T BRACHYURY, T, hTERT, hTRT, iCE, MUC1, MUC1 (VNTR polymorphism), MUC1-c, MUC1n, MUC2, PRAME, P15, RU1, RU2, SART-1, SART-3, WT1, AFP, β-catenin/m, Caspase-8/m, CEA, CDK-4/m, HER3, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, ETV6/AML, LDLR/FUT, Pml/RARα, or TEL/AML1, or a modified variant, a splice variant, a functional epitope, an epitope agonist, or a combination thereof. In some aspects, the one or more target antigens is CEA, Brachyury, MUC1, MUC1-c, or any combination thereof. In some aspects, the one or more target antigens is CEA.

In some aspects, the one or more target antigens is Brachyury.

In some aspects, the one or more target antigens is MUC1 or MUC1-c.

In some aspects, the one or more target antigens is HER3.

In some aspects, CEA comprises a sequence at least 80%, at least 85%, at least 90%, at least 92%, at least 95%, at least 97%, or at least 99% identical to SEQ ID NO: 30, SEQ ID NO: 31, or positions 1057-3165 of SEQ ID NO: 29 In some aspects, MUC1-c comprises a sequence at least 80%, at least 85%, at least 90%, at least 92%, at least 95%, at least 97%, or at least 99% identical to SEQ ID NO: 32 or SEQ ID NO: 33.

In some aspects, Brachyury comprises a sequence at least 80%, at least 85%, at least 90%, at least 92%, at least 95%, at least 97%, or at least 99% identical to SEQ ID NO: 34.

In some aspects, HER3 comprises a sequence at least 80%, at least 85%, at least 90%, at least 92%, at least 95%, at least 97%, or at least 99% identical to SEQ ID NO: 27.

In some aspects, the replication-defective virus vector further comprises a selectable marker. In some aspects, the selectable marker is a lacZ gene, thymidine kinase, gpt, GUS, or a vaccinia K1L host range gene, or a combination thereof.

In various aspects, the present disclosure provides a pharmaceutical composition comprising any composition as decribed herein and a pharmaceutically acceptable carrier.

In various aspects, the present disclosure provides a host cell comprising any composition as described herein.

In various aspects, the present disclosure provides a method of preparing a tumor vaccine, the method comprising preparing any pharmaceutical composition as described herein.

In various aspects, the present disclosure provides a method of enhancing an immune response in a subject in need thereof, the method comprising administering a therapeutically effective amount of any composition as described herein or any pharmaceutical composition as described herein to the subject.

In various aspects, the present disclosure provides a method of treating a cancer in a subject in need thereof, the method comprising administering a therapeutically effective amount of any composition as described herein or any pharmaceutical composition as described herein to the subject.

In some aspects, the method further comprises readministering the pharmaceutical composition to the subject.

In some aspects, the method further comprises administering an immune checkpoint inhibitor to the subject. In some aspects, the immune checkpoint inhibitor inhibits PD1, PDL1, PDL2, CD28, CD80, CD86, CTLA4, B7RP1, ICOS, B7RPI, B7-H3, B7-H4, BTLA, HVEM, KIR, TCR, LAG3, CD137, CD137L, OX40, OX40L, CD27, CD70, CD40, CD40L, TIM3, GAL9, ADORA, CD276, VTCN1, IDO1, KIR3DL1, HAVCR2, VISTA, or CD244. In some aspects, the immune checkpoint inhibitor inhibits PD1 or PDL1. In some aspects, the immune checkpoint inhibitor is an anti-PD1 or anti-PDL1 antibody. In some aspects, the immune checkpoint inhibitor is an anti-PDL1 antibody.

In some aspects, the administering is intravenous, subcutaneous, intralymphatic, intratumoral, intradermal, intramuscular, intraperitoneal, intrarectal, intravaginal, intranasal, oral, via bladder instillation, or via scarification.

In some aspects, the enhanced immune response is a cell-mediated or humoral response. In some aspects, the enhanced immune response is an enhancement of B-cell proliferation, CD4+ T cell proliferation, CD8+ T cell proliferation, or a combination thereof. In some aspects, the enhanced immune response is an enhancement of IL-2 production, IFN-γ production or combination thereof. In some aspects, the enhanced immune response is an enhancement of antigen presenting cell proliferation, function or combination thereof.

In some aspects, the subject has been previously administered an adenovirus vector. In some aspects, the subject has pre-existing immunity to adenovirus vectors. In some aspects, the subject is determined to have pre-existing immunity to adenovirus vectors.

In some aspects, the method further comprises administering to the subject a chemotherapy, radiation, a different immunotherapy, or a combination thereof.

In some aspects, the subject is a human or a non-human animal.

In some aspects, the subject has previously been treated for cancer.

In some aspects, the administering the therapeutically effective amount is repeated at least three times. In some aspects, the administering the therapeutically effective amount comprises from at least 1×10⁹ to at least 5×10¹² virus particles. In some aspects, the administering the therapeutically effective amount comprises 5×10⁹ virus particles per dose. In some aspects, the administering the therapeutically effective amount comprises at least 5×10¹⁰ virus particles per dose. In some aspects, the administering the therapeutically effective amount comprises at least 5×10¹¹ virus particles per dose. In some aspects, the administering the therapeutically effective amount comprises at least 5×10¹² virus particles per dose. In some aspects, the administering the therapeutically effective amount is repeated every two or three weeks.

In some aspects, the administering the therapeutically effective amount is followed by one or more booster immunizations comprising the same composition or pharmaceutical composition. In some aspects, the booster immunization is administered every one, two, three, four, five, six, seven, eight, nine, ten, eleven, or twelve months or more. In some aspects, the booster immunization is repeated three four, five, six, seven, eight, nine, ten, eleven, or twelve or more times. In some aspects, the administering the therapeutically effective amount is a primary immunization repeated every one, two, or three weeks for three four, five, six, seven, eight, nine, ten, eleven, or twelve or more times followed by a booster immunization repeated every one, two, three, four, five, six, seven, eight, nine, ten, eleven, or twelve or more months for three or more times.

In some aspects, the method further comprises administering to the subject a pharmaceutical composition comprising a population of engineered nature killer (NK) cells. In some aspects, the engineered NK cells comprise one or more NK cells that have been modified as essentially lacking the expression of KIR (killer inhibitory receptors), one or more NK cells that have been modified to express a high affinity CD16 variant, and one or more NK cells that have been modified to express one or more CARs (chimeric antigen receptors), or any combinations thereof. In some aspects, the engineered NK cells comprise one or more NK cells that have been modified as essentially lacking the expression KIR. In some aspects, the engineered NK cells comprise one or more NK cells that have been modified to express a high affinity CD16 variant. In some aspects, the engineered NK cells comprise one or more NK cells that have been modified to express one or more CARs. In further aspects, the CAR is a CAR for a tumor neo-antigen, tumor neo-epitope, WT1, p53, MAGE-A1, MAGE-A2, MAGE-A3, MAGE-A4, MAGE-A6, MAGE-A10, MAGE-A12, BAGE, DAM-6, DAM-10, Folate receptor alpha, GAGE-1, GAGE-2, GAGE-8, GAGE-3, GAGE-4, GAGE-5, GAGE-6, GAGE-7B, NA88-A, NY-ESO-1, MART-1, MC1R, Gp100, Tyrosinase, TRP-1, TRP-2, ART-4, CAMEL, CEA, Cyp-B, HER2/neu, HER3, BRCA1, Brachyury, Brachyury (TIVS7-2, polymorphism), Brachyury (IVS7 T/C polymorphism), T Brachyury, T, hTERT, hTRT, iCE, MUC1, MUC1 (VNTR polymorphism), MUC1C, MUC1n, MUC2, PRAME, P15, RU1, RU2, SART-1, SART-3, AFP, β-catenin/m, Caspase-8/m, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, ETV6/AML, LDLR/FUT, Pml/RARα, TEL/AML1, or any combination thereof.

In some aspects, the replication-defective adenovirus vector is comprised in a cell. In some aspects, the cell is a dendritic cells (DC).

In some aspects, the method further comprises administering a pharmaceutical composition comprising a therapeutically effective amount of IL-15 or a replication-defective vector comprising a nucleic acid sequence encoding IL-15.

In some aspects, the subject has HER2/neu-expressing cancer. In some aspects, the subject has HER2/neu expressing breast cancer. In some aspects, the subject has HER2/neu expressing bone cancer. In some aspects, the cancer is osteosarcoma. In some aspects, the subject has HER2/neu expressing gastric cancer. In some aspects, the subject has unresectable, locally advanced or metastatic cancer. In some aspects, the method further comprises administering an additional cancer therapy to the subject.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows an illustrative embodiment of a restriction map of the Ad5 [E1−, E2b−]-HER2/neu vector, pAd5CMV/HER2/neu/Δpp.

FIG. 2 shows an illustrative embodiment of the clinical study design and treatment regimen.

### DETAILED DESCRIPTION

While the making and using of various embodiments are discussed in detail below, it should be appreciated that the many applicable inventive concepts provided herein can be embodied in a wide variety of specific contexts. The specific embodiments discussed herein are merely illustrative of specific ways to make and use the invention and do not delimit the scope of the invention.

To facilitate the understanding of certain aspects, a number of terms are defined below. Terms defined herein have meanings as commonly understood by a person of ordinary skill in the areas relevant to the present invention.

Terms such as “a,” “an” and “the” are not intended to refer to only a singular entity, but include the general class of which a specific example may be used for illustration. The terminology herein is used to describe specific embodiments of the invention, but their usage does not delimit the invention, except as outlined in the claims.

By “individual,” “subject” or “patient” is meant any single subject for which therapy is desired, including but not limited to humans, non-human primates, rodents, dogs, or pigs. Also intended to be included as a subject are any subjects involved in clinical research trials not showing any clinical sign of disease, or subjects involved in epidemiological studies, or subjects used as controls.

As used herein, the term “gene” refers to a functional protein, polypeptide or peptide-encoding unit. As will be understood by those in the art, this functional term includes genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated. The term “allele” or “allelic form” refers to an alternative version of a gene encoding the same functional protein but containing differences in nucleotide sequence relative to another version of the same gene. In certain aspects, the term “gene” means the gene and all currently known variants thereof and any further variants which may be elucidated.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

As used herein, unless otherwise indicated, the article “a” means one or more unless explicitly otherwise provided for.

As used herein, unless otherwise indicated, terms such as “contain,” “containing,” “include,” “including,” and the like mean “comprising.”

As used herein, unless otherwise indicated, the term “or” can be conjunctive or disjunctive.

As used herein, unless otherwise indicated, any embodiment can be combined with any other embodiment.

As used herein, unless otherwise indicated, some inventive embodiments herein contemplate numerical ranges. A variety of aspects can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. When ranges are present, the ranges include the range endpoints.

The term “adenovirus” or “Ad” refers to a group of non-enveloped DNA viruses from the family Adenoviridae. In addition to human hosts, these viruses can be found in, but are not limited to, avian, bovine, porcine and canine species. Certain aspects may contemplate the use of any adenovirus from any of the four genera of the family Adenoviridae (e.g., Aviadenovirus, Mastadenovirus, Atadenovirus and Siadenovirus) as the basis of an E2b deleted virus vector, or vector containing other deletions as described herein. In addition, several serotypes are found in each species. Ad also pertains to genetic derivatives of any of these viral serotypes, including but not limited to, genetic mutation, deletion or transposition of homologous or heterologous DNA sequences.

A “helper adenovirus” or “helper virus” refers to an Ad that can supply viral functions that a particular host cell cannot (the host may provide Ad gene products such as E1 proteins). This virus is used to supply, in trans, functions (e.g., proteins) that are lacking in a second virus, or helper dependent virus (e.g., a gutted or gutless virus, or a virus deleted for a particular region such as E2b or other region as described herein); the first replication-incompetent virus is said to “help” the second, helper dependent virus thereby permitting the production of the second viral genome in a cell.

The term “Adenovirus5 null (Ad5null),” as used herein, refers to a non-replicating Ad that does not contain any heterologous nucleic acid sequences for expression.

The term “First Generation adenovirus,” as used herein, refers to an Ad that has the early region 1 (E1) deleted. In additional cases, the nonessential early region 3 (E3) may also be deleted.

The term “gutted” or “gutless,” as used herein, refers to an adenovirus vector that has been deleted of all viral coding regions.

The term “transfection” as used herein refers to the introduction of foreign nucleic acid into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign nucleic acid, DNA or RNA, into the genome of the transfected cell. The term “stable transfectant” refers to a cell which has stably integrated foreign DNA into the genomic DNA.

The term “reporter gene” indicates a nucleotide sequence that encodes a reporter molecule (including an enzyme). A “reporter molecule” is detectable in any of a variety of detection systems, including, but not limited to enzyme-based detection assays (e.g., ELISA, as well as enzyme-based histochemical assays), fluorescent, radioactive, and luminescent systems.

In one embodiment, there may be provided the E. coli β-galactosidase gene (available from Pharmacia Biotech, Pistacataway, N.J.), green fluorescent protein (GFP) (commercially available from Clontech, Palo Alto, Calif.), the human placental alkaline phosphatase gene, the chloramphenicol acetyltransferase (CAT) gene as reporter genes; other reporter genes are known to the art and may be employed.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The nucleic acid sequence thus codes for the amino acid sequence.

The term “heterologous nucleic acid sequence,” as used herein, refers to a nucleotide sequence that is ligated to, or is manipulated to become ligated to, a nucleic acid sequence to which it is not ligated in nature, or to which it is ligated at a different location in nature. Heterologous nucleic acid may include a nucleotide sequence that is naturally found in the cell into which it is introduced or the heterologous nucleic acid may contain some modification relative to the naturally occurring sequence.

The term “transgene” refers to any gene coding region, either natural or heterologous nucleic acid sequences or fused homologous or heterologous nucleic acid sequences, introduced into the cells or genome of a test subject. In certain aspects, transgenes are carried on any viral vector that is used to introduce the transgenes to the cells of the subject.

The term “Second Generation Adenovirus,” as used herein, refers to an Ad that has all or parts of the E1, E2, E3, and, in certain embodiments, E4 DNA gene sequences deleted (removed) from the virus.

As used herein, the term “fragment or segment,” as applied to a nucleic acid sequence, gene or polypeptide, will ordinarily be at least about 5 contiguous nucleic acid bases (for nucleic acid sequence or gene) or amino acids (for polypeptides), typically at least about 10 contiguous nucleic acid bases or amino acids, more typically at least about 20 contiguous nucleic acid bases or amino acids, usually at least about 30 contiguous nucleic acid bases or amino acids, preferably at least about 40 contiguous nucleic acid bases or amino acids, more preferably at least about 50 contiguous nucleic acid bases or amino acids, and even more preferably at least about 60 to 80 or more contiguous nucleic acid bases or amino acids in length. “Overlapping fragments” as used herein, refer to contiguous nucleic acid or peptide fragments which begin at the amino terminal end of a nucleic acid or protein and end at the carboxy terminal end of the nucleic acid or protein. Each nucleic acid or peptide fragment has at least about one contiguous nucleic acid or amino acid position in common with the next nucleic acid or peptide fragment, more preferably at least about three contiguous nucleic acid bases or amino acid positions in common, most preferably at least about ten contiguous nucleic acid bases amino acid positions in common.

A significant “fragment” in a nucleic acid context is a contiguous segment of at least about 17 nucleotides, generally at least 20 nucleotides, more generally at least 23 nucleotides, ordinarily at least 26 nucleotides, more ordinarily at least 29 nucleotides, often at least 32 nucleotides, more often at least 35 nucleotides, typically at least 38 nucleotides, more typically at least 41 nucleotides, usually at least 44 nucleotides, more usually at least 47 nucleotides, preferably at least 50 nucleotides, more preferably at least 53 nucleotides, and in particularly preferred embodiments will be at least 56 or more nucleotides.

A “vector” is a composition which can transduce, transfect, transform or infect a cell, thereby causing the cell to express nucleic acids and/or proteins other than those native to the cell, or in a manner not native to the cell. A cell is “transduced” by a nucleic acid when the nucleic acid is translocated into the cell from the extracellular environment. Any method of transferring a nucleic acid into the cell may be used; the term, unless otherwise indicated, does not imply any particular method of delivering a nucleic acid into a cell. A cell is “transformed” by a nucleic acid when the nucleic acid is transduced into the cell and stably Teplicated. A vector includes a nucleic acid (ordinarily RNA or DNA) to be expressed by the cell. A vector optionally includes materials to aid in achieving entry of the nucleic acid into the cell, such as a virus particle, liposome, protein coating or the like. A “cell transduction vector” is a vector which encodes a nucleic acid capable of stable replication and expression in a cell once the nucleic acid is transduced into the cell.

The term “variant,” when used in the context of a polynucleotide sequence, may encompass a polynucleotide sequence related to a wild type gene. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type target genes. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

As used herein, “variant” of polypeptides refers to an amino acid sequence that is altered by one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs,) or single base mutations in which the polynucleotide sequence varies by one base.

An “antigen” is any substance that reacts specifically with antibodies or T lymphocytes (T cells). An “antigen-binding site” is the part of an immunoglobulin molecule that specifically binds an antigen. Additionally, an antigen-binding site includes any such site on any antigen-binding molecule, including, but not limited to, an MHC molecule or T cell receptor. “Antigen processing” refers to the degradation of an antigen into fragments (e.g., the degradation of a protein into peptides) and the association of one or more of these fragments (e.g., via binding) with MHC molecules for presentation by “antigen-presenting cells” to specific T cells.

“Dendritic cells” (DC) are potent antigen-presenting cells, capable of triggering a robust adaptive immune response in vivo. It has been shown that activated, mature DCs provide the signals required for T cell activation and proliferation. These signals can be categorized into two types. The first type, which gives specificity to the immune response, is mediated through interaction between the T-cell receptor/CD3 (“TCR/CD3”) complex and an antigenic peptide presented by a major histocompatibility complex (“MHC” defined above) class I or II protein on the surface of APCs. The second type of signal, called a co-stimulatory signal, is neither antigen-specific nor MHC-restricted, and can lead to a full proliferation response of T cells and induction of T cell effector functions in the presence of the first type of signals. This two-fold signaling can, therefore, result in a vigorous immune response. As noted supra, in most non-avian vertebrates, DCs arise from bone marrow-derived precursors. Immature DCs are found in the peripheral blood and cord blood and in the thymus. Additional immature populations may be present elsewhere. DCs of various stages of maturity are also found in the spleen, lymph nodes, tonsils, and human intestine. Avian DC may also be found in the bursa of Fabricius, a primary immune organ unique to avians. In a particular embodiment, the dendritic cells are mammalian, preferably human, mouse, or rat.

A “co-stimulatory molecule” encompasses any single molecule or combination of molecules which, when acting together with a peptide MHC complex bound by a T cell receptor on the surface of a T cell, provides a co-stimulatory effect which achieves activation of the T cell that binds the peptide.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. As used herein, the phrase “consisting essentially of” limits the scope of a claim to the specified materials or steps and those that do not materially affect the basic and novel characteristic(s) of the claimed invention. As used herein, the phrase “consisting of” excludes any element, step, or ingredient not specified in the claim except for, e.g., impurities ordinarily associated with the element or limitation.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. A skilled artisan will understand that typically there is no limit on the number of items or terms in any combination, unless otherwise apparent from the context.

As used herein, words of approximation such as, without limitation, “about,” “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12, or 15%.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety to the same extent as if each individual publication, patent, or patent application was specifically and individually indicated to be incorporated by reference.

Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### I. HER2/Neu Target Antigens

In certain aspects, there may be provided expression constructs or vectors comprising nucleic acid sequences that encode one or more target proteins of interest or target antigens, such as a HER2/neu antigen or epitope as described herein.

HER-2/neu (p185) is the protein product of the HER-2/neu oncogene. In some aspects, the HER-2/neu gene is amplified and the HER-2/neu protein is overexpressed in a variety of cancers including breast, ovarian, gastric, colon, lung, prostate, and bone. In some aspects, HER-2/neu is related to malignant transformation. In some aspects, it is found in 50%-60% of ductal in situ carcinoma and 20%-40% of all breast cancers, as well as a substantial fraction of adenocarcinomas arising in the ovaries, prostate, colon and lung. In some aspects, the HER-2/neu protein is overexpressed in cancers of the bone, including osteosarcoma. In some aspects, HER-2/neu is intimately associated not only with the malignant phenotype, but also with the aggressiveness of the malignancy, being found in one-fourth of all invasive breast cancers. In some aspects, HER-2/neu overexpression is correlated with a poor prognosis in both breast and ovarian cancer.

In some aspects, HER-2/neu is a transmembrane protein with a relative molecular mass of 185 kd that is approximately 1255 amino acids (aa) in length. It has an extracellular binding domain (ECD) of approximately 645 aa, with 40% homology to epidermal growth factor receptor (EGFR), a highly hydrophobic transmembrane domain (TM), and an intracellular domain of approximately 580 aa with 80% homology to EGFR.

In further aspects, there may be provided expression constructs or vectors that may contain nucleic acid encoding at least, at most or about one, two, three, four, five, six, seven, eight, nine, ten, 11, 12, 13, 14, 15, 16, 17, 18, 19, 20, 30, 40, 50, 60, 70, 80, 90, 100, 200, 300, 400, or 500 different target antigens of interest or any number or ranges derived therefrom. The expression constructs or vectors may contain nucleic acid sequences encoding multiple fragments or epitopes from one HER2/neu antigen or may contain one or more fragments or epitopes from numerous different target antigens including a HER2/neu antigen or epitope as described herein.

The HER2/neu antigen may be a full length protein or may be an immunogenic fragment (e.g., an epitope) thereof. Immunogenic fragments may be identified using available techniques, such as those summarized in Paul, Fundamental Immunology, 3rd ed., 243-247 (Raven Press, 1993) and references cited therein. Representative techniques for identifying immunogenic fragments include screening polypeptides for the ability to react with antigen-specific antisera and/or T-cell lines or clones. An immunogenic fragment of a particular target polypeptide may be a fragment that reacts with such antisera and/or T-cells at a level that is not substantially less than the reactivity of the full length target polypeptide (e.g., in an ELISA and/or T-cell reactivity assay). In other words, an immunogenic fragment may react within such assays at a level that is similar to or greater than the reactivity of the full length polypeptide. Such screens may generally be performed using methods available to those of ordinary skill in the art, such as those described in Harlow and Lane, Antibodies: A Laboratory Manual, Cold Spring Harbor Laboratory, 1988.

In some cases an immunogenic epitope such as a HER2/neu epitope can be 8 to 10 amino acids long. In some cases a HER2/neu epitope is four to ten amino acids long or over 10 amino acids long. An immunogenic epitope such as a HER2/neu epitope can comprise a length of or can comprise a length of at least, about, or at most 1, 2, 3, 4, 5, 6, 7, 8, 9, 10, 11, 12, 13, 14, 15, 16, 17, 18, 19, 20 amino acids or any number or ranges derived therefrom. An immunogenic epitope such as a HER2/neu epitope can be any length of amino acids.

In some embodiments, a HER2/neu epitope can have a nucleic acid sequence that is at least 80%, at least 85%, at least 90%, at least 92%, at least 95%, at least 97%, or at least 99% identical to SEQ ID NO: 1 (nucleic acid sequence of a truncated HER2/neu containing the transmembrane and extracellular domains) or positions 1033-3107 of SEQ ID NO: 3. In certain emodiments, a HER2/neu epitope can have a sequence as set forth in SEQ ID NO: 1 or positions 1033-3107 of SEQ ID NO: 3 (nucleic acid sequence of an Ad5 [E1−, E2b−]-HER2/neu vector wherein the HER2/neu is the truncated HER2/neu of SEQ ID NO: 1). In some embodiments, an Ad5 [E1−, E2b−]-HER2/neu vector can have a nucleic acid sequence that is at least 80%, at least 85%, at least 90%, at least 92%, at least 95%, at least 97%, or at least 99% identical to SEQ ID NO: 3. In some embodiments, Ad5 [E1−, E2b−]-HER2/neu vaccines can be combined with Ad5 [E1−, E2b−]-HER3 vaccines in which the HER3 antigen can be a truncated HER3 antigen comprising a transmembrane and extracellular domains. In some embodiments, the HER 3 antigen can have a nucleici acid sequences that is at least 80%, at least 85%, at least 90%, at least 92%, at least 95%, at least 97%, or at least 99% identical to SEQ ID NO: 27 (nucleic acid sequence of a truncated HER3 containing the transmembrane and extracellular domains).

Additional non-limiting examples of target antigens include human epidermal growth factor receptor 2 (HER2/neu), carcinoembryonic antigen (CEA), a tumor neo-antigens or tumor neo-epitope, folate receptor alpha, WT1, brachyury (TIVS7-2, polymorphism), brachyury (IVS7 T/C polymorphism), T brachyury, T, hTERT, hTRT, iCE, BAGE, DAM-6, -10, GAGE-1, -2, -8, GAGE-3, -4, -5, -6, -7B, NA88-A, NY-ESO-1, MART-1, MC1R, Gp100, Tyrosinase, TRP-1, TRP-2, ART-4, CAMEL, Cyp-B, EGFR, HER2/neu, MUC1, MUC1 (VNTR polymorphism), MUC1-c, MUC1-n, MUC2, PRAME, P15, RU1, RU2, SART-1, SART-3, β-catenin/m, Caspase-8/m, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, ETV6/AML, LDLR/FUT, Pml/RARα, TEL/AML1, human epidermal growth factor receptor 3 (HER3), alpha-actinin-4, ARTC1, CAR-ABL fusion protein (b3a2), B-RAF, CASP-5, CASP-8, beta-catenin, Cdc27, CDK4, CDKN2A, COA-1, dek-can fusion protein, EFTUD2, Elongation factor 2, ETV6-AML1 fusion protein, FLT3-ITD, FN1, GPNMB, LDLR-fucosyltransferase fusion protein, HLA-A2d, HLA-A1 ld, hsp70-2, KIAAO205, MART2, ME1, Myosin class I, NFYC, OGT, OS-9, pml-RARalpha fusion protein, PRDX5, PTPRK, K-ras, N-ras, RBAF600, SIRT2, SNRPD1, SYT-SSX1- or -SSX2 fusion protein, TGF-betaRII, triosephosphate isomerase, BAGE-1, GAGE-1, 2, 8, Gage 3, 4, 5, 6, 7, GnTVf, HERV-K-MEL, KK-LC-1, KM-HN-1, LAGE-1, MAGE-A1, MAGE-A2, MAGE-A3, MAGE-A4, MAGE-A6, MAGE-A9, MAGE-A10, MAGE-A12, MAGE-C2, mucin, NA-88, NY-ESO-1/LAGE-2, SAGE, Sp17, SSX-2, SSX-4, TAG-1, TAG-2, TRAG-3, TRP2-INT2g, XAGE-1b, gp100/Pmel17, mammaglobin-A, Melan-A/MART-1, NY-BR-1, OA1, RAB38/NY-MEL-1, TRP-1/gp75, adipophilin, AIM-2, ALDH1A1, BCLX (L), BCMA, BING-4, CPSF, cyclin D1, DKK1, ENAH (hMena), EP-CAM, EphA3, EZH2, FGF5, G250/MN/CAIX, IL13Ralpha2, intestinal carboxyl esterase, alpha fetoprotein, M-CSFT, MCSP, mdm-2, MMP-2, p53, PBF, PRAME, RAGE-1, RGS5, RNF43, RU2AS, secernin 1, SOX10, survivin, Telomerase, VEGF, or any combination thereof.

In some aspects, tumor neo-epitopes as used herein are tumor-specific epitopes, such as EQVWGMAVR (SEQ ID NO: 6) or CQGPEQVWGMAVREL (SEQ ID NO: 7) (R346W mutation of FLRT2), GETVTMPCP (SEQ ID NO: 8) or NVGETVTMPCPKVFS (SEQ ID NO: 9) (V73M mutation of VIPR2), GLGAQCSEA (SEQ ID NO: 10) or NNGLGAQCSEAVTLN (SEQ ID NO: 11) (R286C mutation of FCRL1), RKLTTELTI (SEQ ID NO: 12), LGPERRKLTTELTII (SEQ ID NO: 13), or PERRKLTTE (SEQ ID NO: 14) (S1613L mutation of FAT4), MDWVWMDTT (SEQ ID NO: 15), AVMDWVWMDTTLSLS (SEQ ID NO: 16), or VWMDTTLSL (SEQ ID NO: 17) (T2356M mutation of PIEZO2), GKTLNPSQT (SEQ ID NO: 18), SWFREGKTLNPSQTS (SEQ ID NO: 19), or REGKTLNPS (SEQ ID NO: 20) (A292T mutation of SIGLEC14), VRNATSYRC (SEQ ID NO: 21), LPNVTVRNATSYRCG (SEQ ID NO: 22), or NVTVRNATS (SEQ ID NO: 23) (D1143N mutation of SIGLEC1), FAMAQIPSL (SEQ ID NO: 24), PFAMAQIPSLSLRAV (SEQ ID NO: 25), or AQIPSLSLR (SEQ ID NO: 26) (Q678P mutation of SLC4A11).

Tumor-associated antigens may be antigens not normally expressed by the host; they can be mutated, truncated, misfolded, or otherwise abnormal manifestations of molecules normally expressed by the host; they can be identical to molecules normally expressed but expressed at abnormally high levels; or they can be expressed in a context or environment that is abnormal. Tumor-associated antigens may be, for example, proteins or protein fragments, complex carbohydrates, gangliosides, haptens, nucleic acids, other biological molecules or any combinations thereof.

### II. CEA Target Antigens

Disclosed herein include compositions comprising replication-defective vectors comprising one or more nucleic acid sequences encoding HER2/neu antigen, and/or one or more nucleic acid sequences encoding mucin family antigen such as CEA, and/or one or more nucleic acid sequences encoding Brachyury, and/or one or more nucleic acid sequences encoding MUC1-c in same or separate replication-defective vectors.

CEA represents an attractive target antigen for immunotherapy since it is over expressed in nearly all colorectal cancers and pancreatic cancers, and is also expressed by some lung and breast cancers, and uncommon tumors such as medullary thyroid cancer, but is not expressed in other cells of the body except for low-level expression in gastrointestinal epithelium. CEA contains epitopes that may be recognized in an MHC restricted fashion by T-cells.

It was discovered that multiple homologous immunizations with Ad5 [E1−, E2b−]-CEA(6D), encoding the tumor antigen CEA, induced CEA-specific cell-mediated immune (CMI) responses with antitumor activity in mice despite the presence of pre-existing or induced Ad5-neutralizing antibody. In the present phase I/II study, cohorts of patients with advanced colorectal cancer were immunized with escalating doses of Ad5 [E1−, E2b−]-CEA(6D). CEA-specific CMI responses were observed despite the presence of pre-existing Ad5 immunity in a majority (61.3%) of patients. Importantly, there was minimal toxicity, and overall patient survival (48% at 12 months) was similar regardless of pre-existing Ad5 neutralizing antibody titers. The results demonstrate that, in cancer patients, the novel Ad5 [E1−, E2b−] gene delivery platform generates significant CMI responses to the tumor antigen CEA in the setting of both naturally acquired and immunization-induced Ad5 specific immunity.

CEA antigen specific CMI can be, for example, greater than 10, 20, 30, 40, 50, 100, 200, 300, 400, 500, 600, 700, 800, 900, 1000, 5000, 10000, or more IFN-γ spot forming cells (SFC) per 106 peripheral blood mononuclear cells (PBMC). In some embodiments, the immune response is raised in a human subject with a preexisting inverse Ad5 neutralizing antibody titer of greater than 50, 100, 150, 200, 300, 400, 500, 600, 700, 800, 900, 1000, 1500, 2000, 2500, 3000, 3500, 4000, 4500, 5000, 6000, 7000, 8000, 9000, 1000, 12000, 15000 or higher. The immune response may comprise a cell-mediated immunity and/or a humoral immunity as described herein. The immune response may be measured by one or more of intracellular cytokine staining (ICS), ELISpot, proliferation assays, cytotoxic T-cell assays including chromium release or equivalent assays, and gene expression analysis using any number of polymerase chain reaction (PCR) or RT-PCR based assays, as described herein and to the extent they are available to a person skilled in the art, as well as any other suitable assays known in the art for measuring immune response.

In some embodiments, the replication defective adenovirus vector comprises a modified sequence encoding a subunit with at least 75%, 80%, 85%, 90%, 95%, 98%, 99%, 99.5%, or 99.9% identity to a wild-type subunit of the polypeptide.

The immunogenic polypeptide may be a mutant CEA or a fragment thereof. In some embodiments, the immunogenic polypeptide comprises a mutant CEA with an Asn->Asp substitution at position 610. In some embodiments, the replication defective adenovirus vector comprises a sequence encoding a polypeptide with at least 75%, 80%, 85%, 90%, 95%, 98%, 99%, 99.5%, or 99.9% identity to the immunogenic polypeptide. In some embodiments, the sequence encoding the immunogenic polypeptide comprises the sequence of SEQ ID NO: 30 (nucleic acid sequence for CEA-CAP1(6D)) or SEQ ID NO: 31 (amino acid sequence for the mutated CAP1(6D) epitope).

In some embodiments, the sequence encoding the immunogenic polypeptide comprises a sequence with at least 70% 75%, 80%, 85%, 90%, 95%, 98%, 99%, 99.5%, or 99.9% identity to SEQ ID NO: 30 or SEQ ID NO: 31 or a sequence generated from SEQ ID NO: 30 or SEQ ID NO: 31 by alternative codon replacements. In some embodiments, the immunogenic polypeptide encoded by the adenovirus vectors comprise up to 1, 2, 3, 4, 5, 6, 7, 8, 9, 10, 11, 12, 13, 14, 15, 16, 17, 18, 19, 20, 25, 30, 35, 40, or more point mutations, such as single amino acid substitutions or deletions, as compared to a wild-type human CEA sequence.

In some embodiments, the immunogenic polypeptide comprises a sequence from SEQ ID NO: 30 or SEQ ID NO: 31 or a modified version, e.g., comprising up to 1, 2, 3, 4, 5, 6, 7, 8, 9, 10, 11, 12, 13, 14, 15, 16, 17, 18, 19, 20, 25, 30, 35, 40, or more point mutations, such as single amino acid substitutions or deletions, of SEQ ID NO: 30 or SEQ ID NO: 31.

Members of the CEA gene family are subdivided into three subgroups based on sequence similarity, developmental expression patterns and their biological functions: the CEA-related Cell Adhesion Molecule (CEACAM) subgroup containing twelve genes (CEACAM1, CEACAM3-CEACAM8, CEACAM16 and CEACAM18-CEACAM21), the Pregnancy Specific Glycoprotein (PSG) subgroup containing eleven closely related genes (PSG1-PSG11) and a subgroup of eleven pseudogenes (CEACAMP1-CEACAMP11). Most members of the CEACAM subgroup have similar structures that consist of an extracellular Ig-like domains composed of a single N-terminal V-set domain, with structural homology to the immunoglobulin variable domains, followed by varying numbers of C2-set domains of A or B subtypes, a transmembrane domain and a cytoplasmic domain. There are two members of CEACAM subgroup (CEACAM16 and CEACAM20) that show a few exceptions in the organization of their structures. CEACAM16 contains two Ig-like V-type domains at its N and C termini and CEACAM20 contains a truncated Ig-like V-type 1 domain. The CEACAM molecules can be anchored to the cell surface via their transmembrane domains (CEACAM5 thought CEACAM8) or directly linked to glycophosphatidylinositol (GPI) lipid moiety (CEACAM5, CEACAM18 thought CEACAM21).

CEA family members are expressed in different cell types and have a wide range of biological functions. CEACAMs are found prominently on most epithelial cells and are present on different leucocytes. In humans, CEACAM1, the ancestor member of CEA family, is expressed on the apical side of epithelial and endothelial cells as well as on lymphoid and myeloid cells. CEACAM1 mediates cell-cell adhesion through hemophilic (CEACAM1 to CEACAM1) as well as heterothallic (e.g., CEACAM1 to CEACAM5) interactions. In addition, CEACAM1 is involved in many other biological processes, such as angiogenesis, cell migration, and immune functions. CEACAM3 and CEACAM4 expression is largely restricted to granulocytes, and they are able to convey uptake and destruction of several bacterial pathogens including Neisseria, Moraxella, and Haemophilus species.

Thus, in various embodiments, compositions and methods relate to raising an immune response against a CEA, selected from the group consisting of CEACAM1, CEACAM3, CEACAM4, CEACAM5, CEACAM6, CEACAM7, CEACAM8, CEACAM16, CEACAM18, CEACAM19, CEACAM20, CEACAM21, PSG1, PSG2, PSG3, PSG4, PSG5, PSG6, PSG7, PSG8, PSG9, and PSG11. An immune response may be raised against cells, e.g., cancer cells, expressing or overexpressing one or more of the CEAs, using the methods and compositions. In some embodiments, the overexpression of the one or more CEAs in such cancer cells is over 5, 10, 20, 30, 40, 50, 60, 70, 80, 90, 100 fold or more compared to non-cancer cells.

In certain embodiments, the CEA antigen used herein is a wild-type CEA antigen or a modified CEA antigen having a least a mutation in YLSGANLNL (SEQ ID NO: 28), a CAP1 epitope of CEA. The mutation can be conservative or non-conservative, substitution, addition, or deletion. In certain embodiments, the CEA antigen used herein has an amino acid sequence set forth in YLSGADLNL (SEQ ID NO: 31), a mutated CAP1 epitope. In further embodiments, the first replication-defective vector or a replication-defective vector that express CEA has a nucleotide sequence at least 50%, 60%, 65%, 70%, 75%, 80%, 85%, 90%, 95%, 98%, 99%, 99.5%, 99.9%, or 100% identical to any portion of SEQ ID NO: 29 (the predicted sequence of an adenovirus vector expressing a modified CEA antigen), such as positions 1057 to 3165 of SEQ ID NO: 29 or full-length SEQ ID NO: 29.

### III. Mucin Family Target Antigens

Disclosed herein include compositions comprising replication-defective vectors comprising one or more nucleic acid sequences encoding HER2/neu antigen, and/or one or more nucleic acid sequences encoding mucin family antigen such as MUC1, and/or one or more nucleic acid sequences encoding Brachyury, and/or one or more nucleic acid sequences encoding CEA in same or separate replication-defective vectors.

The human mucin family (MUC1 to MUC21) includes secreted and transmembrane mucins that play a role in forming protective mucous barriers on epithelial surfaces in the body. These proteins function in to protecting the epithelia lining the respiratory, gastrointestinal tracts, and lining ducts in important organs such as, for example the mammary gland, liver, stomach, pancreas, and kidneys.

MUC1 (CD227) is a TAA that is over-expressed on a majority of human carcinomas and several hematologic malignancies. MUC1 (GenBank: X80761.1, NCBI: NM_001204285.1) and activates many important cellular pathways known to be involved in human disease. MUC1 is a heterodimeric protein formed by two subunits that is commonly overexpressed in several human cancers. MUC1 undergoes autoproteolysis to generate two subunits MUC1n and MUC1c that, in turn, form a stable noncovalent heterodimer.

The MUC1 C-terminal subunit (MUC1c) can comprise a 58 aa extracellular domain (ED), a 28 aa transmembrane domain (TM) and a 72 aa cytoplasmic domain (CD). The MUC1c also can contain a “CQC” motif that can allow for dimerization of MUC1 and it can also impart oncogenic function to a cell. In some cases, MUC1 can in part oncogenic function through inducing cellular signaling via MUC1c. MUC1c can interact with EGFR, ErbB2 and other receptor tyrosine kinases and contributing to the activation of the PI3K→AKT and MEK→ERK cellular pathways. In the nucleus, MUC1c activates the Wnt/β-catenin, STAT, and NF-κB RelA cellular pathways. In some cases MUC1 can impart oncogenic function through inducing cellular signaling via MUC1n. The MUC1 N-terminal subunit (MUC1n) can comprise variable numbers of 20 amino acid tandem repeats that can be glycosylated. MUC1 is normally expressed at the surface of glandular epithelial cells and is over-expressed and aberrantly glycosylated in carcinomas. MUC1 is a TAA that can be utilized as a target for tumor immunotherapy. Several clinical trials have been and are being performed to evaluate the use of MUC1 in immunotherapeutic vaccines. Importantly, these trials indicate that immunotherapy with MUC1 targeting is safe and may provide survival benefit.

However, clinical trials have also shown that MUC1 is a relatively poor immunogen. To overcome this, the inventors have identified a T lymphocyte immune enhancer peptide sequence in the C terminus region of the MUC1 oncoprotein (MUC1-C or MUC1c). Compared with the native peptide sequence, the agonist in their modified MUC1-C (a) bound HLA-A2 at lower peptide concentrations, (b) demonstrated a higher avidity for HLA-A2, (c) when used with antigen-presenting cells, induced the production of more IFN-γ by T-cells than with the use of the native peptide, and (d) was capable of more efficiently generating MUC1-specific human T-cell lines from cancer patients. Importantly, T-cell lines generated using the agonist epitope were more efficient than those generated with the native epitope for the lysis of targets pulsed with the native epitope and in the lysis of HLA-A2 human tumor cells expressing MUC1. Additionally, the inventors have identified additional CD8+ cytotoxic T lymphocyte immune enhancer agonist sequence epitopes of MUC1-C.

In certain aspects, there is provided a potent MUC1-C modified for immune enhancer capability (mMUC1-C or MUC1-C or MUC1c). The present disclosure provides a potent MUC1-C modified for immune enhancer capability incorporated it into a recombinant Ad5 [E1−, E2b−] platform to produce a new and more potent immunotherapeutic vaccine. For example, the immunotherapeutic vaccine can be Ad5 [E1−, E2b−]-mMUC1-C for treating MUC1 expressing cancers or infectious diseases.

Post-translational modifications play an important role in controlling protein function in the body and in human disease. For example, in addition to proteolytic cleavage discussed above, MUC1 can have several post-translational modifications such as glycosylation, sialylation, palmitoylation, or a combination thereof at specific amino acid residues. Provided herein are immunotherapies targeting glycosylation, sialylation, phosphorylation, or palmitoylation modifications of MUC1.

MUC1 can be highly glycosylated (N- and O-linked carbohydrates and sialic acid at varying degrees on serine and threonine residues within each tandem repeat, ranging from mono- to penta-glycosylation). Differentially 0-glycosylated in breast carcinomas with 3,4-linked GlcNAc. N-glycosylation consists of high-mannose, acidic complex-type and hybrid glycans in the secreted form MUC1/SEC, and neutral complex-type in the transmembrane form, MUC1/TM.4. The present disclosure provides for immunotherapies targeting differentially 0-glycosylated forms of MUC1.

Further, MUC1 can be sialylated. Membrane-shed glycoproteins from kidney and breast cancer cells have preferentially sialyated core 1 structures, while secreted forms from the same tissues display mainly core 2 structures. The O-glycosylated content is overlapping in both these tissues with terminal fucose and galactose, 2- and 3-linked galactose, 3- and 3,6-linked GalNAc-ol and 4-linked GlcNAc predominating. The present disclosure provides for immunotherapies targeting various sialylation forms of MUC1. Dual palmitoylation on cysteine residues in the CQC motif is required for recycling from endosomes back to the plasma membrane. The present disclosure provides for immunotherapies targeting various palmitoylation forms of MUC1.

Phosphorylation can affect MUC1's ability to induce specific cell signaling responses that are important for human health. The present disclosure provides for immunotherapies targeting various phosphorylated forms of MUC1. For example, MUC1 can be phosphorylated on tyrosine and serine residues in the C-terminal domain. Phosphorylation on tyrosines in the C-terminal domain can increase nuclear location of MUC1 and β-catenin. Phosphorylation by PKC delta can induce binding of MUC1 to β-catenin/CTNNB1 and decrease formation of β-catenin/E-cadherin complexes. Src-mediated phosphorylation of MUC1 can inhibit interaction with GSK3B. Src- and EGFR-mediated phosphorylation of MUC1 on Tyr-1229 can increase binding to β-catenin/CTNNB1. GSK3B-mediated phosphorylation of MUC1 on Ser-1227 can decrease this interaction, but restores the formation of the β-cadherin/E-cadherin complex. PDGFR-mediated phosphorylation of MUC1 can increase nuclear colocalization of MUC1CT and CTNNB1. The present disclosure provides for immunotherapies targeting different phosphorylated forms of MUC1, MUC1c, and MUC1n known to regulate its cell signaling abilities.

The disclosure provides for immunotherapies that modulate MUC1c cytoplasmic domain and its functions in the cell. The disclosure provides for immunotherapies that comprise modulating a CQC motif in MUC1c. The disclosure provides for immunotherapies that comprise modulating the extracellular domain (ED), the transmembrane domain (TM), the cytoplasmic domain (CD) of MUC1c, or a combination thereof. The disclosure provides for immunotherapies that comprise modulating MUC1c's ability to induce cellular signaling through EGFR, ErbB2, or other receptor tyrosine kinases. The disclosure provides for immunotherapies that comprise modulating MUC1c's ability to induce PI3K→AKT, MEK→ERK, Wnt/β-catenin, STAT, NF-κB RelA cellular pathways, or combination thereof.

In some embodiments, the MUC1c immunotherapy can further comprise HER2/neu, CEA, or Brachyury immunotherapy in the same replication-defective virus vectors or separate replication-defective virus vectors.

The disclosure also provides for immunotherapies that modulate MUC1n and its cellular functions. The disclosure also provides for immunotherapies comprising tandem repeats of MUC1n, the glycosylation sites on the tandem repeats of MUC1n, or a combination thereof. In some embodiments, the MUC1n immunotherapy further comprises HER2/neu, CEA, or Brachyury immunotherapy in the same replication-defective virus vectors or separate replication-defective virus vectors.

The disclosure also provides vaccines comprising MUC1n, MUC1c, HER2/neu, brachyury, CEA, or a combination thereof. The disclosure provides vaccines comprising MUC1c and HER2/neu, brachyury, CEA, or a combination thereof. The disclosure also provides vaccines targeting MUC1n and HER2/neu, Brachyury, CEA, or a combination thereof. In some embodiments, the antigen combination is contained in one vector as provided herein. In some embodiments, the antigen combination is contained in a separate vector as provided herein.

The present invention relates to a replication defective adenovirus vector of serotype 5 comprising a sequence encoding an immunogenic polypeptide. The immunogenic polypeptide may be an isoform of MUC1 or a subunit or a fragment thereof. In some embodiments, the replication defective adenovirus vector comprises a sequence encoding a polypeptide with at least 75%, 80%, 85%, 90%, 95%, 98%, 99%, 99.5%, or 99.9% identity to the immunogenic polypeptide. In some embodiments, the immunogenic polypeptide encoded by the adenovirus vectors described herein comprising up to 1, 2, 3, 4, 5, 6, 7, 8, 9, 10, 11, 12, 13, 14, 15, 16, 17, 18, 19, 20, 25, 30, 35, 40, or more point mutations, such as single amino acid substitutions or deletions, as compared to a wild-type human MUC1 sequence.

In some embodiments, a MUC1-c antigen of this disclosure can be a modified MUC1 and can have a nucleotide sequence that is at least 80%, at least 85%, at least 90%, at least 92%, at least 95%, at least 97%, or at least 99% identical to SEQ ID NO: 32. In certain embodiments, a MUC1-c antigen of this disclosure can have a nucleotide sequence as set forth in SEQ ID NO: 32.

In some embodiments, a MUC1-c antigen of this disclosure can be a modified MUC1 and can have an amino sequence that is at least 80%, at least 85%, at least 90%, at least 92%, at least 95%, at least 97%, or at least 99% identical to SEQ ID NO: 33. In certain embodiments, a MUC1-c antigen of this disclosure can have an amino acid sequence as set forth in SEQ ID NO: 33.

### IV. Brachyury Target Antigens

Disclosed herein include compositions comprising replication-defective vectors comprising one or more nucleic acid sequences encoding HER2/neu antigen, and/or one or more nucleic acid sequences encoding mucin family antigen such as MUC1, and/or one or more nucleic acid sequences encoding Brachyury, and/or one or more nucleic acid sequences encoding CEA in same or separate replication-defective vectors.

The disclosure provides for immunotherapies that comprise one or more antigens to Brachyury. Brachyury (also known as the “T” protein in humans) is a member of the T-box family of transcription factors that play key roles during early development, mostly in the formation and differentiation of normal mesoderm and is characterized by a highly conserved DNA-binding domain designated as T-domain. The epithelial to mesenchymal transition (EMT) is a key step during the progression of primary tumors into a metastatic state in which Brachyury plays a crucial role. The expression of Brachyury in human carcinoma cells induces changes characteristic of EMT, including up-regulation of mesenchymal markers, down-regulation of epithelial markers, and an increase in cell migration and invasion. Conversely, inhibition of Brachyury resulted in down-regulation of mesenchymal markers and loss of cell migration and invasion and diminished the ability of human tumor cells to form metastases. Brachyury can function to mediate epithelial-mesenchymal transition and promotes invasion.

The disclosure also provides for immunotherapies that modulate Brachyury effect on epithelial-mesenchymal transition function in cell proliferation diseases, such as cancer. The disclosure also provides immunotherapies that modulate Brachyury's ability to promote invasion in cell proliferation diseases, such as cancer. The disclosure also provides for immunotherapies that modulate the DNA binding function of T-box domain of Brachyury. In some embodiments, the Brachyury immunotherapy can further comprise one or more antigens to HER2/neu, CEA, or MUC1, MUC1c, or MUC1n.

Brachyury expression is nearly undetectable in most normal human tissues and is highly restricted to human tumors and often overexpressed making it an attractive target antigen for immunotherapy. In humans, Brachyury is encoded by the T gene (GenBank: AJ001699.1, NCBI: NM_003181.3). There are at least two different isoforms produced by alternative splicing found in humans. Each isoform has a number of natural variants.

Brachyury is immunogenic and Brachyury-specific CD8+ T-cells expanded in vitro can lyse Brachyury expressing tumor cells. These features of Brachyury make it an attractive tumor associated antigen (TAA) for immunotherapy. The Brachyury protein is a T-box transcription factor. It can bind to a specific DNA element, a near palindromic sequence “TCACACCT” through a region in its N-terminus, called the T-box to activate gene transcription when bound to such a site.

The disclosure also provides vaccines comprising Brachyury, HER2/neu, MUC1, CEA, or a combination thereof. In some embodiments, the antigen combination is contained in one vector as provided herein. In some embodiments, the antigen combination is contained in a separate vector as provided herein.

In particular embodiments, the present invention relates to a replication defective adenovirus vector of serotype 5 comprising a sequence encoding an immunogenic polypeptide. The immunogenic polypeptide may be an isoform of Brachyury or a subunit or a fragment thereof. In some embodiments, the replication defective adenovirus vector comprises a sequence encoding a polypeptide with at least 70%, 75%, 80%, 85%, 90%, 95%, 98%, 99%, 99.5%, or 99.9% identity to the immunogenic polypeptide. In some embodiments, the immunogenic polypeptide encoded by the adenovirus vectors described herein comprising up to 1, 2, 3, 4, 5, 6, 7, 8, 9, 10, 11, 12, 13, 14, 15, 16, 17, 18, 19, 20, 25, 30, 35, 40, or more point mutations, such as single amino acid substitutions or deletions, as compared to a wild-type human Brachyury sequence.

In some embodiments, a Brachyury antigen of this disclosure can have an amino sequence that is at least 80%, at least 85%, at least 90%, at least 92%, at least 95%, at least 97%, or at least 99% identical to SEQ ID NO: 34. In certain embodiments, a Brachyury antigen of this disclosure can have an amino acid sequence as set forth in SEQ ID NO: 34.

### V. Vectors

Certain aspects include transferring into a cell an expression construct comprising one or more nucleic acid sequences encoding one or more target antigens such as a HER2/neu antigen or epitope. In certain embodiments, transfer of an expression construct into a cell may be accomplished using a viral vector. A viral vector may be used to include those constructs containing viral sequences sufficient to express a recombinant gene construct that has been cloned therein.

In particular embodiments, the viral vector is an adenovirus vector. Adenoviruses are a family of DNA viruses characterized by an icosahedral, non-enveloped capsid containing a linear double-stranded genome. Of the human adenoviruses, none are associated with any neoplastic disease, and only cause relatively mild, self-limiting illness in immunocompetent individuals.

Adenovirus vectors may have low capacity for integration into genomic DNA. Adenovirus vectors may result in highly efficient gene transfer. Additional advantages of adenovirus vectors include that they are efficient at gene delivery to both nondividing and dividing cells, and can be produced in large quantities.

In contrast to integrating viruses, the adenoviral infection of host cells may not result in chromosomal integration because adenoviral DNA can replicate in an episomal manner without potential genotoxicity. Also, adenovirus vectors may be structurally stable, and no genome rearrangement has been detected after extensive amplification. Adenovirus is particularly suitable for use as a gene transfer vector because of its mid-sized genome, ease of manipulation, high titer, wide target-cell range, and high infectivity.

The first genes expressed by the virus are the E1 genes, which act to initiate high-level gene expression from the other Ad5 gene promoters present in the wild type genome. Viral DNA replication and assembly of progeny virions occur within the nucleus of infected cells, and the entire life cycle takes about 36 hrs with an output of approximately 104 virions per cell.

The wild type Ad5 genome is approximately 36 kb, and encodes genes that are divided into early and late viral functions, depending on whether they are expressed before or after DNA replication. The early/late delineation is nearly absolute, since it has been demonstrated that super-infection of cells previously infected with an Ad5 results in lack of late gene expression from the super-infecting virus until after it has replicated its own genome. Without being bound by theory, this is likely due to a replication dependent cis-activation of the Ad5 major late promoter (MLP), preventing late gene expression (primarily the Ad5 capsid proteins) until replicated genomes are present to be encapsulated. The composition and methods may take advantage of these features in the development of advanced generation Ad vectors/vaccines.

The adenovirus vector may be replication defective, or at least conditionally defective. The adenovirus may be of any of the 42 different known serotypes or subgroups A-F, and other serotypes or subgroups are envisioned. Adenovirus type 5 of subgroup C may be used in particular embodiments in order to obtain a replication-defective adenovirus vector. This is because Adenovirus type 5 is a human adenovirus about which a great deal of biochemical and genetic information is known, and it has historically been used for most constructs employing adenovirus as a vector.

Adenovirus growth and manipulation is known to those of skill in the art, and exhibits a broad host range in vitro and in vivo. Modified viruses, such as adenoviruses with alteration of the CAR domain, may also be used. Methods for enhancing delivery or evading an immune response, such as liposome encapsulation of the virus, are also envisioned.

The vector may comprise a genetically engineered form of adenovirus, such as an E2 deleted adenoviral vector, or more specifically, an E2b deleted adenoviral vector. The term “E2b deleted,” as used herein, refers to a specific DNA sequence that is mutated in such a way so as to prevent expression and/or function of at least one E2b gene product. Thus, in certain embodiments, “E2b deleted” refers to a specific DNA sequence that is deleted (removed) from the Ad genome. E2b deleted or “containing a deletion within the E2b region” refers to a deletion of at least one base pair within the E2b region of the Ad genome. In certain embodiments, more than one base pair is deleted and in further embodiments, at least 20, 30, 40, 50, 60, 70, 80, 90, 100, 110, 120, 130, 140, or 150 base pairs are deleted. In another embodiment, the deletion is of more than 150, 160, 170, 180, 190, 200, 250, or 300 base pairs within the E2b region of the Ad genome. An E2b deletion may be a deletion that prevents expression and/or function of at least one E2b gene product and therefore, encompasses deletions within exons encoding portions of E2b-specific proteins as well as deletions within promoter and leader sequences. In certain embodiments, an E2b deletion is a deletion that prevents expression and/or function of one or both of the DNA polymerase and the preterminal protein of the E2b region. In a further embodiment, “E2b deleted” refers to one or more point mutations in the DNA sequence of this region of an Ad genome such that one or more encoded proteins is non-functional. Such mutations include residues that are replaced with a different residue leading to a change in the amino acid sequence that result in a nonfunctional protein.

As would be understood by the skilled artisan upon reading the present disclosure, other regions of the Ad genome can be deleted. Thus to be “deleted” in a particular region of the Ad genome, as used herein, refers to a specific DNA sequence that is mutated in such a way so as to prevent expression and/or function of at least one gene product encoded by that region. In certain embodiments, to be “deleted” in a particular region refers to a specific DNA sequence that is deleted (removed) from the Ad genome in such a way so as to prevent the expression and/or the function encoded by that region (e.g., E2b functions of DNA polymerase or preterminal protein function). “Deleted” or “containing a deletion” within a particular region refers to a deletion of at least one base pair within that region of the Ad genome.

Thus, in certain embodiments, more than one base pair is deleted and in further embodiments, at least 20, 30, 40, 50, 60, 70, 80, 90, 100, 110, 120, 130, 140, or 150 base pairs are deleted from a particular region. In another embodiment, the deletion is more than 150, 160, 170, 180, 190, 200, 250, or 300 base pairs within a particular region of the Ad genome. These deletions are such that expression and/or function of the gene product encoded by the region is prevented. Thus deletions encompass deletions within exons encoding portions of proteins as well as deletions within promoter and leader sequences. In a further embodiment, “deleted” in a particular region of the Ad genome refers to one or more point mutations in the DNA sequence of this region of an Ad genome such that one or more encoded proteins is non-functional. Such mutations include residues that are replaced with a different residue leading to a change in the amino acid sequence that result in a nonfunctional protein.

In certain embodiments, the adenovirus vectors contemplated for use include E2b deleted adenovirus vectors that have a deletion in the E2b region of the Ad genome and, optionally, the E1 region. In some cases, such vectors do not have any other regions of the Ad genome deleted.

In another embodiment, the adenovirus vectors contemplated for use include E2b deleted adenovirus vectors that have a deletion in the E2b region of the Ad genome and, optionally, deletions in the E1 and E3 regions. In some cases, such vectors have no other regions deleted.

In a further embodiment, the adenovirus vectors contemplated for use include adenovirus vectors that have a deletion in the E2b region of the Ad genome and, optionally, deletions in the E1, E3, and, also optionally, partial or complete removal of the E4 regions. In some cases, such vectors have no other deletions.

In another embodiment, the adenovirus vectors contemplated for use include adenovirus vectors that have a deletion in the E2b region of the Ad genome and, optionally, deletions in the E1 and/or E4 regions. In some cases, such vectors contain no other deletions.

In an additional embodiment, the adenovirus vectors contemplated for use include adenovirus vectors that have a deletion in the E2a, E2b, and/or E4 regions of the Ad genome. In some cases, such vectors have no other deletions.

In one embodiment, the adenovirus vectors for use herein comprise vectors having the E1 and/or DNA polymerase functions of the E2b region deleted. In some cases, such vectors have no other deletions.

In a further embodiment, the adenovirus vectors for use herein have the E1 and/or the preterminal protein functions of the E2b region deleted. In some cases, such vectors have no other deletions.

In another embodiment, the adenovirus vectors for use herein have the E1, DNA polymerase, and/or the preterminal protein functions deleted. In some cases, such vectors have no other deletions. In one particular embodiment, the adenovirus vectors contemplated for use herein are deleted for at least a portion of the E2b region and/or the E1 region.

In some cases, such vectors are not “gutted” adenovirus vectors. In this regard, the vectors may be deleted for both the DNA polymerase and the preterminal protein functions of the E2b region. In an additional embodiment, the adenovirus vectors for use include adenovirus vectors that have a deletion in the E1, E2b, and/or 100K regions of the adenovirus genome. In certain embodiments, the adenovirus vector may be a “gutted” adenovirus vector.

In one embodiment, the adenovirus vectors for use herein comprise vectors having the E1, E2b, and/or protease functions deleted. In some cases, such vectors have no other deletions.

In a further embodiment, the adenovirus vectors for use herein have the E1 and/or the E2b regions deleted, while the fiber genes have been modified by mutation or other alterations (e.g., to alter Ad tropism). Removal of genes from the E3 or E4 regions may be added to any of the mentioned adenovirus vectors.

The deleted adenovirus vectors can be generated using recombinant techniques known in the art (see e.g., Amalfitano, et al. J. Virol. 1998; 72:926-33; Hodges, et al. J Gene Med 2000; 2:250-59). As would be recognized by a skilled artisan, the adenovirus vectors for use in certain aspects can be successfully grown to high titers using an appropriate packaging cell line that constitutively expresses E2b gene products and products of any of the necessary genes that may have been deleted. In certain embodiments, HEK-293-derived cells that not only constitutively express the E1 and DNA polymerase proteins, but also the Ad-preterminal protein, can be used. In one embodiment, E.C7 cells are used to successfully grow high titer stocks of the adenovirus vectors (see e.g., Amalfitano, et al. J. Virol. 1998; 72:926-33; Hodges, et al. J Gene Med 2000; 2:250-59)

In order to delete critical genes from self-propagating adenovirus vectors, the proteins encoded by the targeted genes may be coexpressed in HEK-293 cells, or similar, along with the E1 proteins. Therefore, only those proteins which are non-toxic when coexpressed constitutively (or toxic proteins inducibly-expressed) can be utilized. Coexpression in HEK-293 cells of the E1 and E4 genes has been demonstrated (utilizing inducible, not constitutive, promoters) (Yeh, et al. J. Virol. 1996; 70:559; Wang et al. Gene Therapy 1995; 2:775; and Gorziglia, et al. J. Virol. 1996; 70:4173). The E1 and protein IX genes (a virion structural protein) have been coexpressed (Caravokyri, et al. J. Virol. 1995; 69: 6627), and coexpression of the E1, E4, and protein IX genes has also been described (Krougliak, et al. Hum. Gene Ther. 1995; 6:1575). The E1 and 100k genes have been successfully expressed in transcomplementing cell lines, as have E1 and protease genes (Oualikene, et al. Hum Gene Ther 2000; 11:1341-53; Hodges, et al. J. Virol 2001; 75:5913-20).

Cell lines coexpressing E1 and E2b gene products for use in growing high titers of E2b deleted Ad particles are described in U.S. Pat. No. 6,063,622. The E2b region encodes the viral replication proteins which are absolutely required for Ad genome replication (Doerfler, et al. Chromosoma 1992; 102:S39-S45). Useful cell lines constitutively express the approximately 140 kDa Ad-DNA polymerase and/or the approximately 90 kDa preterminal protein. In particular, cell lines that have high-level, constitutive coexpression of the E1, DNA polymerase, and preterminal proteins, without toxicity (e.g., E.C7), are desirable for use in propagating Ad for use in multiple vaccinations. These cell lines permit the propagation of adenovirus vectors deleted for the E1, DNA polymerase, and preterminal proteins.

The recombinant Ad can be propagated using techniques known in the art. For example, in certain embodiments, tissue culture plates containing E.C7 cells are infected with the adenovirus vector virus stocks at an appropriate MOI (e.g., 5) and incubated at 37.0° C. for 40-96 hrs. The infected cells are harvested, resuspended in 10 mM Tris-CI (pH 8.0), and sonicated, and the virus is purified by two rounds of cesium chloride density centrifugation. In certain techniques, the virus containing band is desalted over a Sephadex CL-6B column (Pharmacia Biotech, Piscataway, N.J.), sucrose or glycerol is added, and aliquots are stored at −80° C. In some embodiments, the virus is placed in a solution designed to enhance its stability, such as A195 (Evans, et al. J Pharm Sci 2004; 93:2458-75). The titer of the stock is measured (e.g., by measurement of the optical density at 260 nm of an aliquot of the virus after SDS lysis). In another embodiment, plasmid DNA, either linear or circular, encompassing the entire recombinant E2b deleted adenovirus vector can be transfected into E.C7, or similar cells, and incubated at 37.0° C. until evidence of viral production is present (e.g., the cytopathic effect). The conditioned media from these cells can then be used to infect more E.C7, or similar cells, to expand the amount of virus produced, before purification. Purification can be accomplished by two rounds of cesium chloride density centrifugation or selective filtration. In certain embodiments, the virus may be purified by column chromatography, using commercially available products (e.g., Adenopure from Puresyn, Inc., Malvem, Pa.) or custom made chromatographic columns.

In certain embodiments, the recombinant adenovirus vector may comprise enough of the virus to ensure that the cells to be infected are confronted with a certain number of viruses. Thus, there may be provided a stock of recombinant Ad, particularly an RCA-free stock of recombinant Ad. The preparation and analysis of Ad stocks can use any methods available in the art. Viral stocks vary considerably in titer, depending largely on viral genotype and the protocol and cell lines used to prepare them. The viral stocks can have a titer of at least about 10⁶, 10⁷, or 10⁸ virus particles (VPs)/ml, and many such stocks can have higher titers, such as at least about 10⁹, 10¹⁰, 10¹¹, or 10¹² VPs/ml.

Certain aspects contemplate the use of E2b deleted adenovirus vectors, such as those described in U.S. Pat. Nos. 6,063,622; 6,451,596; 6,057,158; 6,083,750; and 8,298,549. The vectors with deletions in the E2b regions in many cases cripple viral protein expression and/or decrease the frequency of generating replication competent Ad (RCA).

Propagation of these E2b deleted adenovirus vectors can be done utilizing cell lines that express the deleted E2b gene products. Certain aspects also provide such packaging cell lines; for example E.C7 (formally called C-7), derived from the HEK-293 cell line.

In further aspects, the E2b gene products, DNA polymerase and preterminal protein, can be constitutively expressed in E.C7, or similar cells along with the E1 gene products. Transfer of gene segments from the Ad genome to the production cell line has immediate benefits: (1) increased carrying capacity; and, (2) a decreased potential of RCA generation, typically requiring two or more independent recombination events to generate RCA. The E1, Ad DNA polymerase and/or preterminal protein expressing cell lines used herein can enable the propagation of adenovirus vectors with a carrying capacity approaching 13 kb, without the need for a contaminating helper virus. In addition, when genes critical to the viral life cycle are deleted (e.g., the E2b genes), a further crippling of Ad to replicate or express other viral gene proteins occurs. This can decrease immune recognition of virally infected cells, and allow for extended durations of foreign transgene expression.

E1, DNA polymerase, and preterminal protein deleted vectors are typically unable to express the respective proteins from the E1 and E2b regions. Further, they may show a lack of expression of most of the viral structural proteins. For example, the major late promoter (MLP) of Ad is responsible for transcription of the late structural proteins L1 through L5. Though the MLP is minimally active prior to Ad genome replication, the highly toxic Ad late genes are primarily transcribed and translated from the MLP only after viral genome replication has occurred. This cis-dependent activation of late gene transcription is a feature of DNA viruses in general, such as in the growth of polyoma and SV-40. The DNA polymerase and preterminal proteins are important for Ad replication (unlike the E4 or protein IX proteins). Deletion of the E1 region can be extremely detrimental to adenovirus vector late gene expression, and can thereby curb the toxic effects of late gene expression in cells such as antigen presenting cells (APCs). Thus, E1-deleted adenovirus vectors are advantagous for use as vaccine backbones to deliver antigens in therapeutic vaccine regimens to APCs, such as those described herein, in order to induce a protective immune response while minimizing APC toxicity.

Certain aspects contemplate the use of E1-deleted adenovirus vectors. First generation, or E1-deleted adenovirus vectors Ad5 [E1−] are constructed such that a transgene replaces only the E1 region of genes. Typically, about 90% of the wild-type Ad5 genome is retained in the vector. Ad5 [E1−] vectors have a decreased ability to replicate and cannot produce infectious virus after infection of cells not expressing the Ad5 E1 genes. The recombinant Ad5 [E1−] vectors are propagated in human cells (typically 293 cells) allowing for Ad5 [E1−] vector replication and packaging. Ad5 [E1−] vectors have a number of positive attributes; one of the most important is their relative ease for scale up and cGMP production. Currently, well over 220 human clinical trials utilize Ad5 [E1−] vectors, with more than two thousand subjects given the virus subcutaneously, intramuscularly, or intravenously.

Additionally, Ad5 vectors do not integrate; their genomes remain episomal. Generally, for vectors that do not integrate into the host genome, the risk for insertional mutagenesis and/or germ-line transmission is extremely low if at all. Conventional Ad5 [E1−] vectors have a carrying capacity that approaches 7 kb.

Studies in humans and animals have demonstrated that pre-existing immunity against Ad5 can be an inhibitory factor to commercial use of Ad-based vaccines. The preponderance of humans have antibody against Ad5, the most widely used subtype for human vaccines, with two-thirds of humans studied having lympho-proliferative responses against Ad5. This pre-existing immunity can inhibit immunization or re-immunization using typical Ad5 vaccines and may preclude the immunization of a vaccine against a second antigen, using an Ad5 vector, at a later time. Overcoming the problem of pre-existing anti-vector immunity has been a subject of intense investigation. Investigations using alternative human (non-Ad5 based) Ad5 subtypes or even non-human forms of Ad5 have been examined. Even if these approaches succeed in an initial immunization, subsequent vaccinations may be problematic due to immune responses to the novel Ad5 subtype.

To avoid the Ad5 immunization barrier, and improve upon the limited efficacy of first generation Ad5 [E1−] vectors to induce optimal immune responses, there are provided certain embodiments related to a next generation Ad5 vector based vaccine platform. The next generation Ad5 platform has additional deletions in the E2b region, removing the DNA polymerase and the preterminal protein genes. The Ad5 [E1−, E2b−] platform has an expanded cloning capacity that is sufficient to allow inclusion of many possible genes. Ad5 [E1−, E2b−] vectors have up to about 12 kb gene-carrying capacity as compared to the 7 kb capacity of Ad5 [E1−] vectors, providing space for multiple genes if needed. In some embodiments, an insert of more than 1, 2, 3, 4, 5, 6, 7, 8, 9, 10, or 11 kb is introduced into an Ad5 vector, such as the Ad5 [E1−, E2b−] vector.

Deletion of the E2b region may confer advantageous immune properties on the Ad5 vectors, often eliciting potent immune responses to target transgene antigens, such as a HER2/neu antigen or epitope, while minimizing the immune responses to Ad viral proteins.

In various embodiments, Ad5 [E1−, E2b−] vectors may induce potent cell-mediated immunity (CMI), as well as antibodies against the vector expressed target antigens, such as a HER2/neu antigen or epitope, even in the presence of Ad immunity.

Ad5 [E1−, E2b−] vectors also have reduced adverse reactions as compared to Ad5 [E1−] vectors, in particular the appearance of hepatotoxicity and tissue damage.

Certain aspects of these Ad5 vectors and expression of Ad late genes are greatly reduced. For example, production of the capsid fiber proteins could be detected in vivo for Ad5 [E1−] vectors, while fiber expression was ablated from Ad5 [E1−, E2b−] vector vaccines. The innate immune response to wild type Ad is complex. Proteins deleted from the Ad5 [E1−, E2b−] vectors generally play an important role. Specifically, Ad5 [E1−, E2b−] vectors with deletions of preterminal protein or DNA polymerase display reduced inflammation during the first 24 to 72 hours following injection compared to Ad5 [E1−] vectors. In various embodiments, the lack of Ad5 gene expression renders infected cells invisible to anti-Ad activity and permits infected cells to express the transgene for extended periods of time, which develops immunity to the target.

Various embodiments contemplate increasing the capability for the Ad5 [E1−, E2b−] vectors to transduce dendritic cells, improving antigen specific immune responses in the vaccine by taking advantage of the reduced inflammatory response against Ad5 [E1−, E2b−] vector viral proteins and the resulting evasion of pre-existing Ad immunity.

In some cases, this immune induction may take months. Ad5 [E1−, E2b−] vectors not only are safer than, but appear to be superior to, Ad5 [E1−] vectors in regard to induction of antigen specific immune responses, making them much better suitable as a platform to deliver tumor vaccines that can result in a clinical response.

In certain embodiments, methods and compositions are provided by taking advantage of an Ad5 [E1−, E2b−], vector system for developing a therapeutic tumor vaccine that overcomes barriers found with other Ad5 systems and permits the immunization of people who have previously been exposed to Ad5.

E2b deleted vectors may have up to a 13 kb gene-carrying capacity as compared to the 5 to 6 kb capacity of First Generation adenovirus vectors, easily providing space for nucleic acid sequences encoding any of a variety of target antigens, such as a HER2/neu antigen or epitope.

The E2b deleted adenovirus vectors also can have reduced adverse reactions as compared to First Generation adenovirus vectors. E2b deleted vectors can have reduced expression of viral genes, and this characteristic can lead to extended transgene expression in vivo.

Compared to first generation adenovirus vectors, certain embodiments of the Second Generation E2b deleted adenovirus vectors contain additional deletions in the DNA polymerase gene (pol) and deletions of the pre-terminal protein (pTP).

It appears that Ad proteins expressed from adenovirus vectors play an important role. Specifically, the deletions of pre-terminal protein and DNA polymerase in the E2b deleted vectors appear to reduce inflammation during the first 24 to 72 hrs following injection, whereas First Generation adenovirus vectors stimulate inflammation during this period.

In addition, it has been reported that the additional replication block created by E2b deletion also leads to a 10,000 fold reduction in expression of Ad late genes, well beyond that afforded by E1, E3 deletions alone. The decreased levels of Ad proteins produced by E2b deleted adenovirus vectors effectively reduce the potential for competitive, undesired, immune responses to Ad antigens, responses that prevent repeated use of the platform in Ad immunized or exposed individuals.

The reduced induction of inflammatory response by second generation E2b deleted vectors results in increased potential for the vectors to express desired vaccine antigens, such as a HER2/neu antigen or epitope, during the infection of antigen presenting cells (i.e., dendritic cells), decreasing the potential for antigenic competition, resulting in greater immunization of the vaccine to the desired antigen relative to identical attempts with First Generation adenovirus vectors.

E2b deleted adenovirus vectors provide an improved Ad-based vaccine candidate that is safer, more effective, and more versatile than previously described vaccine candidates using First Generation adenovirus vectors.

Thus, first generation, E1-deleted Adenovirus subtype 5 (Ad5)-based vectors, although promising platforms for use as vaccines, may be impeded in activity by naturally occurring or induced Ad-specific neutralizing antibodies.

Without being bound by theory, Ad5-based vectors with deletions of the E1 and the E2b regions (Ad5 [E1−, E2b−]), the latter encoding the DNA polymerase and the pre-terminal protein, for example by virtue of diminished late phase viral protein expression, may avoid immunological clearance and induce more potent immune responses against the encoded antigen transgene, such as a HER2/neu antigen or epitope, in Ad-immune hosts.

### VI. Heterologous Nucleic Acids

In some embodiments, vectors, such as adenovirus vectors, may comprise heterologous nucleic acid sequences that encode one or more tumor antigens such as a HER2/neu antigen or epitope, fusions thereof or fragments thereof, which can modulate the immune response. In certain aspects, there may be provided a Second Generation E2b deleted adenovirus vectors that comprise a heterologous nucleic acid sequence encoding one or more tumor antigens such as a HER2/neu antigen or epitope.

As such, there may be provided polynucleotides that encode a HER2/neu antigen or epitope from any source as described further herein, vectors or constructs comprising such polynucleotides and host cells transformed or transfected with such vectors or expression constructs.

The terms “nucleic acid” and “polynucleotide” are used essentially interchangeably herein. As will be also recognized by the skilled artisan, polynucleotides used herein may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide as disclosed herein, and a polynucleotide may, but need not, be linked to other molecules and/or support materials. An isolated polynucleotide, as used herein, means that a polynucleotide is substantially away from other coding sequences. For example, an isolated DNA molecule as used herein does not contain large portions of unrelated coding DNA, such as large chromosomal fragments or other functional genes or polypeptide coding regions. Of course, this refers to the DNA molecule as originally isolated, and does not exclude genes or coding regions later added to the segment through recombination in the laboratory.

As will be understood by those skilled in the art, the polynucleotides can include genomic sequences, extra-genomic and plasmid-encoded sequences and smaller engineered gene segments that express, or may be adapted to express target antigens as described herein, fragments of antigens, peptides and the like. Such segments may be naturally isolated, or modified synthetically by the hand of man.

Polynucleotides may comprise a native sequence (i.e., an endogenous sequence that encodes one or more tumor antigens such as a HER2/neu antigen or epitope or a portion thereof) or may comprise a sequence that encodes a variant or derivative of such a sequence. In certain embodiments, the polynucleotide sequences set forth herein encode one or more mutated tumor antigens such as a HER2/neu antigen or epitope. In some embodiments, polynucleotides represent a novel gene sequence that has been optimized for expression in specific cell types (i.e., human cell lines) that may substantially vary from the native nucleotide sequence or variant but encode a similar protein antigen.

In other related embodiments, there may be provided polynucleotide variants having substantial identity to native sequences encoding one or more tumor antigens such as a HER2/neu antigen or epitope, for example those comprising at least 60, 70, 80, 90, 95, 96, 97, 98, 99, or 100% sequence identity (or any derivable range or value thereof), particularly at least 75% up to 99% or higher sequence identity compared to a native polynucleotide sequence set forth in SEQ ID NO: 1 or a polynclueotide sequence encoding one or more tumor antigens such as a HER2/neu antigen or epitope or an amino acid sequence with at least 60, 70, 80, 90, 95, 96, 97, 98, 99, or 100% (or any derivable range or value thereof), particularly at least 75% up to 99% or higher sequence identity with SEQ ID NO: 2 using the methods described herein (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

Typically, polynucleotide variants will contain one or more substitutions, additions, deletions and/or insertions, preferably such that the immunogenicity of the epitope of the polypeptide encoded by the variant polynucleotide or such that the immunogenicity of the heterologous target protein is not substantially diminished relative to a polypeptide encoded by the native polynucleotide sequence. As described elsewhere herein, the polynucleotide variants preferably encode a variant of one or more tumor antigens such as a HER2/neu antigen or epitope, or a fragment (e.g., an epitope) thereof wherein the propensity of the variant polypeptide or fragment (e.g., epitope) thereof to react with antigen-specific antisera and/or T-cell lines or clones is not substantially diminished relative to the native polypeptide. The term “variants” should also be understood to encompass homologous genes of xenogenic origin.

In certain aspects, there may be provided polynucleotides that comprise or consist of at least about 5 up to a 1000 or more contiguous nucleotides encoding a polypeptide, including target protein antigens, as described herein, as well as all intermediate lengths between. It will be readily understood that “intermediate lengths,” in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers from 200-500; 500-1,000, and the like. A polynucleotide sequence as described herein may be extended at one or both ends by additional nucleotides not found in the native sequence encoding a polypeptide as described herein, such as an epitope or heterologous target protein. This additional sequence may consist of 1 up to 20 nucleotides or more, at either end of the disclosed sequence or at both ends of the disclosed sequence.

The polynucleotides or fragments thereof, regardless of the length of the coding sequence itself, may be combined with other DNA sequences, such as promoters, expression control sequences, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of about 1000, 2000, 3000, 4000, 5000, 6000, 7000, 8000, 9000, 10,000, about 500, about 200, about 100, about 50 base pairs in length, and the like, (including all intermediate lengths) are contemplated to be useful in certain aspects.

When comparing polynucleotide sequences, two sequences are said to be “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff M O (1978) A model of evolutionary change in proteins—Matrices for detecting distant relationships. In Dayhoff M O (ed.) Atlas of Protein Sequence and Structure, National Biomedical Research Foundation, Washington D.C. Vol. 5, Suppl. 3, pp. 345-358; Hein J Unified Approach to Alignment and Phylogenes, pp. 626-645 (1990); Methods in Enzymology vol. 183, Academic Press, Inc., San Diego, Calif.; Higgins, et al. PM CABIOS 1989; 5:151-53; Myers E W, et al. CABIOS 1988; 4:11-17; Robinson E D Comb. Theor 1971; 11A 05; Saitou N, et al. Mol. Biol. Evol. 1987; 4:406-25; Sneath PHA and Sokal RR Numerical Taxonomy—the Principles and Practice of Numerical Taxonomy, Freeman Press, San Francisco, Calif. (1973); Wilbur W J, et al. Proc. Natl. Acad., Sci. USA 1983 80:726-30).

Alternatively, optimal alignment of sequences for comparison may be conducted by the local identity algorithm of Smith, et al. Add. APL. Math 1981; 2:482, by the identity alignment algorithm of Needleman, et al. Mol. Biol. 1970 48:443, by the search for similarity methods of Pearson and Lipman, Proc. Natl. Acad. Sci. USA 1988; 85:2444, by computerized implementations of these algorithms (GAP, BESTFIT, BLAST, FASTA, and TFASTA in the Wisconsin Genetics Software Package, Genetics Computer Group (GCG), 575 Science Dr., Madison, W1), or by inspection.

One example of algorithms that are suitable for determining percent sequence identity and sequence similarity are the BLAST and BLAST 2.0 algorithms, which are described in Altschul et al., Nucl. Acids Res. 1977 25:3389-3402, and Altschul et al. J. MoI. Biol. 1990 215:403-10, respectively. BLAST and BLAST 2.0 can be used, for example with the parameters described herein, to determine percent sequence identity for the polynucleotides. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a word length (W) of 11, and expectation (E) of 10, and the BLOSUM62 scoring matrix (see Henikoff, et al. Proc. Natl. Acad. Sci. USA 1989; 89:10915) alignments, (B) of 50, expectation (E) of 10, M=5, N=−4 and a comparison of both strands.

In certain embodiments, the “percentage of sequence identity” is determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

It is appreciated by those of ordinary skill in the art that, as a result of the degeneracy of the genetic code, there are many nucleotide sequences that encode a particular antigen of interest, or fragment thereof, as described herein. Some of these polynucleotides bear minimal homology to the nucleotide sequence of any native gene. Nonetheless, polynucleotides that vary due to differences in codon usage are specifically contemplated.

Further, alleles of the genes comprising the polynucleotide sequences provided herein may also be contemplated. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Therefore, in another embodiment, a mutagenesis approach, such as site-specific mutagenesis, is employed for the preparation of variants and/or derivatives of nucleic acid sequences encoding one or more tumor antigens such as a HER2/neu antigen or epitope, or fragments thereof, as described herein. By this approach, specific modifications in a polypeptide sequence can be made through mutagenesis of the underlying polynucleotides that encode them. These techniques provide a straightforward approach to prepare and test sequence variants, for example, incorporating one or more of the foregoing considerations, by introducing one or more nucleotide sequence changes into the polynucleotide.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

Polynucleotide segments or fragments encoding the polypeptides may be readily prepared by, for example, directly synthesizing the fragment by chemical means, as is commonly practiced using an automated oligonucleotide synthesizer. Also, fragments may be obtained by application of nucleic acid reproduction technology, such as the PCR™ technology of U.S. Pat. No. 4,683,202, by introducing selected sequences into recombinant vectors for recombinant production, and by other recombinant DNA techniques generally known to those of skill in the art of molecular biology (see for example, Current Protocols in Molecular Biology, John Wiley and Sons, NY, NY).

In order to express a desired tumor antigen such as a HER2/neu antigen or epitope, polypeptide or fragment thereof, or fusion protein comprising any of the above, as described herein, the nucleotide sequences encoding the polypeptide, or functional equivalents, are inserted into an appropriate vector such as a replication-defective adenovirus vector as described herein using recombinant techniques known in the art. The appropriate vector contains the necessary elements for the transcription and translation of the inserted coding sequence and any desired linkers.

Methods that are available to those skilled in the art may be used to construct these vectors containing sequences encoding one or more tumor antigens such as a HER2/neu antigen or epitope and appropriate transcriptional and translational control elements. These methods include in vitro recombinant DNA techniques, synthetic techniques, and in vivo genetic recombination. Such techniques are described, for example, in Amalfitano, et al. J. Virol. 1998; 72:926-33; Hodges, et al. J Gene Med 2000; 2:250-259; Sambrook J, et al. (1989) Molecular Cloning, A Laboratory Manual, Cold Spring Harbor Press, Plainview, N.Y., and Ausubel F M, et al. (1989) Current Protocols in Molecular Biology, John Wiley & Sons, New York. N.Y.

A variety of vector/host systems may be utilized to contain and produce polynucleotide sequences. These include, but are not limited to, microorganisms such as bacteria transformed with recombinant bacteriophage, plasmid, or cosmid DNA vectors; yeast transformed with yeast vectors; insect cell systems infected with virus vectors (e.g., baculovirus); plant cell systems transformed with virus vectors (e.g., cauliflower mosaic virus, CaMV; tobacco mosaic virus, TMV) or with bacterial vectors (e.g., Ti or pBR322 plasmids); or animal cell systems.

The “control elements” or “regulatory sequences” present in a vector, such as an adenovirus vector, are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, sequences encoding one or more tumor antigens such as a HER2/neu antigen or epitope may be ligated into an Ad transcription/translation complex consisting of the late promoter and tripartite leader sequence. Insertion in a non-essential E1 or E3 region of the viral genome may be used to obtain a viable virus that is capable of expressing the polypeptide in infected host cells (Logan J, et al. Proc. Natl. Acad. Sci 1984; 87:3655-59). In addition, transcription enhancers, such as the Rous sarcoma virus (RSV) enhancer, may be used to increase expression in mammalian host cells.

Specific initiation signals may also be used to achieve more efficient translation of sequences encoding one or more tumor antigens such as a HER2/neu antigen or epitope. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers that are appropriate for the particular cell system which is used, such as those described in the literature (Scharf D., et al. Results Probl. Cell Differ. 1994; 20:125-62). Specific termination sequences, either for transcription or translation, may also be incorporated in order to achieve efficient translation of the sequence encoding the polypeptide of choice.

A variety of protocols for detecting and measuring the expression of polynucleotide-encoded products (e.g., one or more tumor antigens such as a HER2/neu antigen or epitope), using either polyclonal or monoclonal antibodies specific for the product are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on a given polypeptide may be preferred for some applications, but a competitive binding assay may also be employed. These and other assays are described, among other places, in Hampton R et al. (1990; Serological Methods, a Laboratory Manual, APS Press, St Paul. Minn.) and Maddox D E, et al. J. Exp. Med. 1983; 758:1211-16).

In certain embodiments, elements that increase the expression of the desired tumor antigens such as a HER2/neu antigen or epitope may be incorporated into the nucleic acid sequence of expression constructs or vectors such as adenovirus vectors described herein. Such elements include internal ribosome binding sites (IRES; Wang, et al. Curr. Top. Microbiol. Immunol 1995; 203:99; Ehrenfeld, et al. Curr. Top. Microbiol. Immunol. 1995; 203:65; Rees, et al. Biotechniques 1996; 20:102; Sugimoto, et al. Biotechnology 1994; 2:694). IRES increase translation efficiency. As well, other sequences may enhance expression. For some genes, sequences especially at the 5′ end inhibit transcription and/or translation. These sequences are usually palindromes that can form hairpin structures. Any such sequences in the nucleic acid to be delivered are generally deleted. Expression levels of the transcript or translated product are assayed to confirm or ascertain which sequences affect expression. Transcript levels may be assayed by any known method, including Northern blot hybridization, RNase probe protection and the like. Protein levels may be assayed by any known method, including ELISA.

As would be recognized by a skilled artisan, vectors, such as adenovirus vectors described herein, that comprise heterologous nucleic acid sequences can be generated using recombinant techniques known in the art, such as those described in Maione, et al. Proc Natl Acad Sci USA 2001; 98:5986-91; Maione, et al. Hum Gene Ther 2000 1:859-68; Sandig, et al. Proc Natl Acad Sci USA, 2000; 97:1002-07; Harui, et al. Gene Therapy 2004; 11:1617-26; Parks et al. Proc Natl Acad Sci USA 1996; 93:13565-570; DelloRusso, et al. Proc Natl Acad Sci USA 2002; 99:12979-984; Current Protocols in Molecular Biology, John Wiley and Sons, NY, NY).

### VII. Pharmaceutical Compositions

In certain aspects, there may be provided pharmaceutical compositions that comprise nucleic acid sequences encoding one or more one or more tumor antigens such as a HER2/neu antigen or epitope against which an immune response is to be generated. For example, tumor antigens may include, but are not limited to, a HER2/neu antigen or epitope or in combination with one or more additional tumor antigens as described herein or available in the art.

For example, the adenovirus vector stock described herein may be combined with an appropriate buffer, physiologically acceptable carrier, excipient or the like. In certain embodiments, an appropriate number of adenovirus vector particles are administered in an appropriate buffer, such as, sterile PBS. In certain circumstances it will be desirable to deliver the adenovirus vector compositions disclosed herein parenterally, intravenously, intramuscularly, or even intraperitoneally.

In certain embodiments, solutions of the pharmaceutical compositions as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. In other embodiments, E2b deleted adenovirus vectors may be delivered in pill form, delivered by swallowing or by suppository.

Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). In all cases the form must be sterile and must be fluid to the extent that easy syringability exists. It must be stable under the conditions of manufacture and storage and must be preserved against the contaminating action of microorganisms, such as bacteria, molds and fungi.

The carrier can be a solvent or dispersion medium containing, for example, water, lipids, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and/or by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

In one embodiment, for parenteral administration in an aqueous solution, the solution may be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. Moreover, for human administration, preparations will of course preferably meet sterility, pyrogenicity, and the general safety and purity standards as required by FDA Office of Biology standards.

The carriers can further comprise any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a human.

Routes and frequency of administration of the therapeutic compositions described herein, as well as dosage, will vary from individual to individual, and from disease to disease, and may be readily established using standard techniques. In general, the pharmaceutical compositions and vaccines may be administered by injection (e.g., intracutaneous, intramuscular, intravenous or subcutaneous), intranasally (e.g., by aspiration), in pill form (e.g., swallowing, suppository for vaginal or rectal delivery). In certain embodiments, between 1 and 3 doses may be administered over a 6 week period and further booster vaccinations may be given periodically thereafter.

For example, a suitable dose is an amount of an adenovirus vector that, when administered as described above, is capable of promoting a target antigen immune response as described elsewhere herein. In certain embodiments, the immune response is at least 10-50% above the basal (i.e., untreated) level. Such response can be monitored by measuring the antibodies against the target antigen in a patient or by vaccine-dependent generation of cytolytic effector cells Capable of killing the target antigen-expressing cells in vitro, or other methods known in the art for monitoring immune responses. The target antigen is a HER2/neu antigen or epitope as described herein

In general, an appropriate dosage and treatment regimen provides the adenovirus vectors in an amount sufficient to provide prophylactic benefit. Protective immune responses may generally be evaluated using standard proliferation, cytotoxicity or cytokine assays, which may be performed using samples obtained from a patient before and after immunization (vaccination).

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

While one advantage of compositions and methods described herein is the capability to administer multiple vaccinations with the same adenovirus vectors, particularly in individuals with preexisting immunity to Ad, the adenoviral vaccines described herein may also be administered as part of a prime and boost regimen. A mixed modality priming and booster inoculation scheme may result in an enhanced immune response. Thus, one aspect is a method of priming a subject with a plasmid vaccine, such as a plasmid vector comprising nucleic acid sequences encoding one or more tumor antigens such as a HER2/neu antigen or epitope, by administering the plasmid vaccine at least one time, allowing a predetermined length of time to pass, and then boosting by administering the adenovirus vector described herein.

Multiple primings, e.g., 1-3, may be employed, although more may be used. The length of time between priming and boost may typically vary from about six months to a year, but other time frames may be used.

In certain embodiments, pharmaceutical compositions may comprise, for example, at least about 0.1% of therapeutic agents, such as the expression constructs or vectors used herein as vaccine, a related lipid nanovesicle, or an exosome or nanovesicle loaded with therapeutic agents. In other embodiments, the therapeutic agent may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 microgram/kg/body weight to about 100 mg/kg/body weight, about 5 microgram/kg/body weight to about 500 milligram/kg/body weight, etc., can be administered.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

In certain aspects, compositions comprising a vaccination regime as described herein can be administered either alone or together with a pharmaceutically acceptable carrier or excipient, by any routes, and such administration can be carried out in both single and multiple dosages. More particularly, the pharmaceutical composition can be combined with various pharmaceutically acceptable inert carriers in the form of tablets, capsules, lozenges, troches, hand candies, powders, sprays, aqueous suspensions, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes. The compositions described throughout can be formulated into a pharmaceutical medicament and be used to treat a human or mammal, in need thereof, diagnosed with a disease, e.g., cancer, or to enhances an immune response.

In certain embodiments, the viral vectors or compositions described herein may be administered in conjunction with one or more immunostimulants, such as an adjuvant. An immunostimulant refers to essentially any substance that enhances or potentiates an immune response (antibody and/or cell-mediated) to an antigen. One type of immunostimulant comprises an adjuvant. Many adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Certain adjuvants are commercially available as, for example, Freund's Incomplete Adjuvant and Complete Adjuvant (Difco Laboratories); Merck Adjuvant 65 (Merck and Company, Inc.) AS-2 (SmithKline Beecham); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, IFN-γ, TNFα, IL-2, IL-8, IL-12, IL-18, IL-7, IL-3, IL-4, IL-5, IL-6, IL-9, IL-10, IL-13, IL-15, IL-16, IL-17, IL-23, and/or IL-32, and others, like growth factors, may also be used as adjuvants.

Within certain embodiments, the adjuvant composition can be one that induces an immune response predominantly of the Th1 type. High levels of Th1-type cytokines (e.g., IFN-γ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient may support an immune response that includes Th1- and/or Th2-type responses. Within certain embodiments, in which a response is predominantly Th1-type, the level of Th1-type cytokines will increase to a greater extent than the level of Th2-type cytokines. The levels of these cytokines may be readily assessed using standard assays. Thus, various embodiments relate to therapies raising an immune response against a target antigen, for example a HER2/neu antigen or epitope, using cytokines, e.g., IFN-γ, TNFα, IL-2, IL-8, IL-12, IL-18, IL-7, IL-3, IL-4, IL-5, IL-6, IL-9, IL-10, IL-13 and/or IL-15 supplied concurrently with a replication defective viral vector treatment. In some embodiments, a cytokine or a nucleic acid encoding a cytokine, is administered together with a replication defective viral described herein. In some embodiments, cytokine administration is performed prior or subsequent to viral vector administration. In some embodiments, a replication defective viral vector capable of raising an immune response against a target antigen, for example a HER2/neu antigen or epitope, further comprises a sequence encoding a cytokine.

Certain illustrative adjuvants for eliciting a predominantly Th1-type response include, for example, a combination of monophosphoryl lipid A, such as 3-de-O-acylated monophosphoryl lipid A, together with an aluminum salt. MPL® adjuvants are commercially available (see, e.g., U.S. Pat. Nos. 4,436,727; 4,877,611; 4,866,034; and 4,912,094). CpG-containing oligonucleotides (in which the CpG dinucleotide is unmethylated) also induce a predominantly Th1 response. (see, e.g., WO 96/02555, WO 99/33488 and U.S. Pat. Nos. 6,008,200 and 5,856,462). Immunostimulatory DNA sequences can also be used.

Another adjuvant for use in some embodiments comprises a saponin, such as Quil A, or derivatives thereof, including QS21 and QS7 (Aquila Biopharmaceuticals Inc.), Escin; Digitonin; or Gypsophila or Chenopodium quinoa saponins. Other formulations may include more than one saponin in the adjuvant combinations, e.g., combinations of at least two of the following group comprising QS21, QS7, Quil A, β-escin, or digitonin.

In some embodiments, the compositions may be delivered by intranasal sprays, inhalation, and/or other aerosol delivery vehicles. The delivery of drugs using intranasal microparticle resins and lysophosphatidyl-glycerol compounds can be employed (see, e.g., U.S. Pat. No. 5,725,871). Likewise, illustrative transmucosal drug delivery in the form of a polytetrafluoroetheylene support matrix can be employed (see, e.g., U.S. Pat. No. 5,780,045).

Liposomes, nanocapsules, microparticles, lipid particles, vesicles, and the like, can be used for the introduction of the compositions as described herein into suitable hot cells/organisms. Compositions as described herein may be formulated for delivery either encapsulated in a lipid particle, a liposome, a vesicle, a nanosphere, or a nanoparticle or the like. Alternatively, compositions as described herein can be bound, either covalently or non-covalently, to the surface of such carrier vehicles. Liposomes can be used effectively to introduce genes, various drugs, radiotherapeutic agents, enzymes, viruses, transcription factors, allosteric effectors and the like, into a variety of cultured cell lines and animals. Furthermore, the use of liposomes does not appear to be associated with autoimmune responses or unacceptable toxicity after systemic delivery. In some embodiments, liposomes are formed from phospholipids dispersed in an aqueous medium and spontaneously form multilamellar concentric bilayer vesicles (i.e., multilamellar vesicles (MLVs)).

In some embodiments, there are provided pharmaceutically-acceptable nanocapsule formulations of the compositions or vectors as described herein. Nanocapsules can generally entrap pharmaceutical compositions in a stable and reproducible way. To avoid side effects due to intracellular polymeric overloading, such ultrafine particles (sized around 0.1 μm) may be designed using polymers able to be degraded in vivo.

In certain aspects, a pharmaceutical composition comprising IL-15 may be administered to an individual in need thereof, in combination with one or more therapy provided herein, particularly one or more adenoviral vectors comprising nucleic acid sequences encoding one or more target antigens such as a HER2/neu antigen or epitope.

Interleukin 15 (IL-15) is a cytokine with structural similarity to IL-2. Like IL-2, IL-15 binds to and signals through a complex composed of IL-2/IL-15 receptor beta chain (CD122) and the common gamma chain (gamma-C, CD132). IL-15 is secreted by mononuclear phagocytes (and some other cells) following infection by virus(es). This cytokine induces cell proliferation of natural killer cells; cells of the innate immune system whose principal role is to kill virally infected cells.

IL-15 can enhance the anti-tumor immunity of CD8+ T cells in pre-clinical models. A phase I clinical trial to evaluate the safety, dosing, and anti-tumor efficacy of IL-15 in patients with metastatic melanoma and renal cell carcinoma (kidney cancer) has begun to enroll patients at the National Institutes of Health.

IL-15 disclosed herein may also include mutants of IL-15 that are modified to maintain the function of its native form.

IL-15 is 14-15 kDa glycoprotein encoded by the 34 kb region 4q31 of chromosome 4, and by the central region of chromosome 8 in mice. The human IL-15 gene comprises nine exons (1-8 and 4A) and eight introns, four of which (exons 5 through 8) code for the mature protein. Two alternatively spliced transcript variants of this gene encoding the same protein have been reported. The originally identified isoform, with long signal peptide of 48 amino acids (IL-15 LSP) consisted of a 316 bp 5′-untranslated region (UTR), 486 bp coding sequence and the C-terminus 400 bp 3′-UTR region. The other isoform (IL-15 SSP) has a short signal peptide of 21 amino acids encoded by exons 4A and 5. Both isoforms shared 11 amino acids between signal sequences of the N-terminus. Although both isoforms produce the same mature protein, they differ in their cellular trafficking. IL-15 LSP isoform was identified in Golgi apparatus (GC), early endosomes and in the endoplasmic reticulum (ER). It exists in two forms, secreted and membrane-bound particularly on dendritic cells. On the other hand, IL-15 SSP isoform is not secreted and it appears to be restricted to the cytoplasm and nucleus where it plays an important role in the regulation of cell cycle.

It has been demonstrated that two isoforms of IL-15 mRNA are generated by alternatively splicing in mice. The isoform which had an alternative exon 5 containing another 3′ splicing site, exhibited a high translational efficiency, and the product lack hydrophobic domains in the signal sequence of the N-terminus. This suggests that the protein derived from this isoform is located intracellularly. The other isoform with normal exon 5, which is generated by integral splicing of the alternative exon 5, may be released extracellularly.

Although IL-15 mRNA can be found in many cells and tissues including mast cells, cancer cells or fibroblasts, this cytokine is produce as a mature protein mainly by dendritic cells, monocytes and macrophages. This discrepancy between the wide appearance of IL-15 mRNA and limited production of protein might be explained by the presence of the twelve in humans and five in mice upstream initiating codons, which can repress translation of IL-15 mRNA. Translational inactive mRNA is stored within the cell and can be induced upon specific signal. Expression of IL-15 can be stimulated by cytokine such as GM-CSF, double-strand mRNA, unmethylated CpG oligonucleotides, lipopolysaccharide (LPS) through Toll-like receptors (TLR), interferon gamma (IFN-γ) or after infection of monocytes herpes virus, Mycobacterium tuberculosis and Candida albicans.

### VIII. Natural Killer (NK) Cells

In certain embodiments, native or engineered NK cells may be provided to be administered to a subject in need thereof, in combination with adenoviral vector-based compositions or immunotherapy as described herein.

The immune system is a tapestry of diverse families of immune cells each with its own distinct role in protecting from infections and diseases. Among these immune cells are the natural killer, or NK, cells as the body's first line of defense. NK cells have the innate ability to rapidly seek and destroy abnormal cells, such as cancer or virally-infected cells, without prior exposure or activation by other support molecules. In contrast to adaptive immune cells such as T cells, NK cells have been utilized as a cell-based “off-the-shelf” treatment in phase 1 clinical trials, and have demonstrated tumor killing abilities for cancer.

1. aNK Cells

In addition to native NK cells, there may be provided NK cells for administering to a patient that has do not express Killer Inhibitory Receptors (KIR), which diseased cells often exploit to evade the killing function of NK cells. This unique activated NK, or aNK, cell lack these inhibitory receptors while retaining the broad array of activating receptors which enable the selective targeting and killing of diseased cells. aNK cells also carry a larger pay load of granzyme and perforin containing granules, thereby enabling them to deliver a far greater payload of lethal enzymes to multiple targets.

2. taNK Cells

Chimeric antigen receptor (CAR) technology is among the most novel cancer therapy approaches currently in development. CARs are proteins that allow immune effector cells to target cancer cells displaying specific surface antigen (target-activated Natural Killer) is a platform in which aNK cells are engineered with one or more CARs to target proteins found on cancers and is then integrated with a wide spectrum of CARs. This strategy has multiple advantages over other CAR approaches using patient or donor sourced effector cells such as autologous T-cells, especially in terms of scalability, quality control and consistency.

Much of the cancer cell killing relies upon ADCC (antibody dependent cell-mediated cytotoxicity) whereupon effector immune cells attach to antibodies, which are in turn bound to the target cancer cell, thereby facilitating killing of the cancer by the effector cell. NK cells are the key effector cell in the body for ADCC and utilize a specialized receptor (CD16) to bind antibodies.

3. haNK Cells

Studies have shown that perhaps only 20% of the human population uniformly expresses the “high-affinity” variant of CD16 (haNK cells), which is strongly correlated with more favorable therapeutic outcomes compared to patients with the “low-affinity” CD16. Additionally, many cancer patients have severely weakened immune systems due to chemotherapy, the disease itself or other factors.

In certain aspects, NK cells are modified to express high-affinity CD16 (haNK cells). As such, haNK cells may potentiate the therapeutic efficacy of a broad spectrum of antibodies directed against cancer cells.

### IX. Combination Therapy

The compositions comprising an adenoviral vector-based vaccination comprising a nucleic acid sequence encoding tumor antigens such as a HER2/neu antigen or epitope described throughout can be formulated into a pharmaceutical medicament and be used to treat a human or mammal in need thereof or diagnosed with a disease, e.g., cancer. These medicaments can be co-administered with one or more additional vaccines or other cancer therapy to a human or mammal.

In certain aspects, the medicaments as described herein can be combined with one or more available therapy for breast cancer, for example, conventional cancer therapy such as surgery, radiation therapy or medications such as hormone blocking therapy, chemotherapy or monoclonal antibodies. In some embodiments, any vaccine described herein (e.g., Ad5[E1−, E2b−]-HER3) can be combined with low dose chemotherapy or low dose radiation. For example, in some embodiment, any vaccine described herein (e.g., Ad5[E1−, E2b−]-HER3) can be combined with chemotherapy, such that the dose of chemotherapy administered is lower than the clinical standard of care. In some embodiments, the chemotherapy can be cyclophosphamide. The cyclophasmade can administered at a dose that is lower than the clinical standard of care dosing. For example, the chemotherapy can be administered at 50 mg twice a day (BID) on days 1-5 and 8-12 every 2 weeks for a total of 8 weeks. In some embodiments, any vaccine described herein (e.g., Ad5[E1−, E2b−]-HER3) can be combined with radiation, such that the dose of radiation administered is lower than the clinical standard of care. For example, in some embodiments, concurrent sterotactic body radiotherapy (SBRT) at 8 Gy can be given on day 8, 22, 36, 50 (every 2 weeks for 4 doses). Radiation can be administered to all feasible tumor sites using SBRT.

In certain aspects, medications used for breast cancer treatment include hormone-blocking agents, chemotherapy, and monoclonal antibodies. Some breast cancers require estrogen to continue growing. They can be identified by the presence of estrogen receptors (ER+) and progesterone receptors (PR+) on their surface (sometimes referred to together as hormone receptors). These ER+ cancers can be treated with drugs that either block the receptors, e.g., tamoxifen, or alternatively block the production of estrogen with an aromatase inhibitor, e.g., anastrozole or letrozole. The use of tamoxifen is recommended for 10 years. Aromatase inhibitors are useful for women after menopause; however, in this group, they appear better than tamoxifen. This is because the active aromatase in postmenopausal women is different from the prevalent form in premenopausal women, and therefore these agents are ineffective in inhibiting the predominant aromatase of premenopausal women.

Chemotherapy is predominantly used for cases of breast cancer in stages 2-4, and is particularly beneficial in estrogen receptor-negative (ER−) disease. The chemotherapy medications are administered in combinations, usually for periods of 3-6 months. One of the most common regimens, known as “AC,” combinescyclophosphamide with doxorubicin. Sometimes a taxane drug, such as docetaxel (Taxotere), is added, and the regime is then known as “CAT.” Another common treatment is cyclophosphamide, methotrexate, and fluorouracil (or “CMF”). Most chemotherapy medications work by destroying fast-growing and/or fast-replicating cancer cells, either by causing DNA damage upon replication or by other mechanisms. However, the medications also damage fast-growing normal cells, which may cause serious side effects. Damage to the heart muscle is the most dangerous complication of doxorubicin, for example.

HER2/neu is the target of the monoclonal antibody trastuzumab (marketed as Herceptin). Trastuzumab, a monoclonal antibody to HER2/neu (a cell receptor that is especially active in some breast cancer cells), has improved the 5-year disease free survival of stage 1-3 HER2/neu-positive breast cancers to about 87% (overall survival 95%). One year of trastuzumab therapy is recommended for all patients with HER2/neu-positive breast cancer who are also receiving chemotherapy.

When stimulated by certain growth factors, HER2/neu causes cellular growth and division; in the absence of stimulation by the growth factor, the cell normally stops growing. Between 25% and 30% of breast cancers overexpress the HER2/neu gene or its protein product, and overexpression of HER2/neu in breast cancer is associated with increased disease recurrence and worse prognosis. When trastuzumab binds to the HER2/neu in breast cancer cells that overexpress the receptor, trastuzumab prevents growth factors from being able to bind to and stimulate the receptors, effectively blocking the growth of the cancer cells. An important downstream effect of trastuzumab binding to HER2/neu is an increase in p27, a protein that halts cell proliferation. Thus, Trastuzumab is useful for breast cancer patients with HER2/neu amplification/overexpression.

Another monoclonal antibody, Pertuzumab, which inhibits dimerisation of HER2/neu and HER3 receptors, was approved by the FDA for use in combination with trastuzumab in June 2012.

Additionally, NeuVax (Galena Biopharma) is a peptide-based immunotherapy that directs “killer” T cells to target and destroy cancer cells that express HER2/neu. It has entered phase 3 clinical trials.

It has been found that patients with ER+(Estrogen receptor positive)/HER2/neu+ compared with ER−/HER2/neu+ breast cancers may actually benefit more from drugs that inhibit the PI3K/AKT molecular pathway.

Over-expression of HER2/neu can also be suppressed by the amplification of other genes. Research is currently being conducted to discover which genes may have this desired effect.

The expression of HER2/neu is regulated by signaling through estrogen receptors. Normally, estradiol and tamoxifen acting through the estrogen receptor down-regulate the expression of HER2/neu. However, when the ratio of the coactivator AIB-3 exceeds that of thecorepressor PAX2, the expression of HER2/neu is upregulated in the presence of tamoxifen, leading to tamoxifen-resistant breast cancer.

In certain aspects, these medicaments as described herein can be combined together with one or more conventional cancer therapies or alternative cancer therapies or immune pathway checkpoint modulators as described herein. The combination therapy involving the adenovirus vector-based medicaments can be used to treat any cancer, particularly, breast cancer, or unresectable, locally advanced, or metastatic cancer.

Conventional cancer therapies include one or more selected from the group of chemical or radiation based treatments and surgery. Chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

Radiation therapy that causes DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

Approximately 60% of persons with cancer will undergo surgery of some type, which includes preventative, diagnostic or staging, curative and palliative surgery. Curative surgery is a cancer treatment that may be used in conjunction with other therapies, such as the treatment described herein, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that treatment methods described herein may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, 7, 8, 9, 10, 11, 12, 13, or 14 days, or every 1, 2, 3, 4, 5, 6, 7, 8, 9, 10, 11, 12, 13, 14, 15, 16, 17, 18, 19, or 20 weeks or every 1, 2, 3, 4, 5, 6, 7, 8, 9, 10, 11, 12, 13, 14, 15, 16, 17, 18, 19, 20, 21, 22, 23, or 24 months. These treatments may be of varying dosages as well.

Alternative cancer therapies include any cancer therapy other than surgery, chemotherapy and radiation therapy, such as immunotherapy, gene therapy, hormonal therapy or a combination thereof. Subjects identified with poor prognosis using the present methods may not have favorable response to conventional treatment(s) alone and may be prescribed or administered one or more alternative cancer therapy per se or in combination with one or more conventional treatments.

Immunotherapeutics generally rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Gene therapy is the insertion of polynucleotides, including DNA or RNA, into a subject's cells and tissues to treat a disease. Antisense therapy is also a form of gene therapy. A therapeutic polynucleotide may be administered before, after, or at the same time of a first cancer therapy. Delivery of a vector encoding a variety of proteins is provided in some embodiments. For example, cellular expression of the exogenous tumor suppressor oncogenes would exert their function to inhibit excessive cellular proliferation, such as p53, p16 and C-CAM.

Additional agents to be used to improve the therapeutic efficacy of treatment include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with pharmaceutical compositions described herein to improve the anti-hyperproliferative efficacy of the treatments. Inhibitors of cell adhesion are contemplated to improve the efficacy of pharmaceutical compositions described herein. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with pharmaceutical compositions described herein to improve the treatment efficacy.

Hormonal therapy may also be used in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

A “Chemotherapeutic agent” or “chemotherapeutic compound” and their grammatical equivalents as used herein, can be a chemical compound useful in the treatment of cancer. The chemotherapeutic cancer agents that can be used in combination with the disclosed T cell include, but are not limited to, mitotic inhibitors (vinca alkaloids). These include vincristine, vinblastine, vindesine and Navelbine™ (vinorelbine,5′-noranhydroblastine). In yet other embodiments, chemotherapeutic cancer agents include topoisomerase I inhibitors, such as camptothecin compounds. As used herein, “camptothecin compounds” include Camptosar™ (irinotecan HCL), Hycamtin™ (topotecan HCL) and other compounds derived from camptothecin and its analogues. Another category of chemotherapeutic cancer agents that can be used in the methods and compositions disclosed herein are podophyllotoxin derivatives, such as etoposide, teniposide and mitopodozide.

In certain aspects, methods or compositions described herein further encompass the use of other chemotherapeutic cancer agents known as alkylating agents, which alkylate the genetic material in tumor cells. These include without limitation cisplatin, cyclophosphamide, nitrogen mustard, trimethylene thiophosphoramide, carmustine, busulfan, chlorambucil, belustine, uracil mustard, chlomaphazin, and dacarbazine. The disclosure encompasses antimetabolites as chemotherapeutic agents. Examples of these types of agents include cytosine arabinoside, fluorouracil, methotrexate, mercaptopurine, azathioprime, and procarbazine.

An additional category of chemotherapeutic cancer agents that may be used in the methods and compositions disclosed herein includes antibiotics. Examples include without limitation doxorubicin, bleomycin, dactinomycin, daunorubicin, mithramycin, mitomycin, mytomycin C, and daunomycin. There are numerous liposomal formulations commercially available for these compounds. In certain aspects, methods or compositions described herein further encompass the use of other chemotherapeutic cancer agents including without limitation anti-tumor antibodies, dacarbazine, azacytidine, amsacrine, melphalan, ifosfamide and mitoxantrone.

The disclosed adenovirus vaccine herein can be administered in combination with other anti-tumor agents, including cytotoxic/antineoplastic agents and anti-angiogenic agents. Cytotoxic/anti-neoplastic agents can be defined as agents who attack and kill cancer cells. Some cytotoxic/anti-neoplastic agents can be alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents can be antimetabolites for tumor cells, e.g., cytosine arabinoside, fluorouracil, methotrexate, mercaptopuirine, azathioprime, and procarbazine. Other cytotoxic/anti-neoplastic agents can be antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents can be mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Additional formulations comprising population(s) of CAR T cells, T cell receptor engineered T cells, B cell receptor engineered cells, can be administered to a subject in conjunction, before, or after the administration of the pharmaceutical compositions described herein. A therapeutically-effective population of adoptively transferred cells can be administered to subjects when the methods described herein are practiced. In general, formulations are administered that comprise from about 1×10⁴ to about 1×10¹⁰ CAR T cells, T cell receptor engineered cells, or B cell receptor engineered cells. In some cases, the formulation comprises from about 1×10⁵ to about 1×10⁹ engineered cells, from about 5×10⁵ to about 5×10⁸ engineered cells, or from about 1×10⁶ to about 1×10⁷ engineered cells. However, the number of engineered cells administered to a subject will vary between wide limits, depending upon the location, source, identity, extent and severity of the cancer, the age and condition of the subject to be treated etc. A physician will ultimately determine appropriate dosages to be used.

Anti-angiogenic agents can also be used. Suitable anti-angiogenic agents for use in the disclosed methods and compositions include anti-VEGF antibodies, including humanized and chimeric antibodies, anti-VEGF aptamers and antisense oligonucleotides. Other inhibitors of angiogenesis include angiostatin, endostatin, interferons, interleukin 1 (including a and β) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

In some cases, for example, in the compositions, formulations and methods of treating cancer, the unit dosage of the composition or formulation administered can be 5, 10, 15, 20, 25, 30, 35, 40, 45, 50, 55, 60, 65, 70, 75, 80, 85, 90, 95, 100 mg, or any intervening value or range derived therefrom. In some cases, the total amount of the composition or formulation administered can be 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25, 30, 40, 50, 60, 70, 80, 90, 100 g, or any intervening value or range derived therefrom.

### X. Immunological Fusion Partner Antigen Targets

The viral vectors or composition described herein may further comprise nucleic acid sequences that encode proteins, or an “immunological fusion partner,” that can increase the immunogenicity of the target antigen such as HER2/neu, or any other target antigen disclosed herein. In this regard, the protein produced following immunization with the viral vector containing such a protein may be a fusion protein comprising the target antigen of interest fused to a protein that increases the immunogenicity of the target antigen of interest. Furthermore, combination therapy with Ad5[E1−, E2b−] vectors encoding for HER2/neu and an immunological fusion partner can result in boosting the immune response, such that the combination of both therapeutic moieties acts to synergistically boost the immune response than either the Ad5[E1−, E2b−] vectors encoding for HER2/neu alone, or the immunological fusion partner alone. For example, combination therapy with Ad5[E1−, E2b−] vectors encoding for HER2/neu and an immunological fusion partner can result in synergistic enhancement of stimulation of antigen-specific effector CD4+ and CD8+ T cells, stimulation of NK cell response directed towards killing infected cells, stimulation of neutrophils or monocyte cell responses directed towards killing infected cells via antibody dependent cell-mediated cytotoxicity (ADCC), antibody dependent cellular phagocytosis (ADCP) mechanisms, or any combination thereof. This synergistic boost can vastly improve survival outcomes after administration to a subject in need thereof. In certain embodiments, combination therapy with Ad5[E1−, E2b−] vectors encoding for HER2/neu and an immunological fusion partner can result in generating an immune response comprises an increase in target antigen-specific CTL activity of about 1.5 to 20, or more fold in a subject administered the adenovirus vectors as compared to a control. In another embodiment, generating an immune response comprises an increase in target-specific CTL activity of about 1.5 to 20, or more fold in a subject administered the Ad5[E1−, E2b−] vectors encoding for HER2/neu antigens and an immunological fusion partner as compared to a control. In a further embodiment, generating an immune response that comprises an increase in target antigen-specific cell-mediated immunity activity as measured by ELISpot assays measuring cytokine secretion, such as interferon-gamma (IFN-γ), interleukin-2 (IL-2), tumor necrosis factor-alpha (TNF-α), or other cytokines, of about 1.5 to 20, or more fold as compared to a control. In a further embodiment, generating an immune response comprises an increase in target-specific antibody production of between 1.5 and 5 fold in a subject administered the Ad5[E1−, E2b−] vectors encoding for HER2/neu antigens and an immunological fusion partner as described herein as compared to an appropriate control. In another embodiment, generating an immune response comprises an increase in target-specific antibody production of about 1.5 to 20, or more fold in a subject administered the adenovirus vector as compared to a control.

As an additional example, combination therapy with Ad5[E1−, E2b−] vectors encoding for target epitope antigens and an immunological fusion partner can result in synergistic enhancement of stimulation of antigen-specific effector CD4+ and CD8+ T cells, stimulation of NK cell response directed towards killing infected cells, stimulation of neutrophils or monocyte cell responses directed towards killing infected cells via antibody dependent cell-mediated cytotoxicity (ADCC), antibody dependent cellular phagocytosis (ADCP) mechanisms, or any combination thereof. This synergistic boost can vastly improve survival outcomes after administration to a subject in need thereof. In certain embodiments, combination therapy with Ad5[E1−, E2b−] vectors encoding for target epitope antigens and an immunological fusion partner can result in generating an immune response comprises an increase in target antigen-specific CTL activity of about 1.5 to 20, or more fold in a subject administered the adenovirus vectors as compared to a control. In another embodiment, generating an immune response comprises an increase in target-specific CTL activity of about 1.5 to 20, or more fold in a subject administered the Ad5[E1−, E2b−] vectors encoding for target epitope antigens and an immunological fusion partner as compared to a control. In a further embodiment, generating an immune response that comprises an increase in target antigen-specific cell-mediated immunity activity as measured by ELISpot assays measuring cytokine secretion, such as interferon-gamma (IFN-γ), interleukin-2 (IL-2), tumor necrosis factor-alpha (TNF-α), or other cytokines, of about 1.5 to 20, or more fold as compared to a control. In a further embodiment, generating an immune response comprises an increase in target-specific antibody production of between 1.5 and 5 fold in a subject administered the adenovirus vectors as described herein as compared to an appropriate control. In another embodiment, generating an immune response comprises an increase in target-specific antibody production of about 1.5 to 20, or more fold in a subject administered the adenovirus vector as compared to a control.

In one embodiment, such an immunological fusion partner is derived from a Mycobacterium sp., such as a Mycobacterium tuberculosis-derived Ra12 fragment. The immunological fusion partner derived from Mycobacterium sp. can be any one of the sequences set forth in SEQ ID NO: 35-SEQ ID NO: 43. Ra12 compositions and methods for their use in enhancing the expression and/or immunogenicity of heterologous polynucleotide/polypeptide sequences are described in U.S. Pat. No. 7,009,042, which is herein incorporated by reference in its entirety. Briefly, Ra12 refers to a polynucleotide region that is a subsequence of a Mycobacterium tuberculosis MTB32A nucleic acid. MTB32A is a serine protease of 32 kDa encoded by a gene in virulent and avirulent strains of M. tuberculosis. The nucleotide sequence and amino acid sequence of MTB32A have been described (see, e.g., U.S. Pat. No. 7,009,042; Skeiky et al., Infection and Immun. 67:3998-4007 (1999), incorporated herein by reference in their entirety). C-terminal fragments of the MTB32A coding sequence can be expressed at high levels and remain as soluble polypeptides throughout the purification process. Moreover, Ra12 may enhance the immunogenicity of heterologous immunogenic polypeptides with which it is fused. A Ra12 fusion polypeptide can comprise a 14 kDa C-terminal fragment corresponding to amino acid residues 192 to 323 of MTB32A. Other Ra12 polynucleotides generally can comprise at least about 15, 30, 60, 100, 200, 300, or more nucleotides that encode a portion of a Ra12 polypeptide. Ra12 polynucleotides may comprise a native sequence (i.e., an endogenous sequence that encodes a Ra12 polypeptide or a portion thereof) or may comprise a variant of such a sequence. Ra12 polynucleotide variants may contain one or more substitutions, additions, deletions and/or insertions such that the biological activity of the encoded fusion polypeptide is not substantially diminished, relative to a fusion polypeptide comprising a native Ra12 polypeptide. Variants can have at least about 70%, 80%, or 90% identity, or more, to a polynucleotide sequence that encodes a native Ra12 polypeptide or a portion thereof.

In certain aspects, an immunological fusion partner can be derived from protein D, a surface protein of the gram-negative bacterium Haemophilus influenzae B. The immunological fusion partner derived from protein D can be the sequence set forth in SEQ ID NO: 44. In some cases, a protein D derivative comprises approximately the first third of the protein (e.g., the first N-terminal 100-110 amino acids). A protein D derivative may be lipidated. Within certain embodiments, the first 109 residues of a Lipoprotein D fusion partner is included on the N-terminus to provide the polypeptide with additional exogenous T-cell epitopes, which may increase the expression level in E. coli and may function as an expression enhancer. The lipid tail may ensure optimal presentation of the antigen to antigen presenting cells. Other fusion partners can include the non-structural protein from influenza virus, NS1 (hemagglutinin). Typically, the N-terminal 81 amino acids are used, although different fragments that include T-helper epitopes may be used.

In certain aspects, the immunological fusion partner can be the protein known as LYTA, or a portion thereof (particularly a C-terminal portion). The immunological fusion partner derived from LYTA can the sequence set forth in SEQ ID NO: 45. LYTA is derived from Streptococcus pneumoniae, which synthesizes an N-acetyl-L-alanine amidase known as amidase LYTA (encoded by the LytA gene). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus can be employed. Within another embodiment, a repeat portion of LYTA may be incorporated into a fusion polypeptide. A repeat portion can, for example, be found in the C-terminal region starting at residue 178. One particular repeat portion incorporates residues 188-305.

In some embodiments, the target antigen is fused to an immunological fusion partner, also referred to herein as an “immunogenic component,” comprising a cytokine selected from the group of IFN-γ, TNFα, IL-2, IL-8, IL-12, IL-18, IL-7, IL-3, IL-4, IL-5, IL-6, IL-9, IL-10, IL-13, IL-15, IL-16, IL-17, IL-23, IL-32, M-CSF (CSF-1), IFN-α, IFN-β, IL-1α, IL-1β, IL-1RA, IL-11, IL-17A, IL-17F, IL-19, IL-20, IL-21, IL-22, IL-24, IL-25, IL-26, IL-27, IL-28A, B, IL-29, IL-30, IL-31, IL-33, IL-34, IL-35, IL-36α,β,λ, IL-36Ra, IL-37, TSLP, LIF, OSM, LT-α, LT-β, CD40 ligand, Fas ligand, CD27 ligand, CD30 ligand, 4-1BBL, Trail, OPG-L, APRIL, LIGHT, TWEAK, BAFF, TGF-β1, and MIF. The target antigen fusion can produce a protein with substantial identity to one or more of IFN-γ, TNFα IL-2, IL-8, IL-12, IL-18, IL-7, IL-3, IL-4, IL-5, IL-6, IL-9, IL-10, IL-13, IL-15, IL-16, IL-17, IL-23, IL-32, M-CSF (CSF-1), IFN-α, IFN-β, IL-1α, IL-1β, IL-1RA, IL-11, IL-17A, IL-17F, IL-19, IL-20, IL-21, IL-22, IL-24, IL-25, IL-26, IL-27, IL-28A, B, IL-29, IL-30, IL-31, IL-33, IL-34, IL-35, IL-36α,β,λ, IL-36Ra, IL-37, TSLP, LIF, OSM, LT-α, LT-β, CD40 ligand, Fas ligand, CD27 ligand, CD30 ligand, 4-1BBL, Trail, OPG-L, APRIL, LIGHT, TWEAK, BAFF, TGF-β1, and MIF. The target antigen fusion can encode a nucleic acid encoding a protein with substantial identity to one or more of IFN-γ, TNFα, IL-2, IL-8, IL-12, IL-18, IL-7, IL-3, IL-4, IL-5, IL-6, IL-9, IL-10, IL-13, IL-15, IL-16, IL-17, IL-23, IL-32, M-CSF (CSF-1), IFN-α, IFN-β, IL-1α, IL-1β, IL-1RA, IL-11, IL-17A, IL-17F, IL-19, IL-20, IL-21, IL-22, IL-24, IL-25, IL-26, IL-27, IL-28A, B, IL-29, IL-30, IL-31, IL-33, IL-34, IL-35, IL-36α,β,λ, IL-36Ra, IL-37, TSLP, LIF, OSM, LT-α, LT-β, CD40 ligand, Fas ligand, CD27 ligand, CD30 ligand, 4-1BBL, Trail, OPG-L, APRIL, LIGHT, TWEAK, BAFF, TGF-β1, and MIF. In some embodiments, the target antigen fusion further comprises one or more immunological fusion partner, also referred to herein as an “immunogenic components,” comprising a cytokine selected from the group of IFN-γ, TNFα, IL-2, IL-8, IL-12, IL-18, IL-7, IL-3, IL-4, IL-5, IL-6, IL-9, IL-10, IL-13, IL-15, IL-16, IL-17, IL-23, IL-32, M-CSF (CSF-1), IFN-α, IFN-β, IL-1α, IL-1β, IL-1RA, IL-11, IL-17A, IL-17F, IL-19, IL-20, IL-21, IL-22, IL-24, IL-25, IL-26, IL-27, IL-28A, B, IL-29, IL-30, IL-31, IL-33, IL-34, IL-35, IL-36α,β,λ, IL-36Ra, IL-37, TSLP, LIF, OSM, LT-α, LT-β, CD40 ligand, Fas ligand, CD27 ligand, CD30 ligand, 4-1BBL, Trail, OPG-L, APRIL, LIGHT, TWEAK, BAFF, TGF-β1, and MW. The sequence of IFN-γ can be, but is not limited to, a sequence as set forth in SEQ ID NO: 46. The sequence of TNFα can be, but is not limited to, a sequence as set forth in SEQ ID NO: 47. The sequence of IL-2 can be, but is not limited to, a sequence as set forth in SEQ ID NO: 48. The sequence of IL-8 can be, but is not limited to, a sequence as set forth in SEQ ID NO: 49. The sequence of IL-12 can be, but is not limited to, a sequence as set forth in SEQ ID NO: 50. The sequence of IL-18 can be, but is not limited to, a sequence as set forth in SEQ ID NO: 51. The sequence of IL-7 can be, but is not limited to, a sequence as set forth in SEQ ID NO: 52. The sequence of IL-3 can be, but is not limited to, a sequence as set forth in SEQ ID NO: 53. The sequence of IL-4 can be, but is not limited to, a sequence as set forth in SEQ ID NO: 54. The sequence of IL-5 can be, but is not limited to, a sequence as set forth in SEQ ID NO: 55. The sequence of IL-6 can be, but is not limited to, a sequence as set forth in SEQ ID NO: 56. The sequence of IL-9 can be, but is not limited to, a sequence as set forth in SEQ ID NO: 57. The sequence of IL-10 can be, but is not limited to, a sequence as set forth in SEQ ID NO: 58. The sequence of IL-13 can be, but is not limited to, a sequence as set forth in SEQ ID NO: 59. The sequence of IL-15 can be, but is not limited to, a sequence as set forth in SEQ ID NO: 60. The sequence of IL-16 can be, but is not limted to, a sequence as set forth in SEQ ID NO: 87. The sequence of IL-17 can be, but is not limited to, a sequence as set forth in SEQ ID NO: 88. The sequence of IL-23 can be, but is not limited to, a sequence as set forth in SEQ ID NO: 89. The sequence of IL-32 can be, but is not limited to, a sequence as set forth in SEQ ID NO: 90.

In some embodiments, the target antigen is fused or linked to an immunological fusion partner, also referred to herein as an “immunogenic component,” comprising a cytokine selected from the group of IFN-γ, TNFα IL-2, IL-8, IL-12, IL-18, IL-7, IL-3, IL-4, IL-5, IL-6, IL-9, IL-10, IL-13, IL-15, IL-16, IL-17, IL-23, IL-32, M-CSF (CSF-1), IFN-α, IFN-β, IL-1α, IL-1β, IL-1RA, IL-11, IL-17A, IL-17F, IL-19, IL-20, IL-21, IL-22, IL-24, IL-25, IL-26, IL-27, IL-28A, B, IL-29, IL-30, IL-31, IL-33, IL-34, IL-35, IL-36α,β,λ, IL-36Ra, IL-37, TSLP, LIF, OSM, LT-α, LT-β, CD40 ligand, Fas ligand, CD27 ligand, CD30 ligand, 4-1BBL, Trail, OPG-L, APRIL, LIGHT, TWEAK, BAFF, TGF-β1, and MIF. In some embodiments, the target antigen is co-expressed in a cell with an immunological fusion partner, also referred to herein as an “immunogenic component,” comprising a cytokine selected from the group of IFN-γ, TNFα IL-2, IL-8, IL-12, IL-18, IL-7, IL-3, IL-4, IL-5, IL-6, IL-9, IL-10, IL-13, IL-15, IL-16, IL-17, IL-23, IL-32, M-CSF (CSF-1), IFN-α, IFN-β, IL-1α, IL-1β, IL-1RA, IL-11, IL-17A, IL-17F, IL-19, IL-20, IL-21, IL-22, IL-24, IL-25, IL-26, IL-27, IL-28A, B, IL-29, IL-30, IL-31, IL-33, IL-34, IL-35, IL-36α,β,λ, IL-36Ra, IL-37, TSLP, LIF, OSM, LT-α, LT-β, CD40 ligand, Fas ligand, CD27 ligand, CD30 ligand, 4-1BBL, Trail, OPG-L, APRIL, LIGHT, TWEAK, BAFF, TGF-β1, and MIF.

In some embodiments, the target antigen is fused or linked to an immunological fusion partner, comprising CpG ODN (a non-limiting example sequence is shown in SEQ ID NO: 61), cholera toxin (a non-limiting example sequence is shown in SEQ ID NO: 62), a truncated A subunit coding region derived from a bacterial ADP-ribosylating exotoxin (a non-limiting example sequence is shown in (a non-limiting example sequence is shown in SEQ ID NO: 63), a truncated B subunit coding region derived from a bacterial ADP-ribosylating exotoxin (a non-limiting example sequence is shown in SEQ ID NO: 64), Hp91 (a non-limiting example sequence is shown in SEQ ID NO: 65), CCL20 (a non-limiting example sequence is shown in SEQ ID NO: 66), CCL3 (a non-limiting example sequence is shown in SEQ ID NO: 67), GM-CSF (a non-limiting example sequence is shown in SEQ ID NO: 68), G-CSF (a non-limiting example sequence is shown in SEQ ID NO: 69), LPS peptide mimic (non-limiting example sequences are shown in SEQ ID NO: 70-SEQ ID NO: 81), shiga toxin (a non-limiting example sequence is shown in SEQ ID NO: 82), diphtheria toxin (a non-limiting example sequence is shown in SEQ ID NO: 83), or CRM₁₉₇ (a non-limiting example sequence is shown in SEQ ID NO: 86).

In some embodiments, the target antigen is fused or linked to an immunological fusion partner, comprising an IL-15 superagonist. Interleukin 15 (IL-15) is a naturally occurring inflammatory cytokine secreted after viral infections. Secreted IL-15 can carry out its function by signaling via its cognate receptor on effector immune cells, and thus, can lead to overall enhancement of effector immune cell activity.

Based on IL-15's broad ability to stimulate and maintain cellular immune responses, it is believed to be a promising immunotherapeutic drug that could potentially cure certain cancers. However, major limitations in clinical development of IL-15 can include low production yields in standard mammalian cell expression systems and short serum half-life. Moreover, the IL-15:IL-15Ra complex, comprising proteins co-expressed by the same cell, rather than the free IL-15 cytokine, can be responsible for stimulating immune effector cells bearing IL-15 βγc receptor.

To contend with these shortcomings, a novel IL-15 superagonist mutant (IL-15N72D) was identified that has increased ability to bind IL-15Rβγc and enhanced biological activity. Addition of either mouse or human IL-15Rα and Fc fusion protein (the Fc region of immunoglobulin) to equal molar concentrations of IL-15N72D can provide a further increase in IL-15 biologic activity, such that IL-15N72D:IL-15Rα/Fc super-agonist complex exhibits a median effective concentration (EC50) for supporting IL-15-dependent cell growth that was greater than 10-fold lower than that of free IL-15 cytokine.

In some embodiments, the IL-15 superagonist can be a novel IL-15 superagonist mutant (IL-15N72D). In certain embodiments, addition of either mouse or human IL-15Rα and Fc fusion protein (the Fc region of immunoglobulin) to equal molar concentrations of IL-15N72D can provide a further increase in IL-15 biologic activity, such that IL-15N72D:IL-15Rα/Fc super-agonist complex exhibits a median effective concentration (EC₅₀) for supporting IL-15-dependent cell growth that can be greater than 10-fold lower than that of free IL-15 cytokine

Thus, in some embodiments, the present disclosure provides a IL-15N72D:IL-15Rα/Fc super-agonist complex with an EC50 for supporting IL-15-dependent cell growth that is greater than 2-fold lower, greater than 3-fold lower, greater than 4-fold lower, greater than 5-fold lower, greater than 6-fold lower, greater than 7-fold lower, greater than 8-fold lower, greater than 9-fold lower, greater than 10-fold lower, greater than 15-fold lower, greater than 20-fold lower, greater than 25-fold lower, greater than 30-fold lower, greater than 35-fold lower, greater than 40-fold lower, greater than 45-fold lower, greater than 50-fold lower, greater than 55-fold lower, greater than 60-fold lower, greater than 65-fold lower, greater than 70-fold lower, greater than 75-fold lower, greater than 80-fold lower, greater than 85-fold lower, greater than 90-fold lower, greater than 95-fold lower, or greater than 100-fold lower than that of free IL-15 cytokine.

In some embodiments, the IL-15 super agonist is a biologically active protein complex of two IL-15N72D molecules and a dimer of soluble IL-15Rα/Fc fusion protein, also known as ALT-803. The composition of ALT-803 and methods of producing and using ALT-803 are described in U.S. Patent Application Publication 2015/0374790, which is herein incorporated by reference. It is known that a soluble IL-15Rα fragment, containing the so-called “sushi” domain at the N terminus (Su), can bear most of the structural elements responsible for high affinity cytokine binding. A soluble fusion protein can be generated by linking the human IL-15RαSu domain (amino acids 1-65 of the mature human IL-15Rα protein) with the human IgG1 CH2-CH3 region containing the Fc domain (232 amino acids). This IL-15RαSu/IgG1 Fc fusion protein can have the advantages of dimer formation through disulfide bonding via IgG1 domains and ease of purification using standard Protein A affinity chromatography methods.

In some embodiments, ALT-803 can have a soluble complex consisting of 2 protein subunits of a human IL-15 variant associated with high affinity to a dimeric IL-15Rα sushi domain/human IgG1 Fc fusion protein. The IL-15 variant is a 114 amino acid polypeptide comprising the mature human IL-15 cytokine sequence with an Asn to Asp substitution at position 72 of helix C N72D). The human IL-15R sushi domain/human IgG1 Fc fusion protein comprises the sushi domain of the IL-15R subunit (amino acids 1-65 of the mature human IL-15Rα protein) linked with the human IgG1 CH2-CH3 region containing the Fc domain (232 amino acids). Aside from the N72D substitution, all of the protein sequences are human. Based on the amino acid sequence of the subunits, the calculated molecular weight of the complex comprising two IL-15N72D polypeptides (an example IL-15N72D sequence is shown in SEQ ID NO: 84) and a disulfide linked homodimeric IL-15RαSu/IgG1 Fc protein (an example IL-15RαSu/Fc domain is shown in SEQ ID NO: 85) is 92.4 kDa. In some embodiments, a recombinant vector encoding for a target antigen and for ALT-803 can have any sequence described herein to encode for the target antigen and can have SEQ ID NO: 84, SEQ ID NO: 84, SEQ ID NO: 85, and SEQ ID NO: 85 in any order, to encode for ALT-803.

Each IL-15N720 polypeptide has a calculated molecular weight of approximately 12.8 kDa and the IL-15RαSu/IgG 1 Fc fusion protein has a calculated molecular weight of approximately 33.4 kDa. Both the IL-15N72D and IL-15RαSu/IgG 1 Fc proteins can be glycosylated resulting in an apparent molecular weight of ALT-803 of approximately 114 kDa by size exclusion chromatography. The isoelectric point (pI) determined for ALT-803 can range from approximately 5.6 to 6.5. Thus, the fusion protein can be negatively charged at pH 7.

Combination therapy with Ad5[E1−, E2b−] vectors encoding for HER2/neu and ALT-803 can result in boosting the immune response, such that the combination of both therapeutic moieties acts to synergistically boost the immune response than either therapy alone. For example, combination therapy with Ad5[E1−, E2b−] vectors encoding for HER2/neu antigens and ALT-803 can result in synergistic enhancement of stimulation of antigen-specific effector CD4+ and CD8+ T cells, stimulation of NK cell response directed towards killing infected cells, stimulation of neutrophils or monocyte cell responses directed towards killing infected cells via antibody dependent cell-mediated cytotoxicity (ADCC), or antibody dependent cellular phagocytosis (ADCP) mechanisms. Combination therapy with Ad5[E1−, E2b−] vectors encoding for HER2/neu antigens and ALT-803 can synergistically boost any one of the above responses, or a combination of the above responses, to vastly improve survival outcomes after administration to a subject in need thereof.

Any of the immunogenicity enhancing agents described herein can be fused or linked to a target antigen by expressing the immunogenicity enhancing agents and the target antigen in the same recombinant vector, using any recombinant vector described herein.

Nucleic acid sequences that encode for such immunogenicity enhancing agents can be any one of SEQ ID NO: 35-SEQ ID NO: 90 and are summarized in TABLE 1.

TABLE 1
Sequences of Immunogenicity Enhancing Agents

| SEQ ID | |
|---|---|
| NO | Sequence |
| SEQ ID | TAASDNFQLSQGGQGFAIPIGQAMAIAGQIRSGGGSPTV |
| NO: 35 | HIGPTAFLGLGVVDNNGNGARVQRVVGSAPAASLGISTG |
| | DVITAVDGAPINSATAMADALNGHHPGDVISVTWQTKSG |
| | GTRTGNVTLAEGPPA |
| SEQ ID | MHHHHHHTAASDNFQLSQGGQGFAIPIGQAMAIAGQIRS |
| NO: 36 | GGGSPTVHIGPTAFLGLGVVDNNGNGARVQRVVGSAPAA |
| | SLGISTGDVITAVDGAPINSATAMADALNGHHPGDVISV |
| | TWQTKSGGTRTGNVTLAEGPPAEFDDDDKDPPDPHQPDM |
| | TKGYCPGGRWGFGDLAVCDGEKYPDGSFWHQWMQTWFTG |
| | PQFYFDCVSGGEPLPGPPPPGGCGGAIPSEQPNAP |
| SEQ ID | MHHHHHHTAASDNFQLSQGGQGFAIPIGQAMAIAGQIRS |
| NO: 37 | GGGSPTVHIGPTAFLGLGVVDNNGNGARVQRVVGSAPAA |
| | SLGISTGDVITAVDGAPINSATAMADALNGHHPGDVISV |
| | TWQTKSGGTRTGNVTLAEGPPAEFPLVPRGSPMGSDVRD |
| | LNALLPAVPSLGGGGGCALPVSGAAQWAPVLDFAPPGAS |
| | AYGSLGGPAPPPAPPPPPPPPPHSFIKQEPSWGGAEPHE |
| | EQCLSAFTVHFSGQFTGTAGACRYGPFGPPPPSQASSGQ |
| | ARMFPNAPYLPSCLESQPAIRNQGYSTVTFDGTPSYGHT |
| | PSHHAAQFPNHSFKHEDPMGQQGSLGEQQYSVPPPVYGC |
| | HTPTDSCTGSQALLLRTPYSSDNLYQMTSQLECMTWNQM |
| | NLGATLKGHSTGYESDNHTTPILCGAQYRIHTHGVFRGI |
| | QDVRRVPGVAPTLVRSASETSEKRPFMCAYSGCNKRYFK |
| | LSHLQMHSRKHTGEKPYQCDFKDCERRFFRSDQLKRHQR |
| | RHTGVKPFQCKTCQRKFSRSDHLKTHTRTHTGEKPFSCR |
| | WPSCQKKFARSDELVRHHNMHQRNMTKLQLAL |
| SEQ ID | MHHHHHHTAASDNFQLSQGGQGFAIPIGQAMAIAGQIRS |
| NO: 38 | GGGSPTVHIGPTAFLGLGVVDNNGNGARVQRVVGSAPAA |
| | SLGISTGDVITAVDGAPINSATAMADALNGHHPGDVISV |
| | TWQTKSGGTRTGNVTLAEGPPAEFIEGRGSGCPLLENVI |
| | SKTINPQVSKTEYKELLQEFIDDNATTNAIDELKECFLN |
| | QTDETLSNVEVFMQLIYDSSLCDLF |
| SEQ ID | MHHHHHHTAASDNFQLSQGGQGFAIPIGQAMAIAGQIRS |
| NO: 39 | GGGSPTVHIGPTAFLGLGVVDNNGNGARVQRVVGSAPAA |
| | SLGISTGDVITAVDGAPINSATAMADALNGHHPGDVISV |
| | TWQTKSGGTRTGNVTLAEGPPAEFMVDFGALPPEINSAR |
| | MYAGPGSASLVAAAQMWDSVASDLFSAASAFQSVVWGLT |
| | VGSWIGSSAGLMVAAASPYVAWMSVTAGQAELTAAQVRV |
| | AAAAYETAYGLTVPPPVIAENRAELMILIATNLLGQNTP |
| | AIAVNEAEYGEMWAQDAAAMFGYAAATATATATLLPFEE |
| | APEMTSAGGLLEQAAAVEEASDTAAANQLMNNVPQALQQ |
| | LAQPTQGTTPSSKLGGLWKTVSPHRSPISNMVSMANNHM |
| | SMTNSGVSMTNTLSSMLKGFAPAAAAQAVQTAAQNGVRA |
| | MSSLGSSLGSSGLGGGVAANLGRAASVGSLSVPQAWAAA |
| | NQAVTPAARALPLTSLTSAAERGPGQMLGGLPVGQMGAR |
| | AGGGLSGVLRVPPRPYVMPHSPAAGDIAPPALSQDRFAD |
| | FPALPLDPSAMVAQVGPQVVNINTKLGYNNAVGAGTGIV |
| | IDPNGVVLTNNHVIAGATDINAFSVGSGQTYGVDVVGYD |
| | RTQDVAVLQLRGAGGLPSAAIGGGVAVGEPVVAMGNSGG |
| | QGGTPRAVPGRVVALGQTVQASDSLTGAEETLNGLIQFD |
| | AAIQPGDSGGPVVNGLGQVVGMNTAAS |
| SEQ ID | TAASDNFQLSQGGQGFAIPIGQAMAIAGQI |
| NO: 40 | |
| SEQ ID | TAASDNFQLSQGGQGFAIPIGQAMAIAGQIKLPTVHIGP |
| NO: 41 | TAFLGLGVVDNNGNGARVQRVVGSAPAASLGISTGDVIT |
| | AVDGAPINSATAMADALNGHHPGDVISVTWQTKSGGTRT |
| | GNVTLAEGPPA |
| SEQ ID | TAASDNFQLSQGGQGFAIPIGQAMAIAGQIRSGGGSPTV |
| NO: 42 | HIGPTAFLGLGVVDNNGNGARVQRVVGSAPAASLGISTG |
| | DVITAVDGAPINSATAMADALNGHHPGDVISVTWQTKSG |
| | GTRTGNVTLAE |
| SEQ ID | MSNSRRRSLRWSWLLSVLAAVGLGLATAPAQAAPPALSQ |
| NO: 43 | DRFADFPALPLDPSAMVAQVGPQVVNINTKLGYNNAVGA |
| | GTGIVIDPNGVVLTNNHVIAGATDINAFSVGSGQTYGVD |
| | VVGYDRTQDVAVLQLRGAGGLPSAAIGGGVAVGEPVVAM |
| | GNSGGQGGTPRAVPGRVVALGQTVQASDSLTGAEETLNG |
| | LIQFDAAIQPGDSGGPVVNGLGQVVGMNTAASDNFQLSQ |
| | GGQGFAIPIGQAMAIAGQIRSGGGSPTVHIGPTAFLGLG |
| | VVDNNGNGARVQRVVGSAPAASLGISTGDVITAVDGAPI |
| | NSATAMADALNGHHPGDVISVTWQTKSGGTRTGNVTLAE |
| | GPPA |
| SEQ ID | MKLKTLALSLLAAGVLAGCSSHSSNMANTQMKSDKIIIA |
| NO: 44 | HRGASGYLPEHTLESKALAFAQQADYLEQDLAMTKDGRL |
| | VVIHDHFLDGLTDVAKKFPHRHRKDGRYYVIDFTLKEIQ |
| | SLEMTENFETKDGKQAQVYPNRFPLWKSHFRIHTFEDEI |
| | EFIQGLEKSTGKKVGIYPEIKAPWFHHQNGKDIAAETLK |
| | VLKKYGYDKKTDMVYLQTFDFNELKRIKTELLPQMGMDL |
| | KLVQLIAYTDWKETQEKDPKGYWVNYNYDWMFKPGAMAE |
| | VVKYADGVGPGWYMLVNKEESKPDNIVYTPLVKELAQYN |
| | VEVHPYTVRKDALPAFFTDVNQMYDVLLNKSGATGVFTD |
| | FPDTGVEFLKGIK |
| SEQ ID | MEINVSKLRTDLPQVGVQPYRQVHAHSTGNPHSTVQNEA |
| NO: 45 | DYHWRKDPELGFFSHIVGNGCIMQVGPVDNGAWDVGGGW |
| | NAETYAAVELIESHSTKEEFMTDYRLYIELLRNLADEAG |
| | LPKTLDTGSLAGIKTHEYCTNNQPNNHSDHVDPYPYLAK |
| | WGISREQFKHDIENGLTIETGWQKNDTGYWYVHSDGSYP |
| | KDKFEKINGTWYYFDSSGYMLADRWRKHTDGNWYWFDNS |
| | GEMATGWKKIADKWYYFNEEGAMKTGWVKYKDTWYYLDA |
| | KEGAMVSNAFIQSADGTGWYYLKPDGTLADRPEFRMSQM |
| | A |
| SEQ ID | MKYTSYILAFQLCIVLGSLGCYCQDPYVKEAENLKKYFN |
| NO: 46 | AGHSDVADNGTLFLGILKNWKEESDRKIMQSQIVSFYFK |
| | LFKNFKDDQSIQKSVETIKEDMNVKFFNSNKKKRDDFEK |
| | LTNYSVTDLNVQRKAIHELIQVMAELSPAAKTGKRKRSQ |
| | MLFRGRRASQ |
| SEQ ID | MSTESMIRDVELAEEALPKKTGGPQGSRRCLFLSLFSFL |
| NO: 47 | IVAGATTLFCLLHFGVIGPQREEFPRDLSLISPLAQAVR |
| | SSSRTPSDKPVAHVVANPQAEGQLQWLNRRANALLANGV |
| | ELRDNQLVVPSEGLYLIYSQVLFKGQGCPSTHVLLTHTI |
| | SRIAVSYQTKVNLLSAIKSPCQRETPEGAEAKPWYEPIY |
| | LGGVFQLEKGDRLSAEINRPDYLDFAESGQVYFGIIAL |
| SEQ ID | MYRMQLLSCIALSLALVTNSAPTSSSTKKTQLQLEHLLL |
| NO: 48 | DLQMILNGINNYKNPKLTRMLTFKFYMPKKATELKHLQC |
| | LEEELKPLEEVLNLAQSKNFHLRPRDLISNINVIVLELK |
| | GSETTFMCEYADETATIVEFLNRWITFCQSIISTLT |
| SEQ ID | MTSKLAVALLAAFLISAALCEGAVLPRSAKELRCQCIKT |
| NO: 49 | YSKPFHPKFIKELRVIESGPHCANTEIIVKLSDGRELCL |
| | DPKENWVQRVVEKFLKRAENS |
| SEQ ID | MEPLVTWVVPLLFLFLLSRQGAACRTSECCFQDPPYPDA |
| NO: 50 | DSGSASGPRDLRCYRISSDRYECSWQYEGPTAGVSHFLR |
| | CCLSSGRCCYFAAGSATRLQFSDQAGVSVLYTVTLWVES |
| | WARNQTEKSPEVTLQLYNSVKYEPPLGDIKVSKLAGQLR |
| | MEWETPDNQVGAEVQFRHRTPSSPWKLGDCGPQDDDTES |
| | CLCPLEMNVAQEFQLRRRQLGSQGSSWSKWSSPVCVPPE |
| | NPPQPQVRFSVEQLGQDGRRRLTLKEQPTQLELPEGCQG |
| | LAPGTEVTYRLQLHMLSCPCKAKATRTLHLGKMPYLSGA |
| | AYNVAVISSNQFGPGLNQTWHIPADTHTEPVALNISVGT |
| | NGTTMYWPARAQSMTYCIEWQPVGQDGGLATCSLTAPQD |
| | PDPAGMATYSWSRESGAMGQEKCYYITIFASAHPEKLTL |
| | WSTVLSTYHFGGNASAAGTPHHVSVKNHSLDSVSVDWAP |
| | SLLSTCPGVLKEYVVRCRDEDSKQVSEHPVQPTETQVTL |
| | SGLRAGVAYTVQVRADTAWLRGVWSQPQRFSIEVQVSDW |
| | LIFFASLGSFLSILLVGVLGYLGLNRAARHLCPPLPTPC |
| | ASSAIEFPGGKETWQWINPVDFQEEASLQEALVVEMSWD |
| | KGERTEPLEKTELPEGAPELALDTELSLEDGDRCKAKM |
| SEQ ID | MAAEPVEDNCINFVAMKFIDNTLYFIAEDDENLESDYFG |
| NO: 51 | KLESKLSVIRNLNDQVLFIDQGNRPLFEDMTDSDCRDNA |
| | PRTIFIISMYKDSQPRGMAVTISVKCEKISTLSCENKII |
| | SFKEMNPPDNIKDTKSDIIFFQRSVPGHDNKMQFESSSY |
| | EGYFLACEKERDLFKLILKKEDELGDRSIMFTVQNED |
| SEQ ID | MFHVSFRYIFGLPPLILVLLPVASSDCDIEGKDGKQYES |
| NO: 52 | VLMVSIDQLLDSMKEIGSNCLNNEFNFFKRHICDANKEG |
| | MFLFRAARKLRQFLKMNSTGDFDLHLLKVSEGTTILLNC |
| | TGQVKGRKPAALGEAQPTKSLEENKSLKEQKKLNDLCFL |
| | KRLLQEIKTCWNKILMGTKEH |
| SEQ ID | MSRLPVLLLLQLLVRPGLQAPMTQTTSLKTSWVNCSNMI |
| NO: 53 | DEIITHLKQPPLPLLDFNNLNGEDQDILMENNLRRPNLE |
| | AFNRAVKSLQNASAIESILKNLLPCLPLATAAPTRHPIH |
| | IKDGDWNEFRRKLTFYLKTLENAQAQQTTLSLAIF |
| SEQ ID | MGLTSQLLPPLFFLLACAGNFVHGHKCDITLQEIIKTLN |
| NO: 54 | SLTEQKTLCTELTVTDIFAASKNTTEKETFCRAATVLRQ |
| | FYSHHEKDTRCLGATAQQFHRHKQLIRFLKRLDRNLWGL |
| | AGLNSCPVKEANQSTLENFLERLKTIMREKYSKCSS |
| SEQ ID | MRMLLHLSLLALGAAYVYAIPTEIPTSALVKETLALLST |
| NO: 55 | HRTLLIANETLRIPVPVHKNHQLCTEEIFQGIGTLESQT |
| | VQGGTVERLFKNLSLIKKYIDGQKKKCGEERRRVNQFLD |
| | YLQEFLGVMNTEWIIES |
| SEQ ID | MNSFSTSAFGPVAFSLGLLLVLPAAFPAPVPPGEDSKDV |
| NO: 56 | AAPHRQPLTSSERIDKQIRYILDGISALRKETCNKSNMC |
| | ESSKEALAENNLNLPKMAEKDGCFQSGFNEETCLVKIIT |
| | GLLEFEVYLEYLQNRFESSEEQARAVQMSTKVLIQFLQK |
| | KAKNLDAITTPDPTTNASLLTKLQAQNQWLQDMTTHLIL |
| | RSFKEFLQSSLRALRQM |
| SEQ ID | MVLTSALLLCSVAGQGCPTLAGILDINFLINKMQEDPAS |
| NO: 57 | KCHCSANVTSCLCLGIPSDNCTRPCFSERLSQMTNTTMQ |
| | TRYPLIFSRVKKSVEVLKNNKCPYFSCEQPCNQTTAGNA |
| | LTFLKSLLEIFQKEKMRGMRGKI |
| SEQ ID | MHSSALLCCLVLLTGVRASPGQGTQSENSCTHFPGNLPN |
| NO: 58 | MLRDLRDAFSRVKTFFQMKDQLDNLLLKESLLEDFKGYL |
| | GCQALSEMIQFYLEEVMPQAENQDPDIKAHVNSLGENLK |
| | TLRLRLRRCHRFLPCENKSKAVEQVKNAFNKLQEKGIYK |
| | AMSEFDIFINYIEAYMTMKIRN |
| SEQ ID | MALLLTTVIALTCLGGFASPGPVPPSTALRELIEELVNI |
| NO: 59 | TQNQKAPLCNGSMVWSINLTAGMYCAALESLINVSGCSA |
| | IEKTQRMLSGFCPHKVSAGQFSSLHVRDTKIEVAQFVKD |
| | LLLHLKKLFREGQFNRNFESIIICRDRT |
| SEQ ID | MDFQVQIFSFLLISASVIMSRANWVNVISDLKKIEDLIQ |
| NO: 60 | SMHIDATLYTESDVHPSCKVTAMKCFLLELQVISLESGD |
| | ASIHDTVENLIILANNSLSSNGNVTESGCKECEELEEKN |
| | IKEFLQSFVHIVQMFINTS |
| SEQ ID | MEGDGSDPEPPDAGEDSKSENGENAPIYCICRKPDINCF |
| NO: 61 | MIGCDNCNEWFHGDCIRITEKMAKAIREWYCRECREKDP |
| | KLEIRYRHKKSRERDGNERDSSEPRDEGGGRKRPVPDPN |
| | LQRRAGSGTGVGAMLARGSASPHKSSPQPLVATPSQHHQ |
| | QQQQQIKRSARMCGECEACRRTEDCGHCDFCRDMKKFGG |
| | PNKIRQKCRLRQCQLRARESYKYFPSSLSPVTPSESLPR |
| | PRRPLPTQQQPQPSQKLGRIREDEGAVASSTVKEPPEAT |
| | ATPEPLSDEDLPLDPDLYQDFCAGAFDDNGLPWMSDTEE |
| | SPFLDPALRKRAVKVKHVKRREKKSEKKKEERYKRHRQK |
| | QKHKDKWKHPERADAKDPASLPQCLGPGCVRPAQPSSKY |
| | CSDDCGMKLAANRIYEILPQRIQQWQQSPCIAEEHGKKL |
| | LERIRREQQSARTRLQEMERRFHELEAIILRAKQQAVRE |
| | DEESNEGDSDDTDLQIFCVSCGHPINPRVALRHMERCYA |
| | KYESQTSFGSMYPTRIEGATRLFCDVYNPQSKTYCKRLQ |
| | VLCPEHSRDPKVPADEVCGCPLVRDVFELTGDFCRLPKR |
| | QCNRHYCWEKLRRAEVDLERVRVWYKLDELFEQERNVRT |
| | AMTNRAGLLALMLHQTIQHDPLTTDLRSSADR |
| SEQ ID | MIKLKFGVFFTVLLSSAYAHGTPQNITDLCAEYHNTQIY |
| NO: 62 | TLNDKIFSYTESLAGKREMAIITFKNGAIFQVEVPGSQH |
| | IDSQKKAIERMKDTLRIAYLTEAKVEKLCVWNNKTPHAI |
| | AAISMAN |
| SEQ ID | MVKIIFVFFIFLSSFSYANDDKLYRADSRPPDEIKQSGG |
| NO: 63 | LMPRGQNEYFDRGTQMNINLYDHARGTQTGFVRHDDGYV |
| | STSISLRSAHLVGQTILSGHSTYYIYVIATAPNMFNVND |
| | VLGAYSPHPDEQEVSALGGIPYSQIYGWYRVHFGVLDEQ |
| | LHRNRGYRDRYYSNLDIAPAADGYGLAGFPPEHRAWREE |
| | PWIHHAPPGCGNAPRSSMSNTCDEKTQSLGVKFLDEYQS |
| | KVKRQIFSGYQSDIDTHNRIKDEL |
| SEQ ID | MIKLKFGVFFTVLLSSAYAHGTPQNITDLCAEYHNTQIH |
| NO: 64 | TLNDKILSYTESLAGNREMAIITFKNGATFQVEVPGSQH |
| | IDSQKKAIERMKDTLRIAYLTEAKVEKLCVWNNKTPHAI |
| | AAISMAN |
| SEQ ID | DPNAPKRPPSAFFLFCSE |
| NO: 65 | |
| SEQ ID | MCCTKSLLLAALMSVLLLHLCGESEAASNFDCCLGYTDR |
| NO: 66 | ILHPKFIVGFTRQLANEGCDINAIIFHTKKKLSVCANPK |
| | QTWVKYIVRLLSKKVKNM |
| SEQ ID | MQVSTAALAVLLCTMALCNQFSASLAADTPTACCFSYTS |
| NO: 67 | RQIPQNFIADYFETSSQCSKPGVIFLTKRSRQVCADPSE |
| | EWVQKYVSDLELSA |
| SEQ ID | MWLQSLLLLGTVACSISAPARSPSPSTQPWEHVNAIQEA |
| NO: 68 | RRLLNLSRDTAAEMNETVEVISEMFDLQEPTCLQTRLEL |
| | YKQGLRGSLTKLKGPLTMMASHYKQHCPPTPETSCATQI |
| | ITFESFKENLKDFLLVIPFDCWEPVQE |
| SEQ ID | MAGPATQSPMKLMALQLLLWHSALWTVQEATPLGPASSL |
| NO: 69 | PQSFLLKCLEQVRKIQGDGAALQEKLCATYKLCHPEELV |
| | LLGHSLGIPWAPLSSCPSQALQLAGCLSQLHSGLFLYQG |
| | LLQALEGISPELGPTLDTLQLDVADFATTIWQQMEELGM |
| | APALQPTQGAMPAFASAFQRRAGGVLVASHLQSFLEVSY |
| | RVLRHLAQP |
| SEQ ID | QEINSSY |
| NO: 70 | |
| SEQ ID | SHPRLSA |
| NO: 71 | |
| SEQ ID | SMPNPMV |
| NO: 72 | |
| SEQ ID | GLQQVLL |
| NO: 73 | |
| SEQ ID | HELSVLL |
| NO: 74 | |
| SEQ ID | YAPQRLP |
| NO: 75 | |
| SEQ ID | TPRTLPT |
| NO: 76 | |
| SEQ ID | APVHSSI |
| NO: 77 | |
| SEQ ID | APPHALS |
| NO: 78 | |
| SEQ ID | TFSNRFI |
| NO: 79 | |
| SEQ ID | VVPTPPY |
| NO: 80 | |
| SEQ ID | ELAPDSP |
| NO: 81 | |
| SEQ ID | TPDCVTGKVEYTKYNDDDTFTVKVGDKELFTNRWNLQSL |
| NO: 82 | LLSAQITGMTVTIKQNACHNGGGFSEVIFR |
| SEQ ID | MSRKLFASILIGALLGIGAPPSAHAGADDVVDSSKSFVM |
| NO: 83 | ENFSSYHGTKPGYVDSIQKGIQKPKSGTQGNYDDDWKGF |
| | YSTDNKYDAAGYSVDNENPLSGKAGGVVKVTYPGLTKVL |
| | ALKVDNAETIKKELGLSLTEPLMEQVGTEEFIKRFGDGA |
| | SRVVLSLPFAEGSSSVEYINNWEQAKALSVELEINFETR |
| | GKRGQDAMYEYMAQACAGNRVRRSVGSSLSCINLDWDVI |
| | RDKTKTKIESLKEHGPIKNKMSESPNKTVSEEKAKQYLE |
| | EFHQTALEHPELSELKTVTGTNPVFAGANYAAWAVNVAQ |
| | VIDSETADNLEKTTAALSILPGIGSVMGIADGAVHHNTE |
| | EIVAQSIALSSLMVAQAIPLVGELVDIGFAAYNFVESII |
| | NLFQVVHNSYNRPAYSPGHKTQPFLHDGYAVSWNTVEDS |
| | IIRTGFQGESGHDIKITAENTPLPIAGVLLPTIPGKLDV |
| | NKSKTHISVNGRKIRMRCRAIDGDVTFCRPKSPVYVGNG |
| | VHANLHVAFHRSSSEKIHSNEISSDSIGVLGYQKTVDHT |
| | KVNSKLSLFFEIKS |
| SEQ ID | NWVNVISDLKKIEDLIQSMHIDATLYTESDVHPSCKVTA |
| NO: 84 | MKCFLLELQVISLESGDASIHDTVENLIILANDSLSSNG |
| | NVTESGCKECEELEEKNIKEFLQSFVHIVQMFINTS |
| SEQ ID | ITCPPPMSVEHADIWVKSYSLYSRERYICNSGFKRKAGT |
| NO: 85 | SSLTECVLNKATNVAHWTTPSLKCIREPKSCDKTHTCPP |
| | CPAPELLGGPSVFLFPPKPKDTLMISRTPEVTCVVVDVS |
| | HEDPEVKFNWYVDGVEVHNAKTKPREEQYNSTYRVVSVL |
| | TVLHQDWLNGKEYKCKVSNKALPAPIEKTISKAKGQPRE |
| | PQVYTLPPSRDELTKNQVSLTCLVKGFYPSDIAVEWESN |
| | GQPENNYKTTPPVLDSDGSFFLYSKLTVDKSRWQQGNVF |
| | SCSVMHEALHNHYTQKSLSLSPGK |
| SEQ ID | GADDVVDSSKSFVMENFSSYHGTKPGYVDSIQKGIQKPK |
| NO: 86 | SGTQGNYDDDWKEFYSTDNKYDAAGYSVDNENPLSGKAG |
| | GVVKVTYPGLTKVLALKVDNAETIKKELGLSLTEPLMEQ |
| | VGTEEFIKRFGDGASRVVLSLPFAEGSSSVEYINNWEQA |
| | KALSVELEINFETRGKRGQDAMYEYMAQACAGNRVRRSV |
| | GSSLSCINLDWDVIRDKTKTKIESLKEHGPIKNKMSESP |
| | NKTVSEEKAKQYLEEFHQTALEHPELSELKTVTGTNPVF |
| | AGANYAAWAVNVAQVIDSETADNLEKTTAALSILPGIGS |
| | VMGIADGAVHHNTEEIVAQSIALSSLMVAQAIPLVGELV |
| | DIGFAAYNFVESIINLFQVVHNSYNRPAYSPGHKTQPFL |
| | HDGYAVSWNTVEDSIIRTGFQGESGHDIKITAENTPLPI |
| | AGVLLPTIPGKLDVNKSKTHISVNGRKIRMRCRAIDGDV |
| | TFCRPKSPVYVGNGVHANLHVAFHRSSSEKIHSNEISSD |
| | SIGVLGYQKTVDHTKVNSKLSLFFEIKS |
| SEQ ID | MESHSRAGKSRKSAKFRSISRSLMLCNAKTSDDGSSPDE |
| NO: 87 | KYPDPFEISLAQGKEGIFHSSVQLADTSEAGPSSVPDLA |
| | LASEAAQLQAAGNDRGKTCRRIFFMKESSTASSREKPGK |
| | LEAQSSNFLFPKACHQRARSNSTSVNPYCTREIDFPMTK |
| | KSAAPTDRQPYSLCSNRKSLSQQLDCPAGKAAGTSRPTR |
| | SLSTAQLVQPSGGLQASVISNIVLMKGQAKGLGFSIVGG |
| | KDSIYGPIGIYVKTIFAGGAAAADGRLQEGDEILELNGE |
| | SMAGLTHQDALQKFKQAKKGLLTLTVRTRLTAPPSLCSH |
| | LSPPLCRSLSSSTCITKDSSSFALESPSAPISTAKPNYR |
| | IMVEVSLQKEAGVGLGIGLCSVPYFQCISGIFVHTLSPG |
| | SVAHLDGRLRCGDEIVEISDSPVHCLTLNEVYTILSRCD |
| | PGPVPIIVSRHPDPQVSEQQLKEAVAQAVENTKFGKERH |
| | QWSLEGVKRLESSWHGRPTLEKEREKNSAPPHRRAQKVM |
| | IRSSSDSSYMSGSPGGSPGSGSAEKPSSDVDISTHSPSL |
| | PLAREPVVLSIASSRLPQESPPLPESRDSHPPLRLKKSF |
| | EILVRKPMSSKPKPPPRKYFKSDSDPQKSLEERENSSCS |
| | SGHTPPTCGQEARELLPLLLPQEDTAGRSPSASAGCPGP |
| | GIGPQTKSSTEGEPGWRRASPVTQTSPIKHPLLKRQARM |
| | DYSFDTTAEDPWVRISDCIKNLFSPIMSENHGHMPLQPN |
| | ASLNEEEGTQGHPDGTPPKLDTANGTPKVYKSADSSTVK |
| | KGPPVAPKPAWFRQSLKGLRNRASDPRGLPDPALSTQPA |
| | PASREHLGSHIRASSSSSSIRQRISSFETFGSSQLPDKG |
| | AQRLSLQPSSGEAAKPLGKHEEGRFSGLLGRGAAPTLVP |
| | QQPEQVLSSGSPAASEARDPGVSESPPPGRQPNQKTLPP |
| | GPDPLLRLLSTQAEESQGPVLKMPSQRARSFPLTRSQSC |
| | ETKLLDEKTSKLYSISSQVSSAVMKSLLCLPSSISCAQT |
| | PCIPKEGASPTSSSNEDSAANGSAETSALDTGFSLNLSE |
| | LREYTEGLTEAKEDDDGDHSSLQSGQSVISLLSSEELKK |
| | LIEEVKVLDEATLKQLDGIHVTILHKEEGAGLGFSLAGG |
| | ADLENKVITVHRVFPNGLASQEGTIQKGNEVLSINGKSL |
| | KGTTHHDALAILRQAREPRQAVIVTRKLTPEAMPDLNSS |
| | TDSAASASAASDVSVESTEATVCTVTLEKMSAGLGFSLE |
| | GGKGSLHGDKPLTINRIFKGAASEQSETVQPGDEILQLG |
| | GTAMQGLTRFEAWNIIKALPDGPVTIVIRRKSLQSKETT |
| | AAGDS |
| SEQ ID | MTPGKTSLVSLLLLLSLEAIVKAGITIPRNPGCPNSEDK |
| NO: 88 | NFPRTVMVNLNIHNRNTNTNPKRSSDYYNRSTSPWNLHR |
| | NEDPERYPSVIWEAKCRHLGCINADGNVDYHMNSVPIQQ |
| | EILVLRREPPHCPNSFRLEKILVSVGCTCVTPIVHHVA |
| SEQ ID | RAVPGGSSPAWTQCQQLSQKLCTLAWSAHPLVGHMDLRE |
| NO: 89 | EGDEETTNDVPHIQCGDGCDPQGLRDNSQFCLQRIHQGL |
| | IFYEKLLGSDIFTGEPSLLPDSPVGQLHASLLGLSQLLQ |
| | PEGHHWETQQIPSLSPSQPWQRLLLRFKILRSLQAFVAV |
| | AARVFAHGAATLSPIWELKKDVYVVELDWYPDAPGEMVV |
| | LTCDTPEEDGITWTLDQSSEVLGSGKTLTIQVKEFGDAG |
| | QYTCHKGGEVLSHSLLLLHKKEDGIWSTDILKDQKEPKN |
| | KTFLRCEAKNYSGRFTCWWLTTISTDLTFSVKSSRGSSD |
| | PQGVTCGAATLSAERVRGDNKEYEYSVECQEDSACPAAE |
| | ESLPIEVMVDAVHKLKYENYTSSFFIRDIIKPDPPKNLQ |
| | LKPLKNSRQVEVSWEYPDTWSTPHSYFSLTFCVQVQGKS |
| | KREKKDRVFTDKTSATVICRKNASISVRAQDRYYSSSWS |
| | EWASVPCS |
| SEQ ID | MCFPKVLSDDMKKLKARMVMLLPTSAQGLGAWVSACDTE |
| NO: 90 | DTVGHLGPWRDKDPALWCQLCLSSQHQAIERFYDKMQNA |
| | ESGRGQVMSSLAELEDDFKEGYLETVAAYYEEQHPELTP |
| | LLEKERDGLRCRGNRSPVPDVEDPATEEPGESFCDKVMR |
| | WFQAMLQRLQTWWHGVLAWVKEKVVALVHAVQALWKQFQ |
| | SFCCSLSELFMSSFQSYGAPRGDKEELTPQKCSEPQSSK |

In some embodiments, the nucleic acid sequences for the target antigen and the immunological fusion partner are not separated by any nucleic acids. In other embodiments, a nucleic acid sequence that encodes for a linker can be inserted between the nucleic acid sequence encoding for any target antigen described herein and the nucleic acid sequence encoding for any immunological fusion partner described herein. Thus, in certain embodiments, the protein produced following immunization with the viral vector containing a target antigen, a linker, and an immunological fusion partner can be a fusion protein comprising the target antigen of interest followed by the linker and ending with the immunological fusion partner, thus linking the target antigen to an immunological fusion partner that increases the immunogenicity of the target antigen of interest via a linker. In some embodiments, the sequence of linker nucleic acids can be from about 1 to about 150 nucleic acids long, from about 5 to about 100 nucleic acids along, or from about 10 to about 50 nucleic acids in length. In some embodiments, the nucleic acid sequences may encode one or more amino acid residues. In some embodiments, the amino acid sequence of the linker can be from about 1 to about 50, or about 5 to about 25 amino acid residues in length. In some embodiments, the sequence of the linker comprises less than 10 amino acids. In some embodiments, the linker can be a polyalanine linker, a polyglycine linker, or a linker with both alanines and glycines.

Nucleic acid sequences that encode for such linkers can be any one of SEQ ID NO: 91-SEQ ID NO: 105 and are summarized in TABLE 2.

TABLE 2
Sequences of Linkers

| SEQ ID NO | Sequence |
|---|---|
| SEQ ID NO: 91 | MAVPMQLSCSR |
| SEQ ID NO: 92 | RSTG |
| SEQ ID NO: 93 | TR |
| SEQ ID NO: 94 | RSQ |
| SEQ ID NO: 95 | RSAGE |
| SEQ ID NO: 96 | RS |
| SEQ ID NO: 97 | GG |
| SEQ ID NO: 98 | GSGGSGGSG |
| SEQ ID NO: 99 | GGSGGSGGSGG |
| SEQ ID NO: 100 | GGSGGSGGSGGSGG |
| SEQ ID NO: 101 | GGSGGSGGSGGSGGSGG |
| SEQ ID NO: 102 | GGSGGSGGSGGSGGSGGSGG |
| SEQ ID NO: 103 | GGSGGSGGSGGSGGSGGSGGSGG |
| SEQ ID NO: 104 | GGSGGSGGSGGSGGSG |
| SEQ ID NO: 105 | GSGGSGGSGGSGGSGG |

### XI. Costimulatory Molecules

In addition to the use of a recombinant adenovirus-based vector vaccine containing target antigens such as a HER2/neu antigen or epitope, co-stimulatory molecules can be incorporated into said vaccine to increase immunogenicity. Initiation of an immune response requires at least two signals for the activation of naive T cells by APCs (Damle, et al. J Immunol 148:1985-92 (1992); Guinan, et al. Blood 84:3261-82 (1994); Hellstrom, et al. Cancer Chemother Pharmacol 38:S40-44 (1996); Hodge, et al. Cancer Res 39:5800-07 (1999)). An antigen specific first signal is delivered through the T cell receptor (TCR) via the peptide/major histocompatability complex (MHC) and causes the T cell to enter the cell cycle. A second, or costimulatory, signal may be delivered for cytokine production and proliferation.

At least three distinct molecules normally found on the surface of professional antigen presenting cells (APCs) have been reported as capable of providing the second signal critical for T cell activation: B7-1 (CD80), ICAM-1 (CD54), and LFA-3 (human CD58) (Damle, et al. J Immunol 148:1985-92 (1992); Guinan, et al. Blood 84: 3261-82 (1994); Wingren, et al. Crit Rev Immunol 15: 235-53 (1995); Parra, et al. Scand. J Immunol 38: 508-14 (1993); Hellstrom, et al. Ann NY Acad Sci 690: 225-30 (1993); Parra, et al. J Immunol 158: 637-42 (1997); Sperling, et al. J Immunol 157: 3909-17 (1996); Dubey, et al. J Immunol 155: 45-57 (1995); Cavallo, et al. Eur J Immunol 25: 1154-62 (1995)).

These costimulatory molecules have distinct T cell ligands. B7-1 interacts with the CD28 and CTLA-4 molecules, ICAM-1 interacts with the CD11a/CD18 (LFA-1β2 integrin) complex, and LFA-3 interacts with the CD2 (LFA-2) molecules. Therefore, in a preferred embodiment, it would be desirable to have a recombinant adenovirus vector that contains B7-1, ICAM-1, and LFA-3, respectively, that, when combined with a recombinant adenovirus-based vector vaccine containing one or more nucleic acids encoding target antigens such as a HER2/neu antigen or epitope, will further increase/enhance anti-tumor immune responses directed to specific target antigens.

### XII. Immune Pathway Checkpoint Modulators

In certain embodiments, immune pathway checkpoint inhibitors are combined with compositions comprising adenoviral vectors disclosed herein. In certain embodiments, a patient received an immune pathway checkpoint inhibitor in conjunction with a vaccine or pharmaceutical compositions described herein. In further embodiments, compositions are administered with one or more immune pathway checkpoint modulators. A balance between activation and inhibitory signals regulates the interaction between T lymphocytes and disease cells, wherein T-cell responses are initiated through antigen recognition by the T-cell receptor (TCR). The inhibitory pathways and signals are referred to as immune pathway checkpoints. In normal circumstances, immune pathway checkpoints play a critical role in control and prevention of autoimmunity and also protect from tissue damage in response to pathogenic infection.

Certain embodiments provide combination immunotherapies comprising viral vector-based vaccines and compositions for modulating immune pathway checkpoint inhibitory pathways for the prevention and/or treatment of cancer and infectious diseases. In some embodiments, modulating is increasing expression or activity of a gene or protein. In some embodiments, modulating is decreasing expression or activity of a gene or protein. In some embodiments, modulating affects a family of genes or proteins.

In general, the immune inhibitory pathways are initiated by ligand-receptor interactions. It is now clear that in diseases, the disease can co-opt immune-checkpoint pathways as mechanism for inducing immune resistance in a subject.

The induction of immune resistance or immune inhibitory pathways in a subject by a given disease can be blocked by molecular compositions such as siRNAs, antisense, small molecules, mimic, a recombinant form of ligand, receptor or protein, or antibodies (which can be an Ig fusion protein) that are known to modulate one or more of the Immune Inhibitory Pathways. For example, preliminary clinical findings with blockers of immune-checkpoint proteins, such as Cytotoxic T-lymphocyte-associated antigen 4 (CTLA4) and programmed cell death protein 1 (PD1) have shown promise for enhancing anti-tumor immunity.

Because diseased cells can express multiple inhibitory ligands, and disease-infiltrating lymphocytes express multiple inhibitory receptors, dual or triple blockade of immune pathway checkpoints proteins may enhance anti-disease immunity. Combination immunotherapies as provide herein can comprise one or more compositions comprising an immune pathway checkpoint modulator that targets one or more of the following immune-checkpoint proteins: PD1, PDL1, PDL2, CD28, CD80, CD86, CTLA4, B7RP1, ICOS, B7RPI, B7-H3 (also known as CD276), B7-H4 (also known as B7-S1, B7x and VCTN1), BTLA (also known as CD272), HVEM, KIR, TCR, LAG3 (also known as CD223), CD137, CD137L, OX40, OX40L, CD27, CD70, CD40, CD40L, TIM3 (also known as HAVcr2), GAL9, A2aR, and Adenosine.

In some embodiments, the molecular composition comprises siRNAs. In some embodiments, the molecular composition comprises a small molecule. In some embodiments, the molecular composition comprises a recombinant form of a ligand. In some embodiments, the molecular composition comprises a recombinant form of a receptor. In some embodiments, the molecular composition comprises an antibody. In some embodiments, the combination therapy comprises more than one molecular composition and/or more than one type of molecular composition. As it will be appreciated by those in the art, future discovered proteins of the immune checkpoint inhibitory pathways are also envisioned to be encompassed by the present disclosure.

In some embodiments, combination immunotherapies comprise molecular compositions for the modulation of CTLA4. In some embodiments, combination immunotherapies comprise molecular compositions for the modulation of PD1. In some embodiments, combination immunotherapies comprise molecular compositions for the modulation of PDL1. In some embodiments, combination immunotherapies comprise molecular compositions for the modulation of LAG3. In some embodiments, combination immunotherapies comprise molecular compositions for the modulation of B7-H3. In some embodiments, combination immunotherapies comprise molecular compositions for the modulation of B7-H4. In some embodiments, combination immunotherapies comprise molecular compositions for the modulation of TIM3. In some embodiments, modulation is an increase or enhancement of expression. In other embodiments, modulation is the decrease of absence of expression.

Two non-limiting exemplary immune pathway checkpoint inhibitors include the cytotoxic T lymphocyte associated antigen-4 (CTLA-4) and the programmed cell death protein-1 (PD1). CTLA-4 can be expressed exclusively on T-cells where it regulates early stages of T-cell activation. CTLA-4 interacts with the co-stimulatory T-cell receptor CD28 which can result in signaling that inhibits T-cell activity. Once TCR antigen recognition occurs, CD28 signaling may enhances TCR signaling, in some cases leading to activated T-cells and CTLA-4 inhibits the signaling activity of CD28. The present disclosure provides immunotherapies as provided herein in combination with anti-CTLA-4 monoclonal antibody for the prevention and/or treatment of cancer and infectious diseases. The present disclosure provides vaccine or immunotherapies as provided herein in combination with CTLA-4 molecular compositions for the prevention and/or treatment of cancer and infectious diseases.

Programmed death cell protein ligand-1 (PDL1) is a member of the B7 family and is distributed in various tissues and cell types. PDL1 can interact with PD1 inhibiting T-cell activation and CTL mediated lysis. Significant expression of PDL1 has been demonstrated on various human tumors and PDL1 expression is one of the key mechanisms in which tumors evade host anti-tumor immune responses. Programmed death-ligand 1 (PDL1) and programmed cell death protein-1 (PD1) interact as immune pathway checkpoints. This interaction can be a major tolerance mechanism which results in the blunting of anti-tumor immune responses and subsequent tumor progression. PD1 is present on activated T cells and PDL1, the primary ligand of PD1, is often expressed on tumor cells and antigen-presenting cells (APC) as well as other cells, including B cells. PDL1 interacts with PD1 on T cells inhibiting T cell activation and cytotoxic T lymphocyte (CTL) mediated lysis. The present disclosure provides immunotherapies as provided herein in combination with anti-PD1 or anti-PDL1 monoclonal antibody for the prevention and/or treatment of cancer and infectious diseases.

Certain embodiments may provide immunotherapies as provided herein in combination with PD1 or anti-PDL1 molecular compositions for the prevention and/or treatment of cancer and infectious diseases. Certain embodiments may provide immunotherapies as provided herein in combination with anti-CTLA-4 and anti-PD1 monoclonal antibodies for the prevention and/or treatment of cancer and infectious diseases. Certain embodiments may provide immunotherapies as provided herein in combination with anti-CTLA-4 and PDL1 monoclonal antibodies. Certain embodiments may provide vaccine or immunotherapies as provided herein in combination with anti-CTLA-4, anti-PD1, anti-PDL1 monoclonal antibodies, or a combination thereof, for the treatment of cancer and infectious diseases.

Immune pathway checkpoint molecules can be expressed by T cells. Immune pathway checkpoint molecules can effectively serve as “brakes” to down-modulate or inhibit an immune response. Immune pathway checkpoint molecules include, but are not limited to Programmed Death 1 (PD1 or PD-1, also known as PDCD1 or CD279, accession number: NM_005018), Cytotoxic T-Lymphocyte Antigen 4 (CTLA-4, also known as CD152, GenBank accession number AF414120.1), LAG3 (also known as CD223, accession number: NM_002286.5), Tim3 (also known as hepatitis A virus cellular receptor 2 (HAVCR2), GenBank accession number: JX049979.1), B and T lymphocyte associated (BTLA) (also known as CD272, accession number: NM_181780.3), BY55 (also known as CD160, GenBank accession number: CR541888.1), TIGIT (also known as IVSTM3, accession number: NM_173799), LAIR1 (also known as CD305, GenBank accession number: CR542051.1), SIGLECIO (GenBank accession number: AY358337.1), natural killer cell receptor 2B4 (also known as CD244, accession number: NM_001166664.1), PPP2CA, PPP2CB, PTPN6, PTPN22, CD96, CRTAM, SIGLEC7, SIGLEC9, TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIFI, ILIORA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3 which directly inhibit immune cells. For example, PD1 can be combined with an adenoviral vector-based composition to treat a patient in need thereof.

Additional immune pathway checkpoints that can be targeted can be adenosine A2a receptor (ADORA), CD276, V-set domain containing T cell activation inhibitor 1 (VTCN1), indoleamine 2,3-dioxygenase 1 (IDO1), killer cell immunoglobulin-like receptor, three domains, long cytoplasmic tail, 1 (KIR3DL1), V-domain immunoglobulin suppressor of T-cell activation (VISTA), cytokine inducible SH2-containing protein (CISH), hypoxanthine phosphoribosyltransferase 1 (HPRT), adeno-associated virus integration site 1 (AAVS1), or chemokine (C-C motif) receptor 5 (gene/pseudogene) (CCR5), or any combination thereof.

TABLE 3, without being exhaustive, shows exemplary immune pathway checkpoint genes that can be inactivated to improve the efficiency of the adenoviral vector-based composition as described herein. Immune pathway checkpoints gene can be selected from such genes listed in TABLE 3 and others involved in co-inhibitory receptor function, cell death, cytokine signaling, arginine tryptophan starvation, TCR signaling, Induced T-reg repression, transcription factors controlling exhaustion or anergy, and hypoxia mediated tolerance.

TABLE 3
Examples of immune pathway checkpoint genes

| Gene | NCBI # | | | Genome |
|---|---|---|---|---|
| Symbol | (GRCh38.p2) | Start | Stop | location |
| ADORA2A | 135 | 24423597 | 24442360 | 22q11.23 |
| CD276 | 80381 | 73684281 | 73714518 | 15q23-q24 |
| VTCN1 | 79679 | 117143587 | 117270368 | 1p13.1 |
| BTLA | 151888 | 112463966 | 112499702 | 3q13.2 |
| CTLA4 | 1493 | 203867788 | 203873960 | 2q33 |
| IDO1 | 3620 | 39913809 | 39928790 | 8p12-p11 |
| KIR3DL1 | 3811 | 54816438 | 54830778 | 19q13.4 |
| LAG3 | 3902 | 6772483 | 6778455 | 12p13.32 |
| PDCD1 | 5133 | 241849881 | 241858908 | 2q37.3 |
| HAVCR2 | 84868 | 157085832 | 157109237 | 5q33.3 |
| VISTA | 64115 | 71747556 | 71773580 | 10q22.1 |
| CD244 | 51744 | 160830158 | 160862902 | 1q23.3 |
| CISH | 1154 | 50606454 | 50611831 | 3p21.3 |

The combination of an adenoviral-based composition and an immune pathway checkpoint modulator may result in reduction in infection, progression, or symptoms of a disease in treated patients, as compared to either agent alone. In another embodiment, the combination of an adenoviral-based composition and an immune pathway checkpoint modulator may result in improved overall survival of treated patients, as compared to either agent alone. In some cases, the combination of an adenoviral-based composition and an immune pathway checkpoint modulator may increase the frequency or intensity of disease-specific T cell responses in treated patients as compared to either agent alone.

Certain embodiments may also provide the use of immune pathway checkpoint inhibition to improve performance of an adenoviral vector-based composition. Certain immune pathway checkpoint inhibitors may be administered at the time of an adenoviral vector-based composition. Certain immune pathway checkpoint inhibitors may also be administered after the administration of an adenoviral vector-based composition. Immune pathway checkpoint inhibition may occur simultaneously to an adenoviral vaccine administration. Immune pathway checkpoint inhibition may occur 1, 2, 3, 4, 5, 6, 7, 8, 9, 10, 15, 20, 30, 40, 50, or 60 minutes after vaccination. Immune pathway checkpoint inhibition may also occur 1, 2, 3, 4, 5, 6, 7, 8, 9, 10, 11, 12, 13, 14, 15, 16, 17, 18, 19, 20, 21, 22, 23, or 24 hours after the administration of an adenoviral vector-based composition. In some cases, immune inhibition may occur 1, 2, 3, 4, 5, 6, or 7 days after vaccination. Immune pathway checkpoint inhibition may occur at any time before or after the administration of an adenoviral vector-based composition.

In another aspect, there is provided methods involving a vaccine comprising one or more nucleic acids encoding an antigen and an immune pathway checkpoint modulator. For example, there is provided a method for treating a subject having a condition that would benefit from downregulation of an immune pathway checkpoint protein, PD1 or PDL1 for example, and its natural binding partner(s) on cells of the subject.

An immune pathway checkpoint modulator may be combined with an adenoviral vector-based composition comprising one or more nucleic acids encoding any antigen. For example, an antigen can be a tumor antigen, such as a HER2/neu antigen or epitope, or any antigen described herein.

An immune pathway checkpoint modulator may produce a synergistic effect when combined with an adenoviral vector-based composition, such as a vaccine. An immune pathway checkpoint modulator may also produce a beneficial effect when combined with an adenoviral vector-based composition.

### XIII. Cancer

In some embodiments, the methods and compositions of the present disclosure are used to treat cancer in a subject in need threof. In particular aspects, these cancers overexpress the HER2/neu target antigen. HER2/neu is overexpressed in a range of different cancers, including breast, ovarian, prostate, gastric, colon, lung, and bone. HER2/neu overexpression may be useful as a prognostic marker in cancer treatment.

It is specifically contemplated that compositions comprising adenoviral vectors described herein can be used to evaluate or treat stages of disease, such as hyperplasia, dysplasia, neoplasia, pre-cancer, cancer, a primary tumor, or a metastasized tumor. In particular embodiments, the subject has, is at risk of having, or is diagnosed as having a breast cancer, more particularly, a metastatic breast cancer or breast cancer that is unresponsive to other cancer therapy, such as standard breast cancer treatment, unresectable, or locally advanced.

As used herein, the terms “neoplastic cells” and “neoplasia” may be used interchangeably and refer to cells which exhibit relatively autonomous growth, so that they exhibit an aberrant growth phenotype characterized by a significant loss of control of cell proliferation. Neoplastic cells can be malignant or benign. In particular aspects, a neoplasia includes both dysplasia and cancer. Neoplasms may be benign, pre-malignant (carcinoma in situ or dysplasia) or malignant (cancer). Neoplastic cells may form a lump (i.e., a tumor) or not.

The term “dysplasia” may be used when the cellular abnormality is restricted to the originating tissue, as in the case of an early, in-situ neoplasm. Dysplasia may be indicative of an early neoplastic process. The term “cancer” may refer to a malignant neoplasm, including a broad group of various diseases involving unregulated cell growth.

Metastasis, or metastatic disease, may refer to the spread of a cancer from one organ or part to another non-adjacent organ or part. The new occurrences of disease thus generated may be referred to as metastases.

Cancers that may be evaluated or treated by the disclosed methods and compositions include cancer cells particularly from the breast, but may also include cells and cancer cells from the bladder, blood, bone, bone marrow, brain, breast, gastric, colon, esophagus, gastrointestine, gum, head, kidney, liver, lung, nasopharynx, neck, ovary, prostate, skin, stomach, tongue, or uterus.

In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

Breast Cancer

In certain aspects, methods and compositions comprising replication defective vectors comprising a HER2/neu antigen or epitope are used to treat a subject that has, is at risk of having, or is diagnosed as having a breast cancer, particularly unresectable, locally advanced, or metastatic breast cancer.

In certain aspects, breast cancer is diagnosed by microscopic analysis of a sample—or biopsy—of the affected area of the breast. Also, there are types of breast cancer that require specialized lab exams.

The two most commonly used screening methods, physical examination of the breasts by a healthcare provider and mammography, can offer an approximate likelihood that a lump is cancer, and may also detect some other lesions, such as a simple cyst. When these examinations are inconclusive, a healthcare provider can remove a sample of the fluid in the lump for microscopic analysis (a procedure known as fine needle aspiration, or fine needle aspiration and cytology—FNAC) to help establish the diagnosis. A finding of clear fluid makes the lump highly unlikely to be cancerous, but bloody fluid may be sent off for inspection under a microscope for cancerous cells. Together, physical examination of the breasts, mammography, and FNAC can be used to diagnose breast cancer with a good degree of accuracy.

Other options for biopsy include a core biopsy or vacuum-assisted breast biopsy, which are procedures in which a section of the breast lump is removed; or an excisional biopsy, in which the entire lump is removed. Very often the results of physical examination by a healthcare provider, mammography, and additional tests that may be performed in special circumstances (such as imaging by ultrasound or MRI) are sufficient to warrant excisional biopsy as the definitive diagnostic and primary treatment method.

Breast cancers can be classified by different schemata. Each of these aspects influences treatment response and prognosis. Description of a breast cancer would optimally include all of these classification aspects, as well as other findings, such as signs found on physical exam. A full classification includes histopathological type, grade, stage (TNM), receptor status, and the presence or absence of genes as determined by DNA testing:

Histopathology.

The considerable majority of breast cancers are derived from the epithelium lining the ducts or lobules, and are classified as mammary ductal carcinoma. Carcinoma in situ is proliferation of cancer cells within the epithelial tissue without invasion of the surrounding tissue. In contrast, invasive carcinoma invades the surrounding tissue. Perineural and/or lymphovascular space invasion is usually considered as part of the histological description of a breast cancer, and when present may be associated with more aggressive disease.

Grade.

Grading focuses on the appearance of the breast cancer cells compared to the appearance of normal breast tissue. Normal cells in an organ like the breast become differentiated, meaning that they take on specific shapes and forms that reflect their function as part of that organ. Cancerous cells lose that differentiation. In cancer, the cells that would normally line up in an orderly way to make up the milk ducts become disorganized. Cell division becomes uncontrolled. Cell nuclei become less uniform. Pathologists describe cells as well differentiated (low-grade), moderately differentiated (intermediate-grade), and poorly differentiated (high-grade) as the cells progressively lose the features seen in normal breast cells. Poorly differentiated cancers have a worse prognosis.

Stage.

The TNM classification for staging breast cancer is based on the size of the cancer where it originally started in the body and the locations to which it has travelled. These cancer characteristics are described as the size of the tumor (T), whether or not the tumor has spread to the lymph nodes (N) in the armpits, neck, and inside the chest, and whether the tumor has metastasized (M) (i.e., spread to a more distant part of the body). Larger size, nodal spread, and metastasis have a larger stage number and a worse prognosis.

The main stages are Stage 0, Stage 1, Stage 2, Stage 3, and Stage 4.

Stage 0 which is in situ disease or Paget's disease of the nipple. Stage 0 is a pre-cancerous or marker condition, either ductal carcinoma in situ (DCIS) orlobular carcinoma in situ (LCIS).

Stages 1-3 are within the breast or regional lymph nodes.

Stage 4 is a metastatic cancer. Metastatic breast cancer has a less favorable prognosis.

Receptor Status.

Cells have receptors on their surface and in their cytoplasm and nucleus. Chemical messengers such as hormones bind to receptors, and this causes changes in the cell. Breast cancer cells may or may not have many different types of receptors, the three most important in the present classification being: estrogen receptor (ER), progesterone receptor (PR), and HER2/neu. Cells with or without these receptors are called ER positive (ER+), ER negative (ER−), PR positive (PR+), PR negative (PR−), HER2/neu positive (HER2/neu+), and HER2/neu negative (HER2/neu−). Cells with none of these receptors are called basal-like or triple negative.

Osteosarcoma

In some embodiments, methods and compositions comprising replication-defective vectors that comprise a HER2/neu antigen or epitope are used to treat a subject that has, is at risk of having, or is diagnosed as having a bone cancer, particularly osteosarcoma. In certain embodiments, the osteosarcoma can be a high-grade osteosarcoma, an intermediate grade osteosarcoma, or a low-grade osteosarcoma. Osteosarcoma is a cancer of the bone that most commonly is found in subjects in their youth. These cancers most commonly originate in areas of new bone growth. In some embodiments, the methods and compositions of the present disclosure can be administered to treat a subject with any grade or type of osteosarcoma.

Gastric Cancer

In some embodiments, methods and compositions comprising replication-defective vectors that comprise a HER2/neu antigen or epitope are used to treat a subject that has, is at risk of having, or is diagnosed as having gastric cancer. Gastric cancer is a cancer that originates in the stomach, of which nearly 90-95% are adenocarcinomas. In certain embodiments, the gastric cancer can be an adenocarcinoma, lymphoma, gastrointestinal stromal tumor, or a carcinoid tumor. Gastric cancer can also originate from infection by Helicobacter pylori. In some embodiments, the methods and compositions of the present disclosure can be administered to treat a subject with any grade or type of osteosarcoma.

### XIV. Methods of Treatment

The replication-defective adenovirus vectors comprising a target antigen such as a HER2/neu antigen or epitope described herein can be used in a number of vaccine settings for generating an immune response against one or more target antigens as described herein. In some embodiments, there are provided methods of generating an immune response against any target antigen such as a HER2/neu antigen or epitope.

The adenovirus vectors are of particular importance because of the unexpected finding that they can be used to generate immune responses in subjects who have preexisting immunity to Ad and can be used in vaccination regimens that include multiple rounds of immunization using the adenovirus vectors, regimens not possible using previous generation adenovirus vectors.

Generally, generating an immune response comprises an induction of a humoral response and/or a cell-mediated response. It may be desirable to increase an immune response against a target antigen of interest.

Generating an immune response may involve a decrease in the activity and/or number of certain cells of the immune system or a decrease in the level and/or activity of certain cytokines or other effector molecules. A variety of methods for detecting alterations in an immune response (e.g., cell numbers, cytokine expression, cell activity) are available and are useful in some aspects. Illustrative methods useful in this context include intracellular cytokine staining (ICS), ELISpot, proliferation assays, cytotoxic T-cell assays including chromium release or equivalent assays, and gene expression analysis using any number of polymerase chain reaction (PCR) or RT-PCR based assays.

Generating an immune response can comprise an increase in target antigen-specific CTL activity of from 1.5 to 5 fold in a subject administered the adenovirus vectors as described herein as compared to a control. In another embodiment, generating an immune response comprises an increase in target-specific CTL activity of about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 15, 16, 17, 18, 19, 20, or more fold in a subject administered the adenovirus vectors as compared to a control.

Generating an immune response can comprise an increase in target antigen-specific HTL activity, such as proliferation of helper T-cells, of from 1.5 to 5 fold in a subject administered the adenovirus vectors as described herein that comprise nucleic acid encoding the target antigen as compared to an appropriate control. In another embodiment, generating an immune response comprises an increase in target-specific HTL activity of about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 15, 16, 17, 18, 19, 20, or more fold as compared to a control. In this context, HTL activity may comprise an increase as described above, or decrease, in production of a particular cytokine, such as interferon-γ (IFN-γ), interleukin-1 (IL-1), IL-2, IL-3, IL-6, IL-7, IL-12, IL-15, tumor necrosis factor-α (TNF-α), granulocyte macrophage colony-stimulating factor (GM-CSF), granulocyte-colony stimulating factor (G-CSF), or other cytokine. In this regard, generating an immune response may comprise a shift from a Th2 type response to a Th1 type response or in certain embodiments a shift from a Th1 type response to a Th2 type response. In other embodiments, generating an immune response may comprise the stimulation of a predominantly Th1 or a Th2 type response.

Generating an immune response can comprise an increase in target-specific antibody production of between 1.5 and 5 fold in a subject administered the adenovirus vectors as described herein as compared to an appropriate control. In another embodiment, generating an immune response comprises an increase in target-specific antibody production of about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 15, 16, 17, 18, 19, 20, or more fold in a subject administered the adenovirus vector as compared to a control.

Thus, in certain embodiments, there are provided methods for generating an immune response against a target antigen of interest such as a HER2/neu antigen or epitope comprising administering to the individual an adenovirus vector comprising: a) a replication defective adenovirus vector, wherein the adenovirus vector has a deletion in the E2b region, and b) a nucleic acid encoding the target antigen such as a HER2/neu antigen or epitope; and readministering the adenovirus vector at least once to the individual; thereby generating an immune response against the target antigen. In certain embodiments, there are provided methods wherein the vector administered is not a gutted vector. In particular embodiments, the target antigen may be a wild-type protein, a fragment, a variant, or a variant fragment thereof. In some embodiments, the target antigen comprises a tumor antigen such as a HER2/neu antigen or epitope, a fragment, a variant, or a variant fragment thereof.

In a further embodiment, there are provided methods for generating an immune response against a target antigen in an individual, wherein the individual has preexisting immunity to Ad, by administering to the individual an adenovirus vector comprising: a) a replication defective adenovirus vector, wherein the adenovirus vector has a deletion in the E2b region, and b) a nucleic acid encoding the target antigen; and readministering the adenovirus vector at least once to the individual; thereby generating an immune response against the target antigen. In particular embodiments, the target antigen may be a wild-type protein, a fragment, a variant, or a variant fragment thereof. In some embodiments, the target antigen comprises such as a HER2/neu antigen or epitope, a fragment, a variant, or a variant fragment thereof.

With regard to preexisting immunity to Ad, this can be determined using methods known in the art, such as antibody-based assays to test for the presence of Ad antibodies. Further, in certain embodiments, the methods as described herein include first determining that an individual has preexisting immunity to Ad then administering the E2b deleted adenovirus vectors as described herein.

One embodiment provides a method of generating an immune response against one or more target antigens in an individual comprising administering to the individual a first adenovirus vector comprising a replication defective adenovirus vector, wherein the adenovirus vector has a deletion in the E2b region, and a nucleic acid encoding at least one target antigen; administering to the individual a second adenovirus vector comprising a replication defective adenovirus vector, wherein the adenovirus vector has a deletion in the E2b region, and a nucleic acid encoding at least one target antigen, wherein the at least one target antigen of the second adenovirus vector is the same or different from the at least one target antigen of the first adenovirus vector. In particular embodiments, the target antigen may be a wild-type protein, a fragment, a variant, or a variant fragment thereof. In some embodiments, the target antigen comprises a tumor antigen such as a HER2/neu antigen or epitope, a fragment, a variant, or a variant fragment thereof.

Thus, certain embodiments contemplate multiple immunizations with the same E2b deleted adenovirus vector or multiple immunizations with different E2b deleted adenovirus vectors. In each case, the adenovirus vectors may comprise nucleic acid sequences that encode one or more target antigens as described elsewhere herein. In certain embodiments, the methods comprise multiple immunizations with an E2b deleted adenovirus encoding one target antigen, and re-administration of the same adenovirus vector multiple times, thereby inducing an immune response against the target antigen. In some embodiments, the target antigen comprises a tumor antigen such as a HER2/neu antigen or epitope, a fragment, a variant, or a variant fragment thereof.

In a further embodiment, the methods comprise immunization with a first adenovirus vector that encodes one or more target antigens, and then administration with a second adenovirus vector that encodes one or more target antigens that may be the same or different from those antigens encoded by the first adenovirus vector. In this regard, one of the encoded target antigens may be different or all of the encoded antigens may be different, or some may be the same and some may be different. Further, in certain embodiments, the methods include administering the first adenovirus vector multiple times and administering the second adenovirus multiple times. In this regard, the methods comprise administering the first adenovirus vector 1, 2, 3, 4, 5, 6, 7, 8, 9, 10, 11, 12, 13, 14, 15, or more times and administering the second adenovirus vector 1, 2, 3, 4, 5, 6, 7, 8, 9, 10, 11, 12, 13, 14, 15, or more times. The order of administration may comprise administering the first adenovirus one or multiple times in a row followed by administering the second adenovirus vector one or multiple times in a row. In certain embodiments, the methods include alternating administration of the first and the second adenovirus vectors as one administration each, two administrations each, three administrations each, and so on. In certain embodiments, the first and the second adenovirus vectors are administered simultaneously. In other embodiments, the first and the second adenovirus vectors are administered sequentially. In some embodiments, the target antigen comprises a tumor antigen such as a HER2/neu antigen or epitope, a fragment, a variant, or a variant fragment thereof.

As would be readily understood by the skilled artisan, more than two adenovirus vectors may be used in the methods as described herein. Three, 4, 5, 6, 7, 8, 9, 10, or more different adenovirus vectors may be used in the methods as described herein. In certain embodiments, the methods comprise administering more than one E2b deleted adenovirus vector at a time. In this regard, immune responses against multiple target antigens of interest can be generated by administering multiple different adenovirus vectors simultaneously, each comprising nucleic acid sequences encoding one or more target antigens.

The adenovirus vectors can be used to generate an immune response against a cancer, such as carcinomas or sarcomas (e.g., solid tumors, lymphomas and leukemia). The adenovirus vectors can be used to generate an immune response against a cancer, such as neurologic cancers, melanoma, non-Hodgkin's lymphoma, Hodgkin's disease, leukemia, plasmocytomas, adenomas, gliomas, thymomas, breast cancer, prostate cancer, colorectal cancer, kidney cancer, renal cell carcinoma, uterine cancer, pancreatic cancer, esophageal cancer, lung cancer, ovarian cancer, cervical cancer, gastric cancer, multiple myeloma, hepatoma, acute lymphoblastic leukemia (ALL), acute myelogenous leukemia (AML), chronic myelogenous leukemia (CML), and chronic lymphocytic leukemia (CLL), or other cancers.

Methods are also provided for treating or ameliorating the symptoms of any of the infectious diseases or cancers as described herein. The methods of treatment comprise administering the adenovirus vectors one or more times to individuals suffering from or at risk from suffering from an infectious disease or cancer as described herein. As such, certain embodiments provide methods for vaccinating against infectious diseases or cancers in individuals who are at risk of developing such a disease. Individuals at risk may be individuals who may be exposed to an infectious agent at some time or have been previously exposed but do not yet have symptoms of infection or individuals having a genetic predisposition to developing a cancer or being particularly susceptible to an infectious agent. Individuals suffering from an infectious disease or cancer described herein may be determined to express and/or present a target antigen, which may be use to guide the therapies herein. For example, an example can be found to express and/or present a target antigen and an adenovirus vector encoding the target antigen, a variant, a fragment or a variant fragment thereof may be administered subsequently.

Certain embodiments contemplate the use of adenovirus vectors for the in vivo delivery of nucleic acids encoding a target antigen, or a fragment, a variant, or a variant fragment thereof. Once injected into a subject, the nucleic acid sequence is expressed resulting in an immune response against the antigen encoded by the sequence. The adenovirus vector vaccine can be administered in an “effective amount,” that is, an amount of adenovirus vector that is effective in a selected route or routes of administration to elicit an immune response as described elsewhere herein. An effective amount can induce an immune response effective to facilitate protection or treatment of the host against the target infectious agent or cancer. The amount of vector in each vaccine dose is selected as an amount which induces an immune, immunoprotective, or other immunotherapeutic response without significant adverse effects generally associated with typical vaccines. Once vaccinated, subjects may be monitored to determine the efficacy of the vaccine treatment. Monitoring the efficacy of vaccination may be performed by any method known to a person of ordinary skill in the art. In some embodiments, blood or fluid samples may be assayed to detect levels of antibodies. In other embodiments, ELISpot assays may be performed to detect a cell-mediated immune response from circulating blood cells or from lymphoid tissue cells.

In certain embodiments, between 1 and 10 doses may be administered over a 52 week period. In certain embodiments, 6 doses are administered, at intervals of 1, 2, 3, 4, 5, 6, 7, 8, 9, 10, 11, 12, 13, 14, 15, 16, 17, 18, 19, or 20 weeks, 1, 2, 3, 4, 5, 6, 7, 8, 9, 11, 12, 13, 14, 15, 16, 17, 18, 19, 20, 21, 22, 23, or 24 months, or any range or value derivable therefrom, and further booster vaccinations may be given periodically thereafter, at intervals of 1, 2, 3, 4, 5, 6, 7, 8, 9, 10, 11, 12, 13, 14, 15, 16, 17, 18, 19, or 20 weeks, 1, 2, 3, 4, 5, 6, 7, 8, 9, 11, 12, 13, 14, 15, 16, 16, 18, 19, 20, 21, 22, 23, or 24 months, or any range or value derivable therefrom. Alternate protocols may be appropriate for individual patients. As such, 1, 2, 3, 4, 5, 6, 7, 8, 9, 10, 11, 12, 13, 14, 15, 16, 17, 18, 19, 20, or more doses may be administered over a 1 year period or over shorter or longer periods, such as over 35, 40, 45, 50, 55, 60, 65, 70, 75, 80, 85, 90, 95, or 100 week periods. Doses may be administered at 1, 2, 3, 4, 5, or 6 week intervals or longer intervals.

A vaccine can be infused over a period of less than about 4 hours, and more preferably, over a period of less than about 3 hours. For example, the first 25-50 mg could be infused within 30 minutes, preferably even 15 min, and the remainder infused over the next 2-3 hrs. More generally, the dosage of an administered vaccine construct may be administered as one dosage every 2, 3, 4, 5, 6, 7, 8, 9, 10, 11, 12, 13, 14, 15, 16, 17, 18, 19, or 20 weeks, repeated for a total of at least 3 dosages. Or, the construct may be administered twice per week for 4-6 weeks. The dosing schedule can optionally be repeated at other intervals and dosage may be given through various parenteral routes, with appropriate adjustment of the dose and schedule. Compositions as described herein can be administered to a patient in conjunction with (e.g., before, simultaneously, or following) any number of relevant treatment modalities.

A suitable dose is an amount of an adenovirus vector that, when administered as described above, is capable of promoting a target antigen immune response as described elsewhere herein. In certain embodiments, the immune response is at least 10-50% above the basal (i.e., untreated) level. In certain embodiments, the immune response is at least 2, 3, 4, 5, 6, 7, 8, 9, 10, 12, 15, 20, 25, 30, 35, 40, 45, 50, 55, 60, 65, 70, 75, 80, 85, 90, 100, 110, 125, 150, 200, 250, 300, 400, 500 or more over the basal level. Such response can be monitored by measuring the target antigen(s) antibodies in a patient or by vaccine-dependent generation of cytolytic effector cells capable of killing patient tumor or infected cells in vitro, or other methods known in the art for monitoring immune responses. Such vaccines should also be capable of causing an immune response that leads to an improved clinical outcome of the disease in question in vaccinated patients as compared to non-vaccinated patients. In some embodiments, the improved clinical outcome comprises treating disease, reducing the symptoms of a disease, changing the progression of a disease, or extending life.

Any of the compositions provided herein may be administered to an individual. “Individual” may be used interchangeably with “subject” or “patient.” An individual may be a mammal, for example a human or animal such as a non-human primate, a rodent, a rabbit, a rat, a mouse, a horse, a donkey, a goat, a cat, a dog, a cow, a pig, or a sheep. In embodiments, the individual is a human. In embodiments, the individual is a fetus, an embryo, or a child. In some cases, the compositions provided herein are administered to a cell ex vivo. In some cases, the compositions provided herein are administered to an individual as a method of treating a disease or disorder. In some embodiments, the individual has a genetic disease. In some cases, the individual is at risk of having the disease, such as any of the diseases described herein. In some embodiments, the individual is at increased risk of having a disease or disorder caused by insufficient amount of a protein or insufficient activity of a protein. If an individual is “at an increased risk” of having a disease or disorder, the method involves preventative or prophylactic treatment. For example, an individual can be at an increased risk of having such a disease or disorder because of family history of the disease. Typically, individuals at an increased risk of having such a disease or disorder benefit from prophylactic treatment (e.g., by preventing or delaying the onset or progression of the disease or disorder).

In some cases, a subject does not have a disease. In some cases, the treatment as described herein is administered before onset of a disease. A subject may have undetected disease. A subject may have a low disease burden. A subject may also have a high disease burden. In certain cases, a subject may be administered a treatment as described herein according to a grading scale. A grading scale can be a Gleason classification. A Gleason classification reflects how different tumor tissue is from normal prostate tissue. It uses a scale from 1 to 5. A physician gives a cancer a number based on the patterns and growth of the cancer cells. The lower the number, the less normal the cancer cells look and the higher the grade. In certain cases, a treatment may be administered to a patient with a low Gleason score. Preferably, a patient with a Gleason score of 3 or below may be administered a treatment as described herein.

Various embodiments relate to compositions and methods for raising an immune response against one or more particular target antigens such as a HER2/neu antigen or epitope in selected patient populations. Accordingly, methods and compositions as described herein may target patients with a cancer including but not limited to carcinomas or sarcomas such as neurologic cancers, melanoma, non-Hodgkin's lymphoma, Hodgkin's disease, leukemia, plasmocytomas, adenomas, gliomas, thymomas, breast cancer, prostate cancer, colorectal cancer, kidney cancer, renal cell carcinoma, uterine cancer, pancreatic cancer, esophageal cancer, lung cancer, ovarian cancer, cervical cancer, gastric cancer, multiple myeloma, hepatoma, acute lymphoblastic leukemia (ALL), acute myelogenous leukemia (AML), chronic myelogenous leukemia (CML), and chronic lymphocytic leukemia (CLL), or other cancers can be targeted for therapy. In some cases, the targeted patient population may be limited to individuals having colorectal adenocarcinoma, metastatic colorectal cancer, advanced MUC1, MUC1C, MUC1n, T, or CEA expressing colorectal cancer, head and neck cancer, liver cancer, breast cancer, lung cancer, bladder cancer, or pancreas cancer. A histologically confirmed diagnosis of a selected cancer, for example colorectal adenocarcinoma, may be used. A particular disease stage or progression may be selected, for example, patients with one or more of a metastatic, recurrent, stage III, or stage IV cancer may be selected for therapy with the methods and compositions as described herein. In some embodiments, patients may be required to have received and, optionally, progressed through other therapies including but not limited to fluoropyrimidine, irinotecan, oxaliplatin, bevacizumab, cetuximab, or panitumumab containing therapies. In some cases, individual's refusal to accept such therapies may allow the patient to be included in a therapy eligible pool with methods and compositions as described herein. In some embodiments, individuals to receive therapy using the methods and compositions as described herein may be required to have an estimated life expectancy of at least, 1, 2, 3, 4, 5, 6, 7, 8, 9, 10, 11, 12, 14, 15, 18, 21, or 24 months. The patient pool to receive a therapy using the methods and compositions as described herein may be limited by age. For example, individuals who are older than 2, 3, 4, 5, 6, 7, 8, 9, 10, 11, 12, 13, 14, 15, 16, 17, 18, 19, 20, 21, 25, 30, 35, 40, 50, 60, or more years old can be eligible for therapy with methods and compositions as described herein. For another example, individuals who are younger than 75, 70, 65, 60, 55, 50, 40, 35, 30, 25, 20, or fewer years old can be eligible for therapy with methods and compositions as described herein.

In some embodiments, patients receiving therapy using the methods and compositions as described herein are limited to individuals with adequate hematologic function, for example with one or more of a white blood cell (WBC) count of at least 1000, 1500, 2000, 2500, 3000, 3500, 4000, 4500, 5000 or more per microliter, a hemoglobin level of at least 5, 6, 7, 8, 9, 10, 11, 12, 13, 14 or higher g/dL, a platelet count of at least 50,000; 60,000; 70,000; 75,000; 90,000; 100,000; 110,000; 120,000; 130,000; 140,000; 150,000 or more per microliter; with a PT-INR value of less than or equal to 0.8, 1.0, 1.2, 1.3, 1.4, 1.5, 1.6, 1.8, 2.0, 2.5, 3.0, or higher, a PTT value of less than or equal to 1.2, 1.4, 1.5, 1.6, 1.8, 2.0×ULN or more. In various embodiments, hematologic function indicator limits are chosen differently for individuals in different gender and age groups, for example 0-5, 5-10, 10-15, 15-18, 18-21, 21-30, 30-40, 40-50, 50-60, 60-70, 70-80, or older than 80.

In some embodiments, patients receiving therapy using the methods and compositions as described herein are limited to individuals with adequate renal and/or hepatic function, for example with one or more of a serum creatinine level of less than or equal to 0.8, 0.9, 1.0, 1.1, 1.2, 1.3, 1.4, 1.5, 1.6, 1.7, 1.8, 1.9, 2.0, 2.1, 2.2 mg/dL or more, a bilirubin level of 0.8, 0.9, 1.0, 1.1, 1.2, 1.3, 1.4, 1.5, 1.6, 1.7, 1.8, 1.9, 2.0, 2.1, 2.2 mg/dL or more, while allowing a higher limit for Gilbert's syndrome, for example, less than or equal to 1.5, 1.6, 1.8, 1.9, 2.0, 2.1, 2.2, 2.3, or 2.4 mg/dL, an ALT and AST value of less than or equal to less than or equal to 1.5, 2.0, 2.5, 3.0× upper limit of normal (ULN) or more. In various embodiments, renal or hepatic function indicator limits are chosen differently for individuals in different gender and age groups, for example 0-5, 5-10, 10-15, 15-18, 18-21, 21-30, 30-40, 40-50, 50-60, 60-70, 70-80, or older than 80.

In some embodiments, the K-ras mutation status of individuals who are candidates for a therapy using the methods and compositions as described herein can be determined. Individuals with a preselected K-ras mutational status can be included in an eligible patient pool for therapies using the methods and compositions as described herein.

In various embodiments, patients receiving therapy using the methods and compositions as described herein are limited to individuals without concurrent cytotoxic chemotherapy or radiation therapy, a history of, or current, brain metastases, a history of autoimmune disease, such as but not restricted to, inflammatory bowel disease, systemic lupus erythematosus, ankylosing spondylitis, scleroderma, multiple sclerosis, thyroid disease and vitiligo, serious intercurrent chronic or acute illness, such as cardiac disease (NYHA class III or IV), or hepatic disease, a medical or psychological impediment to probable compliance with the protocol, concurrent (or within the last 5 years) second malignancy other than non-melanoma skin cancer, cervical carcinoma in situ, controlled superficial bladder cancer, or other carcinoma in situ that has been treated, an active acute or chronic infection including: a urinary tract infection, HIV (e.g., as determined by ELISA and confirmed by Western Blot), and chronic hepatitis, or concurrent steroid therapy (or other immuno-suppressives, such as azathioprine or cyclosporin A). In some cases, patients with at least 3, 4, 5, 6, 7, 8, 9, or 10 weeks of discontinuation of any steroid therapy (except that used as pre-medication for chemotherapy or contrast-enhanced studies) may be included in a pool of eligible individuals for therapy using the methods and compositions as described herein. In some embodiments, patients receiving therapy using the methods and compositions o as described herein include individuals with thyroid disease and vitiligo.

In various embodiments, samples, for example serum or urine samples, from the individuals or candidate individuals for a therapy using the methods and compositions as described herein may be collected. Samples may be collected before, during, and/or after the therapy for example, within 2, 4, 6, 8, 10 weeks prior to the start of the therapy, within 1 week, 10 day, 2 weeks, 3 weeks, 4 weeks, 6 weeks, 8 weeks, or 12 weeks from the start of the therapy, within 2, 4, 6, 8, 10 weeks prior to the start of the therapy, within 1 week, 10 days, 2 weeks, 3 weeks, 4 weeks, 6 weeks, 8 weeks, 9 weeks, or 12 weeks from the start of the therapy, in 1 week, 10 day, 2 weeks, 3 weeks, 4 weeks, 6 weeks, 8 weeks, 9 weeks, or 12 weeks intervals during the therapy, in 1 month, 3 month, 6 month, 1 year, 2 year intervals after the therapy, within 1 month, 3 months, 6 months, 1 year, 2 years, or longer after the therapy, for a duration of 6 months, 1, 2, 3, 4, 5, 6, 7, 8, 9, 10 years or longer. The samples may be tested for any of the hematologic, renal, or hepatic function indicators described herein as well as suitable others known in the art, for example a 13-HCG for women with childbearing potential. In that regard, hematologic and biochemical tests, including cell blood counts with differential, PT, INR and PTT, tests measuring Na, K, Cl, CO₂, BUN, creatinine, Ca, total protein, albumin, total bilirubin, alkaline phosphatase, AST, ALT and glucose are contemplated in certain aspects. In some embodiments, the presence or the amount of HIV antibody, Hepatitis BsAg, or Hepatitis C antibody are determined in a sample from individuals or candidate individuals for a therapy using the methods and compositions described herein.

Biological markers, such as antibodies to target antigens or the neutralizing antibodies to Ad5 vector can be tested in a sample, such as serum, from individuals or candidate individuals for a therapy using the methods and compositions described herein. In some cases, one or more samples, such as a blood sample can be collected and archived from an individuals or candidate individuals for a therapy using the methods and compositions described herein. Collected samples can be assayed for immunologic evaluation. Individuals or candidate individuals for a therapy using the methods and compositions described herein can be evaluated in imaging studies, for example using CT scans or MRI of the chest, abdomen, or pelvis. Imaging studies can be performed before, during, or after therapy using the methods and compositions described herein, during, and/or after the therapy, for example, within 2, 4, 6, 8, 10 weeks prior to the start of the therapy, within 1 week, 10 days, 2 weeks, 3 weeks, 4 weeks, 6 weeks, 8 weeks, or 12 weeks from the start of the therapy, within 2, 4, 6, 8, 10 weeks prior to the start of the therapy, within 1 week, 10 day, 2 weeks, 3 weeks, 4 weeks, 6 weeks, 8 weeks, 9 weeks, or 12 weeks from the start of the therapy, in 1 week, 10 day, 2 week, 3 week, 4 week, 6 week, 8 week, 9 week, or 12 week intervals during the therapy, in 1 month, 3 month, 6 month, 1 year, 2 year intervals after the therapy, within 1 month, 3 months, 6 months, 1 year, 2 years, or longer after the therapy, for a duration of 6 months, 1, 2, 3, 4, 5, 6, 7, 8, 9, 10 years, or longer.

Compositions and methods described herein contemplate various dosage and administration regimens during therapy. Patients may receive one or more replication defective adenovirus or adenovirus vector, for example Ad5 [E1−, E2b−]-vectors comprising a target antigen that is capable of raising an immune response in an individual against a target antigen described herein.

In various embodiments, the replication defective adenovirus is administered at a dose that suitable for effecting such immune response. In some embodiments, the replication defective adenovirus is administered at a dose from about 1×10⁸ virus particles to about 5×10¹³ virus particles per immunization. In some cases, the replication defective adenovirus is administered at a dose from about 1×10⁹ to about 5×10¹² virus particles per immunization. In some embodiments, the replication defective adenovirus is administered at a dose from about 1×10⁸ virus particles to about 5×10⁸ virus particles per immunization. In some embodiments, the replication defective adenovirus is administered at a dose from about 5×10⁸ virus particles to about 1×10⁹ virus particles per immunization. In some embodiments, the replication defective adenovirus is administered at a dose from about 1×10⁹ virus particles to about 5×10⁹ virus particles per immunization. In some embodiments, the replication defective adenovirus is administered at a dose from about 5×10⁹ virus particles to about 1×10¹⁰ virus particles per immunization. In some embodiments, the replication defective adenovirus is administered at a dose from about 1×10¹⁰ virus particles to about 5×10¹⁰ virus particles per immunization. In some embodiments, the replication defective adenovirus is administered at a dose from about 5×10¹⁰ virus particles to about 1×10¹¹ virus particles per immunization. In some embodiments, the replication defective adenovirus is administered at a dose from about 1×10¹¹ virus particles to about 5×10¹¹ virus particles per immunization. In some embodiments, the replication defective adenovirus is administered at a dose from about 5×10¹¹ virus particles to about 1×10¹² virus particles per immunization. In some embodiments, the replication defective adenovirus is administered at a dose from about 1×10¹² virus particles to about 5×10¹² virus particles per immunization. In some embodiments, the replication defective adenovirus is administered at a dose from about 5×10¹² virus particles to about 1×10¹³ virus particles per immunization. In some embodiments, the replication defective adenovirus is administered at a dose from about 1×10¹³ virus particles to about 5×10¹³ virus particles per immunization. In some embodiments, the replication defective adenovirus is administered at a dose from about 1×10⁸ virus particles to about 5×10¹⁰ virus particles per immunization. In some embodiments, the replication defective adenovirus is administered at a dose from about 1×10¹⁰ virus particles to about 5×10¹² virus particles per immunization. In some embodiments, the replication defective adenovirus is administered at a dose from about 1×10¹¹ virus particles to about 5×10¹³ virus particles per immunization. In some embodiments, the replication defective adenovirus is administered at a dose from about 1×10⁸ virus particles to about 1×10¹⁰ virus particles per immunization. In some embodiments, the replication defective adenovirus is administered at a dose from about 1×10¹⁰ virus particles to about 1×10¹² virus particles per immunization. In some embodiments, the replication defective adenovirus is administered at a dose from about 1×10¹¹ virus particles to about 5×10¹³ virus particles per immunization. In some cases, the replication defective adenovirus is administered at a dose that is greater than or equal to 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰ ′ 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 1.5×10¹², 2×10¹², 3×10¹², 4×10¹², 5×10¹², or more virus particles (VP) per immunization. In some cases, the replication defective adenovirus is administered at a dose that is less than or equal to 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 1.5×10¹², 2×10¹², 3×10¹², 4×10¹², 5×10¹², or more virus particles per immunization. In various embodiments, a desired dose described herein is administered in a suitable volume of formulation buffer, for example a volume of about 0.1-10 mL, 0.2-8 mL, 0.3-7 mL, 0.4-6 mL, 0.5-5 mL, 0.6-4 mL, 0.7-3 mL, 0.8-2 mL, 0.9-1.5 mL, 0.95-1.2 mL, or 1.0-1.1 mL. Those of skill in the art appreciate that the volume may fall within any range bounded by any of these values (e.g., about 0.5 mL to about 1.1 mL). Administration of virus particles can be through a variety of suitable paths for delivery, for example it can be by injection (e.g., intracutaneously, intramuscularly, intravenously or subcutaneously), intranasally (e.g., by aspiration), in pill form (e.g., swallowing, suppository for vaginal or rectal delivery. In some embodiments, a subcutaneous delivery may be preferred and can offer greater access to dendritic cells.

Administration of virus particles to an individual may be repeated. Repeated deliveries of virus particles may follow a schedule or alternatively, may be performed on an as needed basis. For example, an individual's immunity against a target antigen, for example a tumor antigen such as a HER2/neu antigen or epitope, a fragment, a variant, or a variant fragment thereof, may be tested and replenished as necessary with additional deliveries. In some embodiments, schedules for delivery include administrations of virus particles at regular intervals. Joint delivery regimens may be designed comprising one or more of a period with a schedule and/or a period of need based administration assessed prior to administration. For example, a therapy regimen may include an administration, such as subcutaneous administration once every three weeks then another immunotherapy treatment every three months until removed from therapy for any reason including death. Another example regimen comprises three administrations every three weeks then another set of three immunotherapy treatments every three months.

Another example regimen comprises a first period with a first number of administrations at a first frequency, a second period with a second number of administrations at a second frequency, a third period with a third number of administrations at a third frequency, etc., and optionally one or more periods with undetermined number of administrations on an as needed basis. The number of administrations in each period can be independently selected and can for example be 1, 2, 3, 4, 5, 6, 7, 8, 9, 10, 11, 12, 13, 14, 15, 16, 17, 18, 19, 20, or more. The frequency of the administration in each period can also be independently selected, can for example be about every day, every other day, every third day, twice a week, once a week, once every other week, every three weeks, every month, every six weeks, every other month, every third month, every fourth month, every fifth month, every sixth month, once a year etc. The therapy can take a total period of up to 1, 2, 3, 4, 5, 6, 7, 8, 9, 10, 11, 12, 13, 14, 15, 16, 17, 18, 19, 20, 21, 22, 23, 24, 30, 36 months, or more.

The scheduled interval between immunizations may be modified so that the interval between immunizations is revised by up to a fifth, a fourth, a third, or half of the interval. For example, for a 3-week interval schedule, an immunization may be repeated between 20 and 28 days (3 weeks−1 day to 3 weeks+7 days). For the first 3 immunizations, if the second and/or third immunization is delayed, the subsequent immunizations may be shifted allowing a minimum amount of buffer between immunizations. For example, for a three week interval schedule, if an immunization is delayed, the subsequent immunization may be scheduled to occur no earlier than 17, 18, 19, or 20 days after the previous immunization.

Compositions described herein can be provided in various states, for example, at room temperature, on ice, or frozen. Compositions may be provided in a container of a suitable size, for example a vial of 2 mL vial. In one embodiment, one 2 ml vial with 1.0 mL of extractable vaccine contains 5×10¹¹ total virus particles/mL. Storage conditions including temperature and humidity may vary. For example, compositions for use in therapy may be stored at room temperature, 4° C., −20° C., or lower.

In various embodiments, general evaluations are performed on the individuals receiving treatment according to the methods and compositions as described herein. One or more of any tests may be performed as needed or in a scheduled basis, such as on weeks 0, 3, 6 etc. A different set of tests may be performed concurrent with immunization vs. at time points without immunization.

General evaluations may include one or more of medical history, ECOG Performance Score, Karnofsky performance status, and complete physical examination with weight by the attending physician. Any other treatments, medications, biologics, or blood products that the patient is receiving or has received since the last visit may be recorded. Patients may be followed at the clinic for a suitable period, for example approximately 30 minutes, following receipt of vaccine to monitor for any adverse reactions.

In certain embodiments, local and systemic reactogenicity after each dose of vaccine may be assessed daily for a selected time, for example for 3 days (on the day of immunization and 2 days thereafter). Diary cards may be used to report symptoms and a ruler may be used to measure local reactogenicity. Immunization injection sites may be assessed. CT scans or MRI of the chest, abdomen, and pelvis may be performed.

In various embodiments, hematological and biochemical evaluations are performed on the individuals receiving treatment according to the methods and compositions as described herein. One or more of any tests may be performed as needed or in a scheduled basis, such as on weeks 0, 3, 6 etc. A different set of tests may be performed concurrent with immunization vs. at time points without immunization. Hematological and biochemical evaluations may include one or more of blood test for chemistry and hematology, CBC with differential, Na, K, Cl, CO₂, BUN, creatinine, Ca, total protein, albumin, total bilirubin, alkaline phosphatase, AST, ALT, glucose, and ANA.

In various embodiments, biological markers are evaluated on individuals receiving treatment according to the methods and compositions as described herein. One or more of any tests may be performed as needed or in a scheduled basis, such as on weeks 0, 3, 6, etc. A different set of tests may be performed concurrent with immunization vs. at time points without immunization.

Biological marker evaluations may include one or more of measuring antibodies to target antigens or viral vectors described herein, from a serum sample of adequate volume, for example about 5 ml biomarkers may be reviewed if determined and available.

In various embodiments, an immunological assessment is performed on individuals receiving treatment according to the methods and compositions as described herein. One or more of any tests may be performed as needed or in a scheduled basis, such as on weeks 0, 3, 6, etc. A different set of tests may be performed concurrent with immunization vs. at time points without immunization.

Peripheral blood, for example about 90 mL may be drawn prior to each immunization and at a time after at least some of the immunizations, to determine whether there is an effect on the immune response at specific time points during the study and/or after a specific number of immunizations. Immunological assessment may include one or more of assaying peripheral blood mononuclear cells (PBMC) for T-cell responses to target antigens such as a HER2/neu antigen or epitope using ELISpot, proliferation assays, multi-parameter flow cytometric analysis, and cytoxicity assays. Serum from each blood draw may be archived and sent and determined.

In various embodiments, a tumor assessment is performed on individuals receiving treatment according to the methods and compositions as described herein. One or more of any tests may be performed as needed or in a scheduled basis, such as prior to treatment, on weeks 0, 3, 6, etc. A different set of tests may be performed concurrent with immunization vs. at time points without immunization. Tumor assessment may include one or more of CT or MRI scans of chest, abdomen, or pelvis performed prior to treatment, at a time after at least some of the immunizations and at approximately every three months following the completion of a selected number, for example 2, 3, or 4, of first treatments and for example until removal from treatment.

Immune responses against a target antigen such as a HER2/neu antigen or epitope may be evaluated from a sample, such as a peripheral blood sample of an individual using one or more suitable tests for immune response, such as ELISpot, cytokine flow cytometry, or antibody response. A positive immune response can be determined by measuring a T-cell response. A T-cell response can be considered positive if the mean number of spots adjusted for background in six wells with antigen exceeds the number of spots in six control wells by 10 and the difference between single values of the six wells containing antigen and the six control wells is statistically significant at a level of p≤0.05 using the Student's t-test. Immunogenicity assays may occur prior to each immunization and at scheduled time points during the period of the treatment. For example, a time point for an immunogenicity assay at around week 1, 2, 3, 4, 5, 6, 7, 8, 9, 10, 11, 12, 13, 14, 15, 18, 20, 24, 30, 36, or 48 of a treatment may be scheduled even without a scheduled immunization at this time. In some cases, an individual may be considered evaluable for immune response if they receive at least a minimum number of immunizations, for example 1, 2, 3, 4, 5, 6, 7, 8, 9, or more immunizations.

In some embodiments, disease progression or clinical response determination is made according to the RECIST 1.1 criteria among patients with measurable/evaluable disease. In some embodiments, therapies using the methods and compositions as described herein affect a Complete Response (CR; disappearance of all target lesions for target lesions or disappearance of all non-target lesions and normalization of tumor marker level for non-target lesions) in an individual receiving the therapy. In some embodiments, therapies using the methods and compositions as described herein affect a Partial Response (PR; at least a 30% decrease in the sum of the LD of target lesions, taking as reference the baseline sum LD for target lesions) in an individual receiving the therapy.

In some embodiments, therapies using the methods and compositions as described herein affect a Stable Disease (SD; neither sufficient shrinkage to qualify for PR nor sufficient increase to qualify for PD, taking as reference the smallest sum LD since the treatment started for target lesions) in an individual receiving the therapy. In some embodiments, therapies using the methods and compositions described herein affect an Incomplete Response/Stable Disease (SD; persistence of one or more non-target lesion(s) or/and maintenance of tumor marker level above the normal limits for non-target lesions) in an individual receiving the therapy. In some embodiments, therapies using the methods and compositions as described herein affect a Progressive Disease (PD; at least a 20% increase in the sum of the LD of target lesions, taking as reference the smallest sum LD recorded since the treatment started or the appearance of one or more new lesions for target lesions or persistence of one or more non-target lesion(s) or/and maintenance of tumor marker level above the normal limits for non-target lesions) in an individual receiving the therapy.

### XV. Kits

The compositions, immunotherapy, or vaccines described herein may be supplied in the form of a kit. The kits of the present disclosure may further comprise instructions regarding the dosage and or administration including treatment regimen information.

In some embodiments, kits comprise the compositions and methods for providing immunotherapy or vaccines described. In some embodiments, kits may further comprise components useful in administering the kit components and instructions on how to prepare the components. In some embodiments, the kit can further comprise software for conducting monitoring patient before and after treatment with appropriate laboratory tests, or communicating results and patient data with medical staff.

The components comprising the kit may be in dry or liquid form. If they are in dry form, the kit may include a solution to solubilize the dried material. The kit may also include transfer factor in liquid or dry form. If the transfer factor is in dry form, the kit will include a solution to solubilize the transfer factor. The kit may also include containers for mixing and preparing the components. The kit may also include instrument for assisting with the administration such for example needles, tubing, applicator, inhalant, syringe, pipette, forceps, measured spoon, eye dropper or any such medically approved delivery vehicle. The kits or drug delivery systems as described herein also will typically include a means for containing compositions of the present disclosure in close confinement for commercial sale and distribution.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Construction of Ad5 [E1−, E2b−] Vector

This example describes the construction of the Ad5 [E1−, E2b−] vector. The construction of the Ad5 [E1−, E2b−] vector backbone has previously been described. The approximately 20 kb Xba-BamHI subfragment of pBHG11 was subcloned into pBluescriptKSII+(Stratagene, La Jolla, Calif.), yielding pAXB. Plasmid pAXB was digested with BspEI, T4 DNA polymerase end filled, and BamHI digested, and the approximately 9.0 kb fragment was isolated. Plasmid pAXB was also digested with BspHI, T4 DNA polymerase end filled, and BamHI digested, and the approximately 13.7 kb fragment was ligated to the previously isolated 9.0 kb fragment, generating pAXB-Δpol.

This subcloning strategy deleted 608 bp (Δpol; Ad5 nucleotides 7274 to 7881) within the amino terminus of the polymerase gene. This deletion also effectively removed open reading frame 9.4 present on the rightward reading strand in this region of the Ad genome. The Xba-BamHI subfragment of pAXB-Δpol was reintroduced into Xba-BamHI-digested pBHG11, to generate pBHG11-Δpol.

### Example 2

### Construction of the Ad5 [E1−, E2b−]-HER2/Neu Vaccine

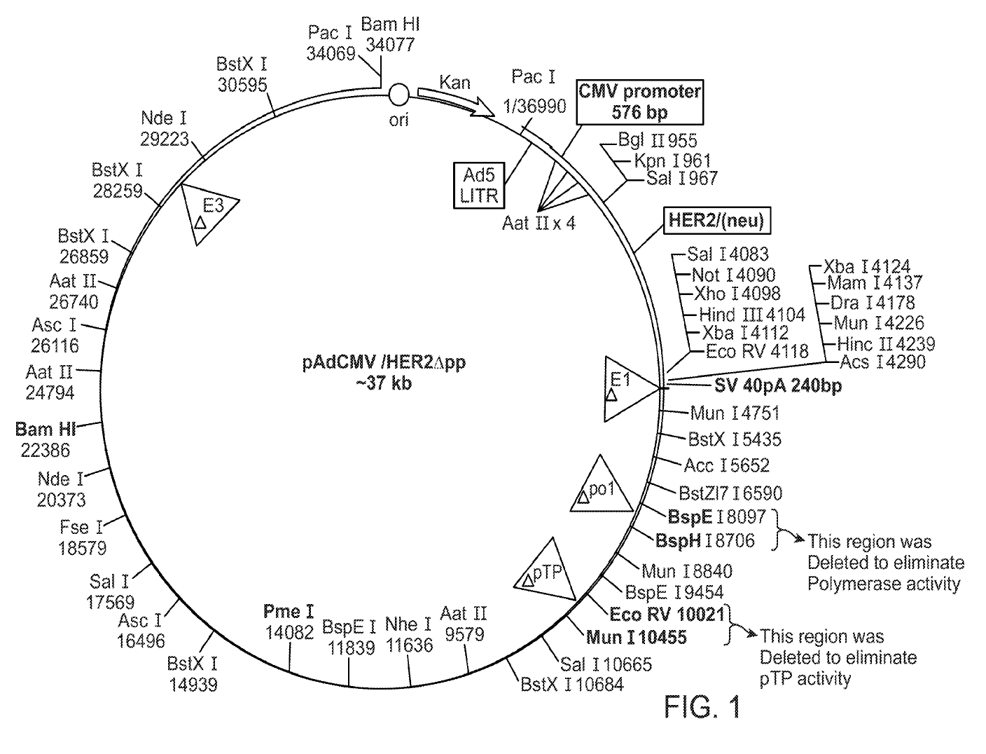

This example describes construction of the Ad5 [E1−, E2b−]-HER2/neu vaccine. A truncated HER2/neu transgene flanked by a minimal cytomegalovirus promoter/enhancer element and the SV40 derived poly adenylation signal was subcloned into the shuttle pShuttleCMV, generating the shuttle plasmid pShuttle CMV/HER2/neu. The shuttle plasmid was linearized with Pmel and homologously recombined (in E. coli bacteria) with the plasmid pAdΔpp to generate pAdCMV/HER2/neu/Δpp (FIG. 1).

Ten micrograms of pAdCMV/HER2/neu/Δpp linearized with PacI was CaPO4 cotransfected into Ad E1, polymerase (E2b) and pTP-expressing (E.C7 cells). Sixteen hours after transfection, the cells were harvested and the cell mixture was distributed into nine 24-well tissue culture cluster plates and incubated at 37° C. for 5 to 9 days. Individual wells demonstrating viral cytopathic effects were harvested, and the isolated virus was amplified by repeated infection of greater numbers of E.C7 cells. Isolation of the Ad5 [E1−, E2b−]-HER2/neu recombinant vector was subsequently confirmed by (1) DNA restriction mapping of the vector genome, (2) confirmation of expression of HER2/neu and (3) multiple functional studies. A complete sequence of the Ad5 [E1−, E2b−]-HER2/neu vector is found in SEQ ID NO: 3. The CMV promoter sequence in the complete sequence of the Ad5 [E1−, E2b−]-HER2/neu vector (SEQ ID NO: 3) is found in SEQ ID NO: 4. The SV40 polyA tail sequence in the complete sequence of the Ad5 [E1−, E2b−]-HER2/neu vector (SEQ ID NO: 3) is found in SEQ ID NO: 5.

### Example 3

### Assessment of Preclinical Toxicology of Ad5 [E1−, E2b−]-HER2/Neu

This example describes assessment of preclinical toxicology of Ad5 [E1−, E2b−]-HER2/neu. The repeat-dose toxicity of Ad5 [E1−, E2b−]-HER2/neu was evaluated in a GLP study in BALB/c mice. The study consisted of eight groups: four vehicle control groups (Groups 1 to 4) and four test article treated groups (Groups 5 to 8). Mice were immunized on days 1, 22, and 43 with Ad5 [E1−, E2b−]-HER2/neu at 1.7×10⁸ virus particles (VP)/dose. The dose of 1.7×10⁸ VP/dose (8.3×10⁹ VP/kg) of Ad5 [E1−, E2b−]-HER2/neu is the mouse-to-human equivalent of the highest proposed dose of 5×10¹¹ VP/dose (8.3×10⁹ VP/kg) in humans, assuming a human weighs 60 kg and a mouse weighs 0.02 kg. Ad5 [E1−, E2b−]-HER2/neu was given subcutaneously to mice, which is also the intended route of administration for patients.

Overall, Ad5 [E1−, E2b−]-HER2/neu was well tolerated in mice. One mouse died, considered not related to the Ad5 [E1−, E2b−]-HER2/neu vaccine. None of the clinical signs observed cage-side and during hands-on observations were considered related to the Ad5 [E1−, E2b−]-HER2/neu vaccine. All other animals survived until scheduled sacrifice. Erythema and edema was evident in some of the Ad5 [E1−, E2b−]-HER2/neu-treated animals, but the erythema generally occurred on a single day. Due to the low incidence and severity of the erythema, it is not considered toxicologically significant. Treatment with Ad5 [E1−, E2b−]-HER2/neu did not have any toxicologically significant effects on body weights, body weight gain, or food consumption. There was no evidence in the clinical pathology, organ weight, or histopathology data at any interval of an effect from the subcutaneous injection of the Ad5 [E1−, E2b−]-HER2/neu vaccine.

Treatment with the Ad5 [E1−, E2b−]-HER2/neu vaccine had no biologically significant effects on blood counts; prothrombin time (PT); activated partial thromboplastin time; levels of sodium, potassium, chloride, calcium, creatine phosphokinase, aspartate aminotransferase, alanine aminotransferase, alkaline phosphatase, glucose, blood urea nitrogen, creatinine, cholesterol, total bilirubin, total protein, albumin, and globulin; and albumin/globulin ratios (TABLES 4-5).

TABLE 4
Summary of Clinical Pathology For Male Mice
Significant Changes in Clinical Chemistry and Hematology Parameters for Male Mice

| | Day 3 | | Day 14 | | Day 28 | | Day 45 | | Day 67 | |
|---|---|---|---|---|---|---|---|---|---|---|
| | | Ad5 [E1−, | | Ad5 [E1−, | | Ad5 [E1−, | | Ad5 [E1−, | | Ad5 [E1−, |
| | Vehicle | E2b−]- | Vehicle | E2b−]- | Vehicle | E2b−]- | Vehicle | E2b−]- | Vehicle | E2b−]- |
| Parameter | Control | HER2/neu | Control | HER2/neu | Control | HER2/neu | Control | HER2/neu | Control | HER2/neu |

Clinical Chemistry

| | | | | | | | | | | |
|---|---|---|---|---|---|---|---|---|---|---|
| Glucose | 199.20 | ↓156.80 | — | — | — | — | — | — | — | — |
| Globulin | — | — | — | — | 2.17 | ↑2.43 | — | — | — | — |
| Potassium | — | — | — | — | — | — | 10.60 | ↓9.50 | — | — |
| Blood Urea Nitrogen | — | — | — | — | — | — | — | — | 28.20 | ↓20.80 |
| Sodium | 151.80 | ↑155.60 | — | — | — | — | — | — | — | — |
| Chloride | — | — | — | — | — | — | — | — | 106.58 | ↑109.12 |

Hematology

| | | | | | | | | | | |
|---|---|---|---|---|---|---|---|---|---|---|
| % Large Unstained | 1.56 | ↓0.84 | — | — | — | — | — | — | — | — |
| Cells | | | | | | | | | | |
| Mean Platelet Volume | — | — | 4.90 | ↑5.24 | — | — | — | — | — | — |
| White Blood Cells | — | — | — | — | — | — | 4.83 | ↑10.87 | — | — |
| Neutrophils | — | — | — | — | — | — | 0.99 | ↑1.97 | — | — |
| Lymphocytes | — | — | — | — | — | — | 3.55 | ↑8.42 | — | — |
| Mean Corpuscular | — | — | — | — | — | — | — | — | 14.78 | ↓14.58 |
| Hemoglobin | | | | | | | | | | |
| Platelets | — | — | — | — | — | — | — | — | 1052.75 | ↓918.80 |
| Reticulocytes | — | — | — | — | — | — | — | — | 242.30 | ↓210.26 |
| % Reticulocytes | — | — | — | — | — | — | — | — | 2.26 | ↓1.87 |
| Prothrombin Time | — | — | 9.7 | ↓9.4 | — | — | — | — | — | — |

Group 1: ARM buffer control treated at Day 1; blood collected at Day 14 for hematology, clinical chemistry, and coagulation parameters; sacrificed at Day 14.
Group 2: ARM buffer control treated at Days 1 and 22; blood collected at Day 28 for coagulation parameters and antibody analysis; sacrificed at Day 28.
Group 3: ARM buffer control treated at Days 1, 22 and 43; blood collected at Day 3 for hematology and clinical chemistry; blood collected at Day 45 for hematology, clinical chemistry, and coagulation parameters; sacrificed at Day 45.
Group 4: ARM buffer control treated at Days 1, 22 and 43; blood collected at Day 28 for hematology and clinical chemistry; blood collected at Day 67 for hematology, clinical chemistry, and coagulation parameters; sacrificed at Day 67.
Group 5: Ad5 [E1−E2b−]- HER2/neu treated at Day 1; blood collected at Day 14 for hematology, clinical chemistry, and coagulation parameters; sacrificed at Day 14
Group 6: Ad5 [E1−E2b−]- HER2/neu treated at Days 1 and 22; blood collected at Day 28 for coagulation parameters and antibody analysis; sacrificed at Day 28.
Group 7: Ad5 [E1−E2b−]- HER2/neu treated at Days 1, 22 and 43; blood collected at Day 3 for hematology and clinical chemistry; blood collected at Day 45 for hematology, clinical chemistry, and coagulation parameters; sacrificed at Day 45.
Group 8: Ad5 [E1−E2b−]- HER2/neu treated at Days 1, 22 and 43; blood collected at Day 28 for hematology and clinical chemistry; blood collected at Day 67 for hematology, clinical chemistry, and coagulation parameters; sacrificed at Day 67.
↑The increase was statistically significant (p < 0.05).
↓The decrease was statistically significant (p < 0.05).
—, not statistically significant.
Source: NantBioScience, data on file.

TABLE 5
Summary of Clinical Pathology For Female Mice
Significant Changes in Clinical Chemistry and Hematology Parameters for Female Mice

| | Day 3 | | Day 14 | | Day 28 | | Day 45 | | Day 67 | |
|---|---|---|---|---|---|---|---|---|---|---|
| | | Ad5 [E1−, | | Ad5 [E1−, | | Ad5 [E1−, | | Ad5 [E1−, | | Ad5 [E1−, |
| | Vehicle | E2b−]- | Vehicle | E2b−]- | Vehicle | E2b−]- | Vehicle | E2b−]- | Vehicle | E2b−]- |
| Parameter | Control | HER2/neu | Control | HER2/neu | Control | HER2/neu | Control | HER2/neu | Control | HER2/neu |

Clinical Chemistry

| | | | | | | | | | | |
|---|---|---|---|---|---|---|---|---|---|---|
| Alanine Aminotransferase | 33.60 | ↓27.20 | — | — | — | — | — | — | — | — |
| Alkaline Phosphatase | 190.00 | ↓152.00 | — | — | — | — | — | — | — | — |
| Blood Urea Nitrogen | — | — | — | — | — | — | 19.80 | ↓17.25 | — | — |
| Sodium | — | — | 152.40 | ↑155.00 | — | — | — | — | 151.20 | ↑153.00 |
| Creatinine | — | — | — | — | 0.16 | ↑0.21 | — | — | — | — |
| Chloride | — | — | — | — | — | — | 111.02 | ↓108.88 | — | — |

Hematology

| | | | | | | | | | | |
|---|---|---|---|---|---|---|---|---|---|---|
| Mean Platelet Volume | 4.58 | ↑4.80 | — | — | 6.32 | ↓5.38 | 5.18 | ↓4.50 | — | — |
| White Blood Cells | — | — | 6.51 | ↑10.88 | 14.89 | ↓11.58 | — | — | — | — |
| Lymphocytes | — | — | 4.69 | ↑8.72 | — | — | — | — | — | — |
| Hematocrit | — | — | 52.80 | ↑56.88 | — | — | — | — | — | — |
| Mean Corpuscular Hemoglobin | — | — | — | — | — | — | 15.38 | ↓15.07 | — | — |
| Monocytes | — | — | — | — | — | — | — | — | 0.09 | ↑0.23 |
| Red Cell Distribution Width | — | — | — | — | — | — | — | — | 13.24 | ↑13.75 |
| Activated Partial Thromboplastin | — | — | — | — | — | — | 34.12 | ↓23.38 | 30.20 | ↓24.73 |
| Time | | | | | | | | | | |

Group 1: ARM buffer control treated at Day 1; blood collected at Day 14 for hematology, clinical chemistry, and coagulation parameters; sacrificed at Day 14.
Group 2: ARM buffer control treated at Days 1 and 22; blood collected at Day 28 for coagulation parameters and antibody analysis; sacrificed at Day 28.
Group 3: ARM buffer control treated at Days 1, 22 and 43; blood collected at Day 3 for hematology and clinical chemistry; blood collected at Day 45 for hematology, clinical chemistry, and coagulation parameters; sacrificed at Day 45.
Group 4: ARM buffer control treated at Days 1, 22 and 43; blood collected at Day 28 for hematology and clinical chemistry; blood collected at Day 67 for hematology, clinical chemistry, and coagulation parameters; sacrificed at Day 67.
Group 5: Ad5 [E1−E2b−]- HER2/neu treated at Day 1; blood collected at Day 14 for hematology, clinical chemistry, and coagulation parameters; sacrificed at Day 14
Group 6: Ad5 [E1−E2b−]- HER2/neu treated at Days 1 and 22; blood collected at Day 28 for coagulation parameters and antibody analysis; sacrificed at Day 28.
Group 7: Ad5 [E1−E2b−]- HER2/neu treated at Days 1, 22 and 43; blood collected at Day 3 for hematology and clinical chemistry; blood collected at Day 45 for hematology, clinical chemistry, and coagulation parameters; sacrificed at Day 45.
Group 8: Ad5 [E1−E2b−]- HER2/neu treated at Days 1, 22 and 43; blood collected at Day 28 for hematology and clinical chemistry; blood collected at Day 67 for hematology, clinical chemistry, and coagulation parameters; sacrificed at Day 67.
↑The increase was statistically significant (p < 0.05).
↓ The decrease was statistically significant (p < 0.05).
—, not statistically significant.
Source: NantBioScience, data on file.

### Example 4

### Preparation of the Ad5 [E1−, E2b−]-HER2/Neu Vaccine (Suspension for Injection)

This example describes preparation of the Ad5 [E1−, E2b−]-HER2/neu vaccine (suspension for injection). Ad5 [E1−, E2b−]-HER2/neu vaccine (suspension for injection) is a replication defective, adenovirus vector system. Ad5 [E1−, E2b−]-HER2/neu is a HER2/neu-targeting vaccine comprising the Ad5 [E1−, E2b−] vector and a modified HER2/neu gene insert. The HER2/neu gene insert encodes a truncated human HER2/neu protein, consisting of the extracellular domain and transmembrane regions. The entire intracellular domain, containing the kinase domain that leads to oncogenic activity, was removed.

### Pharmaceutical Properties

Ad5 [E1−, E2b−]-HER2/neu is a recombinant replication-defective Ad5 vector that was modified by removal of the E1 gene, deletions in the E2b and E3 genes, and insertion of a truncated gene for human HER2/neu, consisting of the extracellular domain and transmembrane regions. The entire intracellular domain, containing the kinase domain that leads to oncogenic activity, was removed (Gabitzsch E S and Jones F R. J Clin Cell Immunol. 2011a; S4:001, Hartman Z C, Wei J, Osada T, et al. An adenoviral vaccine encoding full-length inactivated human HER2/neu exhibits potent immunogenicty and enhanced therapeutic efficacy without oncogenicity. Clin Cancer Res. 2010; 16:1466-1477).

### Evaluation of Adventitious Safety Agents

Ad5 [E1−, E2b−]-HER2/neu was modified by significant deletions in the E1, E2b, and E3 regions and insertion of a human HER2/neu gene. The resulting replication-defective viral vector can be propagated in a proprietary human embryonic kidney 293 cell line (E.C7) that can supply the deleted E1 and E2b gene products in trans. There is, however, a theoretical possibility that a replication competent adenovirus could be formed during manufacturing of the adenoviral virus particles by recombination with the E1 and E2b sequences residing in the E.C7 (293) cell line. Therefore, a sensitive test for replication competent adenovirus was incorporated into release testing for this vaccine.

The E.C7 Master Cell Bank (MCB) and the Master Viral Bank (MVB) were tested for a broad panel of viruses, and all results were negative. In addition, no bacterial, fungal, or mycoplasma contaminations were detected in the MCB or the MVB.

One animal-derived component, fetal bovine serum (FBS), was used in the growth medium for the E.C7 cell expansion. The Australian-sourced FBS was certified to be in compliance with 9 CFR 113.53 Requirements for ingredients of animal origin used for production of biologics.

Ad5 [E1−, E2b−]-HER2/neu was supplied as a sterile, clear suspension in a 2-mL single-dose vial. The vaccine was provided at a concentration of 5×10¹¹ VP per 1 mL and contained ARM formulation buffer (20 mM TRIS, 25 mM NaCl, 2.5% glycerol, pH 8.0). Each vial contained approximately 1.1 mL of the vaccine.

Ad5 [E1−, E2b−]-HER2/neu was stored in the pharmacy at ≤−20° C. until ready for use. Prior to injection, the appropriate vial was removed from the freezer and allowed to thaw at controlled room temperature 20-25° C. (68-77° F.) for 20-30, after which it should be kept at 2-8° C. (35-46° F.).

### Example 5

### Preclinical Studies of an Ad5 [E1−, E2b−]-HER2/Neu Cancer Vaccine

This example describes preclinical studies of an Ad5 [E1−, E2b−]-HER2/neu cancer vaccine. Studies were performed to evaluate Ad5 [E1−, E2b−]-HER2/neu as a cancer vaccine in a BALB/c mouse model. Ad5 [E1−, E2b−]-HER2/neu induced potent CMI against HER2/neu in Ad5-naïve and Ad5-immune mice. Humoral responses were induced, and antibodies demonstrated the ability to lyse HER2/neu-expressing tumor cells in the presence of complement in vitro. Ad5 [E1−, E2b−]-HER2/neu prevented the establishment of HER2/neu-expressing tumors and significantly inhibited progression of established tumors in Ad5-naïve and Ad5-immune murine models. These data indicate that in vivo delivery of Ad5 [E1−, E2b−]-HER2/neu can induce anti-HER2/neu immunity and inhibit progression of HER2/neu-expressing cancers.

Preclinical studies and noteworthy findings are presented in TABLE 6

TABLE 6
Ad5 [E1−, E2b−]−HER2/neu Pre-clinical Animal Studies
Test Article: Ad5 [E1−, E2b−]−HER2/neu

| | | | | |
|---|---|---|---|---|
| | | | Method of | |
| Type of Study | GLP | Test System | Administration | Noteworthy Findings |

Immunogenicity

| | | | | |
|---|---|---|---|---|
| Dose response of Ad5 | No | Ad5-naïve | Subcutaneous | A dose-response effect was observed for the CMI response when dosed |
| [E1−, E2b−]− | | BALB/c mice | injection | with 1 × 10⁸, 1 × 10⁹, or 1 × 10¹⁰ VP. The greatest induction of CMI was |
| HER2/neu | | | | achieved using 1 × 10¹⁰ VP of test article. |
| Induction of CMI | No | Ad5-naïve and | Subcutaneous | In Ad5-naïve mice, significantly elevated CMI response was observed |
| responses by Ad5 | | Ad5-immune | injection | after 2 or 3 immunizations when compared to 1 injection. Levels were |
| [E1−, E2b−]− | | BALB/c mice | | not significantly different between 2 and 3 immunizations. In |
| HER2/neu after 1, 2, | | | | Ad5 immune mice, levels were significantly higher after 3 |
| or 3 immunizations | | | | immunizations when compared to 1 or 2 injections. |
| Induction of humoral | No | Ad5-naïve and | Subcutaneous | HER2/neu antibody was detected in Ad5-naïve and Ad5-immune mice |
| immune responses by | | Ad5-immune | injection | after 1, 2, and 3 injections, with the greatest quantities of antibody |
| Ad5 [E1−, E2b−]− | | BALB/c mice | | detected after 3 immunizations. |
| HER2/neu | | | | |

Antitumor Activity

| | | | | |
|---|---|---|---|---|
| Prevention of | No | Ad5-naïve and | Subcutaneous | Ad5 naïve and immune mice were immunized with 3 injections of test |
| HER2/neu-expressing | | Ad5-immune | injection | article and then inoculated with 10⁶ CT26-HER2/neu tumor cells. Mice |
| tumor establishment | | BALB/c mice | | treated with test article had significant reduction in tumor progression |
| in vivo | | | | after 8 and 9 days, respectively, compared to tumor control and vector |
| | | | | control mice. |

Antitumor Activity

| | | | | |
|---|---|---|---|---|
| Dose response of Ad5 | No | Ad5-naïve | Subcutaneous | A dose-response effect was observed for the CMI response when dosed |
| [E1−, E2b−]− | | BALB/c mice | injection | with 1 × 10⁸, 1 × 10⁹, or 1 × 10¹⁰ VP. The greatest induction of CMI was |
| HER2/neu | | | | achieved using 1 × 10¹⁰ VP of test article. |
| Treatment of | No | Ad5-naïve and | Subcutaneous | Ad5 naïve and immune mice were inoculated with 10⁶ CT26-HER2/neu |
| established | | Ad5-immune | injection | tumor cells and tumors were palpable by days 4-6. Mice were then |
| HER2/neu-expressing | | BALB/c mice | | treated with test article, vector control, or buffer only on days 7 and 14. |
| tumors | | | | Test article immunized mice had significantly smaller tumors by days 11, |
| | | | | 14, and 16 compared to control mice. |

Toxicology

| | | | | |
|---|---|---|---|---|
| 67-Day Subcutaneous | Yes | BALB/c mice | Subcutaneous | Treatment with the test article did not have any toxicologically |
| Repeat-Dose Toxicity | | | injection | significant effects on body weights, body weight gain, or food |
| Study of Ad5 [E1−, | | | | consumption. There was no evidence in the clinical pathology, organ |
| E2b−]−HER2/neu in | | | | weight, or histopathology data at any interval of an effect from the |
| BALB/c Mice | | | | subcutaneous injection of the test article. |

### Example 6

### Phase I Study of Ad5 [E1−, E2b−]-HER2/Neu Vaccination in Subjects with Unresectable Locally Advanced or Metastatic HER2/Neu-Expressing Breast Cancer

This example describes a Phase I study of Ad5 [E1−, E2b−]-HER2/neu vaccination in subjects with unresectable, locally advanced or metastatic HER2/neu-expressing (IHC 1+ or 2+) breast cancer. The Ad5 [E1−, E2b−]-HER2/neu vaccine is administered subcutaneously (SC) once a week for three weeks (three injections total) and is followed by three booster injections at three-month intervals to subjects with HER2/neu-expressing breast cancer. The overall safety of this vaccine regimen is determined and the recommended dose in Phase 2 of the Ad5 [E1−, E2b−]-HER2/neu vaccine is identified. Preliminary assessments of objective response rate (ORR), disease control rate (DCR), duration of response, progression-free survival (PFS), and overall survival (OS) in subjects with HER2/neu-expressing breast cancer treated with Ad5 [E1−, E2b−]-HER2/neu are made. The immunogenicity of Ad5 [E1−, E2b−]-HER2/neu is evaluated and the genomic and proteomic profile of subjects' tumors are determined to identify gene mutations, gene amplifications, RNA-expression levels, and protein-expression levels. Correlations between genomic/proteomic profiles and efficacy outcomes are also assessed.

A summary of clinical studies is provided in TABLE 7.

TABLE 7
Ad5 [E1−, E2b−]−HER2/neu Clinical Studies

| | | Number | | | |
|---|---|---|---|---|---|
| Study | | of | | Dosage and | |
| Design | Indication | Subjects | Age | Dosage Regimen | Primary Endpoints |
| Phase 1 | HER2/neu- | Up to 30 | Age ≥18 | Ad5 [E1−, E2b−]− | Dose-limiting |
| | expressing | | years | HER2/neu (5 × | toxicities and |
| | breast | | | 10⁹ VP, 5 × 10¹⁰ | maximum tolerated |
| | cancer | | | VP or 5 × 10¹¹ | dose or highest |
| | | | | VP) is | tested dose. |
| | | | | administered by | Treatment-emergent |
| | | | | subcutaneous | AEs and SAEs. |
| | | | | injection every 3 | Clinically |
| | | | | weeks for 3 | significant changes |
| | | | | immunizations, | in safety laboratory |
| | | | | followed by 3 | tests, physical |
| | | | | booster | examinations, |
| | | | | immunizations at | electrocardiograms, |
| | | | | 3-month | LVEF, and vital |
| | | | | intervals. | signs. |

Secondary endpoints include ORR (confirmed complete or partial response) according to the Response Evaluation Criteria in Solid Tumors (RECIST) Version 1.1., DCR (confirmed response or stable disease lasting for at least 6 months), duration of response, progression-free survival (PFS), and overall survival (OS).

The immunogenicity of Ad5 [E1−, E2b−]-HER2/neu is assessed by flow cytometric analysis of T-cell frequency, activation status, cytokine profiles, and antibody levels. Genetic and proteomic profiling is conducted and correlated with efficacy.

### Study Design

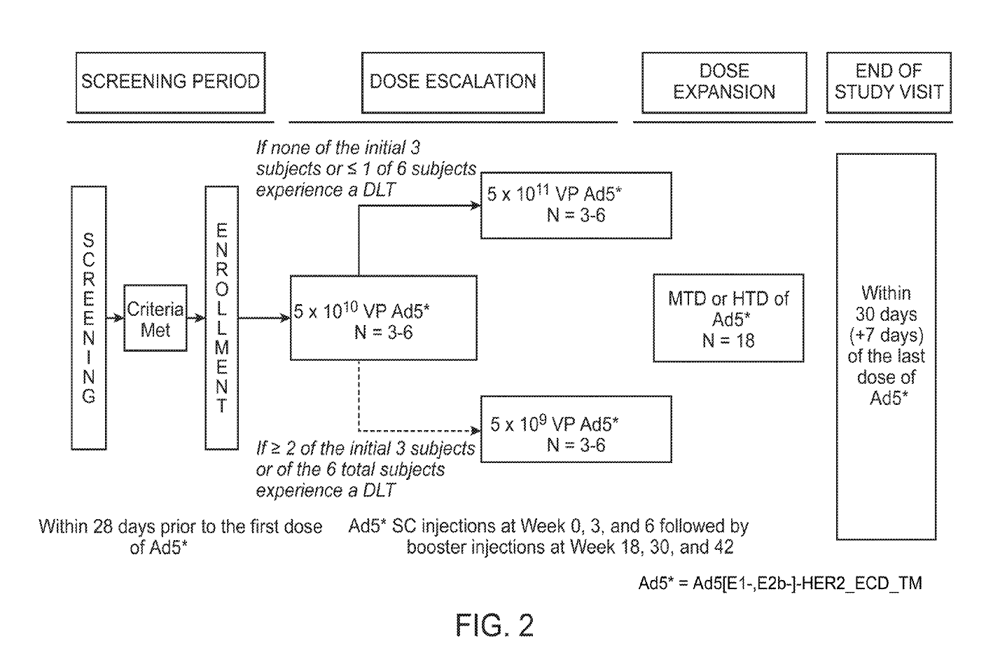

A Phase I trial is conducted including subjects with unresectable locally advanced or metastatic HER2/neu-low expressing (IHC 1+ or 2+) breast cancer. The study is conducted in two parts: the first part involves dose escalation using a 3+3 design, and the second part involves the expansion of the maximum tolerated dose (MTD) or highest tested dose (HTD) to further evaluate safety, preliminary efficacy, and immunogenicity. In the first part, 3 to 6 subjects are sequentially enrolled starting at dose Cohort 1. Cohort 1 receives 5×10¹⁰ virus particles (VP), Cohort 2 receives 5×10¹¹ VP, and if needed, the dose de-escalation cohort (Cohort-1) receives 5×10⁹ VP. Subjects are assessed for dose-limiting toxicities (DLTs). Dose expansion occurs when the MTD or HTD has been determined. An additional 12 subjects are enrolled in the dose expansion component of the trial, for a total of 18 subjects at the MTD or HTD. A schematic of the proposed study is shown in FIG. 2.

In the dose-escalation component, 3 to 6 subjects are sequentially enrolled starting at dose Cohort 1 (TABLE 8). During specific cohort enrollment, there is a minimum of 7 days between enrolling successive subjects. DLTs are monitored continuously.

A DLT is defined as any Grade 3 or greater toxicity as defined by National Cancer Institute (NCI) Common Terminology Criteria for Adverse Events (CTCAE) Version 4.03 or any Grade 2 or higher autoimmune reaction or immediate hypersensitivity reaction. Dose escalation is performed as shown in TABLE 8. No intra-patient dose escalations are permitted.

TABLE 8
Dose Levels

| Cohort | Ad5 [E1−, E2b−]−HER2/neu (VP) |
|---|---|
| −1 | 5 × 10⁹ |
| 1 | 5 × 10¹⁰ |
| 2 | 5 × 10¹¹ |

In Cohort 1, if none of the initial three subjects experience a DLT, dose escalation to Cohort 2 commences. If one of the initial three subjects experiences a DLT, three additional subjects are enrolled into Cohort 1 for a total of six subjects. If ≤one of six subjects experience a DLT, escalation to Cohort 2 commences. If ≥two of the initial three subjects or of the six total subjects experience a DLT, enrollment into the de-escalation Cohort-1 commences.

In Cohort 2, if ≤one of the initial three subjects experience a DLT, three additional subjects are enrolled into Cohort 2 for a total of six subjects. If ≤one of six subjects experience a DLT, this dose level is defined as the HTD. If ≥two of the initial three subjects, or if ≥two of a total six subjects experience a DLT, enrollment into the next lower dose level may be resumed as follows. If three subjects are treated in Cohort 1, three additional subjects are enrolled at this dose level for a total of six subjects. If ≤one of six subjects experiences a DLT, that dose is defined as the MTD. If ≥two of six subjects experience a DLT, enrollment into the de-escalation Cohort-1 commences. Additionally, if six subjects are treated in Cohort 1, that dose is defined as the MTD.

In the dose de-escalation Cohort-1, if ≤one of the initial three subjects experiences a DLT, three additional subjects are enrolled into de-escalation Cohort-1 for a total of six subjects. If ≤one of six subjects experiences a DLT, this dose level is defined as the MTD. If ≥two of the initial three subjects, or if ≥two of a total six subjects experience a DLT, dosing is suspended, and the study is re-evaluated.

Dose expansion occurs after all the available safety and laboratory results are reviewed by the safety review committee (SRC) and when the MTD or HTD has been determined. An additional 12 subjects are enrolled in the dose expansion component of the study, for a total of 18 subjects at the MTD or HTD.

Safety events that trigger a temporary suspension of the study injections include death possibly related to the study agent, two Grade 4 toxicity events that are possibly related to the study agent, if more than one of the first six enrolled subjects in the de-escalation Cohort-1 experience a DLT, or if at any time during the expansion phase greater than 33% of subjects experience a Grade 3 or 4 major organ toxicity possibly related to study injections.

### Subjects

Up to 30 subjects are enrolled in the study. Subjects have histologically confirmed unresectable locally advanced or metastatic breast cancer that expresses HER2/neu (IHC 1+ or 2+). Subjects with HER2/neu IHC 3+ tumors are excluded. In the dose escalation component, 3 to 6 subjects are sequentially enrolled starting at dose Cohort 1. In the dose expansion component (i.e., once the MTD or HTD has been identified), an additional 12 subjects are enrolled for a total of 18 subjects in the MTD/HTD cohort to obtain further safety, preliminary efficacy, and immunogenicity data.

### Duration of Treatment

It is anticipated that each subject is on approximately 42 weeks of treatment (injections occur at 0, 3, and 6 weeks with booster injections at 18, 30, and 42 weeks) or until they experience progressive disease or unacceptable toxicity, withdraw consent, or if the Investigator feels it is no longer in their best interest to continue treatment. The estimated duration of treatment for subjects may be longer or shorter depending on the subject's disease, ability to tolerate Ad5 [E1−, E2b−]-HER2/neu, willingness to participate in the study, or if the Investigator feels it is no longer in their best interest to continue treatment.

### Dose Modification

Ad5 [E1−, E2b−]-HER2/neu is withheld for any of the following reasons: any Grade 3 or greater toxicity as defined by CTCAE Version 4.03, any Grade 2 or higher autoimmune reaction or immediate hypersensitivity reaction, less than a 16%, or a 16%, absolute decrease in the left ventricular ejection fraction (LVEF) from pretreatment values, an LVEF below institutional defined lower limits of normal (LLN) and greater than a 10%, or a 10%, absolute decrease in the LVEF from pretreatment values.

HER2/neu is permanently discontinued for any of the following reasons: any hypersensitivity reaction that is possibly related to Ad5 [E1−, E2b−]-HER2/neu, life-threatening anaphylactic reactions, subjects that develop symptomatic congestive heart failure with decreased LVEF, any life-threatening adverse reaction, Grade 3 or higher injection site reaction (e.g., ulceration, necrosis), Grade 4 toxicity (except fever) attributed to the injections, or Grade 4 fever lasting over 48 hours.

The following are acceptable conditions for dose delays. First, dosing of the first three vaccines should be given on schedule every 3 weeks (Week 0, 3, and 6) and in the event of conflicts, a 5-day window is acceptable. Second, for unrelated acute illnesses present at the time of a scheduled vaccination, dosing can be delayed until symptoms subside, or the subject may be withdrawn at the discretion of the Investigator and delays up to 3 weeks are considered acceptable in this setting. There are no dose reductions for Ad5 [E1−, E2b−]-HER2/neu. Concomitant medications permitted are concurrent bisphosphonate therapy.

### Inclusion Criteria

Subject eligibility for the Phase I clinical trial is defined by inclusion criteria and exclusion criteria. Inclusion criteria include the following: age ≥18 years, male or female, ability to understand and provide signed informed consent that fulfills Institutional Review Board (IRB)'s guidelines, histologically confirmed unresectable locally advanced or metastatic breast cancer that expresses HER2/neu (IHC 1+ or 2+), derived from the most recent metastatic biopsy sample available, tumor tissue (block or slides) and whole blood sample available for analysis (archival tissue is permitted), and an Eastern Cooperative Oncology Group (ECOG) performance status of 0 or 1.

Further, subjects who have received prior HER2/neu-targeted immunotherapy (vaccine) are eligible for this trial if this treatment was discontinued at least 3 months prior to enrollment. All toxic side effects of prior chemotherapy, radiotherapy, or surgical procedures are resolevd to NCI CTCAE Grade ≤1. Subjects who are taking medications that do not have a known history of immunosuppression are eligible for this trial. Additionally, adequate hematologic function at screening is defined as follows: a white blood count ≥3000/microliter, hemoglobin ≥9 g/dL (may not transfuse or use erythropoietin to achieve this level), platelets ≥75,000/microliter, a prothrombin (PT)-international normalized ratio (INR) <1.5, and a partial thromboplastin time (PTT)<1.5× upper limit of normal (ULN). Adequate renal and hepatic function at screening is defined as follows: a serum creatinine <2.0 mg/dL, bilirubin <1.5 mg/dL (except for Gilbert's syndrome which allows bilirubin ≤2.0 mg/dL), alanine aminotransferase (ALT)≤2.5×ULN, and aspartate aminotransferase (AST) ≤2.5×ULN.

Additionally for eligibility, inclusion criteria also includes a multigated acquisition (MUGA) scan or echocardiogram with an LVEF ≥institutional LLN (same imaging modality is to be used throughout the study). Female subjects of childbearing potential and women <12 months since the onset of menopause must agree to use acceptable contraceptive methods for the duration of the study and for four months following the last injection of study medication. If employing contraception, two of the following precautions must be used: vasectomy of partner, tubal ligation, vaginal diaphragm, intrauterine device, condom and spermicidal (gel/foam/cream/vaginal suppository), or total abstinence. Male subjects must be surgically sterile or must agree to use a condom and acceptable contraceptive method with their partner. Female subjects who are post-menopausal are defined as those with an absence of menses for >12 consecutive months. Finally, inclusion criteria include the ability to attend required study visits and return for adequate follow up.

### Exclusion Criteria

Subject eligibility for the Phase I clinical trial is defined by inclusion criteria and exclusion criteria. Exclusion criteria include the following: subjects with HER2/neu IHC 3+ tumors, subjects with ongoing HER2/neu-directed therapy, including trastuzumab, pertuzumab, T-DM1, and lapatinib, participation in an investigational drug or device study within 30 days of screening for this study, pregnant and nursing women, and subjects with ongoing palbociclib, everolimus, or other breast cancer therapy that interferes with the induction of immune responses.

Additional criteria for exclusion include subjects with concurrent cytotoxic chemotherapy or radiation therapy. There must be at least 1 month between any other prior chemotherapy (or radiotherapy) and study treatment. Any prior HER2/neu-targeted immunotherapy (vaccine) must have been discontinued at least 3 months before initiation of study treatment. Subjects must have recovered from all acute toxicities from prior treatment prior to screening for this study.

Further criteria for exclusion are subjects with active brain or central nervous system metastasis, seizures requiring anticonvulsant treatment, cerebrovascular accident (<6 months), or transient ischemic attack, subjects with a history of autoimmune disease (active or past), such as but not restricted to inflammatory bowel disease, systemic lupus erythematosus, ankylosing spondylitis, scleroderma, or multiple sclerosis (autoimmune-related thyroid disease and vitiligo are permitted), subjects with serious intercurrent chronic or acute illness, such as cardiac or pulmonary disease, hepatic disease, or other illness considered as high risk for investigational drug treatment, subjects with a history of heart disease, such as congestive heart failure (class II, III, or IV defined by the New York Heart Association functional classification), history of unstable or poorly controlled angina, or history (<1 year) of ventricular arrhythmia, and subjects with a medical or psychological impediment that would impair the ability of the subject to receive therapy per protocol or impact ability to comply with the protocol or protocol-required visits and procedures.

History of malignancy is also criteria for exclusion except for the following: adequately treated non-melanoma skin cancer, cervical carcinoma in situ, superficial bladder cancer, or other carcinoma that has been in complete remission without treatment for more than 5 years. Presence of a known active acute or chronic infection, including human immunodeficiency virus (HIV, as determined by enzyme-linked immunosorbent assay [ELISA] and confirmed by western blot) and hepatitis B and hepatitis C virus (HBV/HCV, as determined by HBsAg and hepatitis C serology) is considered criteria for exclusion. Subjects on systemic intravenous or oral steroid therapy (or other immunosuppressives, such as azathioprine or cyclosporin A) are excluded on the basis of potential immune suppression. Subjects must have had at least 6 weeks of discontinuation of any steroid therapy (except that used as premedication for chemotherapy or contrast-enhanced studies) prior to enrollment.

Subjects with known allergy or hypersensitivity to any component of the investigational product are excluded. Subjects with acute or chronic skin disorders that interfere with injection into the skin of the extremities or subsequent assessment of potential skin reactions are excluded. Finally, subjects vaccinated with a live (attenuated) vaccine (e.g., FluMist®) or a killed (inactivated)/subunit vaccine (e.g., PNEUMOVAX®, Fluzone®) within 28 days or 14 days, respectively, of the first planned dose of Ad5 [E1−, E2b−]-HER2/neu.

### Ad5 [E1−, E2b−]-HER2/Neu Dose Preparation

The product name, dosage form, unit dose, route of administration, physical description, and manufacturer for the Ad5 [E1−, E2b−]-HER2/neu vaccine is summarized in TABLE 9.

TABLE 9
Ad5 [E1−, E2b−]−HER2/neu

| | |
|---|---|
| Product Name: | Ad5 [E1−, E2b−]−HER2/neu Vaccine |
| Dosage Form: | Suspension for injection |
| Unit Dose | 5 × 10⁹ VP, 5 × 10¹⁰ VP, or 5 × 10¹¹ VP |
| Route of | SC injection |
| Administration | |
| Physical | Ad5 [E1−, E2b−]−HER2/neu is supplied as a sterile, |
| Description | clear solution in a 2-mL single-dose vial. The vaccine |
| | is provided at a concentration of 5 × 10¹¹ VP per 1 mL |
| | and contains ARM formulation buffer (20 mM TRIS, |
| | 25 mM NaCl, 2.5% glycerol, pH 8.0). Each vial contains |
| | approximately 1.1 mL of the vaccine. The product should |
| | be stored at ≤−20° C. |
| Manufacturer | SAFC Pharma |

The injected dose of Ad5 [E1−, E2b−]-HER2/neu is 5×10⁹ VP (for de-escalation Cohort-1), 5×10¹⁰ VP (Cohort 1), or 5×10¹¹ VP (Cohort 2) per 1 mL. Prior to injection, the appropriate vial is removed from the freezer and allowed to thaw at controlled room temperature (20-25° C., 68-77° F.) for at least 20 minutes and not more than 30 minutes, after which it is kept at 2-8° C. (35-46° F.).

Each vial is sealed with a rubber stopper and has a white flip-off seal. The end user of the product flips the white plastic portion of the cap up/off with their thumb to expose the rubber stopper and then punctures the stopper with an injection needle to withdraw the liquid. The rubber stopper is secured to the vial with an aluminum-crimped seal. The thawed vial is swirled and then, using aseptic technique, the pharmacist withdraws the appropriate volume from the appropriate vial using a 1-mL syringe.

The vaccine dose is injected as soon as possible using a 1 to ½ inch, 20 to 25 gauge needle. If the vaccine cannot be injected immediately, the syringe is returned to the pharmacy and properly disposed in accordance with institutional policy and procedure, and disposition is recorded on the investigational product accountability record.

Storage of the vaccine in the vial at 2-8° C. (35-46° F.) does not exceed 8 hours. Also, once the vaccine is thawed, it is not refrozen.

Dose preparation for Cohort 2 (5×10¹¹ VP) is as follows. 1 mL of contents from the vial is withdrawn, the injection site is prepared with alcohol, and the dose is administered to the subject by subcutaneous injection in the thigh without any further manipulation.

Dose preparation for Cohort 1 (5×10¹⁰ VP) is as follows. Using a 1.0 mL tuberculin syringe, 0.50 mL of fluid is removed from a 5.0-mL vial of 0.9% sterile saline, leaving 4.50 mL. Using another 1.0 mL tuberculin syringe, 0.50 mL is removed from the vial labeled Ad5 [E1−, E2b−]-HER2/neu, and delivered into the 4.5 mL of sterile saline remaining in the 5-mL sterile saline vial. The contents are mixed by inverting the 5 mL solution of diluted Ad5 [E1-, E2b-]-HER2/neu. 1 mL of the diluted Ad5 [E1−, E2b−]-HER2/neu is withdrawn, the injection site is prepared with alcohol, and the dose is administered to the subject by subcutaneous injection in the thigh.

Dose preparation for Cohort-1 (5×10⁹ VP, Dose De-escalation) is as follows. A 0.50 mL tuberculin syringe is used to remove 0.05 mL of fluid from a 5.0-mL vial of 0.9% sterile saline, leaving 4.95 mL. Using another 0.50 mL tuberculin syringe, 0.05 mL is removed from the vial labeled Ad5 [E1−, E2b−]-HER2/neu, and delivered into the 4.95 mL of sterile saline remaining in the 5-mL sterile saline vial. The contents are mixed by inverting the 5 mL of diluted Ad5 [E1−, E2b−]-HER2/neu. 1 mL of the diluted Ad5 [E1−, E2b−]-HER2/neu is withdrawn, the injection site is prepared with alcohol, and the dose is administered to the subject by subcutaneous injection in the thigh.

### Administration

Ad5 [E1−, E2b−]-HER2/neu is administered on Week 0, 3, and 6 for a total of three injections followed by three booster injections at 3-month intervals (Week 18, 30, and 42). All study drug administration treatment occurs within ±5 days of the planned visit date. All injections of the vaccine should be given as a volume of 1 mL by SC injection in the thigh after preparation of the site with alcohol. Either thigh may be used for the initial injection. Subsequent injections must be given in the same thigh as the initial injection and must be separated by at least 5 cm.

The Ad5 [E1−, E2b−] vector is non-replicating and its genome does not integrate into the human genome. Since the vector is a non-replicating recombinant virus, it is handled under Biosafety Level-2 conditions. Any vialed Ad5 [E1−, E2b−]-HER2/neu material used is autoclaved after use.

### Criteria for Evaluation

Safety endpoints include assessments of DLT, MTD or HTD, treatment-emergent AEs, SAES, and clinically significant changes in safety laboratory tests, physical examinations, ECGs, LVEF, and vital signs. Toxicities are graded using the National Cancer Institute (NCI) Common Terminology Criteria for Adverse Events (CTCAE) Version 4.03. To assess efficacy, tumor response (ORR and DCR) is evaluated according to RECIST Version 1.1; duration of response, PFS, and OS.

### Efficacy Assessments

Efficacy of the Ad5 [E1−, E2b−]-HER2/neu vaccine is assessed by evaluating survival and antitumor response. After the subject completes or withdraws from the study, all subjects are followed for survival every 3 months for 12 months and then approximately every 6 months thereafter for 12 months.

Tumor assessments may include the following evaluations: physical examination (with photograph and measurement of skin lesions, as applicable); cross-sectional imaging using computed tomography (CT) or magnetic resonance imaging (MRI) scan of the chest, abdomen, and pelvis (pelvis scan is optional unless known pelvic disease is present at baseline); nuclear bone scan for subjects with known/suspected bone lesions; and CT or MRI scan of the brain (only as clinically warranted based on symptoms/findings). The preferred method of disease assessment is CT with contrast. If CT with contrast is contraindicated, CT of the chest without contrast and MRI scan of the abdomen/pelvis with contrast is preferred.

At baseline, tumor lesions are selected and categorized as target or non-target lesions. Target lesions include those lesions that can be accurately measured in at least 1 dimension as ≥20 mm with conventional techniques or ≥10 mm with CT scan. Malignant lymph nodes with a short axis diameter ≥15 mm can be considered target lesions. Up to a maximum of 2 target lesions per organ and 5 target lesions in total are identified at baseline. These lesions should be representative of all involved organs and selected based on their size (those with the longest diameter) and their suitability for accurate repeated measurements. A sum of the longest lesion diameter (LLD) for all target lesions is calculated and reported as the baseline sum LLD. For malignant lymph nodes identified as target lesions, the short axis diameter is used in the sum of LLD calculation. All other lesions (or sites of disease) are identified as non-target lesions (including bone lesions).

All post-baseline response assessments follow the same lesions identified at baseline. The same mode of assessment (e.g., CT) used to identify/evaluate lesions at baseline are used throughout the course of the study unless subject safety necessitates a change (e.g., allergic reaction to contrast media).

### RECIST Response Criteria

Antitumor activity is evaluated with target and/or non-target lesions according to RECIST Version 1.1 (Eisenhauer E A, Therasse P, Bogaerts J, et al. Eur J Cancer. 2009; 45:228-247) as summarized below.

The target response is defined as the percentage change in target lesion size is evaluated by the following two formulae. First, when determining complete response or partial response, the formula [(Post value−Baseline value)/Baseline value]×100 is used to calculate the target response. Second, when determining progressive disease, the formula [(Post value −Smallest value since treatment started)/(Smallest value since treatment started)]×100 is used to calculate the target response.

Target responses are classified according to the RECIST Version 1.1 Target Lesion Response Criteria in TABLE 10.

TABLE 10
RECIST Target Response Criteria

| Target | |
|---|---|
| Response | |
| Criteria | Definition |
| Complete | Disappearance of all target lesions. Any |
| Response (CR) | pathological lymph nodes (whether target or non-target) |
| | must have reduction in short axis to <10 mm. |
| Partial | At least a 30% decrease in the sum of diameters of |
| Response (PR) | target lesions, taking as reference the baseline sum |
| | diameters. |
| Stable | Neither sufficient shrinkage to qualify for PR nor |
| Disease (SD) | sufficient increase to qualify for PD, taking as reference |
| | the smallest sum diameters while on study. |
| Progressive | At least a 20% increase in the sum of diameters |
| Disease (PD) | of target lesions, taking as reference the smallest sum |
| | diameters while on study (this includes the baseline sum |
| | if that is the smallest on study). In addition to the relative |
| | increase of 20%, the sum must also demonstrate an |
| | absolute increase of 5 mm. (Note: the appearance of one |
| | or more lesions is also considered progression). |

Non-target responses are classified according to the RECIST Version 1.1 Non-Target Lesion Response Criteria in TABLE 11.

TABLE 11
RECIST Non-Target Response Criteria

| Non-Target | |
|---|---|
| Response | |
| Criteria | Definition |
| CR | Disappearance of all non-target lesions and normalization |
| | of tumor marker level. All lymph nodes must be non- |
| | pathological in size (<10 mm short axis). |
| Non-CR/ | Persistence of one or more non-target lesion(s) and/or |
| Non-PD | maintenance of tumor marker level above the normal |
| | limits. |
| PD | Unequivocal progression of existing non-target lesions. |
| | (Note: the appearance of one or more new lesions is also |
| | considered progression). |

TABLE 12
RECIST Overall Response Criteria

| Target Lesions | Non-Target Lesions | New Lesions | Overall Response |
|---|---|---|---|
| CR | CR | No | CR |
| CR | Non-CR/Non-PD | No | PR |
| CR | Not Evaluated | No | PR |
| PR | Non-PD or not all | No | PR |
| | evaluated | | |
| SD | Non-PD or not all | No | SD |
| | evaluated | | |
| Not all | Non-PD | No | Inevaluable |
| evaluated | | | |
| PD | Any | Yes or No | PD |
| Any | PD | Yes or No | PD |
| Any | Any | Yes | PD |

Overall responses are classified according to the RECIST Version 1.1 Overall Response Criteria in TABLE 12.

### Exploratory Endpoints Analysis

Immune responses are detected and quantified in flow cytometry-based and serum assays. Immunogenicity of Ad5 [E1−, E2b−]-HER2/neu is detected by flow cytometric analysis of T-cell frequency, activation status, cytokine profiles, and antibody levels.

Genomic sequencing of tumor cells relative to non-tumor cells from whole blood is profiled to identify the genomic variances that may contribute to response or disease progression and provide an understanding of molecular abnormalities. RNA sequencing is conducted to provide expression data and give relevance to DNA mutations. Quantitative proteomics analysis is conducted to determine the exact amounts of specific proteins and to confirm expression of genes that are correlative of response to vaccine immunotherapy and disease progression.

### Pharmacodynamic Assessments

Pharmacodynamics of the Ad5 [E1−, E2b−]-HER2/neu vaccine is assessed by peripheral blood collection and immune assessments of the collected samples. Approximately 80 mL of peripheral blood is drawn from subjects to evaluate the study drug's effect on the immune response at specific time points during the study and/or after a specified injection. Blood draws are done at baseline, prior to each injection, and approximately 3 weeks after the third injection (Week 9); and prior to each booster injection (Week 18, 30, and 42), and 3 weeks after each booster injection (Week 21, 33, and 45). Six, 10-mL green top sodium heparin tubes for PBMC samples and two 8-mL serum-separating tubes for serum samples are drawn. Immune assessments include flow cytometry-based and serum assays.

PBMCs are analyzed as follows. Pre- and post-therapy PBMCs, separated by Ficoll-Hypaque density gradient separation, are analyzed for antigen-specific immune responses using an intracellular cytokine staining assay. PBMCs are stimulated in vitro with overlapping 15-mer peptide pools encoding the tumor-associated antigen HER2/neu. Control peptide pools involve the use of human leukocyte antigen peptide as a negative control and CEFT peptide mix as a positive control. CEFT is a mixture of peptides of CMV, Epstein-Barr virus, influenza, and tetanus toxin. Post-stimulation analyses of CD4 and CD8 T cells involve the production of IFN-γ, IL-2, tumor necrosis factor, and CD107a. If sufficient PBMCs are available, assays are performed for the development of T cells to other tumor-associated antigens. PBMCs are evaluated for changes in standard immune cell types (CD4 and CD8 T cells, natural killer [NK] cells, regulatory T cells [Tregs], myeloid-derived suppressor cells [MDSCs], and dendritic cells) as well as 123 immune cell subsets. If sufficient PBMCs are available, PBMCs from selected subjects are analyzed for function of specific immune cell subsets, including CD4 and CD8 T cells, NK cells, Tregs, and MDSCs.

Soluble factors are analyzed as follows. Sera are analyzed pre- and post-therapy for the following soluble factors: soluble CD27, soluble CD40 ligand, and antibodies to HER2/neu and other tumor-associated antigens.

### Genomics and Proteomics Molecular Analysis and Analysis of Tumor and Whole Blood

Genomic sequencing of tumor cells from tissue relative to non-tumor cells from whole blood is profiled to identify the genomic variances that may contribute to response or disease progression and provide an understanding of molecular abnormalities. RNA sequencing is conducted to provide expression data and give relevance to DNA mutations. Quantitative proteomics analysis is conducted to determine the exact amounts of specific proteins and to confirm expression of genes that are correlative of response to vaccine immunotherapy and disease progression.

Genomics and proteomics molecular profiling are performed on formalin-fixed, paraffin embedded (FFPE) tumor tissue and whole blood (subject matched normal comparator against the tumor tissue) by next-generation sequencing and mass spectrometry-based quantitative proteomics. Collection of tumor tissue and whole blood is mandatory for this study. Tumor tissue and whole blood are obtained at screening.

A single FFPE tumor tissue block or slides are used for the extraction of tumor DNA, tumor RNA, and tumor protein. A whole blood sample is used for the extraction of subject normal DNA. Tumor tissue and whole blood are processed in the NantOmics, LLC CLIA-registered and CAP-accredited/CLIA-certified laboratories.

### Statistical Methods

The rate of DLTs and the MTD or HTD is assessed. Overall safety is assessed by descriptive analyses using tabulated frequencies of AEs by grade using CTCAE Version 4.03 within dose cohorts and for the overall study population in terms of treatment-emergent AEs, SAEs, and clinically significant changes in safety laboratory tests, physical examinations, ECGs, LVEF, and vital signs. ORR and DCR are evaluated according to RECIST Version 1.1 by dose cohort and overall; duration of response is also evaluated. PFS and OS are analyzed using Kaplan-Meier methods by dose cohort and overall.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

SEQUENCES

| SEQ | |
|---|---|
| ID | |
| NO | Sequence |
| SEQ | CTAGAATGGAGCTGGCGGCCTTGTGCCGCTGGGGGCTCCTCC |
| ID | TCGCCCTCTTGCCCCCCGGAGCCGCGAGCACCCAAGTGTGCA |
| NO: | CCGGCACAGACATGAAGCTGCGGCTCCCTGCCAGTCCCGAGA |
| 1 | CCCACCTGGACATGCTCCGCCACCTCTACCAGGGCTGCCAGG |
| | TGGTGCAGGGAAACCTGGAACTCACCTACCTGCCCACCAATG |
| | CCAGCCTGTCCTTCCTGCAGGATATCCAGGAGGTGCAGGGCT |
| | ACGTGCTCATCGCTCACAACCAAGTGAGGCAGGTCCCACTGC |
| | AGAGGCTGCGGATTGTGCGAGGCACCCAGCTCTTTGAGGACA |
| | ACTATGCCCTGGCCGTGCTAGACAATGGAGACCCGCTGAACA |
| | ATACCACCCCTGTCACAGGGGCCTCCCCAGGAGGCCTGCGGG |
| | AGCTGCAGCTTCGAAGCCTCACAGAGATCTTGAAAGGAGGGG |
| | TCTTGATCCAGCGGAACCCCCAGCTCTGCTACCAGGACACGA |
| | TTTTGTGGAAGGACATCTTCCACAAGAACAACCAGCTGGCTC |
| | TCACACTGATAGACACCAACCGCTCTCGGGCCTGCCACCCCT |
| | GTTCTCCGATGTGTAAGGGCTCCCGCTGCTGGGGAGAGAGTT |
| | CTGAGGATTGTCAGAGCCTGACGCGCACTGTCTGTGCCGGTG |
| | GCTGTGCCCGCTGCAAGGGGCCACTGCCCACTGACTGCTGCC |
| | ATGAGCAGTGTGCTGCCGGCTGCACGGGCCCCAAGCACTCTG |
| | ACTGCCTGGCCTGCCTCCACTTCAACCACAGTGGCATCTGTGA |
| | GCTGCACTGCCCAGCCCTGGTCACCTACAACACAGACACGTT |
| | TGAGTCCATGCCCAATCCCGAGGGCCGGTATACATTCGGCGC |
| | CAGCTGTGTGACTGCCTGTCCCTACAACTACCTTTCTACGGAC |
| | GTGGGATCCTGCACCCTCGTCTGCCCCCTGCACAACCAAGAG |
| | GTGACAGCAGAGGATGGAACACAGCGGTGTGAGAAGTGCAG |
| | CAAGCCCTGTGCCCGAGTGTGCTATGGTCTGGGCATGGAGCA |
| | CTTGCGAGAGGTGAGGGCAGTTACCAGTGCCAATATCCAGGA |
| | GTTTGCTGGCTGCAAGAAGATCTTTGGGAGCCTGGCATTTCTG |
| | CCGGAGAGCTTTGATGGGGACCCAGCCTCCAACACTGCCCCG |
| | CTCCAGCCAGAGCAGCTCCAAGTGTTTGAGACTCTGGAAGAG |
| | ATCACAGGTTACCTATACATCTCAGCATGGCCGGACAGCCTG |
| | CCTGACCTCAGCGTCTTCCAGAACCTGCAAGTAATCCGGGGA |
| | CGAATTCTGCACAATGGCGCCTACTCGCTGACCCTGCAAGGG |
| | CTGGGCATCAGCTGGCTGGGGCTGCGCTCACTGAGGGAACTG |
| | GGCAGTGGACTGGCCCTCATCCACCATAACACCCACCTCTGC |
| | TTCGTGCACACGGTGCCCTGGGACCAGCTCTTTCGGAACCCG |
| | CACCAAGCTCTGCTCCACACTGCCAACCGGCCAGAGGACGAG |
| | TGTGTGGGCGAGGGCCTGGCCTGCCACCAGCTGTGCGCCCGA |
| | GGGCACTGCTGGGGTCCAGGGCCCACCCAGTGTGTCAACTGC |
| | AGCCAGTTCCTTCGGGGCCAGGAGTGCGTGGAGGAATGCCGA |
| | GTACTGCAGGGGCTCCCCAGGGAGTATGTGAATGCCAGGCAC |
| | TGTTTGCCGTGCCACCCTGAGTGTCAGCCCCAGAATGGCTCA |
| | GTGACCTGTTTTGGACCGGAGGCTGACCAGTGTGTGGCCTGT |
| | GCCCACTATAAGGACCCTCCCTTCTGCGTGGCCCGCTGCCCCA |
| | GCGGTGTGAAACCTGACCTCTCCTACATGCCCATCTGGAAGT |
| | TTCCAGATGAGGAGGGCGCATGCCAGCCTTGCCCCATCAACT |
| | GCACCCACTCCTGTGTGGACCTGGATGACAAGGGCTGCCCCG |
| | CCGAGCAGAGAGCCAGCCCTCTGACGTCCATCGTCTCTGCGG |
| | TGGTTGGCATTCTGCTGGTCGTGGTCTTGGGGGTGGTCTTTGG |
| | GATCCTCATCAAGCGACGGCAGCAGAAGATCCGGAAGTACA |
| | CGTAATCTAGATAA |
| SEQ | MRMELAALCRWGLLLALLPPGAASTQVCTGTDMKLRLPASPET |
| ID | HLDMLRHLYQGCQVVQGNLELTYLPTNASLSFLQDIQEVQGYV |
| NO: | LIAHNQVRQVPLQRLRIVRGTQLFEDNYALAVLDNGDPLNNTTP |
| 2 | VTGASPGGLRELQLRSLTEILKGGVLIQRNPQLCYQDTILWKDIF |
| | HKNNQLALTLIDTNRSRACHPCSPMCKGSRCWGESSEDCQSLTR |
| | TVCAGGCARCKGPLPTDCCHEQCAAGCTGPKHSDCLACLHFNH |
| | SGICELHCPALVTYNTDTFESMPNPEGRYTFGASCVTACPYNYL |
| | STDVGSCTLVCPLHNQEVTAEDGTQRCEKCSKPCARVCYGLGM |
| | EHLREVRAVTSANIQEFAGCKKIFGSLAFLPESFDGDPASNTAPL |
| | QPEQLQVFETLEEITGYLYISAWPDSLPDLSVFQNLQVIRGRILHN |
| | GAYSLTLQGLGISWLGLRSLRELGSGLALIHHNTHLCFVHTVPW |
| | DQLFRNPHQALLHTANRPEDECVGEGLACHQLCARGHCWGPGP |
| | TQCVNCSQFLRGQECVEECRVLQGLPREYVNARHCLPCHPECQP |
| | QNGSVTCFGPEADQCVACAHYKDPPFCVARCPSGVKPDLSYMPI |
| | WKFPDEEGACQPCPINCTHSCVDLDDKGCPAEQRASPLTSIVSA |
| | VVGILLVVVLGVVFGILIKRRQQKIRKYT |
| SEQ | CATCATCAATAATATACCTTATTTTGGATTGAAGCCAATATGA |
| ID | TAATGAGGGGGTGGAGTTTGTGACGTGGCGCGGGGCGTGGG |
| NO: | AACGGGGCGGGTGACGTAGTAGTGTGGCGGAAGTGTGATGTT |
| 3 | GCAAGTGTGGCGGAACACATGTAAGCGACGGATGTGGCAAA |
| | AGTGACGTTTTTGGTGTGCGCCGGTGTACACAGGAAGTGACA |
| | ATTTTCGCGCGGTTTTAGGCGGATGTTGTAGTAAATTTGGGCG |
| | TAACCGAGTAAGATTTGGCCATTTTCGCGGGAAAACTGAATA |
| | AGAGGAAGTGAAATCTGAATAATTTTGTGTTACTCATAGCGC |
| | GTAATACTGTAATAGTAATCAATTACGGGGTCATTAGTTCAT |
| | AGCCCATATATGGAGTTCCGCGTTACATAACTTACGGTAAAT |
| | GGCCCGCCTGGCTGACCGCCCAACGACCCCCGCCCATTGACG |
| | TCAATAATGACGTATGTTCCCATAGTAACGCCAATAGGGACT |
| | TTCCATTGACGTCAATGGGTGGAGTATTTACGGTAAACTGCC |
| | CACTTGGCAGTACATCAAGTGTATCATATGCCAAGTACGCCC |
| | CCTATTGACGTCAATGACGGTAAATGGCCCGCCTGGCATTAT |
| | GCCCAGTACATGACCTTATGGGACTTTCCTACTTGGCAGTACA |
| | TCTACGTATTAGTCATCGCTATTACCATGGTGATGCGGTTTTG |
| | GCAGTACATCAATGGGCGTGGATAGCGGTTTGACTCACGGGG |
| | ATTTCCAAGTCTCCACCCCATTGACGTCAATGGGAGTTTGTTT |
| | TGGCACCAAAATCAACGGGACTTTCCAAAATGTCGTAACAAC |
| | TCCGCCCCATTGACGCAAATGGGCGGTAGGCGTGTACGGTGG |
| | GAGGTCTATATAAGCAGAGCTGGTTTAGTGAACCGTCAGATC |
| | CGCTAGAGATCTGGTACCGTCGACGCGGCCGCTCGAGCCTAA |
| | GCTTCTAGATGCATGCTCGAGCGGCCGCCAGTGTGATGGATA |
| | TCTGCAGAATTCGCCCTTGCTCTAGAATGGAGCTGGCGGCCTT |
| | GTGCCGCTGGGGGCTCCTCCTCGCCCTCTTGCCCCCCGGAGCC |
| | GCGAGCACCCAAGTGTGCACCGGCACAGACATGAAGCTGCG |
| | GCTCCCTGCCAGTCCCGAGACCCACCTGGACATGCTCCGCCA |
| | CCTCTACCAGGGCTGCCAGGTGGTGCAGGGAAACCTGGAACT |
| | CACCTACCTGCCCACCAATGCCAGCCTGTCCTTCCTGCAGGAT |
| | ATCCAGGAGGTGCAGGGCTACGTGCTCATCGCTCACAACCAA |
| | GTGAGGCAGGTCCCACTGCAGAGGCTGCGGATTGTGCGAGGC |
| | ACCCAGCTCTTTGAGGACAACTATGCCCTGGCCGTGCTAGAC |
| | AATGGAGACCCGCTGAACAATACCACCCCTGTCACAGGGGCC |
| | TCCCCAGGAGGCCTGCGGGAGCTGCAGCTTCGAAGCCTCACA |
| | GAGATCTTGAAAGGAGGGGTCTTGATCCAGCGGAACCCCCAG |
| | CTCTGCTACCAGGACACGATTTTGTGGAAGGACATCTTCCAC |
| | AAGAACAACCAGCTGGCTCTCACACTGATAGACACCAACCGC |
| | TCTCGGGCCTGCCACCCCTGTTCTCCGATGTGTAAGGGCTCCC |
| | GCTGCTGGGGAGAGAGTTCTGAGGATTGTCAGAGCCTGACGC |
| | GCACTGTCTGTGCCGGTGGCTGTGCCCGCTGCAAGGGGCCAC |
| | TGCCCACTGACTGCTGCCATGAGCAGTGTGCTGCCGGCTGCA |
| | CGGGCCCCAAGCACTCTGACTGCCTGGCCTGCCTCCACTTCA |
| | ACCACAGTGGCATCTGTGAGCTGCACTGCCCAGCCCTGGTCA |
| | CCTACAACACAGACACGTTTGAGTCCATGCCCAATCCCGAGG |
| | GCCGGTATACATTCGGCGCCAGCTGTGTGACTGCCTGTCCCTA |
| | CAACTACCTTTCTACGGACGTGGGATCCTGCACCCTCGTCTGC |
| | CCCCTGCACAACCAAGAGGTGACAGCAGAGGATGGAACACA |
| | GCGGTGTGAGAAGTGCAGCAAGCCCTGTGCCCGAGTGTGCTA |
| | TGGTCTGGGCATGGAGCACTTGCGAGAGGTGAGGGCAGTTAC |
| | CAGTGCCAATATCCAGGAGTTTGCTGGCTGCAAGAAGATCTT |
| | TGGGAGCCTGGCATTTCTGCCGGAGAGCTTTGATGGGGACCC |
| | AGCCTCCAACACTGCCCCGCTCCAGCCAGAGCAGCTCCAAGT |
| | GTTTGAGACTCTGGAAGAGATCACAGGTTACCTATACATCTC |
| | AGCATGGCCGGACAGCCTGCCTGACCTCAGCGTCTTCCAGAA |
| | CCTGCAAGTAATCCGGGGACGAATTCTGCACAATGGCGCCTA |
| | CTCGCTGACCCTGCAAGGGCTGGGCATCAGCTGGCTGGGGCT |
| | GCGCTCACTGAGGGAACTGGGCAGTGGACTGGCCCTCATCCA |
| | CCATAACACCCACCTCTGCTTCGTGCACACGGTGCCCTGGGA |
| | CCAGCTCTTTCGGAACCCGCACCAAGCTCTGCTCCACACTGCC |
| | AACCGGCCAGAGGACGAGTGTGTGGGCGAGGGCCTGGCCTG |
| | CCACCAGCTGTGCGCCCGAGGGCACTGCTGGGGTCCAGGGCC |
| | CACCCAGTGTGTCAACTGCAGCCAGTTCCTTCGGGGCCAGGA |
| | GTGCGTGGAGGAATGCCGAGTACTGCAGGGGCTCCCCAGGG |
| | AGTATGTGAATGCCAGGCACTGTTTGCCGTGCCACCCTGAGT |
| | GTCAGCCCCAGAATGGCTCAGTGACCTGTTTTGGACCGGAGG |
| | CTGACCAGTGTGTGGCCTGTGCCCACTATAAGGACCCTCCCTT |
| | CTGCGTGGCCCGCTGCCCCAGCGGTGTGAAACCTGACCTCTC |
| | CTACATGCCCATCTGGAAGTTTCCAGATGAGGAGGGCGCATG |
| | CCAGCCTTGCCCCATCAACTGCACCCACTCCTGTGTGGACCTG |
| | GATGACAAGGGCTGCCCCGCCGAGCAGAGAGCCAGCCCTCTG |
| | ACGTCCATCGTCTCTGCGGTGGTTGGCATTCTGCTGGTCGTGG |
| | TCTTGGGGGTGGTCTTTGGGATCCTCATCAAGCGACGGCAGC |
| | AGAAGATCCGGAAGTACACGTAATCTAGATAAGATATCCGAT |
| | CCACCGGATCTAGATAACTGATCATAATCAGCCATACCACAT |
| | TTGTAGAGGTTTTACTTGCTTTAAAAAACCTCCCACACCTCCC |
| | CCTGAACCTGAAACATAAAATGAATGCAATTGTTGTTGTTAA |
| | CTTGTTTATTGCAGCTTATAATGGTTACAAATAAAGCAATAGC |
| | ATCACAAATTTCACAAATAAAGCATTTTTTTCACTGCATTCTA |
| | GTTGTGGTTTGTCCAAACTCATCAATGTATCTTAACGCGGATC |
| | TGGAAGGTGCTGAGGTACGATGAGACCCGCACCAGGTGCAG |
| | ACCCTGCGAGTGTGGCGGTAAACATATTAGGAACCAGCCTGT |
| | GATGCTGGATGTGACCGAGGAGCTGAGGCCCGATCACTTGGT |
| | GCTGGCCTGCACCCGCGCTGAGTTTGGCTCTAGCGATGAAGA |
| | TACAGATTGAGGTACTGAAATGTGTGGGCGTGGCTTAAGGGT |
| | GGGAAAGAATATATAAGGTGGGGGTCTTATGTAGTTTTGTAT |
| | CTGTTTTGCAGCAGCCGCCGCCGCCATGAGCACCAACTCGTTT |
| | GATGGAAGCATTGTGAGCTCATATTTGACAACGCGCATGCCC |
| | CCATGGGCCGGGGTGCGTCAGAATGTGATGGGCTCCAGCATT |
| | GATGGTCGCCCCGTCCTGCCCGCAAACTCTACTACCTTGACCT |
| | ACGAGACCGTGTCTGGAACGCCGTTGGAGACTGCAGCCTCCG |
| | CCGCCGCTTCAGCCGCTGCAGCCACCGCCCGCGGGATTGTGA |
| | CTGACTTTGCTTTCCTGAGCCCGCTTGCAAGCAGTGCAGCTTC |
| | CCGTTCATCCGCCCGCGATGACAAGTTGACGGCTCTTTTGGCA |
| | CAATTGGATTCTTTGACCCGGGAACTTAATGTCGTTTCTCAGC |
| | AGCTGTTGGATCTGCGCCAGCAGGTTTCTGCCCTGAAGGCTTC |
| | CTCCCCTCCCAATGCGGTTTAAAACATAAATAAAAAACCAGA |
| | CTCTGTTTGGATTTGGATCAAGCAAGTGTCTTGCTGTCTTTAT |
| | TTAGGGGTTTTGCGCGCGCGGTAGGCCCGGGACCAGCGGTCT |
| | CGGTCGTTGAGGGTCCTGTGTATTTTTTCCAGGACGTGGTAAA |
| | GGTGACTCTGGATGTTCAGATACATGGGCATAAGCCCGTCTC |
| | TGGGGTGGAGGTAGCACCACTGCAGAGCTTCATGCTGCGGGG |
| | TGGTGTTGTAGATGATCCAGTCGTAGCAGGAGCGCTGGGCGT |
| | GGTGCCTAAAAATGTCTTTCAGTAGCAAGCTGATTGCCAGGG |
| | GCAGGCCCTTGGTGTAAGTGTTTACAAAGCGGTTAAGCTGGG |
| | ATGGGTGCATACGTGGGGATATGAGATGCATCTTGGACTGTA |
| | TTTTTAGGTTGGCTATGTTCCCAGCCATATCCCTCCGGGGATT |
| | CATGTTGTGCAGAACCACCAGCACAGTGTATCCGGTGCACTT |
| | GGGAAATTTGTCATGTAGCTTAGAAGGAAATGCGTGGAAGAA |
| | CTTGGAGACGCCCTTGTGACCTCCAAGATTTTCCATGCATTCG |
| | TCCATAATGATGGCAATGGGCCCACGGGCGGCGGCCTGGGCG |
| | AAGATATTTCTGGGATCACTAACGTCATAGTTGTGTTCCAGG |
| | ATGAGATCGTCATAGGCCATTTTTACAAAGCGCGGGCGGAGG |
| | GTGCCAGACTGCGGTATAATGGTTCCATCCGGCCCAGGGGCG |
| | TAGTTACCCTCACAGATTTGCATTTCCCACGCTTTGAGTTCAG |
| | ATGGGGGGATCATGTCTACCTGCGGGGCGATGAAGAAAACG |
| | GTTTCCGGGGTAGGGGAGATCAGCTGGGAAGAAAGCAGGTT |
| | CCTGAGCAGCTGCGACTTACCGCAGCCGGTGGGCCCGTAAAT |
| | CACACCTATTACCGGCTGCAACTGGTAGTTAAGAGAGCTGCA |
| | GCTGCCGTCATCCCTGAGCAGGGGGGCCACTTCGTTAAGCAT |
| | GTCCCTGACTCGCATGTTTTCCCTGACCAAATCCGCCAGAAG |
| | GCGCTCGCCGCCCAGCGATAGCAGTTCTTGCAAGGAAGCAAA |
| | GTTTTTCAACGGTTTGAGACCGTCCGCCGTAGGCATGCTTTTG |
| | AGCGTTTGACCAAGCAGTTCCAGGCGGTCCCACAGCTCGGTC |
| | ACCTGCTCTACGGCATCTCGATCCAGCATATCTCCTCGTTTCG |
| | CGGGTTGGGGCGGCTTTCGCTGTACGGCAGTAGTCGGTGCTC |
| | GTCCAGACGGGCCAGGGTCATGTCTTTCCACGGGCGCAGGGT |
| | CCTCGTCAGCGTAGTCTGGGTCACGGTGAAGGGGTGCGCTCC |
| | GGGCTGCGCGCTGGCCAGGGTGCGCTTGAGGCTGGTCCTGCT |
| | GGTGCTGAAGCGCTGCCGGTCTTCGCCCTGCGCGTCGGCCAG |
| | GTAGCATTTGACCATGGTGTCATAGTCCAGCCCCTCCGCGGC |
| | GTGGCCCTTGGCGCGCAGCTTGCCCTTGGAGGAGGCGCCGCA |
| | CGAGGGGCAGTGCAGACTTTTGAGGGCGTAGAGCTTGGGCGC |
| | GAGAAATACCGATTCCGGGGAGTAGGCATCCGCGCCGCAGG |
| | CCCCGCAGACGGTCTCGCATTCCACGAGCCAGGTGAGCTCTG |
| | GCCGTTCGGGGTCAAAAACCAGGTTTCCCCCATGCTTTTTGAT |
| | GCGTTTCTTACCTCTGGTTTCCATGAGCCGGTGTCCACGCTCG |
| | GTGACGAAAAGGCTGTCCGTGTCCCCGTATACAGACTTGAGA |
| | GGCCTGTCCTCGAGCGGTGTTCCGCGGTCCTCCTCGTATAGAA |
| | ACTCGGACCACTCTGAGACAAAGGCTCGCGTCCAGGCCAGCA |
| | CGAAGGAGGCTAAGTGGGAGGGGTAGCGGTCGTTGTCCACTA |
| | GGGGGTCCACTCGCTCCAGGGTGTGAAGACACATGTCGCCCT |
| | CTTCGGCATCAAGGAAGGTGATTGGTTTGTAGGTGTAGGCCA |
| | CGTGACCGGGTGTTCCTGAAGGGGGGCTATAAAAGGGGGTG |
| | GGGGCGCGTTCGTCCTCACTCTCTTCCGCATCGCTGTCTGCGA |
| | GGGCCAGCTGTTGGGGTGAGTACTCCCTCTGAAAAGCGGGCA |
| | TGACTTCTGCGCTAAGATTGTCAGTTTCCAAAAACGAGGAGG |
| | ATTTGATATTCACCTGGCCCGCGGTGATGCCTTTGAGGGTGGC |
| | CGCATCCATCTGGTCAGAAAAGACAATCTTTTTGTTGTCAAGC |
| | TTGGTGGCAAACGACCCGTAGAGGGCGTTGGACAGCAACTTG |
| | GCGATGGAGCGCAGGGTTTGGTTTTTGTCGCGATCGGCGCGC |
| | TCCTTGGCCGCGATGTTTAGCTGCACGTATTCGCGCGCAACGC |
| | ACCGCCATTCGGGAAAGACGGTGGTGCGCTCGTCGGGCACCA |
| | GGTGCACGCGCCAACCGCGGTTGTGCAGGGTGACAAGGTCAA |
| | CGCTGGTGGCTACCTCTCCGCGTAGGCGCTCGTTGGTCCAGC |
| | AGAGGCGGCCGCCCTTGCGCGAGCAGAATGGCGGTAGGGGG |
| | TCTAGCTGCGTCTCGTCCGGGGGGTCTGCGTCCACGGTAAAG |
| | ACCCCGGGCAGCAGGCGCGCGTCGAAGTAGTCTATCTTGCAT |
| | CCTTGCAAGTCTAGCGCCTGCTGCCATGCGCGGGCGGCAAGC |
| | GCGCGCTCGTATGGGTTGAGTGGGGGACCCCATGGCATGGGG |
| | TGGGTGAGCGCGGAGGCGTACATGCCGCAAATGTCGTAAACG |
| | TAGAGGGGCTCTCTGAGTATTCCAAGATATGTAGGGTAGCAT |
| | CTTCCACCGCGGATGCTGGCGCGCACGTAATCGTATAGTTCG |
| | TGCGAGGGAGCGAGGAGGTCGGGACCGAGGTTGCTACGGGC |
| | GGGCTGCTCTGCTCGGAAGACTATCTGCCTGAAGATGGCATG |
| | TGAGTTGGATGATATGGTTGGACGCTGGAAGACGTTGAAGCT |
| | GGCGTCTGTGAGACCTACCGCGTCACGCACGAAGGAGGCGTA |
| | GGAGTCGCGCAGCTTGTTGACCAGCTCGGCGGTGACCTGCAC |
| | GTCTAGGGCGCAGTAGTCCAGGGTTTCCTTGATGATGTCATA |
| | CTTATCCTGTCCCTTTTTTTTCCACAGCTCGCGGTTGAGGACA |
| | AACTCTTCGCGGTCTTTCCAGTACTCTTGGATCGGAAACCCGT |
| | CGGCCTCCGAACGGTAAGAGCCTAGCATGTAGAACTGGTTGA |
| | CGGCCTGGTAGGCGCAGCATCCCTTTTCTACGGGTAGCGCGT |
| | ATGCCTGCGCGGCCTTCCGGCATGACCAGCATGAAGGGCACG |
| | AGCTGCTTCCCAAAGGCCCCCATCCAAGTATAGGTCTCTACA |
| | TCGTAGGTGACAAAGAGACGCTCGGTGCGAGGATGCGAGCC |
| | GATCGGGAAGAACTGGATCTCCCGCCACCAATTGGAGGAGTG |
| | GCTATTGATGTGGTGAAAGTAGAAGTCCCTGCGACGGGCCGA |
| | ACACTCGTGCTGGCTTTTGTAAAAACGTGCGCAGTACTGGCA |
| | GCGGTGCACGGGCTGTACATCCTGCACGAGGTTGACCTGACG |
| | ACCGCGCACAAGGAAGCAGAGTGGGAATTTGAGCCCCTCGCC |
| | TGGCGGGTTTGGCTGGTGGTCTTCTACTTCGGCTGCTTGTCCT |
| | TGACCGTCTGGCTGCTCGAGGGGAGTTACGGTGGATCGGACC |
| | ACCACGCCGCGCGAGCCCAAAGTCCAGATGTCCGCGCGCGGC |
| | GGTCGGAGCTTGATGACAACATCGCGCAGATGGGAGCTGTCC |
| | ATGGTCTGGAGCTCCCGCGGCGTCAGGTCAGGCGGGAGCTCC |
| | TGCAGGTTTACCTCGCATAGACGGGTCAGGGCGCGGGCTAGA |
| | TCCAGGTGATACCTAATTTCCAGGGGCTGGTTGGTGGCGGCG |
| | TCGATGGCTTGCAAGAGGCCGCATCCCCGCGGCGCGACTACG |
| | GTACCGCGCGGCGGGCGGTGGGCCGCGGGGGTGTCCTTGGAT |
| | GATGCATCTAAAAGCGGTGACGCGGGCGAGCCCCCGGAGGT |
| | AGGGGGGGCTCCGGACCCGCCGGGAGAGGGGGCAGGGGCAC |
| | GTCGGCGCCGCGCGCGGGCAGGAGCTGGTGCTGCGCGCGTAG |
| | GTTGCTGGCGAACGCGACGACGCGGCGGTTGATCTCCTGAAT |
| | CTGGCGCCTCTGCGTGAAGACGACGGGCCCGGTGAGCTTGAA |
| | CCTGAAAGAGAGTTCGACAGAATCAATTTCGGTGTCGTTGAC |
| | GGCGGCCTGGCGCAAAATCTCCTGCACGTCTCCTGAGTTGTCT |
| | TGATAGGCGATCTCGGCCATGAACTGCTCGATCTCTTCCTCCT |
| | GGAGATCTCCGCGTCCGGCTCGCTCCACGGTGGCGGCGAGGT |
| | CGTTGGAAATGCGGGCCATGAGCTGCGAGAAGGCGTTGAGG |
| | CCTCCCTCGTTCCAGACGCGGCTGTAGACCACGCCCCCTTCGG |
| | CATCGCGGGCGCGCATGACCACCTGCGCGAGATTGAGCTCCA |
| | CGTGCCGGGCGAAGACGGCGTAGTTTCGCAGGCGCTGAAAG |
| | AGGTAGTTGAGGGTGGTGGCGGTGTGTTCTGCCACGAAGAAG |
| | TACATAACCCAGCGTCGCAACGTGGATTCGTTGATAATTGTT |
| | GTGTAGGTACTCCGCCGCCGAGGGACCTGAGCGAGTCCGCAT |
| | CGACCGGATCGGAAAACCTCTCGAGAAAGGCGTCTAACCAGT |
| | CACAGTCGCAAGGTAGGCTGAGCACCGTGGCGGGCGGCAGC |
| | GGGCGGCGGTCGGGGTTGTTTCTGGCGGAGGTGCTGCTGATG |
| | ATGTAATTAAAGTAGGCGGTCTTGAGACGGCGGATGGTCGAC |
| | AGAAGCACCATGTCCTTGGGTCCGGCCTGCTGAATGCGCAGG |
| | CGGTCGGCCATGCCCCAGGCTTCGTTTTGACATCGGCGCAGG |
| | TCTTTGTAGTAGTCTTGCATGAGCCTTTCTACCGGCACTTCTT |
| | CTTCTCCTTCCTCTTGTCCTGCATCTCTTGCATCTATCGCTGCG |
| | GCGGCGGCGGAGTTTGGCCGTAGGTGGCGCCCTCTTCCTCCC |
| | ATGCGTGTGACCCCGAAGCCCCTCATCGGCTGAAGCAGGGCT |
| | AGGTCGGCGACAACGCGCTCGGCTAATATGGCCTGCTGCACC |
| | TGCGTGAGGGTAGACTGGAAGTCATCCATGTCCACAAAGCGG |
| | TGGTATGCGCCCGTGTTGATGGTGTAAGTGCAGTTGGCCATA |
| | ACGGACCAGTTAACGGTCTGGTGACCCGGCTGCGAGAGCTCG |
| | GTGTACCTGAGACGCGAGTAAGCCCTCGAGTCAAATACGTAG |
| | TCGTTGCAAGTCCGCACCAGGTACTGGTATCCCACCAAAAAG |
| | TGCGGCGGCGGCTGGCGGTAGAGGGGCCAGCGTAGGGTGGC |
| | CGGGGCTCCGGGGGCGAGATCTTCCAACATAAGGCGATGATA |
| | TCCGTAGATGTACCTGGACATCCAGGTGATGCCGGCGGCGGT |
| | GGTGGAGGCGCGCGGAAAGTCGCGGACGCGGTTCCAGATGTT |
| | GCGCAGCGGCAAAAAGTGCTCCATGGTCGGGACGCTCTGGCC |
| | GGTCAGGCGCGCGCAATCGTTGACGCTCTAGCGTGCAAAAGG |
| | AGAGCCTGTAAGCGGGCACTCTTCCGTGGTCTGGTGGATAAA |
| | TTCGCAAGGGTATCATGGCGGACGACCGGGGTTCGAGCCCCG |
| | TATCCGGCCGTCCGCCGTGATCCATGCGGTTACCGCCCGCGT |
| | GTCGAACCCAGGTGTGCGACGTCAGACAACGGGGGAGTGCTC |
| | CTTTTGGCTTCCTTCCAGGCGCGGCGGCTGCTGCGCTAGCTTT |
| | TTTGGCCACTGGCCGCGCGCAGCGTAAGCGGTTAGGCTGGAA |
| | AGCGAAAGCATTAAGTGGCTCGCTCCCTGTAGCCGGAGGGTT |
| | ATTTTCCAAGGGTTGAGTCGCGGGACCCCCGGTTCGAGTCTC |
| | GGACCGGCCGGACTGCGGCGAACGGGGGTTTGCCTCCCCGTC |
| | ATGCAAGACCCCGCTTGCAAATTCCTCCGGAAACAGGGACGA |
| | GCCCCTTTTTTGCTTTTCCCAGATGCATCCGGTGCTGCGGCAG |
| | ATGCGCCCCCCTCCTCAGCAGCGGCAAGAGCAAGAGCAGCG |
| | GCAGACATGCAGGGCACCCTCCCCTCCTCCTACCGCGTCAGG |
| | AGGGGCGACATCCGCGGTTGACGCGGCAGCAGATGGTGATTA |
| | CGAACCCCCGCGGCGCCGGGCCCGGCACTACCTGGACTTGGA |
| | GGAGGGCGAGGGCCTGGCGCGGCTAGGAGCGCCCTCTCCTGA |
| | GCGGCACCCAAGGGTGCAGCTGAAGCGTGATACGCGTGAGG |
| | CGTACGTGCCGCGGCAGAACCTGTTTCGCGACCGCGAGGGAG |
| | AGGAGCCCGAGGAGATGCGGGATCGAAAGTTCCACGCAGGG |
| | CGCGAGCTGCGGCATGGCCTGAATCGCGAGCGGTTGCTGCGC |
| | GAGGAGGACTTTGAGCCCGACGCGCGAACCGGGATTAGTCCC |
| | GCGCGCGCACACGTGGCGGCCGCCGACCTGGTAACCGCATAC |
| | GAGCAGACGGTGAACCAGGAGATTAACTTTCAAAAAAGCTTT |
| | AACAACCACGTGCGTACGCTTGTGGCGCGCGAGGAGGTGGCT |
| | ATAGGACTGATGCATCTGTGGGACTTTGTAAGCGCGCTGGAG |
| | CAAAACCCAAATAGCAAGCCGCTCATGGCGCAGCTGTTCCTT |
| | ATAGTGCAGCACAGCAGGGACAACGAGGCATTCAGGGATGC |
| | GCTGCTAAACATAGTAGAGCCCGAGGGCCGCTGGCTGCTCGA |
| | TTTGATAAACATCCTGCAGAGCATAGTGGTGCAGGAGCGCAG |
| | CTTGAGCCTGGCTGACAAGGTGGCCGCCATCAACTATTCCAT |
| | GCTTAGCCTGGGCAAGTTTTACGCCCGCAAGATATACCATAC |
| | CCCTTACGTTCCCATAGACAAGGAGGTAAAGATCGAGGGGTT |
| | CTACATGCGCATGGCGCTGAAGGTGCTTACCTTGAGCGACGA |
| | CCTGGGCGTTTATCGCAACGAGCGCATCCACAAGGCCGTGAG |
| | CGTGAGCCGGCGGCGCGAGCTCAGCGACCGCGAGCTGATGC |
| | ACAGCCTGCAAAGGGCCCTGGCTGGCACGGGCAGCGGCGAT |
| | AGAGAGGCCGAGTCCTACTTTGACGCGGGCGCTGACCTGCGC |
| | TGGGCCCCAAGCCGACGCGCCCTGGAGGCAGCTGGGGCCGG |
| | ACCTGGGCTGGCGGTGGCACCCGCGCGCGCTGGCAACGTCGG |
| | CGGCGTGGAGGAATATGACGAGGACGATGAGTACGAGCCAG |
| | AGGACGGCGAGTACTAAGCGGTGATGTTTCTGATCAGATGAT |
| | GCAAGACGCAACGGACCCGGCGGTGCGGGCGGCGCTGCAGA |
| | GCCAGCCGTCCGGCCTTAACTCCACGGACGACTGGCGCCAGG |
| | TCATGGACCGCATCATGTCGCTGACTGCGCGCAATCCTGACG |
| | CGTTCCGGCAGCAGCCGCAGGCCAACCGGCTCTCCGCAATTC |
| | TGGAAGCGGTGGTCCCGGCGCGCGCAAACCCCACGCACGAG |
| | AAGGTGCTGGCGATCGTAAACGCGCTGGCCGAAAACAGGGC |
| | CATCCGGCCCGACGAGGCCGGCCTGGTCTACGACGCGCTGCT |
| | TCAGCGCGTGGCTCGTTACAACAGCGGCAACGTGCAGACCAA |
| | CCTGGACCGGCTGGTGGGGGATGTGCGCGAGGCCGTGGCGCA |
| | GCGTGAGCGCGCGCAGCAGCAGGGCAACCTGGGCTCCATGGT |
| | TGCACTAAACGCCTTCCTGAGTACACAGCCCGCCAACGTGCC |
| | GCGGGGACAGGAGGACTACACCAACTTTGTGAGCGCACTGCG |
| | GCTAATGGTGACTGAGACACCGCAAAGTGAGGTGTACCAGTC |
| | TGGGCCAGACTATTTTTTCCAGACCAGTAGACAAGGCCTGCA |
| | GACCGTAAACCTGAGCCAGGCTTTCAAAAACTTGCAGGGGCT |
| | GTGGGGGGTGCGGGCTCCCACAGGCGACCGCGCGACCGTGTC |
| | TAGCTTGCTGACGCCCAACTCGCGCCTGTTGCTGCTGCTAATA |
| | GCGCCCTTCACGGACAGTGGCAGCGTGTCCCGGGACACATAC |
| | CTAGGTCACTTGCTGACACTGTACCGCGAGGCCATAGGTCAG |
| | GCGCATGTGGACGAGCATACTTTCCAGGAGATTACAAGTGTC |
| | AGCCGCGCGCTGGGGCAGGAGGACACGGGCAGCCTGGAGGC |
| | AACCCTAAACTACCTGCTGACCAACCGGCGGCAGAAGATCCC |
| | CTCGTTGCACAGTTTAAACAGCGAGGAGGAGCGCATTTTGCG |
| | CTACGTGCAGCAGAGCGTGAGCCTTAACCTGATGCGCGACGG |
| | GGTAACGCCCAGCGTGGCGCTGGACATGACCGCGCGCAACAT |
| | GGAACCGGGCATGTATGCCTCAAACCGGCCGTTTATCAACCG |
| | CCTAATGGACTACTTGCATCGCGCGGCCGCCGTGAACCCCGA |
| | GTATTTCACCAATGCCATCTTGAACCCGCACTGGCTACCGCCC |
| | CCTGGTTTCTACACCGGGGGATTCGAGGTGCCCGAGGGTAAC |
| | GATGGATTCCTCTGGGACGACATAGACGACAGCGTGTTTTCC |
| | CCGCAACCGCAGACCCTGCTAGAGTTGCAACAGCGCGAGCAG |
| | GCAGAGGCGGCGCTGCGAAAGGAAAGCTTCCGCAGGCCAAG |
| | CAGCTTGTCCGATCTAGGCGCTGCGGCCCCGCGGTCAGATGC |
| | TAGTAGCCCATTTCCAAGCTTGATAGGGTCTCTTACCAGCACT |
| | CGCACCACCCGCCCGCGCCTGCTGGGCGAGGAGGAGTACCTA |
| | AACAACTCGCTGCTGCAGCCGCAGCGCGAAAAAAACCTGCCT |
| | CCGGCATTTCCCAACAACGGGATAGAGAGCCTAGTGGACAAG |
| | ATGAGTAGATGGAAGACGTACGCGCAGGAGCACAGGGACGT |
| | GCCAGGCCCGCGCCCGCCCACCCGTCGTCAAAGGCACGACCG |
| | TCAGCGGGGTCTGGTGTGGGAGGACGATGACTCGGCAGACG |
| | ACAGCAGCGTCCTGGATTTGGGAGGGAGTGGCAACCCGTTTG |
| | CGCACCTTCGCCCCAGGCTGGGGAGAATGTTTTAAAAAAAAA |
| | AAAGCATGATGCAAAATAAAAAACTCACCAAGGCCATGGCA |
| | CCGAGCGTTGGTTTTCTTGTATTCCCCTTAGTATGCGGCGCGC |
| | GGCGATGTATGAGGAAGGTCCTCCTCCCTCCTACGAGAGTGT |
| | GGTGAGCGCGGCGCCAGTGGCGGCGGCGCTGGGTTCTCCCTT |
| | CGATGCTCCCCTGGACCCGCCGTTTGTGCCTCCGCGGTACCTG |
| | CGGCCTACCGGGGGGAGAAACAGCATCCGTTACTCTGAGTTG |
| | GCACCCCTATTCGACACCACCCGTGTGTACCTGGTGGACAAC |
| | AAGTCAACGGATGTGGCATCCCTGAACTACCAGAACGACCAC |
| | AGCAACTTTCTGACCACGGTCATTCAAAACAATGACTACAGC |
| | CCGGGGGAGGCAAGCACACAGACCATCAATCTTGACGACCG |
| | GTCGCACTGGGGCGGCGACCTGAAAACCATCCTGCATACCAA |
| | CATGCCAAATGTGAACGAGTTCATGTTTACCAATAAGTTTAA |
| | GGCGCGGGTGATGGTGTCGCGCTTGCCTACTAAGGACAATCA |
| | GGTGGAGCTGAAATACGAGTGGGTGGAGTTCACGCTGCCCGA |
| | GGGCAACTACTCCGAGACCATGACCATAGACCTTATGAACAA |
| | CGCGATCGTGGAGCACTACTTGAAAGTGGGCAGACAGAACG |
| | GGGTTCTGGAAAGCGACATCGGGGTAAAGTTTGACACCCGCA |
| | ACTTCAGACTGGGGTTTGACCCCGTCACTGGTCTTGTCATGCC |
| | TGGGGTATATACAAACGAAGCCTTCCATCCAGACATCATTTT |
| | GCTGCCAGGATGCGGGGTGGACTTCACCCACAGCCGCCTGAG |
| | CAACTTGTTGGGCATCCGCAAGCGGCAACCCTTCCAGGAGGG |
| | CTTTAGGATCACCTACGATGATCTGGAGGGTGGTAACATTCC |
| | CGCACTGTTGGATGTGGACGCCTACCAGGCGAGCTTGAAAGA |
| | TGACACCGAACAGGGCGGGGGTGGCGCAGGCGGCAGCAACA |
| | GCAGTGGCAGCGGCGCGGAAGAGAACTCCAACGCGGCAGCC |
| | GCGGCAATGCAGCCGGTGGAGGACATGAACGATCATGCCATT |
| | CGCGGCGACACCTTTGCCACACGGGCTGAGGAGAAGCGCGCT |
| | GAGGCCGAAGCAGCGGCCGAAGCTGCCGCCCCCGCTGCGCA |
| | ACCCGAGGTCGAGAAGCCTCAGAAGAAACCGGTGATCAAAC |
| | CCCTGACAGAGGACAGCAAGAAACGCAGTTACAACCTAATA |
| | AGCAATGACAGCACCTTCACCCAGTACCGCAGCTGGTACCTT |
| | GCATACAACTACGGCGACCCTCAGACCGGAATCCGCTCATGG |
| | ACCCTGCTTTGCACTCCTGACGTAACCTGCGGCTCGGAGCAG |
| | GTCTACTGGTCGTTGCCAGACATGATGCAAGACCCCGTGACC |
| | TTCCGCTCCACGCGCCAGATCAGCAACTTTCCGGTGGTGGGC |
| | GCCGAGCTGTTGCCCGTGCACTCCAAGAGCTTCTACAACGAC |
| | CAGGCCGTCTACTCCCAACTCATCCGCCAGTTTACCTCTCTGA |
| | CCCACGTGTTCAATCGCTTTCCCGAGAACCAGATTTTGGCGCG |
| | CCCGCCAGCCCCCACCATCACCACCGTCAGTGAAAACGTTCC |
| | TGCTCTCACAGATCACGGGACGCTACCGCTGCGCAACAGCAT |
| | CGGAGGAGTCCAGCGAGTGACCATTACTGACGCCAGACGCCG |
| | CACCTGCCCCTACGTTTACAAGGCCCTGGGCATAGTCTCGCC |
| | GCGCGTCCTATCGAGCCGCACTTTTTGAGCAAGCATGTCCATC |
| | CTTATATCGCCCAGCAATAACACAGGCTGGGGCCTGCGCTTC |
| | CCAAGCAAGATGTTTGGCGGGGCCAAGAAGCGCTCCGACCA |
| | ACACCCAGTGCGCGTGCGCGGGCACTACCGCGCGCCCTGGGG |
| | CGCGCACAAACGCGGCCGCACTGGGCGCACCACCGTCGATGA |
| | CGCCATCGACGCGGTGGTGGAGGAGGCGCGCAACTACACGC |
| | CCACGCCGCCACCAGTGTCCACAGTGGACGCGGCCATTCAGA |
| | CCGTGGTGCGCGGAGCCCGGCGCTATGCTAAAATGAAGAGAC |
| | GGCGGAGGCGCGTAGCACGTCGCCACCGCCGCCGACCCGGC |
| | ACTGCCGCCCAACGCGCGGCGGCGGCCCTGCTTAACCGCGCA |
| | CGTCGCACCGGCCGACGGGCGGCCATGCGGGCCGCTCGAAG |
| | GCTGGCCGCGGGTATTGTCACTGTGCCCCCCAGGTCCAGGCG |
| | ACGAGCGGCCGCCGCAGCAGCCGCGGCCATTAGTGCTATGAC |
| | TCAGGGTCGCAGGGGCAACGTGTATTGGGTGCGCGACTCGGT |
| | TAGCGGCCTGCGCGTGCCCGTGCGCACCCGCCCCCCGCGCAA |
| | CTAGATTGCAAGAAAAAACTACTTAGACTCGTACTGTTGTAT |
| | GTATCCAGCGGCGGCGGCGCGCAACGAAGCTATGTCCAAGCG |
| | CAAAATCAAAGAAGAGATGCTCCAGGTCATCGCGCCGGAGA |
| | TCTATGGCCCCCCGAAGAAGGAAGAGCAGGATTACAAGCCCC |
| | GAAAGCTAAAGCGGGTCAAAAAGAAAAAGAAAGATGATGAT |
| | GATGAACTTGACGACGAGGTGGAACTGCTGCACGCTACCGCG |
| | CCCAGGCGACGGGTACAGTGGAAAGGTCGACGCGTAAAACG |
| | TGTTTTGCGACCCGGCACCACCGTAGTCTTTACGCCCGGTGAG |
| | CGCTCCACCCGCACCTACAAGCGCGTGTATGATGAGGTGTAC |
| | GGCGACGAGGACCTGCTTGAGCAGGCCAACGAGCGCCTCGG |
| | GGAGTTTGCCTACGGAAAGCGGCATAAGGACATGCTGGCGTT |
| | GCCGCTGGACGAGGGCAACCCAACACCTAGCCTAAAGCCCGT |
| | AACACTGCAGCAGGTGCTGCCCGCGCTTGCACCGTCCGAAGA |
| | AAAGCGCGGCCTAAAGCGCGAGTCTGGTGACTTGGCACCCAC |
| | CGTGCAGCTGATGGTACCCAAGCGCCAGCGACTGGAAGATGT |
| | CTTGGAAAAAATGACCGTGGAACCTGGGCTGGAGCCCGAGGT |
| | CCGCGTGCGGCCAATCAAGCAGGTGGCGCCGGGACTGGGCGT |
| | GCAGACCGTGGACGTTCAGATACCCACTACCAGTAGCACCAG |
| | TATTGCCACCGCCACAGAGGGCATGGAGACACAAACGTCCCC |
| | GGTTGCCTCAGCGGTGGCGGATGCCGCGGTGCAGGCGGTCGC |
| | TGCGGCCGCGTCCAAGACCTCTACGGAGGTGCAAACGGACCC |
| | GTGGATGTTTCGCGTTTCAGCCCCCCGGCGCCCGCGCCGTTCG |
| | AGGAAGTACGGCGCCGCCAGCGCGCTACTGCCCGAATATGCC |
| | CTACATCCTTCCATTGCGCCTACCCCCGGCTATCGTGGCTACA |
| | CCTACCGCCCCAGAAGACGAGCAACTACCCGACGCCGAACCA |
| | CCACTGGAACCCGCCGCCGCCGTCGCCGTCGCCAGCCCGTGC |
| | TGGCCCCGATTTCCGTGCGCAGGGTGGCTCGCGAAGGAGGCA |
| | GGACCCTGGTGCTGCCAACAGCGCGCTACCACCCCAGCATCG |
| | TTTAAAAGCCGGTCTTTGTGGTTCTTGCAGATATGGCCCTCAC |
| | CTGCCGCCTCCGTTTCCCGGTGCCGGGATTCCGAGGAAGAAT |
| | GCACCGTAGGAGGGGCATGGCCGGCCACGGCCTGACGGGCG |
| | GCATGCGTCGTGCGCACCACCGGCGGCGGCGCGCGTCGCACC |
| | GTCGCATGCGCGGCGGTATCCTGCCCCTCCTTATTCCACTGAT |
| | CGCCGCGGCGATTGGCGCCGTGCCCGGAATTGCATCCGTGGC |
| | CTTGCAGGCGCAGAGACACTGATTAAAAACAAGTTGCATGTG |
| | GAAAAATCAAAATAAAAAGTCTGGACTCTCACGCTCGCTTGG |
| | TCCTGTAACTATTTTGTAGAATGGAAGACATCAACTTTGCGTC |
| | TCTGGCCCCGCGACACGGCTCGCGCCCGTTCATGGGAAACTG |
| | GCAAGATATCGGCACCAGCAATATGAGCGGTGGCGCCTTCAG |
| | CTGGGGCTCGCTGTGGAGCGGCATTAAAAATTTCGGTTCCAC |
| | CGTTAAGAACTATGGCAGCAAGGCCTGGAACAGCAGCACAG |
| | GCCAGATGCTGAGGGATAAGTTGAAAGAGCAAAATTTCCAAC |
| | AAAAGGTGGTAGATGGCCTGGCCTCTGGCATTAGCGGGGTGG |
| | TGGACCTGGCCAACCAGGCAGTGCAAAATAAGATTAACAGTA |
| | AGCTTGATCCCCGCCCTCCCGTAGAGGAGCCTCCACCGGCCG |
| | TGGAGACAGTGTCTCCAGAGGGGCGTGGCGAAAAGCGTCCG |
| | CGCCCCGACAGGGAAGAAACTCTGGTGACGCAAATAGACGA |
| | GCCTCCCTCGTACGAGGAGGCACTAAAGCAAGGCCTGCCCAC |
| | CACCCGTCCCATCGCGCCCATGGCTACCGGAGTGCTGGGCCA |
| | GCACACACCCGTAACGCTGGACCTGCCTCCCCCCGCCGACAC |
| | CCAGCAGAAACCTGTGCTGCCAGGCCCGACCGCCGTTGTTGT |
| | AACCCGTCCTAGCCGCGCGTCCCTGCGCCGCGCCGCCAGCGG |
| | TCCGCGATCGTTGCGGCCCGTAGCCAGTGGCAACTGGCAAAG |
| | CACACTGAACAGCATCGTGGGTCTGGGGGTGCAATCCCTGAA |
| | GCGCCGACGATGCTTCTGATAGCTAACGTGTCGTATGTGTGTC |
| | ATGTATGCGTCCATGTCGCCGCCAGAGGAGCTGCTGAGCCGC |
| | CGCGCGCCCGCTTTCCAAGATGGCTACCCCTTCGATGATGCC |
| | GCAGTGGTCTTACATGCACATCTCGGGCCAGGACGCCTCGGA |
| | GTACCTGAGCCCCGGGCTGGTGCAGTTTGCCCGCGCCACCGA |
| | GACGTACTTCAGCCTGAATAACAAGTTTAGAAACCCCACGGT |
| | GGCGCCTACGCACGACGTGACCACAGACCGGTCCCAGCGTTT |
| | GACGCTGCGGTTCATCCCTGTGGACCGTGAGGATACTGCGTA |
| | CTCGTACAAGGCGCGGTTCACCCTAGCTGTGGGTGATAACCG |
| | TGTGCTGGACATGGCTTCCACGTACTTTGACATCCGCGGCGTG |
| | CTGGACAGGGGCCCTACTTTTAAGCCCTACTCTGGCACTGCCT |
| | ACAACGCCCTGGCTCCCAAGGGTGCCCCAAATCCTTGCGAAT |
| | GGGATGAAGCTGCTACTGCTCTTGAAATAAACCTAGAAGAAG |
| | AGGACGATGACAACGAAGACGAAGTAGACGAGCAAGCTGAG |
| | CAGCAAAAAACTCACGTATTTGGGCAGGCGCCTTATTCTGGT |
| | ATAAATATTACAAAGGAGGGTATTCAAATAGGTGTCGAAGGT |
| | CAAACACCTAAATATGCCGATAAAACATTTCAACCTGAACCT |
| | CAAATAGGAGAATCTCAGTGGTACGAAACAGAAATTAATCAT |
| | GCAGCTGGGAGAGTCCTAAAAAAGACTACCCCAATGAAACC |
| | ATGTTACGGTTCATATGCAAAACCCACAAATGAAAATGGAGG |
| | GCAAGGCATTCTTGTAAAGCAACAAAATGGAAAGCTAGAAA |
| | GTCAAGTGGAAATGCAATTTTTCTCAACTACTGAGGCAGCCG |
| | CAGGCAATGGTGATAACTTGACTCCTAAAGTGGTATTGTACA |
| | GTGAAGATGTAGATATAGAAACCCCAGACACTCATATTTCTT |
| | ACATGCCCACTATTAAGGAAGGTAACTCACGAGAACTAATGG |
| | GCCAACAATCTATGCCCAACAGGCCTAATTACATTGCTTTTAG |
| | GGACAATTTTATTGGTCTAATGTATTACAACAGCACGGGTAA |
| | TATGGGTGTTCTGGCGGGCCAAGCATCGCAGTTGAATGCTGT |
| | TGTAGATTTGCAAGACAGAAACACAGAGCTTTCATACCAGCT |
| | TTTGCTTGATTCCATTGGTGATAGAACCAGGTACTTTTCTATG |
| | TGGAATCAGGCTGTTGACAGCTATGATCCAGATGTTAGAATT |
| | ATTGAAAATCATGGAACTGAAGATGAACTTCCAAATTACTGC |
| | TTTCCACTGGGAGGTGTGATTAATACAGAGACTCTTACCAAG |
| | GTAAAACCTAAAACAGGTCAGGAAAATGGATGGGAAAAAGA |
| | TGCTACAGAATTTTCAGATAAAAATGAAATAAGAGTTGGAAA |
| | TAATTTTGCCATGGAAATCAATCTAAATGCCAACCTGTGGAG |
| | AAATTTCCTGTACTCCAACATAGCGCTGTATTTGCCCGACAAG |
| | CTAAAGTACAGTCCTTCCAACGTAAAAATTTCTGATAACCCA |
| | AACACCTACGACTACATGAACAAGCGAGTGGTGGCTCCCGGG |
| | CTAGTGGACTGCTACATTAACCTTGGAGCACGCTGGTCCCTTG |
| | ACTATATGGACAACGTCAACCCATTTAACCACCACCGCAATG |
| | CTGGCCTGCGCTACCGCTCAATGTTGCTGGGCAATGGTCGCT |
| | ATGTGCCCTTCCACATCCAGGTGCCTCAGAAGTTCTTTGCCAT |
| | TAAAAACCTCCTTCTCCTGCCGGGCTCATACACCTACGAGTG |
| | GAACTTCAGGAAGGATGTTAACATGGTTCTGCAGAGCTCCCT |
| | AGGAAATGACCTAAGGGTTGACGGAGCCAGCATTAAGTTTGA |
| | TAGCATTTGCCTTTACGCCACCTTCTTCCCCATGGCCCACAAC |
| | ACCGCCTCCACGCTTGAGGCCATGCTTAGAAACGACACCAAC |
| | GACCAGTCCTTTAACGACTATCTCTCCGCCGCCAACATGCTCT |
| | ACCCTATACCCGCCAACGCTACCAACGTGCCCATATCCATCC |
| | CCTCCCGCAACTGGGCGGCTTTCCGCGGCTGGGCCTTCACGC |
| | GCCTTAAGACTAAGGAAACCCCATCACTGGGCTCGGGCTACG |
| | ACCCTTATTACACCTACTCTGGCTCTATACCCTACCTAGATGG |
| | AACCTTTTACCTCAACCACACCTTTAAGAAGGTGGCCATTACC |
| | TTTGACTCTTCTGTCAGCTGGCCTGGCAATGACCGCCTGCTTA |
| | CCCCCAACGAGTTTGAAATTAAGCGCTCAGTTGACGGGGAGG |
| | GTTACAACGTTGCCCAGTGTAACATGACCAAAGACTGGTTCC |
| | TGGTACAAATGCTAGCTAACTATAACATTGGCTACCAGGGCT |
| | TCTATATCCCAGAGAGCTACAAGGACCGCATGTACTCCTTCTT |
| | TAGAAACTTCCAGCCCATGAGCCGTCAGGTGGTGGATGATAC |
| | TAAATACAAGGACTACCAACAGGTGGGCATCCTACACCAACA |
| | CAACAACTCTGGATTTGTTGGCTACCTTGCCCCCACCATGCGC |
| | GAAGGACAGGCCTACCCTGCTAACTTCCCCTATCCGCTTATA |
| | GGCAAGACCGCAGTTGACAGCATTACCCAGAAAAAGTTTCTT |
| | TGCGATCGCACCCTTTGGCGCATCCCATTCTCCAGTAACTTTA |
| | TGTCCATGGGCGCACTCACAGACCTGGGCCAAAACCTTCTCT |
| | ACGCCAACTCCGCCCACGCGCTAGACATGACTTTTGAGGTGG |
| | ATCCCATGGACGAGCCCACCCTTCTTTATGTTTTGTTTGAAGT |
| | CTTTGACGTGGTCCGTGTGCACCAGCCGCACCGCGGCGTCAT |
| | CGAAACCGTGTACCTGCGCACGCCCTTCTCGGCCGGCAACGC |
| | CACAACATAAAGAAGCAAGCAACATCAACAACAGCTGCCGC |
| | CATGGGCTCCAGTGAGCAGGAACTGAAAGCCATTGTCAAAGA |
| | TCTTGGTTGTGGGCCATATTTTTTGGGCACCTATGACAAGCGC |
| | TTTCCAGGCTTTGTTTCTCCACACAAGCTCGCCTGCGCCATAG |
| | TCAATACGGCCGGTCGCGAGACTGGGGGCGTACACTGGATGG |
| | CCTTTGCCTGGAACCCGCACTCAAAAACATGCTACCTCTTTGA |
| | GCCCTTTGGCTTTTCTGACCAGCGACTCAAGCAGGTTTACCAG |
| | TTTGAGTACGAGTCACTCCTGCGCCGTAGCGCCATTGCTTCTT |
| | CCCCCGACCGCTGTATAACGCTGGAAAAGTCCACCCAAAGCG |
| | TACAGGGGCCCAACTCGGCCGCCTGTGGACTATTCTGCTGCA |
| | TGTTTCTCCACGCCTTTGCCAACTGGCCCCAAACTCCCATGGA |
| | TCACAACCCCACCATGAACCTTATTACCGGGGTACCCAACTC |
| | CATGCTCAACAGTCCCCAGGTACAGCCCACCCTGCGTCGCAA |
| | CCAGGAACAGCTCTACAGCTTCCTGGAGCGCCACTCGCCCTA |
| | CTTCCGCAGCCACAGTGCGCAGATTAGGAGCGCCACTTCTTTT |
| | TGTCACTTGAAAAACATGTAAAAATAATGTACTAGAGACACT |
| | TTCAATAAAGGCAAATGCTTTTATTTGTACACTCTCGGGTGAT |
| | TATTTACCCCCACCCTTGCCGTCTGCGCCGTTTAAAAATCAAA |
| | GGGGTTCTGCCGCGCATCGCTATGCGCCACTGGCAGGGACAC |
| | GTTGCGATACTGGTGTTTAGTGCTCCACTTAAACTCAGGCACA |
| | ACCATCCGCGGCAGCTCGGTGAAGTTTTCACTCCACAGGCTG |
| | CGCACCATCACCAACGCGTTTAGCAGGTCGGGCGCCGATATC |
| | TTGAAGTCGCAGTTGGGGCCTCCGCCCTGCGCGCGCGAGTTG |
| | CGATACACAGGGTTGCAGCACTGGAACACTATCAGCGCCGGG |
| | TGGTGCACGCTGGCCAGCACGCTCTTGTCGGAGATCAGATCC |
| | GCGTCCAGGTCCTCCGCGTTGCTCAGGGCGAACGGAGTCAAC |
| | TTTGGTAGCTGCCTTCCCAAAAAGGGCGCGTGCCCAGGCTTT |
| | GAGTTGCACTCGCACCGTAGTGGCATCAAAAGGTGACCGTGC |
| | CCGGTCTGGGCGTTAGGATACAGCGCCTGCATAAAAGCCTTG |
| | ATCTGCTTAAAAGCCACCTGAGCCTTTGCGCCTTCAGAGAAG |
| | AACATGCCGCAAGACTTGCCGGAAAACTGATTGGCCGGACAG |
| | GCCGCGTCGTGCACGCAGCACCTTGCGTCGGTGTTGGAGATC |
| | TGCACCACATTTCGGCCCCACCGGTTCTTCACGATCTTGGCCT |
| | TGCTAGACTGCTCCTTCAGCGCGCGCTGCCCGTTTTCGCTCGT |
| | CACATCCATTTCAATCACGTGCTCCTTATTTATCATAATGCTT |
| | CCGTGTAGACACTTAAGCTCGCCTTCGATCTCAGCGCAGCGG |
| | TGCAGCCACAACGCGCAGCCCGTGGGCTCGTGATGCTTGTAG |
| | GTCACCTCTGCAAACGACTGCAGGTACGCCTGCAGGAATCGC |
| | CCCATCATCGTCACAAAGGTCTTGTTGCTGGTGAAGGTCAGC |
| | TGCAACCCGCGGTGCTCCTCGTTCAGCCAGGTCTTGCATACG |
| | GCCGCCAGAGCTTCCACTTGGTCAGGCAGTAGTTTGAAGTTC |
| | GCCTTTAGATCGTTATCCACGTGGTACTTGTCCATCAGCGCGC |
| | GCGCAGCCTCCATGCCCTTCTCCCACGCAGACACGATCGGCA |
| | CACTCAGCGGGTTCATCACCGTAATTTCACTTTCCGCTTCGCT |
| | GGGCTCTTCCTCTTCCTCTTGCGTCCGCATACCACGCGCCACT |
| | GGGTCGTCTTCATTCAGCCGCCGCACTGTGCGCTTACCTCCTT |
| | TGCCATGCTTGATTAGCACCGGTGGGTTGCTGAAACCCACCA |
| | TTTGTAGCGCCACATCTTCTCTTTCTTCCTCGCTGTCCACGATT |
| | ACCTCTGGTGATGGCGGGCGCTCGGGCTTGGGAGAAGGGCGC |
| | TTCTTTTTCTTCTTGGGCGCAATGGCCAAATCCGCCGCCGAGG |
| | TCGATGGCCGCGGGCTGGGTGTGCGCGGCACCAGCGCGTCTT |
| | GTGATGAGTCTTCCTCGTCCTCGGACTCGATACGCCGCCTCAT |
| | CCGCTTTTTTGGGGGCGCCCGGGGAGGCGGCGGCGACGGGGA |
| | CGGGGACGACACGTCCTCCATGGTTGGGGGACGTCGCGCCGC |
| | ACCGCGTCCGCGCTCGGGGGTGGTTTCGCGCTGCTCCTCTTCC |
| | CGACTGGCCATTTCCTTCTCCTATAGGCAGAAAAAGATCATG |
| | GAGTCAGTCGAGAAGAAGGACAGCCTAACCGCCCCCTCTGAG |
| | TTCGCCACCACCGCCTCCACCGATGCCGCCAACGCGCCTACC |
| | ACCTTCCCCGTCGAGGCACCCCCGCTTGAGGAGGAGGAAGTG |
| | ATTATCGAGCAGGACCCAGGTTTTGTAAGCGAAGACGACGAG |
| | GACCGCTCAGTACCAACAGAGGATAAAAAGCAAGACCAGGA |
| | CAACGCAGAGGCAAACGAGGAACAAGTCGGGCGGGGGGACG |
| | AAAGGCATGGCGACTACCTAGATGTGGGAGACGACGTGCTGT |
| | TGAAGCATCTGCAGCGCCAGTGCGCCATTATCTGCGACGCGT |
| | TGCAAGAGCGCAGCGATGTGCCCCTCGCCATAGCGGATGTCA |
| | GCCTTGCCTACGAACGCCACCTATTCTCACCGCGCGTACCCCC |
| | CAAACGCCAAGAAAACGGCACATGCGAGCCCAACCCGCGCC |
| | TCAACTTCTACCCCGTATTTGCCGTGCCAGAGGTGCTTGCCAC |
| | CTATCACATCTTTTTCCAAAACTGCAAGATACCCCTATCCTGC |
| | CGTGCCAACCGCAGCCGAGCGGACAAGCAGCTGGCCTTGCGG |
| | CAGGGCGCTGTCATACCTGATATCGCCTCGCTCAACGAAGTG |
| | CCAAAAATCTTTGAGGGTCTTGGACGCGACGAGAAGCGCGCG |
| | GCAAACGCTCTGCAACAGGAAAACAGCGAAAATGAAAGTCA |
| | CTCTGGAGTGTTGGTGGAACTCGAGGGTGACAACGCGCGCCT |
| | AGCCGTACTAAAACGCAGCATCGAGGTCACCCACTTTGCCTA |
| | CCCGGCACTTAACCTACCCCCCAAGGTCATGAGCACAGTCAT |
| | GAGTGAGCTGATCGTGCGCCGTGCGCAGCCCCTGGAGAGGGA |
| | TGCAAATTTGCAAGAACAAACAGAGGAGGGCCTACCCGCAG |
| | TTGGCGACGAGCAGCTAGCGCGCTGGCTTCAAACGCGCGAGC |
| | CTGCCGACTTGGAGGAGCGACGCAAACTAATGATGGCCGCAG |
| | TGCTCGTTACCGTGGAGCTTGAGTGCATGCAGCGGTTCTTTGC |
| | TGACCCGGAGATGCAGCGCAAGCTAGAGGAAACATTGCACT |
| | ACACCTTTCGACAGGGCTACGTACGCCAGGCCTGCAAGATCT |
| | CCAACGTGGAGCTCTGCAACCTGGTCTCCTACCTTGGAATTTT |
| | GCACGAAAACCGCCTTGGGCAAAACGTGCTTCATTCCACGCT |
| | CAAGGGCGAGGCGCGCCGCGACTACGTCCGCGACTGCGTTTA |
| | CTTATTTCTATGCTACACCTGGCAGACGGCCATGGGCGTTTGG |
| | CAGCAGTGCTTGGAGGAGTGCAACCTCAAGGAGCTGCAGAA |
| | ACTGCTAAAGCAAAACTTGAAGGACCTATGGACGGCCTTCAA |
| | CGAGCGCTCCGTGGCCGCGCACCTGGCGGACATCATTTTCCC |
| | CGAACGCCTGCTTAAAACCCTGCAACAGGGTCTGCCAGACTT |
| | CACCAGTCAAAGCATGTTGCAGAACTTTAGGAACTTTATCCT |
| | AGAGCGCTCAGGAATCTTGCCCGCCACCTGCTGTGCACTTCCT |
| | AGCGACTTTGTGCCCATTAAGTACCGCGAATGCCCTCCGCCG |
| | CTTTGGGGCCACTGCTACCTTCTGCAGCTAGCCAACTACCTTG |
| | CCTACCACTCTGACATAATGGAAGACGTGAGCGGTGACGGTC |
| | TACTGGAGTGTCACTGTCGCTGCAACCTATGCACCCCGCACC |
| | GCTCCCTGGTTTGCAATTCGCAGCTGCTTAACGAAAGTCAAA |
| | TTATCGGTACCTTTGAGCTGCAGGGTCCCTCGCCTGACGAAA |
| | AGTCCGCGGCTCCGGGGTTGAAACTCACTCCGGGGCTGTGGA |
| | CGTCGGCTTACCTTCGCAAATTTGTACCTGAGGACTACCACGC |
| | CCACGAGATTAGGTTCTACGAAGACCAATCCCGCCCGCCTAA |
| | TGCGGAGCTTACCGCCTGCGTCATTACCCAGGGCCACATTCTT |
| | GGCCAATTGCAAGCCATCAACAAAGCCCGCCAAGAGTTTCTG |
| | CTACGAAAGGGACGGGGGGTTTACTTGGACCCCCAGTCCGGC |
| | GAGGAGCTCAACCCAATCCCCCCGCCGCCGCAGCCCTATCAG |
| | CAGCAGCCGCGGGCCCTTGCTTCCCAGGATGGCACCCAAAAA |
| | GAAGCTGCAGCTGCCGCCGCCACCCACGGACGAGGAGGAAT |
| | ACTGGGACAGTCAGGCAGAGGAGGTTTTGGACGAGGAGGAG |
| | GAGGACATGATGGAAGACTGGGAGAGCCTAGACGAGGAAGC |
| | TTCCGAGGTCGAAGAGGTGTCAGACGAAACACCGTCACCCTC |
| | GGTCGCATTCCCCTCGCCGGCGCCCCAGAAATCGGCAACCGG |
| | TTCCAGCATGGCTACAACCTCCGCTCCTCAGGCGCCGCCGGC |
| | ACTGCCCGTTCGCCGACCCAACCGTAGATGGGACACCACTGG |
| | AACCAGGGCCGGTAAGTCCAAGCAGCCGCCGCCGTTAGCCCA |
| | AGAGCAACAACAGCGCCAAGGCTACCGCTCATGGCGCGGGC |
| | ACAAGAACGCCATAGTTGCTTGCTTGCAAGACTGTGGGGGCA |
| | ACATCTCCTTCGCCCGCCGCTTTCTTCTCTACCATCACGGCGT |
| | GGCCTTCCCCCGTAACATCCTGCATTACTACCGTCATCTCTAC |
| | AGCCCATACTGCACCGGCGGCAGCGGCAGCAACAGCAGCGG |
| | CCACACAGAAGCAAAGGCGACCGGATAGCAAGACTCTGACA |
| | AAGCCCAAGAAATCCACAGCGGCGGCAGCAGCAGGAGGAGG |
| | AGCGCTGCGTCTGGCGCCCAACGAACCCGTATCGACCCGCGA |
| | GCTTAGAAACAGGATTTTTCCCACTCTGTATGCTATATTTCAA |
| | CAGAGCAGGGGCCAAGAACAAGAGCTGAAAATAAAAAACAG |
| | GTCTCTGCGATCCCTCACCCGCAGCTGCCTGTATCACAAAAG |
| | CGAAGATCAGCTTCGGCGCACGCTGGAAGACGCGGAGGCTCT |
| | CTTCAGTAAATACTGCGCGCTGACTCTTAAGGACTAGTTTCGC |
| | GCCCTTTCTCAAATTTAAGCGCGAAAACTACGTCATCTCCAGC |
| | GGCCACACCCGGCGCCAGCACCTGTTGTCAGCGCCATTATGA |
| | GCAAGGAAATTCCCACGCCCTACATGTGGAGTTACCAGCCAC |
| | AAATGGGACTTGCGGCTGGAGCTGCCCAAGACTACTCAACCC |
| | GAATAAACTACATGAGCGCGGGACCCCACATGATATCCCGGG |
| | TCAACGGAATACGCGCCCACCGAAACCGAATTCTCCTGGAAC |
| | AGGCGGCTATTACCACCACACCTCGTAATAACCTTAATCCCC |
| | GTAGTTGGCCCGCTGCCCTGGTGTACCAGGAAAGTCCCGCTC |
| | CCACCACTGTGGTACTTCCCAGAGACGCCCAGGCCGAAGTTC |
| | AGATGACTAACTCAGGGGCGCAGCTTGCGGGCGGCTTTCGTC |
| | ACAGGGTGCGGTCGCCCGGGCAGGGTATAACTCACCTGACAA |
| | TCAGAGGGCGAGGTATTCAGCTCAACGACGAGTCGGTGAGCT |
| | CCTCGCTTGGTCTCCGTCCGGACGGGACATTTCAGATCGGCG |
| | GCGCCGGCCGCTCTTCATTCACGCCTCGTCAGGCAATCCTAAC |
| | TCTGCAGACCTCGTCCTCTGAGCCGCGCTCTGGAGGCATTGG |
| | AACTCTGCAATTTATTGAGGAGTTTGTGCCATCGGTCTACTTT |
| | AACCCCTTCTCGGGACCTCCCGGCCACTATCCGGATCAATTTA |
| | TTCCTAACTTTGACGCGGTAAAGGACTCGGCGGACGGCTACG |
| | ACTGAATGTTAAGTGGAGAGGCAGAGCAACTGCGCCTGAAA |
| | CACCTGGTCCACTGTCGCCGCCACAAGTGCTTTGCCCGCGACT |
| | CCGGTGAGTTTTGCTACTTTGAATTGCCCGAGGATCATATCGA |
| | GGGCCCGGCGCACGGCGTCCGGCTTACCGCCCAGGGAGAGCT |
| | TGCCCGTAGCCTGATTCGGGAGTTTACCCAGCGCCCCCTGCTA |
| | GTTGAGCGGGACAGGGGACCCTGTGTTCTCACTGTGATTTGC |
| | AACTGTCCTAACCCTGGATTACATCAAGATCCTCTAGTTAATG |
| | TCAGGTCGCCTAAGTCGATTAACTAGAGTACCCGGGGATCTT |
| | ATTCCCTTTAACTAATAAAAAAAAATAATAAAGCATCACTTA |
| | CTTAAAATCAGTTAGCAAATTTCTGTCCAGTTTATTCAGCAGC |
| | ACCTCCTTGCCCTCCTCCCAGCTCTGGTATTGCAGCTTCCTCC |
| | TGGCTGCAAACTTTCTCCACAATCTAAATGGAATGTCAGTTTC |
| | CTCCTGTTCCTGTCCATCCGCACCCACTATCTTCATGTTGTTGC |
| | AGATGAAGCGCGCAAGACCGTCTGAAGATACCTTCAACCCCG |
| | TGTATCCATATGACACGGAAACCGGTCCTCCAACTGTGCCTTT |
| | TCTTACTCCTCCCTTTGTATCCCCCAATGGGTTTCAAGAGAGT |
| | CCCCCTGGGGTACTCTCTTTGCGCCTATCCGAACCTCTAGTTA |
| | CCTCCAATGGCATGCTTGCGCTCAAAATGGGCAACGGCCTCT |
| | CTCTGGACGAGGCCGGCAACCTTACCTCCCAAAATGTAACCA |
| | CTGTGAGCCCACCTCTCAAAAAAACCAAGTCAAACATAAACC |
| | TGGAAATATCTGCACCCCTCACAGTTACCTCAGAAGCCCTAA |
| | CTGTGGCTGCCGCCGCACCTCTAATGGTCGCGGGCAACACAC |
| | TCACCATGCAATCACAGGCCCCGCTAACCGTGCACGACTCCA |
| | AACTTAGCATTGCCACCCAAGGACCCCTCACAGTGTCAGAAG |
| | GAAAGCTAGCCCTGCAAACATCAGGCCCCCTCACCACCACCG |
| | ATAGCAGTACCCTTACTATCACTGCCTCACCCCCTCTAACTAC |
| | TGCCACTGGTAGCTTGGGCATTGACTTGAAAGAGCCCATTTA |
| | TACACAAAATGGAAAACTAGGACTAAAGTACGGGGCTCCTTT |
| | GCATGTAACAGACGACCTAAACACTTTGACCGTAGCAACTGG |
| | TCCAGGTGTGACTATTAATAATACTTCCTTGCAAACTAAAGTT |
| | ACTGGAGCCTTGGGTTTTGATTCACAAGGCAATATGCAACTT |
| | AATGTAGCAGGAGGACTAAGGATTGATTCTCAAAACAGACGC |
| | CTTATACTTGATGTTAGTTATCCGTTTGATGCTCAAAACCAAC |
| | TAAATCTAAGACTAGGACAGGGCCCTCTTTTTATAAACTCAG |
| | CCCACAACTTGGATATTAACTACAACAAAGGCCTTTACTTGTT |
| | TACAGCTTCAAACAATTCCAAAAAGCTTGAGGTTAACCTAAG |
| | CACTGCCAAGGGGTTGATGTTTGACGCTACAGCCATAGCCAT |
| | TAATGCAGGAGATGGGCTTGAATTTGGTTCACCTAATGCACC |
| | AAACACAAATCCCCTCAAAACAAAAATTGGCCATGGCCTAGA |
| | ATTTGATTCAAACAAGGCTATGGTTCCTAAACTAGGAACTGG |
| | CCTTAGTTTTGACAGCACAGGTGCCATTACAGTAGGAAACAA |
| | AAATAATGATAAGCTAACTTTGTGGACCACACCAGCTCCATC |
| | TCCTAACTGTAGACTAAATGCAGAGAAAGATGCTAAACTCAC |
| | TTTGGTCTTAACAAAATGTGGCAGTCAAATACTTGCTACAGTT |
| | TCAGTTTTGGCTGTTAAAGGCAGTTTGGCTCCAATATCTGGAA |
| | CAGTTCAAAGTGCTCATCTTATTATAAGATTTGACGAAAATG |
| | GAGTGCTACTAAACAATTCCTTCCTGGACCCAGAATATTGGA |
| | ACTTTAGAAATGGAGATCTTACTGAAGGCACAGCCTATACAA |
| | ACGCTGTTGGATTTATGCCTAACCTATCAGCTTATCCAAAATC |
| | TCACGGTAAAACTGCCAAAAGTAACATTGTCAGTCAAGTTTA |
| | CTTAAACGGAGACAAAACTAAACCTGTAACACTAACCATTAC |
| | ACTAAACGGTACACAGGAAACAGGAGACACAACTCCAAGTG |
| | CATACTCTATGTCATTTTCATGGGACTGGTCTGGCCACAACTA |
| | CATTAATGAAATATTTGCCACATCCTCTTACACTTTTTCATAC |
| | ATTGCCCAAGAATAAAGAATCGTTTGTGTTATGTTTCAACGTG |
| | TTTATTTTTCAATTGCAGAAAATTTCAAGTCATTTTTCATTCA |
| | GTAGTATAGCCCCACCACCACATAGCTTATACAGATCACCGT |
| | ACCTTAATCAAACTCACAGAACCCTAGTATTCAACCTGCCAC |
| | CTCCCTCCCAACACACAGAGTACACAGTCCTTTCTCCCCGGCT |
| | GGCCTTAAAAAGCATCATATCATGGGTAACAGACATATTCTT |
| | AGGTGTTATATTCCACACGGTTTCCTGTCGAGCCAAACGCTCA |
| | TCAGTGATATTAATAAACTCCCCGGGCAGCTCACTTAAGTTC |
| | ATGTCGCTGTCCAGCTGCTGAGCCACAGGCTGCTGTCCAACTT |
| | GCGGTTGCTTAACGGGCGGCGAAGGAGAAGTCCACGCCTACA |
| | TGGGGGTAGAGTCATAATCGTGCATCAGGATAGGGCGGTGGT |
| | GCTGCAGCAGCGCGCGAATAAACTGCTGCCGCCGCCGCTCCG |
| | TCCTGCAGGAATACAACATGGCAGTGGTCTCCTCAGCGATGA |
| | TTCGCACCGCCCGCAGCATAAGGCGCCTTGTCCTCCGGGCAC |
| | AGCAGCGCACCCTGATCTCACTTAAATCAGCACAGTAACTGC |
| | AGCACAGCACCACAATATTGTTCAAAATCCCACAGTGCAAGG |
| | CGCTGTATCCAAAGCTCATGGCGGGGACCACAGAACCCACGT |
| | GGCCATCATACCACAAGCGCAGGTAGATTAAGTGGCGACCCC |
| | TCATAAACACGCTGGACATAAACATTACCTCTTTTGGCATGTT |
| | GTAATTCACCACCTCCCGGTACCATATAAACCTCTGATTAAAC |
| | ATGGCGCCATCCACCACCATCCTAAACCAGCTGGCCAAAACC |
| | TGCCCGCCGGCTATACACTGCAGGGAACCGGGACTGGAACAA |
| | TGACAGTGGAGAGCCCAGGACTCGTAACCATGGATCATCATG |
| | CTCGTCATGATATCAATGTTGGCACAACACAGGCACACGTGC |
| | ATACACTTCCTCAGGATTACAAGCTCCTCCCGCGTTAGAACC |
| | ATATCCCAGGGAACAACCCATTCCTGAATCAGCGTAAATCCC |
| | ACACTGCAGGGAAGACCTCGCACGTAACTCACGTTGTGCATT |
| | GTCAAAGTGTTACATTCGGGCAGCAGCGGATGATCCTCCAGT |
| | ATGGTAGCGCGGGTTTCTGTCTCAAAAGGAGGTAGACGATCC |
| | CTACTGTACGGAGTGCGCCGAGACAACCGAGATCGTGTTGGT |
| | CGTAGTGTCATGCCAAATGGAACGCCGGACGTAGTCATATTT |
| | CCTGAAGCAAAACCAGGTGCGGGCGTGACAAACAGATCTGC |
| | GTCTCCGGTCTCGCCGCTTAGATCGCTCTGTGTAGTAGTTGTA |
| | GTATATCCACTCTCTCAAAGCATCCAGGCGCCCCCTGGCTTCG |
| | GGTTCTATGTAAACTCCTTCATGCGCCGCTGCCCTGATAACAT |
| | CCACCACCGCAGAATAAGCCACACCCAGCCAACCTACACATT |
| | CGTTCTGCGAGTCACACACGGGAGGAGCGGGAAGAGCTGGA |
| | AGAACCATGTTTTTTTTTTTATTCCAAAAGATTATCCAAAACC |
| | TCAAAATGAAGATCTATTAAGTGAACGCGCTCCCCTCCGGTG |
| | GCGTGGTCAAACTCTACAGCCAAAGAACAGATAATGGCATTT |
| | GTAAGATGTTGCACAATGGCTTCCAAAAGGCAAACGGCCCTC |
| | ACGTCCAAGTGGACGTAAAGGCTAAACCCTTCAGGGTGAATC |
| | TCCTCTATAAACATTCCAGCACCTTCAACCATGCCCAAATAAT |
| | TCTCATCTCGCCACCTTCTCAATATATCTCTAAGCAAATCCCG |
| | AATATTAAGTCCGGCCATTGTAAAAATCTGCTCCAGAGCGCC |
| | CTCCACCTTCAGCCTCAAGCAGCGAATCATGATTGCAAAAAT |
| | TCAGGTTCCTCACAGACCTGTATAAGATTCAAAAGCGGAACA |
| | TTAACAAAAATACCGCGATCCCGTAGGTCCCTTCGCAGGGCC |
| | AGCTGAACATAATCGTGCAGGTCTGCACGGACCAGCGCGGCC |
| | ACTTCCCCGCCAGGAACCATGACAAAAGAACCCACACTGATT |
| | ATGACACGCATACTCGGAGCTATGCTAACCAGCGTAGCCCCG |
| | ATGTAAGCTTGTTGCATGGGCGGCGATATAAAATGCAAGGTG |
| | CTGCTCAAAAAATCAGGCAAAGCCTCGCGCAAAAAAGAAAG |
| | CACATCGTAGTCATGCTCATGCAGATAAAGGCAGGTAAGCTC |
| | CGGAACCACCACAGAAAAAGACACCATTTTTCTCTCAAACAT |
| | GTCTGCGGGTTTCTGCATAAACACAAAATAAAATAACAAAAA |
| | AACATTTAAACATTAGAAGCCTGTCTTACAACAGGAAAAACA |
| | ACCCTTATAAGCATAAGACGGACTACGGCCATGCCGGCGTGA |
| | CCGTAAAAAAACTGGTCACCGTGATTAAAAAGCACCACCGAC |
| | AGCTCCTCGGTCATGTCCGGAGTCATAATGTAAGACTCGGTA |
| | AACACATCAGGTTGATTCACATCGGTCAGTGCTAAAAAGCGA |
| | CCGAAATAGCCCGGGGGAATACATACCCGCAGGCGTAGAGA |
| | CAACATTACAGCCCCCATAGGAGGTATAACAAAATTAATAGG |
| | AGAGAAAAACACATAAACACCTGAAAAACCCTCCTGCCTAG |
| | GCAAAATAGCACCCTCCCGCTCCAGAACAACATACAGCGCTT |
| | CCACAGCGGCAGCCATAACAGTCAGCCTTACCAGTAAAAAAG |
| | AAAACCTATTAAAAAAACACCACTCGACACGGCACCAGCTCA |
| | ATCAGTCACAGTGTAAAAAAGGGCCAAGTGCAGAGCGAGTA |
| | TATATAGGACTAAAAAATGACGTAACGGTTAAAGTCCACAAA |
| | AAACACCCAGAAAACCGCACGCGAACCTACGCCCAGAAACG |
| | AAAGCCAAAAAACCCACAACTTCCTCAAATCGTCACTTCCGT |
| | TTTCCCACGTTACGTCACTTCCCATTTTAAGAAAACTACAATT |
| | CCCAACACATACAAGTTACTCCGCCCTAAAACCTACGTCACC |
| | CGCCCCGTTCCCACGCCCCGCGCCACGTCACAAACTCCACCC |
| | CCTCATTATCATATTGGCTTCAATCCAAAATAAGGTATATTAT |
| | TGATGAT |
| SEQ | TAGTAATCAATTACGGGGTCATTAGTTCATAGCCCATATATG |
| ID | GAGTTCCGCGTTACATAACTTACGGTAAATGGCCCGCCTGGC |
| NO: | TGACCGCCCAACGACCCCCGCCCATTGACGTCAATAATGACG |
| 4 | TATGTTCCCATAGTAACGCCAATAGGGACTTTCCATTGACGTC |
| | AATGGGTGGAGTATTTACGGTAAACTGCCCACTTGGCAGTAC |
| | ATCAAGTGTATCATATGCCAAGTACGCCCCCTATTGACGTCA |
| | ATGACGGTAAATGGCCCGCCTGGCATTATGCCCAGTACATGA |
| | CCTTATGGGACTTTCCTACTTGGCAGTACATCTACGTATTAGT |
| | CATCGCTATTACCATGGTGATGCGGTTTTGGCAGTACATCAAT |
| | GGGCGTGGATAGCGGTTTGACTCACGGGGATTTCCAAGTCTC |
| | CACCCCATTGACGTCAATGGGAGTTTGTTTTGGCACCAAAAT |
| | CAACGGGACTTTCCAAAATGTCGTAACAACTCCGCCCCATTG |
| | ACGCAAATGGGCGGTAGGCGTGTACGGTGGGAGGTCTATATA |
| | AGCAGAGCTGGTTTAGTGAACCGTCAG |
| SEQ | ATCTAGATAACTGATCATAATCAGCCATACCACATTTGTAGA |
| ID | GGTTTTACTTGCTTTAAAAAACCTCCCACACCTCCCCCTGAAC |
| NO: | CTGAAACATAAAATGAATGCAATTGTTGTTGTTAACTTGTTTA |
| 5 | TTGCAGCTTATAATGGTTACAAATAAAGCAATAGCATCACAA |
| | ATTTCACAAATAAAGCATTTTTTTCACTGCATTCTAGTTGTGG |
| | TTTGTCCAAACTCATCAATGTATCTTA |
| SEQ | ATGAGGGCGAACGACGCTCTGCAGGTGCTGGGCTTGCTTTTC |
| ID | AGCCTGGCCCGGGGCTCCGAGGTGGGCAACTCTCAGGCAGTG |
| NO: | TGTCCTGGGACTCTGAATGGCCTGAGTGTGACCGGCGATGCT |
| 27 | GAGAACCAATACCAGACACTGTACAAGCTCTACGAGAGGTGT |
| | GAGGTGGTGATGGGGAACCTTGAGATTGTGCTCACGGGACAC |
| | AATGCCGACCTCTCCTTCCTGCAGTGGATTCGAGAAGTGACA |
| | GGCTATGTCCTCGTGGCCATGAATGAATTCTCTACTCTACCAT |
| | TGCCCAACCTCCGCGTGGTGCGAGGGACCCAGGTCTACGATG |
| | GGAAGTTTGCCATCTTCGTCATGTTGAACTATAACACCAACTC |
| | CAGCCACGCTCTGCGCCAGCTCCGCTTGACTCAGCTCACCGA |
| | GATTCTGTCAGGGGGTGTTTATATTGAGAAGAACGATAAGCT |
| | TTGTCACATGGACACAATTGACTGGAGGGACATCGTGAGGGA |
| | CCGAGATGCTGAGATAGTGGTGAAGGACAATGGCAGAAGCT |
| | GTCCCCCCTGTCATGAGGTTTGCAAGGGGCGATGCTGGGGTC |
| | CTGGATCAGAAGACTGCCAGACATTGACCAAGACCATCTGTG |
| | CTCCTCAGTGTAATGGTCACTGCTTTGGGCCCAACCCCAACCA |
| | GTGCTGCCATGATGAGTGTGCCGGGGGCTGCTCAGGCCCTCA |
| | GGACACAGACTGCTTTGCCTGCCGGCACTTCAATGACAGTGG |
| | AGCCTGTGTACCTCGCTGTCCACAGCCTCTTGTCTACAACAAG |
| | CTAACTTTCCAGCTGGAACCCAATCCCCACACCAAGTATCAG |
| | TATGGAGGAGTTTGTGTAGCCAGCTGTCCCCATAACTTTGTGG |
| | TGGATCAAACATCCTGTGTCAGGGCCTGTCCTCCTGACAAGA |
| | TGGAAGTAGATAAAAATGGGCTCAAGATGTGTGAGCCTTGTG |
| | GGGGACTATGTCCCAAAGCCTGTGAGGGAACAGGCTCTGGGA |
| | GCCGCTTCCAGACTGTGGACTCGAGCAACATTGATGGATTTG |
| | TGAACTGCACCAAGATCCTGGGCAACCTGGACTTTCTGATCA |
| | CCGGCCTCAATGGAGACCCCTGGCACAAGATCCCTGCCCTGG |
| | ACCCAGAGAAGCTCAATGTCTTCCGGACAGTACGGGAGATCA |
| | CAGGTTACCTGAACATCCAGTCCTGGCCGCCCCACATGCACA |
| | ACTTCAGTGTTTTTTCCAATTTGACAACCATTGGAGGCAGAAG |
| | CCTCTACAACCGGGGCTTCTCATTGTTGATCATGAAGAACTTG |
| | AATGTCACATCTCTGGGCTTCCGATCCCTGAAGGAAATTAGT |
| | GCTGGGCGTATCTATATAAGTGCCAATAGGCAGCTCTGCTAC |
| | CACCACTCTTTGAACTGGACCAAGGTGCTTCGGGGGCCTACG |
| | GAAGAGCGACTAGACATCAAGCATAATCGGCCGCGCAGAGA |
| | CTGCGTGGCAGAGGGCAAAGTGTGTGACCCACTGTGCTCCTC |
| | TGGGGGATGCTGGGGCCCAGGCCCTGGTCAGTGCTTGTCCTG |
| | TCGAAATTATAGCCGAGGAGGTGTCTGTGTGACCCACTGCAA |
| | CTTTCTGAATGGGGAGCCTCGAGAATTTGCCCATGAGGCCGA |
| | ATGCTTCTCCTGCCACCCGGAATGCCAACCCATGGAGGGCAC |
| | TGCCACATGCAATGGCTCGGGCTCTGATACTTGTGCTCAATGT |
| | GCCCATTTTCGAGATGGGCCCCACTGTGTGAGCAGCTGCCCC |
| | CATGGAGTCCTAGGTGCCAAGGGCCCAATCTACAAGTACCCA |
| | GATGTTCAGAATGAATGTCGGCCCTGCCATGAGAACTGCACC |
| | CAGGGGTGTAAAGGACCAGAGCTTCAAGACTGTTTAGGACAA |
| | ACACTGGTGCTGATCGGCAAAACCCATCTGACAATGGCTTTG |
| | ACAGTGATAGCAGGATTGGTAGTGATTTTCATGATGCTGGGC |
| | GGCACTTTTTAA |
| SEQ | YLSGANLNL |
| ID | |
| NO: | |
| 28 | |
| SEQ | CATCATCAATAATATACCTTATTTTGGATTGAAGCCAATATGA |
| ID | TAATGAGGGGGTGGAGTTTGTGACGTGGCGCGGGGCGTGGG |
| NO: | AACGGGGCGGGTGACGTAGTAGTGTGGCGGAAGTGTGATGTT |
| 29 | GCAAGTGTGGCGGAACACATGTAAGCGACGGATGTGGCAAA |
| | AGTGACGTTTTTGGTGTGCGCCGGTGTACACAGGAAGTGACA |
| | ATTTTCGCGCGGTTTTAGGCGGATGTTGTAGTAAATTTGGGCG |
| | TAACCGAGTAAGATTTGGCCATTTTCGCGGGAAAACTGAATA |
| | AGAGGAAGTGAAATCTGAATAATTTTGTGTTACTCATAGCGC |
| | GTAATACTGTAATAGTAATCAATTACGGGGTCATTAGTTCAT |
| | AGCCCATATATGGAGTTCCGCGTTACATAACTTACGGTAAAT |
| | GGCCCGCCTGGCTGACCGCCCAACGACCCCCGCCCATTGACG |
| | TCAATAATGACGTATGTTCCCATAGTAACGCCAATAGGGACT |
| | TTCCATTGACGTCAATGGGTGGAGTATTTACGGTAAACTGCC |
| | CACTTGGCAGTACATCAAGTGTATCATATGCCAAGTACGCCC |
| | CCTATTGACGTCAATGACGGTAAATGGCCCGCCTGGCATTAT |
| | GCCCAGTACATGACCTTATGGGACTTTCCTACTTGGCAGTACA |
| | TCTACGTATTAGTCATCGCTATTACCATGGTGATGCGGTTTTG |
| | GCAGTACATCAATGGGCGTGGATAGCGGTTTGACTCACGGGG |
| | ATTTCCAAGTCTCCACCCCATTGACGTCAATGGGAGTTTGTTT |
| | TGGCACCAAAATCAACGGGACTTTCCAAAATGTCGTAACAAC |
| | TCCGCCCCATTGACGCAAATGGGCGGTAGGCGTGTACGGTGG |
| | GAGGTCTATATAAGCAGAGCTGGTTTAGTGAACCGTCAGATC |
| | CGCTAGAGATCTGGTACCGTCGACGCGGCCGCTCGAGCCTAA |
| | GCTTGGTACCGAGCTCGGATCCACTAGTAACGGCCGCCAGTG |
| | TGCTGGAATTCGGCTTAAAGGTACCCAGAGCAGACAGCCGCC |
| | ACCATGGAGTCTCCCTCGGCCCCTCCCCACAGATGGTGCATC |
| | CCCTGGCAGAGGCTCCTGCTCACAGCCTCACTTCTAACCTTCT |
| | GGAACCCGCCCACCACTGCCAAGCTCACTATTGAATCCACGC |
| | CGTTCAATGTCGCAGAGGGGAAGGAGGTGCTTCTACTTGTCC |
| | ACAATCTGCCCCAGCATCTTTTTGGCTACAGCTGGTACAAAG |
| | GTGAAAGAGTGGATGGCAACCGTCAAATTATAGGATATGTAA |
| | TAGGAACTCAACAAGCTACCCCAGGGCCCGCATACAGTGGTC |
| | GAGAGATAATATACCCCAATGCATCCCTGCTGATCCAGAACA |
| | TCATCCAGAATGACACAGGATTCTACACCCTACACGTCATAA |
| | AGTCAGATCTTGTGAATGAAGAAGCAACTGGCCAGTTCCGGG |
| | TATACCCGGAGCTGCCCAAGCCCTCCATCTCCAGCAACAACT |
| | CCAAACCCGTGGAGGACAAGGATGCTGTGGCCTTCACCTGTG |
| | AACCTGAGACTCAGGACGCAACCTACCTGTGGTGGGTAAACA |
| | ATCAGAGCCTCCCGGTCAGTCCCAGGCTGCAGCTGTCCAATG |
| | GCAACAGGACCCTCACTCTATTCAATGTCACAAGAAATGACA |
| | CAGCAAGCTACAAATGTGAAACCCAGAACCCAGTGAGTGCC |
| | AGGCGCAGTGATTCAGTCATCCTGAATGTCCTCTATGGCCCG |
| | GATGCCCCCACCATTTCCCCTCTAAACACATCTTACAGATCAG |
| | GGGAAAATCTGAACCTCTCCTGCCACGCAGCCTCTAACCCAC |
| | CTGCACAGTACTCTTGGTTTGTCAATGGGACTTTCCAGCAATC |
| | CACCCAAGAGCTCTTTATCCCCAACATCACTGTGAATAATAG |
| | TGGATCCTATACGTGCCAAGCCCATAACTCAGACACTGGCCT |
| | CAATAGGACCACAGTCACGACGATCACAGTCTATGCAGAGCC |
| | ACCCAAACCCTTCATCACCAGCAACAACTCCAACCCCGTGGA |
| | GGATGAGGATGCTGTAGCCTTAACCTGTGAACCTGAGATTCA |
| | GAACACAACCTACCTGTGGTGGGTAAATAATCAGAGCCTCCC |
| | GGTCAGTCCCAGGCTGCAGCTGTCCAATGACAACAGGACCCT |
| | CACTCTACTCAGTGTCACAAGGAATGATGTAGGACCCTATGA |
| | GTGTGGAATCCAGAACGAATTAAGTGTTGACCACAGCGACCC |
| | AGTCATCCTGAATGTCCTCTATGGCCCAGACGACCCCACCATT |
| | TCCCCCTCATACACCTATTACCGTCCAGGGGTGAACCTCAGCC |
| | TCTCCTGCCATGCAGCCTCTAACCCACCTGCACAGTATTCTTG |
| | GCTGATTGATGGGAACATCCAGCAACACACACAAGAGCTCTT |
| | TATCTCCAACATCACTGAGAAGAACAGCGGACTCTATACCTG |
| | CCAGGCCAATAACTCAGCCAGTGGCCACAGCAGGACTACAGT |
| | CAAGACAATCACAGTCTCTGCGGAGCTGCCCAAGCCCTCCAT |
| | CTCCAGCAACAACTCCAAACCCGTGGAGGACAAGGATGCTGT |
| | GGCCTTCACCTGTGAACCTGAGGCTCAGAACACAACCTACCT |
| | GTGGTGGGTAAATGGTCAGAGCCTCCCAGTCAGTCCCAGGCT |
| | GCAGCTGTCCAATGGCAACAGGACCCTCACTCTATTCAATGT |
| | CACAAGAAATGACGCAAGAGCCTATGTATGTGGAATCCAGA |
| | ACTCAGTGAGTGCAAACCGCAGTGACCCAGTCACCCTGGATG |
| | TCCTCTATGGGCCGGACACCCCCATCATTTCCCCCCCAGACTC |
| | GTCTTACCTTTCGGGAGCGGACCTCAACCTCTCCTGCCACTCG |
| | GCCTCTAACCCATCCCCGCAGTATTCTTGGCGTATCAATGGGA |
| | TACCGCAGCAACACACACAAGTTCTCTTTATCGCCAAAATCA |
| | CGCCAAATAATAACGGGACCTATGCCTGTTTTGTCTCTAACTT |
| | GGCTACTGGCCGCAATAATTCCATAGTCAAGAGCATCACAGT |
| | CTCTGCATCTGGAACTTCTCCTGGTCTCTCAGCTGGGGCCACT |
| | GTCGGCATCATGATTGGAGTGCTGGTTGGGGTTGCTCTGATAT |
| | AGCAGCCCTGGTGTAGTTTCTTCATTTCAGGAAGACTGACAG |
| | TTGTTTTGCTTCTTCCTTAAAGCATTTGCAACAGCTACAGTCT |
| | AAAATTGCTTCTTTACCAAGGATATTTACAGAAAAGACTCTG |
| | ACCAGAGATCGAGACCATCCTCTAGATAAGATATCCGATCCA |
| | CCGGATCTAGATAACTGATCATAATCAGCCATACCACATTTG |
| | TAGAGGTTTTACTTGCTTTAAAAAACCTCCCACACCTCCCCCT |
| | GAACCTGAAACATAAAATGAATGCAATTGTTGTTGTTAACTT |
| | GTTTATTGCAGCTTATAATGGTTACAAATAAAGCAATAGCAT |
| | CACAAATTTCACAAATAAAGCATTTTTTTCACTGCATTCTAGT |
| | TGTGGTTTGTCCAAACTCATCAATGTATCTTAACGCGGATCTG |
| | GGCGTGGTTAAGGGTGGGAAAGAATATATAAGGTGGGGGTC |
| | TTATGTAGTTTTGTATCTGTTTTGCAGCAGCCGCCGCCGCCAT |
| | GAGCACCAACTCGTTTGATGGAAGCATTGTGAGCTCATATTT |
| | GACAACGCGCATGCCCCCATGGGCCGGGGTGCGTCAGAATGT |
| | GATGGGCTCCAGCATTGATGGTCGCCCCGTCCTGCCCGCAAA |
| | CTCTACTACCTTGACCTACGAGACCGTGTCTGGAACGCCGTTG |
| | GAGACTGCAGCCTCCGCCGCCGCTTCAGCCGCTGCAGCCACC |
| | GCCCGCGGGATTGTGACTGACTTTGCTTTCCTGAGCCCGCTTG |
| | CAAGCAGTGCAGCTTCCCGTTCATCCGCCCGCGATGACAAGT |
| | TGACGGCTCTTTTGGCACAATTGGATTCTTTGACCCGGGAACT |
| | TAATGTCGTTTCTCAGCAGCTGTTGGATCTGCGCCAGCAGGTT |
| | TCTGCCCTGAAGGCTTCCTCCCCTCCCAATGCGGTTTAAAACA |
| | TAAATAAAAAACCAGACTCTGTTTGGATTTGGATCAAGCAAG |
| | TGTCTTGCTGTCTTTATTTAGGGGTTTTGCGCGCGCGGTAGGC |
| | CCGGGACCAGCGGTCTCGGTCGTTGAGGGTCCTGTGTATTTTT |
| | TCCAGGACGTGGTAAAGGTGACTCTGGATGTTCAGATACATG |
| | GGCATAAGCCCGTCTCTGGGGTGGAGGTAGCACCACTGCAGA |
| | GCTTCATGCTGCGGGGTGGTGTTGTAGATGATCCAGTCGTAG |
| | CAGGAGCGCTGGGCGTGGTGCCTAAAAATGTCTTTCAGTAGC |
| | AAGCTGATTGCCAGGGGCAGGCCCTTGGTGTAAGTGTTTACA |
| | AAGCGGTTAAGCTGGGATGGGTGCATACGTGGGGATATGAG |
| | ATGCATCTTGGACTGTATTTTTAGGTTGGCTATGTTCCCAGCC |
| | ATATCCCTCCGGGGATTCATGTTGTGCAGAACCACCAGCACA |
| | GTGTATCCGGTGCACTTGGGAAATTTGTCATGTAGCTTAGAA |
| | GGAAATGCGTGGAAGAACTTGGAGACGCCCTTGTGACCTCCA |
| | AGATTTTCCATGCATTCGTCCATAATGATGGCAATGGGCCCA |
| | CGGGCGGCGGCCTGGGCGAAGATATTTCTGGGATCACTAACG |
| | TCATAGTTGTGTTCCAGGATGAGATCGTCATAGGCCATTTTTA |
| | CAAAGCGCGGGCGGAGGGTGCCAGACTGCGGTATAATGGTTC |
| | CATCCGGCCCAGGGGCGTAGTTACCCTCACAGATTTGCATTTC |
| | CCACGCTTTGAGTTCAGATGGGGGGATCATGTCTACCTGCGG |
| | GGCGATGAAGAAAACGGTTTCCGGGGTAGGGGAGATCAGCT |
| | GGGAAGAAAGCAGGTTCCTGAGCAGCTGCGACTTACCGCAGC |
| | CGGTGGGCCCGTAAATCACACCTATTACCGGCTGCAACTGGT |
| | AGTTAAGAGAGCTGCAGCTGCCGTCATCCCTGAGCAGGGGGG |
| | CCACTTCGTTAAGCATGTCCCTGACTCGCATGTTTTCCCTGAC |
| | CAAATCCGCCAGAAGGCGCTCGCCGCCCAGCGATAGCAGTTC |
| | TTGCAAGGAAGCAAAGTTTTTCAACGGTTTGAGACCGTCCGC |
| | CGTAGGCATGCTTTTGAGCGTTTGACCAAGCAGTTCCAGGCG |
| | GTCCCACAGCTCGGTCACCTGCTCTACGGCATCTCGATCCAGC |
| | ATATCTCCTCGTTTCGCGGGTTGGGGCGGCTTTCGCTGTACGG |
| | CAGTAGTCGGTGCTCGTCCAGACGGGCCAGGGTCATGTCTTT |
| | CCACGGGCGCAGGGTCCTCGTCAGCGTAGTCTGGGTCACGGT |
| | GAAGGGGTGCGCTCCGGGCTGCGCGCTGGCCAGGGTGCGCTT |
| | GAGGCTGGTCCTGCTGGTGCTGAAGCGCTGCCGGTCTTCGCC |
| | CTGCGCGTCGGCCAGGTAGCATTTGACCATGGTGTCATAGTC |
| | CAGCCCCTCCGCGGCGTGGCCCTTGGCGCGCAGCTTGCCCTT |
| | GGAGGAGGCGCCGCACGAGGGGCAGTGCAGACTTTTGAGGG |
| | CGTAGAGCTTGGGCGCGAGAAATACCGATTCCGGGGAGTAG |
| | GCATCCGCGCCGCAGGCCCCGCAGACGGTCTCGCATTCCACG |
| | AGCCAGGTGAGCTCTGGCCGTTCGGGGTCAAAAACCAGGTTT |
| | CCCCCATGCTTTTTGATGCGTTTCTTACCTCTGGTTTCCATGAG |
| | CCGGTGTCCACGCTCGGTGACGAAAAGGCTGTCCGTGTCCCC |
| | GTATACAGACTTGAGAGGCCTGTCCTCGAGCGGTGTTCCGCG |
| | GTCCTCCTCGTATAGAAACTCGGACCACTCTGAGACAAAGGC |
| | TCGCGTCCAGGCCAGCACGAAGGAGGCTAAGTGGGAGGGGT |
| | AGCGGTCGTTGTCCACTAGGGGGTCCACTCGCTCCAGGGTGT |
| | GAAGACACATGTCGCCCTCTTCGGCATCAAGGAAGGTGATTG |
| | GTTTGTAGGTGTAGGCCACGTGACCGGGTGTTCCTGAAGGGG |
| | GGCTATAAAAGGGGGTGGGGGCGCGTTCGTCCTCACTCTCTT |
| | CCGCATCGCTGTCTGCGAGGGCCAGCTGTTGGGGTGAGTACT |
| | CCCTCTGAAAAGCGGGCATGACTTCTGCGCTAAGATTGTCAG |
| | TTTCCAAAAACGAGGAGGATTTGATATTCACCTGGCCCGCGG |
| | TGATGCCTTIGAGGGTGGCCGCATCCATCTGGTCAGAAAAGA |
| | CAATCTTTTTGTTGTCAAGCTTGGTGGCAAACGACCCGTAGA |
| | GGGCGTTGGACAGCAACTTGGCGATGGAGCGCAGGGTTTGGT |
| | TTTTGTCGCGATCGGCGCGCTCCTTGGCCGCGATGTTTAGCTG |
| | CACGTATTCGCGCGCAACGCACCGCCATTCGGGAAAGACGGT |
| | GGTGCGCTCGTCGGGCACCAGGTGCACGCGCCAACCGCGGTT |
| | GTGCAGGGTGACAAGGTCAACGCTGGTGGCTACCTCTCCGCG |
| | TAGGCGCTCGTTGGTCCAGCAGAGGCGGCCGCCCTTGCGCGA |
| | GCAGAATGGCGGTAGGGGGTCTAGCTGCGTCTCGTCCGGGGG |
| | GTCTGCGTCCACGGTAAAGACCCCGGGCAGCAGGCGCGCGTC |
| | GAAGTAGTCTATCTTGCATCCTTGCAAGTCTAGCGCCTGCTGC |
| | CATGCGCGGGCGGCAAGCGCGCGCTCGTATGGGTTGAGTGGG |
| | GGACCCCATGGCATGGGGTGGGTGAGCGCGGAGGCGTACAT |
| | GCCGCAAATGTCGTAAACGTAGAGGGGCTCTCTGAGTATTCC |
| | AAGATATGTAGGGTAGCATCTTCCACCGCGGATGCTGGCGCG |
| | CACGTAATCGTATAGTTCGTGCGAGGGAGCGAGGAGGTCGGG |
| | ACCGAGGTTGCTACGGGCGGGCTGCTCTGCTCGGAAGACTAT |
| | CTGCCTGAAGATGGCATGTGAGTTGGATGATATGGTTGGACG |
| | CTGGAAGACGTTGAAGCTGGCGTCTGTGAGACCTACCGCGTC |
| | ACGCACGAAGGAGGCGTAGGAGTCGCGCAGCTTGTTGACCA |
| | GCTCGGCGGTGACCTGCACGTCTAGGGCGCAGTAGTCCAGGG |
| | TTTCCTTGATGATGTCATACTTATCCTGTCCCTTTTTTTTCCAC |
| | AGCTCGCGGTTGAGGACAAACTCTTCGCGGTCTTTCCAGTACT |
| | CTTGGATCGGAAACCCGTCGGCCTCCGAACGGTAAGAGCCTA |
| | GCATGTAGAACTGGTTGACGGCCTGGTAGGCGCAGCATCCCT |
| | TTTCTACGGGTAGCGCGTATGCCTGCGCGGCCTTCCGGCATG |
| | ACCAGCATGAAGGGCACGAGCTGCTTCCCAAAGGCCCCCATC |
| | CAAGTATAGGTCTCTACATCGTAGGTGACAAAGAGACGCTCG |
| | GTGCGAGGATGCGAGCCGATCGGGAAGAACTGGATCTCCCGC |
| | CACCAATTGGAGGAGTGGCTATTGATGTGGTGAAAGTAGAAG |
| | TCCCTGCGACGGGCCGAACACTCGTGCTGGCTTTTGTAAAAA |
| | CGTGCGCAGTACTGGCAGCGGTGCACGGGCTGTACATCCTGC |
| | ACGAGGTTGACCTGACGACCGCGCACAAGGAAGCAGAGTGG |
| | GAATTTGAGCCCCTCGCCTGGCGGGTTTGGCTGGTGGTCTTCT |
| | ACTTCGGCTGCTTGTCCTTGACCGTCTGGCTGCTCGAGGGGAG |
| | TTACGGTGGATCGGACCACCACGCCGCGCGAGCCCAAAGTCC |
| | AGATGTCCGCGCGCGGCGGTCGGAGCTTGATGACAACATCGC |
| | GCAGATGGGAGCTGTCCATGGTCTGGAGCTCCCGCGGCGTCA |
| | GGTCAGGCGGGAGCTCCTGCAGGTTTACCTCGCATAGACGGG |
| | TCAGGGCGCGGGCTAGATCCAGGTGATACCTAATTTCCAGGG |
| | GCTGGTTGGTGGCGGCGTCGATGGCTTGCAAGAGGCCGCATC |
| | CCCGCGGCGCGACTACGGTACCGCGCGGCGGGCGGTGGGCC |
| | GCGGGGGTGTCCTTGGATGATGCATCTAAAAGCGGTGACGCG |
| | GGCGAGCCCCCGGAGGTAGGGGGGGCTCCGGACCCGCCGGG |
| | AGAGGGGGCAGGGGCACGTCGGCGCCGCGCGCGGGCAGGAG |
| | CTGGTGCTGCGCGCGTAGGTTGCTGGCGAACGCGACGACGCG |
| | GCGGTTGATCTCCTGAATCTGGCGCCTCTGCGTGAAGACGAC |
| | GGGCCCGGTGAGCTTGAACCTGAAAGAGAGTTCGACAGAATC |
| | AATTTCGGTGTCGTTGACGGCGGCCTGGCGCAAAATCTCCTG |
| | CACGTCTCCTGAGTTGTCTTGATAGGCGATCTCGGCCATGAAC |
| | TGCTCGATCTCTTCCTCCTGGAGATCTCCGCGTCCGGCTCGCT |
| | CCACGGTGGCGGCGAGGTCGTTGGAAATGCGGGCCATGAGCT |
| | GCGAGAAGGCGTTGAGGCCTCCCTCGTTCCAGACGCGGCTGT |
| | AGACCACGCCCCCTTCGGCATCGCGGGCGCGCATGACCACCT |
| | GCGCGAGATTGAGCTCCACGTGCCGGGCGAAGACGGCGTAGT |
| | TTCGCAGGCGCTGAAAGAGGTAGTTGAGGGTGGTGGCGGTGT |
| | GTTCTGCCACGAAGAAGTACATAACCCAGCGTCGCAACGTGG |
| | ATTCGTTGATAATTGTTGTGTAGGTACTCCGCCGCCGAGGGA |
| | CCTGAGCGAGTCCGCATCGACCGGATCGGAAAACCTCTCGAG |
| | AAAGGCGTCTAACCAGTCACAGTCGCAAGGTAGGCTGAGCAC |
| | CGTGGCGGGCGGCAGCGGGCGGCGGTCGGGGTTGTTTCTGGC |
| | GGAGGTGCTGCTGATGATGTAATTAAAGTAGGCGGTCTTGAG |
| | ACGGCGGATGGTCGACAGAAGCACCATGTCCTTGGGTCCGGC |
| | CTGCTGAATGCGCAGGCGGTCGGCCATGCCCCAGGCTTCGTT |
| | TTGACATCGGCGCAGGTCTTTGTAGTAGTCTTGCATGAGCCTT |
| | TCTACCGGCACTTCTTCTTCTCCTTCCTCTTGTCCTGCATCTCT |
| | TGCATCTATCGCTGCGGCGGCGGCGGAGTTTGGCCGTAGGTG |
| | GCGCCCTCTTCCTCCCATGCGTGTGACCCCGAAGCCCCTCATC |
| | GGCTGAAGCAGGGCTAGGTCGGCGACAACGCGCTCGGCTAAT |
| | ATGGCCTGCTGCACCTGCGTGAGGGTAGACTGGAAGTCATCC |
| | ATGTCCACAAAGCGGTGGTATGCGCCCGTGTTGATGGTGTAA |
| | GTGCAGTTGGCCATAACGGACCAGTTAACGGTCTGGTGACCC |
| | GGCTGCGAGAGCTCGGTGTACCTGAGACGCGAGTAAGCCCTC |
| | GAGTCAAATACGTAGTCGTTGCAAGTCCGCACCAGGTACTGG |
| | TATCCCACCAAAAAGTGCGGCGGCGGCTGGCGGTAGAGGGG |
| | CCAGCGTAGGGTGGCCGGGGCTCCGGGGGCGAGATCTTCCAA |
| | CATAAGGCGATGATATCCGTAGATGTACCTGGACATCCAGGT |
| | GATGCCGGCGGCGGTGGTGGAGGCGCGCGGAAAGTCGCGGA |
| | CGCGGTTCCAGATGTTGCGCAGCGGCAAAAAGTGCTCCATGG |
| | TCGGGACGCTCTGGCCGGTCAGGCGCGCGCAATCGTTGACGC |
| | TCTAGCGTGCAAAAGGAGAGCCTGTAAGCGGGCACTCTTCCG |
| | TGGTCTGGTGGATAAATTCGCAAGGGTATCATGGCGGACGAC |
| | CGGGGTTCGAGCCCCGTATCCGGCCGTCCGCCGTGATCCATG |
| | CGGTTACCGCCCGCGTGTCGAACCCAGGTGTGCGACGTCAGA |
| | CAACGGGGGAGTGCTCCTTTTGGCTTCCTTCCAGGCGCGGCG |
| | GCTGCTGCGCTAGCTTTTTTGGCCACTGGCCGCGCGCAGCGTA |
| | AGCGGTTAGGCTGGAAAGCGAAAGCATTAAGTGGCTCGCTCC |
| | CTGTAGCCGGAGGGTTATTTTCCAAGGGTTGAGTCGCGGGAC |
| | CCCCGGTTCGAGTCTCGGACCGGCCGGACTGCGGCGAACGGG |
| | GGTTTGCCTCCCCGTCATGCAAGACCCCGCTTGCAAATTCCTC |
| | CGGAAACAGGGACGAGCCCCTTTTTTGCTTTTCCCAGATGCAT |
| | CCGGTGCTGCGGCAGATGCGCCCCCCTCCTCAGCAGCGGCAA |
| | GAGCAAGAGCAGCGGCAGACATGCAGGGCACCCTCCCCTCCT |
| | CCTACCGCGTCAGGAGGGGCGACATCCGCGGTTGACGCGGCA |
| | GCAGATGGTGATTACGAACCCCCGCGGCGCCGGGCCCGGCAC |
| | TACCTGGACTTGGAGGAGGGCGAGGGCCTGGCGCGGCTAGG |
| | AGCGCCCTCTCCTGAGCGGCACCCAAGGGTGCAGCTGAAGCG |
| | TGATACGCGTGAGGCGTACGTGCCGCGGCAGAACCTGTTTCG |
| | CGACCGCGAGGGAGAGGAGCCCGAGGAGATGCGGGATCGAA |
| | AGTTCCACGCAGGGCGCGAGCTGCGGCATGGCCTGAATCGCG |
| | AGCGGTTGCTGCGCGAGGAGGACTTTGAGCCCGACGCGCGAA |
| | CCGGGATTAGTCCCGCGCGCGCACACGTGGCGGCCGCCGACC |
| | TGGTAACCGCATACGAGCAGACGGTGAACCAGGAGATTAACT |
| | TTCAAAAAAGCTTTAACAACCACGTGCGTACGCTTGTGGCGC |
| | GCGAGGAGGTGGCTATAGGACTGATGCATCTGTGGGACTTTG |
| | TAAGCGCGCTGGAGCAAAACCCAAATAGCAAGCCGCTCATG |
| | GCGCAGCTGTTCCTTATAGTGCAGCACAGCAGGGACAACGAG |
| | GCATTCAGGGATGCGCTGCTAAACATAGTAGAGCCCGAGGGC |
| | CGCTGGCTGCTCGATTTGATAAACATCCTGCAGAGCATAGTG |
| | GTGCAGGAGCGCAGCTTGAGCCTGGCTGACAAGGTGGCCGCC |
| | ATCAACTATTCCATGCTTAGCCTGGGCAAGTTTTACGCCCGCA |
| | AGATATACCATACCCCTTACGTTCCCATAGACAAGGAGGTAA |
| | AGATCGAGGGGTTCTACATGCGCATGGCGCTGAAGGTGCTTA |
| | CCTTGAGCGACGACCTGGGCGTTTATCGCAACGAGCGCATCC |
| | ACAAGGCCGTGAGCGTGAGCCGGCGGCGCGAGCTCAGCGAC |
| | CGCGAGCTGATGCACAGCCTGCAAAGGGCCCTGGCTGGCACG |
| | GGCAGCGGCGATAGAGAGGCCGAGTCCTACTTTGACGCGGGC |
| | GCTGACCTGCGCTGGGCCCCAAGCCGACGCGCCCTGGAGGCA |
| | GCTGGGGCCGGACCTGGGCTGGCGGTGGCACCCGCGCGCGCT |
| | GGCAACGTCGGCGGCGTGGAGGAATATGACGAGGACGATGA |
| | GTACGAGCCAGAGGACGGCGAGTACTAAGCGGTGATGTTTCT |
| | GATCAGATGATGCAAGACGCAACGGACCCGGCGGTGCGGGC |
| | GGCGCTGCAGAGCCAGCCGTCCGGCCTTAACTCCACGGACGA |
| | CTGGCGCCAGGTCATGGACCGCATCATGTCGCTGACTGCGCG |
| | CAATCCTGACGCGTTCCGGCAGCAGCCGCAGGCCAACCGGCT |
| | CTCCGCAATTCTGGAAGCGGTGGTCCCGGCGCGCGCAAACCC |
| | CACGCACGAGAAGGTGCTGGCGATCGTAAACGCGCTGGCCG |
| | AAAACAGGGCCATCCGGCCCGACGAGGCCGGCCTGGTCTACG |
| | ACGCGCTGCTTCAGCGCGTGGCTCGTTACAACAGCGGCAACG |
| | TGCAGACCAACCTGGACCGGCTGGTGGGGGATGTGCGCGAG |
| | GCCGTGGCGCAGCGTGAGCGCGCGCAGCAGCAGGGCAACCT |
| | GGGCTCCATGGTTGCACTAAACGCCTTCCTGAGTACACAGCC |
| | CGCCAACGTGCCGCGGGGACAGGAGGACTACACCAACTTTGT |
| | GAGCGCACTGCGGCTAATGGTGACTGAGACACCGCAAAGTG |
| | AGGTGTACCAGTCTGGGCCAGACTATTTTTTCCAGACCAGTA |
| | GACAAGGCCTGCAGACCGTAAACCTGAGCCAGGCTTTCAAAA |
| | ACTTGCAGGGGCTGTGGGGGGTGCGGGCTCCCACAGGCGACC |
| | GCGCGACCGTGTCTAGCTTGCTGACGCCCAACTCGCGCCTGTT |
| | GCTGCTGCTAATAGCGCCCTTCACGGACAGTGGCAGCGTGTC |
| | CCGGGACACATACCTAGGTCACTTGCTGACACTGTACCGCGA |
| | GGCCATAGGTCAGGCGCATGTGGACGAGCATACTTTCCAGGA |
| | GATTACAAGTGTCAGCCGCGCGCTGGGGCAGGAGGACACGG |
| | GCAGCCTGGAGGCAACCCTAAACTACCTGCTGACCAACCGGC |
| | GGCAGAAGATCCCCTCGTTGCACAGTTTAAACAGCGAGGAGG |
| | AGCGCATTTTGCGCTACGTGCAGCAGAGCGTGAGCCTTAACC |
| | TGATGCGCGACGGGGTAACGCCCAGCGTGGCGCTGGACATGA |
| | CCGCGCGCAACATGGAACCGGGCATGTATGCCTCAAACCGGC |
| | CGTTTATCAACCGCCTAATGGACTACTTGCATCGCGCGGCCG |
| | CCGTGAACCCCGAGTATTTCACCAATGCCATCTTGAACCCGC |
| | ACTGGCTACCGCCCCCTGGTTTCTACACCGGGGGATTCGAGG |
| | TGCCCGAGGGTAACGATGGATTCCTCTGGGACGACATAGACG |
| | ACAGCGTGTTTTCCCCGCAACCGCAGACCCTGCTAGAGTTGC |
| | AACAGCGCGAGCAGGCAGAGGCGGCGCTGCGAAAGGAAAGC |
| | TTCCGCAGGCCAAGCAGCTTGTCCGATCTAGGCGCTGCGGCC |
| | CCGCGGTCAGATGCTAGTAGCCCATTTCCAAGCTTGATAGGG |
| | TCTCTTACCAGCACTCGCACCACCCGCCCGCGCCTGCTGGGC |
| | GAGGAGGAGTACCTAAACAACTCGCTGCTGCAGCCGCAGCGC |
| | GAAAAAAACCTGCCTCCGGCATTTCCCAACAACGGGATAGAG |
| | AGCCTAGTGGACAAGATGAGTAGATGGAAGACGTACGCGCA |
| | GGAGCACAGGGACGTGCCAGGCCCGCGCCCGCCCACCCGTCG |
| | TCAAAGGCACGACCGTCAGCGGGGTCTGGTGTGGGAGGACG |
| | ATGACTCGGCAGACGACAGCAGCGTCCTGGATTTGGGAGGGA |
| | GTGGCAACCCGTTTGCGCACCTTCGCCCCAGGCTGGGGAGAA |
| | TGTTTTAAAAAAAAAAAAGCATGATGCAAAATAAAAAACTC |
| | ACCAAGGCCATGGCACCGAGCGTTGGTTTTCTTGTATTCCCCT |
| | TAGTATGCGGCGCGCGGCGATGTATGAGGAAGGTCCTCCTCC |
| | CTCCTACGAGAGTGTGGTGAGCGCGGCGCCAGTGGCGGCGGC |
| | GCTGGGTTCTCCCTTCGATGCTCCCCTGGACCCGCCGTTTGTG |
| | CCTCCGCGGTACCTGCGGCCTACCGGGGGGAGAAACAGCATC |
| | CGTTACTCTGAGTTGGCACCCCTATTCGACACCACCCGTGTGT |
| | ACCTGGTGGACAACAAGTCAACGGATGTGGCATCCCTGAACT |
| | ACCAGAACGACCACAGCAACTTTCTGACCACGGTCATTCAAA |
| | ACAATGACTACAGCCCGGGGGAGGCAAGCACACAGACCATC |
| | AATCTTGACGACCGGTCGCACTGGGGCGGCGACCTGAAAACC |
| | ATCCTGCATACCAACATGCCAAATGTGAACGAGTTCATGTTT |
| | ACCAATAAGTTTAAGGCGCGGGTGATGGTGTCGCGCTTGCCT |
| | ACTAAGGACAATCAGGTGGAGCTGAAATACGAGTGGGTGGA |
| | GTTCACGCTGCCCGAGGGCAACTACTCCGAGACCATGACCAT |
| | AGACCTTATGAACAACGCGATCGTGGAGCACTACTTGAAAGT |
| | GGGCAGACAGAACGGGGTTCTGGAAAGCGACATCGGGGTAA |
| | AGTTTGACACCCGCAACTTCAGACTGGGGTTTGACCCCGTCA |
| | CTGGTCTTGTCATGCCTGGGGTATATACAAACGAAGCCTTCC |
| | ATCCAGACATCATTTTGCTGCCAGGATGCGGGGTGGACTTCA |
| | CCCACAGCCGCCTGAGCAACTTGTTGGGCATCCGCAAGCGGC |
| | AACCCTTCCAGGAGGGCTTTAGGATCACCTACGATGATCTGG |
| | AGGGTGGTAACATTCCCGCACTGTTGGATGTGGACGCCTACC |
| | AGGCGAGCTTGAAAGATGACACCGAACAGGGCGGGGGTGGC |
| | GCAGGCGGCAGCAACAGCAGTGGCAGCGGCGCGGAAGAGAA |
| | CTCCAACGCGGCAGCCGCGGCAATGCAGCCGGTGGAGGACA |
| | TGAACGATCATGCCATTCGCGGCGACACCTTTGCCACACGGG |
| | CTGAGGAGAAGCGCGCTGAGGCCGAAGCAGCGGCCGAAGCT |
| | GCCGCCCCCGCTGCGCAACCCGAGGTCGAGAAGCCTCAGAAG |
| | AAACCGGTGATCAAACCCCTGACAGAGGACAGCAAGAAACG |
| | CAGTTACAACCTAATAAGCAATGACAGCACCTTCACCCAGTA |
| | CCGCAGCTGGTACCTTGCATACAACTACGGCGACCCTCAGAC |
| | CGGAATCCGCTCATGGACCCTGCTTTGCACTCCTGACGTAACC |
| | TGCGGCTCGGAGCAGGTCTACTGGTCGTTGCCAGACATGATG |
| | CAAGACCCCGTGACCTTCCGCTCCACGCGCCAGATCAGCAAC |
| | TTTCCGGTGGTGGGCGCCGAGCTGTTGCCCGTGCACTCCAAG |
| | AGCTTCTACAACGACCAGGCCGTCTACTCCCAACTCATCCGC |
| | CAGTTTACCTCTCTGACCCACGTGTTCAATCGCTTTCCCGAGA |
| | ACCAGATTTTGGCGCGCCCGCCAGCCCCCACCATCACCACCG |
| | TCAGTGAAAACGTTCCTGCTCTCACAGATCACGGGACGCTAC |
| | CGCTGCGCAACAGCATCGGAGGAGTCCAGCGAGTGACCATTA |
| | CTGACGCCAGACGCCGCACCTGCCCCTACGTTTACAAGGCCC |
| | TGGGCATAGTCTCGCCGCGCGTCCTATCGAGCCGCACTTTTTG |
| | AGCAAGCATGTCCATCCTTATATCGCCCAGCAATAACACAGG |
| | CTGGGGCCTGCGCTTCCCAAGCAAGATGTTTGGCGGGGCCAA |
| | GAAGCGCTCCGACCAACACCCAGTGCGCGTGCGCGGGCACTA |
| | CCGCGCGCCCTGGGGCGCGCACAAACGCGGCCGCACTGGGC |
| | GCACCACCGTCGATGACGCCATCGACGCGGTGGTGGAGGAG |
| | GCGCGCAACTACACGCCCACGCCGCCACCAGTGTCCACAGTG |
| | GACGCGGCCATTCAGACCGTGGTGCGCGGAGCCCGGCGCTAT |
| | GCTAAAATGAAGAGACGGCGGAGGCGCGTAGCACGTCGCCA |
| | CCGCCGCCGACCCGGCACTGCCGCGCAACGCGCGGCGGCGGC |
| | CCTGCTTAACCGCGCACGTCGCACCGGCCGACGGGCGGCCAT |
| | GCGGGCCGCTCGAAGGCTGGCCGCGGGTATTGTCACTGTGCC |
| | CCCCAGGTCCAGGCGACGAGCGGCCGCCGCAGCAGCCGCGG |
| | CCATTAGTGCTATGACTCAGGGTCGCAGGGGCAACGTGTATT |
| | GGGTGCGCGACTCGGTTAGCGGCCTGCGCGTGCCCGTGCGCA |
| | CCCGCCCCCCGCGCAACTAGATTGCAAGAAAAAACTACTTAG |
| | ACTCGTACTGTTGTATGTATCCAGCGGCGGCGGCGCGCAACG |
| | AAGCTATGTCCAAGCGCAAAATCAAAGAAGAGATGCTCCAG |
| | GTCATCGCGCCGGAGATCTATGGCCCCCCGAAGAAGGAAGA |
| | GCAGGATTACAAGCCCCGAAAGCTAAAGCGGGTCAAAAAGA |
| | AAAAGAAAGATGATGATGATGAACTTGACGACGAGGTGGAA |
| | CTGCTGCACGCTACCGCGCCCAGGCGACGGGTACAGTGGAAA |
| | GGTCGACGCGTAAAACGTGTTTTGCGACCCGGCACCACCGTA |
| | GTCTTTACGCCCGGTGAGCGCTCCACCCGCACCTACAAGCGC |
| | GTGTATGATGAGGTGTACGGCGACGAGGACCTGCTTGAGCAG |
| | GCCAACGAGCGCCTCGGGGAGTTTGCCTACGGAAAGCGGCAT |
| | AAGGACATGCTGGCGTTGCCGCTGGACGAGGGCAACCCAAC |
| | ACCTAGCCTAAAGCCCGTAACACTGCAGCAGGTGCTGCCCGC |
| | GCTTGCACCGTCCGAAGAAAAGCGCGGCCTAAAGCGCGAGTC |
| | TGGTGACTTGGCACCCACCGTGCAGCTGATGGTACCCAAGCG |
| | CCAGCGACTGGAAGATGTCTTGGAAAAAATGACCGTGGAACC |
| | TGGGCTGGAGCCCGAGGTCCGCGTGCGGCCAATCAAGCAGGT |
| | GGCGCCGGGACTGGGCGTGCAGACCGTGGACGTTCAGATACC |
| | CACTACCAGTAGCACCAGTATTGCCACCGCCACAGAGGGCAT |
| | GGAGACACAAACGTCCCCGGTTGCCTCAGCGGTGGCGGATGC |
| | CGCGGTGCAGGCGGTCGCTGCGGCCGCGTCCAAGACCTCTAC |
| | GGAGGTGCAAACGGACCCGTGGATGTTTCGCGTTTCAGCCCC |
| | CCGGCGCCCGCGCCGTTCGAGGAAGTACGGCGCCGCCAGCGC |
| | GCTACTGCCCGAATATGCCCTACATCCTTCCATTGCGCCTACC |
| | CCCGGCTATCGTGGCTACACCTACCGCCCCAGAAGACGAGCA |
| | ACTACCCGACGCCGAACCACCACTGGAACCCGCCGCCGCCGT |
| | CGCCGTCGCCAGCCCGTGCTGGCCCCGATTTCCGTGCGCAGG |
| | GTGGCTCGCGAAGGAGGCAGGACCCTGGTGCTGCCAACAGC |
| | GCGCTACCACCCCAGCATCGTTTAAAAGCCGGTCTTTGTGGTT |
| | CTTGCAGATATGGCCCTCACCTGCCGCCTCCGTTTCCCGGTGC |
| | CGGGATTCCGAGGAAGAATGCACCGTAGGAGGGGCATGGCC |
| | GGCCACGGCCTGACGGGCGGCATGCGTCGTGCGCACCACCGG |
| | CGGCGGCGCGCGTCGCACCGTCGCATGCGCGGCGGTATCCTG |
| | CCCCTCCTTATTCCACTGATCGCCGCGGCGATTGGCGCCGTGC |
| | CCGGAATTGCATCCGTGGCCTTGCAGGCGCAGAGACACTGAT |
| | TAAAAACAAGTTGCATGTGGAAAAATCAAAATAAAAAGTCT |
| | GGACTCTCACGCTCGCTTGGTCCTGTAACTATTTTGTAGAATG |
| | GAAGACATCAACTTTGCGTCTCTGGCCCCGCGACACGGCTCG |
| | CGCCCGTTCATGGGAAACTGGCAAGATATCGGCACCAGCAAT |
| | ATGAGCGGTGGCGCCTTCAGCTGGGGCTCGCTGTGGAGCGGC |
| | ATTAAAAATTTCGGTTCCACCGTTAAGAACTATGGCAGCAAG |
| | GCCTGGAACAGCAGCACAGGCCAGATGCTGAGGGATAAGTT |
| | GAAAGAGCAAAATTTCCAACAAAAGGTGGTAGATGGCCTGG |
| | CCTCTGGCATTAGCGGGGTGGTGGACCTGGCCAACCAGGCAG |
| | TGCAAAATAAGATTAACAGTAAGCTTGATCCCCGCCCTCCCG |
| | TAGAGGAGCCTCCACCGGCCGTGGAGACAGTGTCTCCAGAGG |
| | GGCGTGGCGAAAAGCGTCCGCGCCCCGACAGGGAAGAAACT |
| | CTGGTGACGCAAATAGACGAGCCTCCCTCGTACGAGGAGGCA |
| | CTAAAGCAAGGCCTGCCCACCACCCGTCCCATCGCGCCCATG |
| | GCTACCGGAGTGCTGGGCCAGCACACACCCGTAACGCTGGAC |
| | CTGCCTCCCCCCGCCGACACCCAGCAGAAACCTGTGCTGCCA |
| | GGCCCGACCGCCGTTGTTGTAACCCGTCCTAGCCGCGCGTCC |
| | CTGCGCCGCGCCGCCAGCGGTCCGCGATCGTTGCGGCCCGTA |
| | GCCAGTGGCAACTGGCAAAGCACACTGAACAGCATCGTGGGT |
| | CTGGGGGTGCAATCCCTGAAGCGCCGACGATGCTTCTGATAG |
| | CTAACGTGTCGTATGTGTGTCATGTATGCGTCCATGTCGCCGC |
| | CAGAGGAGCTGCTGAGCCGCCGCGCGCCCGCTTTCCAAGATG |
| | GCTACCCCTTCGATGATGCCGCAGTGGTCTTACATGCACATCT |
| | CGGGCCAGGACGCCTCGGAGTACCTGAGCCCCGGGCTGGTGC |
| | AGTTTGCCCGCGCCACCGAGACGTACTTCAGCCTGAATAACA |
| | AGTTTAGAAACCCCACGGTGGCGCCTACGCACGACGTGACCA |
| | CAGACCGGTCCCAGCGTTTGACGCTGCGGTTCATCCCTGTGG |
| | ACCGTGAGGATACTGCGTACTCGTACAAGGCGCGGTTCACCC |
| | TAGCTGTGGGTGATAACCGTGTGCTGGACATGGCTTCCACGT |
| | ACTTTGACATCCGCGGCGTGCTGGACAGGGGCCCTACTTTTA |
| | AGCCCTACTCTGGCACTGCCTACAACGCCCTGGCTCCCAAGG |
| | GTGCCCCAAATCCTTGCGAATGGGATGAAGCTGCTACTGCTC |
| | TTGAAATAAACCTAGAAGAAGAGGACGATGACAACGAAGAC |
| | GAAGTAGACGAGCAAGCTGAGCAGCAAAAAACTCACGTATT |
| | TGGGCAGGCGCCTTATTCTGGTATAAATATTACAAAGGAGGG |
| | TATTCAAATAGGTGTCGAAGGTCAAACACCTAAATATGCCGA |
| | TAAAACATTTCAACCTGAACCTCAAATAGGAGAATCTCAGTG |
| | GTACGAAACAGAAATTAATCATGCAGCTGGGAGAGTCCTAAA |
| | AAAGACTACCCCAATGAAACCATGTTACGGTTCATATGCAAA |
| | ACCCACAAATGAAAATGGAGGGCAAGGCATTCTTGTAAAGC |
| | AACAAAATGGAAAGCTAGAAAGTCAAGTGGAAATGCAATTT |
| | TTCTCAACTACTGAGGCAGCCGCAGGCAATGGTGATAACTTG |
| | ACTCCTAAAGTGGTATTGTACAGTGAAGATGTAGATATAGAA |
| | ACCCCAGACACTCATATTTCTTACATGCCCACTATTAAGGAA |
| | GGTAACTCACGAGAACTAATGGGCCAACAATCTATGCCCAAC |
| | AGGCCTAATTACATTGCTTTTAGGGACAATTTTATTGGTCTAA |
| | TGTATTACAACAGCACGGGTAATATGGGTGTTCTGGCGGGCC |
| | AAGCATCGCAGTTGAATGCTGTTGTAGATTTGCAAGACAGAA |
| | ACACAGAGCTTTCATACCAGCTTTTGCTTGATTCCATTGGTGA |
| | TAGAACCAGGTACTTTTCTATGTGGAATCAGGCTGTTGACAG |
| | CTATGATCCAGATGTTAGAATTATTGAAAATCATGGAACTGA |
| | AGATGAACTTCCAAATTACTGCTTTCCACTGGGAGGTGTGATT |
| | AATACAGAGACTCTTACCAAGGTAAAACCTAAAACAGGTCAG |
| | GAAAATGGATGGGAAAAAGATGCTACAGAATTTTCAGATAA |
| | AAATGAAATAAGAGTTGGAAATAATTTTGCCATGGAAATCAA |
| | TCTAAATGCCAACCTGTGGAGAAATTTCCTGTACTCCAACAT |
| | AGCGCTGTATTTGCCCGACAAGCTAAAGTACAGTCCTTCCAA |
| | CGTAAAAATTTCTGATAACCCAAACACCTACGACTACATGAA |
| | CAAGCGAGTGGTGGCTCCCGGGCTAGTGGACTGCTACATTAA |
| | CCTTGGAGCACGCTGGTCCCTTGACTATATGGACAACGTCAA |
| | CCCATTTAACCACCACCGCAATGCTGGCCTGCGCTACCGCTC |
| | AATGTTGCTGGGCAATGGTCGCTATGTGCCCTTCCACATCCAG |
| | GTGCCTCAGAAGTTCTTTGCCATTAAAAACCTCCTTCTCCTGC |
| | CGGGCTCATACACCTACGAGTGGAACTTCAGGAAGGATGTTA |
| | ACATGGTTCTGCAGAGCTCCCTAGGAAATGACCTAAGGGTTG |
| | ACGGAGCCAGCATTAAGTTTGATAGCATTTGCCTTTACGCCA |
| | CCTTCTTCCCCATGGCCCACAACACCGCCTCCACGCTTGAGGC |
| | CATGCTTAGAAACGACACCAACGACCAGTCCTTTAACGACTA |
| | TCTCTCCGCCGCCAACATGCTCTACCCTATACCCGCCAACGCT |
| | ACCAACGTGCCCATATCCATCCCCTCCCGCAACTGGGCGGCT |
| | TTCCGCGGCTGGGCCTTCACGCGCCTTAAGACTAAGGAAACC |
| | CCATCACTGGGCTCGGGCTACGACCCTTATTACACCTACTCTG |
| | GCTCTATACCCTACCTAGATGGAACCTTTTACCTCAACCACAC |
| | CTTTAAGAAGGTGGCCATTACCTTTGACTCTTCTGTCAGCTGG |
| | CCTGGCAATGACCGCCTGCTTACCCCCAACGAGTTTGAAATT |
| | AAGCGCTCAGTTGACGGGGAGGGTTACAACGTTGCCCAGTGT |
| | AACATGACCAAAGACTGGTTCCTGGTACAAATGCTAGCTAAC |
| | TATAACATTGGCTACCAGGGCTTCTATATCCCAGAGAGCTAC |
| | AAGGACCGCATGTACTCCTTCTTTAGAAACTTCCAGCCCATG |
| | AGCCGTCAGGTGGTGGATGATACTAAATACAAGGACTACCAA |
| | CAGGTGGGCATCCTACACCAACACAACAACTCTGGATTTGTT |
| | GGCTACCTTGCCCCCACCATGCGCGAAGGACAGGCCTACCCT |
| | GCTAACTTCCCCTATCCGCTTATAGGCAAGACCGCAGTTGAC |
| | AGCATTACCCAGAAAAAGTTTCTTTGCGATCGCACCCTTTGGC |
| | GCATCCCATTCTCCAGTAACTTTATGTCCATGGGCGCACTCAC |
| | AGACCTGGGCCAAAACCTTCTCTACGCCAACTCCGCCCACGC |
| | GCTAGACATGACTTTTGAGGTGGATCCCATGGACGAGCCCAC |
| | CCTTCTTTATGTTTTGTTTGAAGTCTTTGACGTGGTCCGTGTGC |
| | ACCAGCCGCACCGCGGCGTCATCGAAACCGTGTACCTGCGCA |
| | CGCCCTTCTCGGCCGGCAACGCCACAACATAAAGAAGCAAGC |
| | AACATCAACAACAGCTGCCGCCATGGGCTCCAGTGAGCAGGA |
| | ACTGAAAGCCATTGTCAAAGATCTTGGTTGTGGGCCATATTTT |
| | TTGGGCACCTATGACAAGCGCTTTCCAGGCTTTGTTTCTCCAC |
| | ACAAGCTCGCCTGCGCCATAGTCAATACGGCCGGTCGCGAGA |
| | CTGGGGGCGTACACTGGATGGCCTTTGCCTGGAACCCGCACT |
| | CAAAAACATGCTACCTCTTTGAGCCCTTTGGCTTTTCTGACCA |
| | GCGACTCAAGCAGGTTTACCAGTTTGAGTACGAGTCACTCCT |
| | GCGCCGTAGCGCCATTGCTTCTTCCCCCGACCGCTGTATAACG |
| | CTGGAAAAGTCCACCCAAAGCGTACAGGGGCCCAACTCGGCC |
| | GCCTGTGGACTATTCTGCTGCATGTTTCTCCACGCCTTTGCCA |
| | ACTGGCCCCAAACTCCCATGGATCACAACCCCACCATGAACC |
| | TTATTACCGGGGTACCCAACTCCATGCTCAACAGTCCCCAGG |
| | TACAGCCCACCCTGCGTCGCAACCAGGAACAGCTCTACAGCT |
| | TCCTGGAGCGCCACTCGCCCTACTTCCGCAGCCACAGTGCGC |
| | AGATTAGGAGCGCCACTTCTTTTTGTCACTTGAAAAACATGTA |
| | AAAATAATGTACTAGAGACACTTTCAATAAAGGCAAATGCTT |
| | TTATTTGTACACTCTCGGGTGATTATTTACCCCCACCCTTGCC |
| | GTCTGCGCCGTTTAAAAATCAAAGGGGTTCTGCCGCGCATCG |
| | CTATGCGCCACTGGCAGGGACACGTTGCGATACTGGTGTTTA |
| | GTGCTCCACTTAAACTCAGGCACAACCATCCGCGGCAGCTCG |
| | GTGAAGTTTTCACTCCACAGGCTGCGCACCATCACCAACGCG |
| | TTTAGCAGGTCGGGCGCCGATATCTTGAAGTCGCAGTTGGGG |
| | CCTCCGCCCTGCGCGCGCGAGTTGCGATACACAGGGTTGCAG |
| | CACTGGAACACTATCAGCGCCGGGTGGTGCACGCTGGCCAGC |
| | ACGCTCTTGTCGGAGATCAGATCCGCGTCCAGGTCCTCCGCG |
| | TTGCTCAGGGCGAACGGAGTCAACTTTGGTAGCTGCCTTCCC |
| | AAAAAGGGCGCGTGCCCAGGCTTTGAGTTGCACTCGCACCGT |
| | AGTGGCATCAAAAGGTGACCGTGCCCGGTCTGGGCGTTAGGA |
| | TACAGCGCCTGCATAAAAGCCTTGATCTGCTTAAAAGCCACC |
| | TGAGCCTTTGCGCCTTCAGAGAAGAACATGCCGCAAGACTTG |
| | CCGGAAAACTGATTGGCCGGACAGGCCGCGTCGTGCACGCAG |
| | CACCTTGCGTCGGTGTTGGAGATCTGCACCACATTTCGGCCCC |
| | ACCGGTTCTTCACGATCTTGGCCTTGCTAGACTGCTCCTTCAG |
| | CGCGCGCTGCCCGTTTTCGCTCGTCACATCCATTTCAATCACG |
| | TGCTCCTTATTTATCATAATGCTTCCGTGTAGACACTTAAGCT |
| | CGCCTTCGATCTCAGCGCAGCGGTGCAGCCACAACGCGCAGC |
| | CCGTGGGCTCGTGATGCTTGTAGGTCACCTCTGCAAACGACT |
| | GCAGGTACGCCTGCAGGAATCGCCCCATCATCGTCACAAAGG |
| | TCTTGTTGCTGGTGAAGGTCAGCTGCAACCCGCGGTGCTCCTC |
| | GTTCAGCCAGGTCTTGCATACGGCCGCCAGAGCTTCCACTTG |
| | GTCAGGCAGTAGTTTGAAGTTCGCCTTTAGATCGTTATCCACG |
| | TGGTACTTGTCCATCAGCGCGCGCGCAGCCTCCATGCCCTTCT |
| | CCCACGCAGACACGATCGGCACACTCAGCGGGTTCATCACCG |
| | TAATTTCACTTTCCGCTTCGCTGGGCTCTTCCTCTTCCTCTTGC |
| | GTCCGCATACCACGCGCCACTGGGTCGTCTTCATTCAGCCGCC |
| | GCACTGTGCGCTTACCTCCTTTGCCATGCTTGATTAGCACCGG |
| | TGGGTTGCTGAAACCCACCATTTGTAGCGCCACATCTTCTCTT |
| | TCTTCCTCGCTGTCCACGATTACCTCTGGTGATGGCGGGCGCT |
| | CGGGCTTGGGAGAAGGGCGCTTCTTTTTCTTCTTGGGCGCAAT |
| | GGCCAAATCCGCCGCCGAGGTCGATGGCCGCGGGCTGGGTGT |
| | GCGCGGCACCAGCGCGTCTTGTGATGAGTCTTCCTCGTCCTCG |
| | GACTCGATACGCCGCCTCATCCGCTTTTTTGGGGGCGCCCGG |
| | GGAGGCGGCGGCGACGGGGACGGGGACGACACGTCCTCCAT |
| | GGTTGGGGGACGTCGCGCCGCACCGCGTCCGCGCTCGGGGGT |
| | GGTTTCGCGCTGCTCCTCTTCCCGACTGGCCATTTCCTTCTCCT |
| | ATAGGCAGAAAAAGATCATGGAGTCAGTCGAGAAGAAGGAC |
| | AGCCTAACCGCCCCCTCTGAGTTCGCCACCACCGCCTCCACC |
| | GATGCCGCCAACGCGCCTACCACCTTCCCCGTCGAGGCACCC |
| | CCGCTTGAGGAGGAGGAAGTGATTATCGAGCAGGACCCAGG |
| | TTTTGTAAGCGAAGACGACGAGGACCGCTCAGTACCAACAGA |
| | GGATAAAAAGCAAGACCAGGACAACGCAGAGGCAAACGAGG |
| | AACAAGTCGGGCGGGGGGACGAAAGGCATGGCGACTACCTA |
| | GATGTGGGAGACGACGTGCTGTTGAAGCATCTGCAGCGCCAG |
| | TGCGCCATTATCTGCGACGCGTTGCAAGAGCGCAGCGATGTG |
| | CCCCTCGCCATAGCGGATGTCAGCCTTGCCTACGAACGCCAC |
| | CTATTCTCACCGCGCGTACCCCCCAAACGCCAAGAAAACGGC |
| | ACATGCGAGCCCAACCCGCGCCTCAACTTCTACCCCGTATTTG |
| | CCGTGCCAGAGGTGCTTGCCACCTATCACATCTTTTTCCAAAA |
| | CTGCAAGATACCCCTATCCTGCCGTGCCAACCGCAGCCGAGC |
| | GGACAAGCAGCTGGCCTTGCGGCAGGGCGCTGTCATACCTGA |
| | TATCGCCTCGCTCAACGAAGTGCCAAAAATCTTTGAGGGTCT |
| | TGGACGCGACGAGAAGCGCGCGGCAAACGCTCTGCAACAGG |
| | AAAACAGCGAAAATGAAAGTCACTCTGGAGTGTTGGTGGAA |
| | CTCGAGGGTGACAACGCGCGCCTAGCCGTACTAAAACGCAGC |
| | ATCGAGGTCACCCACTTTGCCTACCCGGCACTTAACCTACCCC |
| | CCAAGGTCATGAGCACAGTCATGAGTGAGCTGATCGTGCGCC |
| | GTGCGCAGCCCCTGGAGAGGGATGCAAATTTGCAAGAACAA |
| | ACAGAGGAGGGCCTACCCGCAGTTGGCGACGAGCAGCTAGC |
| | GCGCTGGCTTCAAACGCGCGAGCCTGCCGACTTGGAGGAGCG |
| | ACGCAAACTAATGATGGCCGCAGTGCTCGTTACCGTGGAGCT |
| | TGAGTGCATGCAGCGGTTCTTTGCTGACCCGGAGATGCAGCG |
| | CAAGCTAGAGGAAACATTGCACTACACCTTTCGACAGGGCTA |
| | CGTACGCCAGGCCTGCAAGATCTCCAACGTGGAGCTCTGCAA |
| | CCTGGTCTCCTACCTTGGAATTTTGCACGAAAACCGCCTTGGG |
| | CAAAACGTGCTTCATTCCACGCTCAAGGGCGAGGCGCGCCGC |
| | GACTACGTCCGCGACTGCGTTTACTTATTTCTATGCTACACCT |
| | GGCAGACGGCCATGGGCGTTTGGCAGCAGTGCTTGGAGGAGT |
| | GCAACCTCAAGGAGCTGCAGAAACTGCTAAAGCAAAACTTG |
| | AAGGACCTATGGACGGCCTTCAACGAGCGCTCCGTGGCCGCG |
| | CACCTGGCGGACATCATTTTCCCCGAACGCCTGCTTAAAACC |
| | CTGCAACAGGGTCTGCCAGACTTCACCAGTCAAAGCATGTTG |
| | CAGAACTTTAGGAACTTTATCCTAGAGCGCTCAGGAATCTTG |
| | CCCGCCACCTGCTGTGCACTTCCTAGCGACTTTGTGCCCATTA |
| | AGTACCGCGAATGCCCTCCGCCGCTTTGGGGCCACTGCTACC |
| | TTCTGCAGCTAGCCAACTACCTTGCCTACCACTCTGACATAAT |
| | GGAAGACGTGAGCGGTGACGGTCTACTGGAGTGTCACTGTCG |
| | CTGCAACCTATGCACCCCGCACCGCTCCCTGGTTTGCAATTCG |
| | CAGCTGCTTAACGAAAGTCAAATTATCGGTACCTTTGAGCTG |
| | CAGGGTCCCTCGCCTGACGAAAAGTCCGCGGCTCCGGGGTTG |
| | AAACTCACTCCGGGGCTGTGGACGTCGGCTTACCTTCGCAAA |
| | TTTGTACCTGAGGACTACCACGCCCACGAGATTAGGTTCTAC |
| | GAAGACCAATCCCGCCCGCCTAATGCGGAGCTTACCGCCTGC |
| | GTCATTACCCAGGGCCACATTCTTGGCCAATTGCAAGCCATC |
| | AACAAAGCCCGCCAAGAGTTTCTGCTACGAAAGGGACGGGG |
| | GGTTTACTTGGACCCCCAGTCCGGCGAGGAGCTCAACCCAAT |
| | CCCCCCGCCGCCGCAGCCCTATCAGCAGCAGCCGCGGGCCCT |
| | TGCTTCCCAGGATGGCACCCAAAAAGAAGCTGCAGCTGCCGC |
| | CGCCACCCACGGACGAGGAGGAATACTGGGACAGTCAGGCA |
| | GAGGAGGTTTTGGACGAGGAGGAGGAGGACATGATGGAAGA |
| | CTGGGAGAGCCTAGACGAGGAAGCTTCCGAGGTCGAAGAGG |
| | TGTCAGACGAAACACCGTCACCCTCGGTCGCATTCCCCTCGC |
| | CGGCGCCCCAGAAATCGGCAACCGGTTCCAGCATGGCTACAA |
| | CCTCCGCTCCTCAGGCGCCGCCGGCACTGCCCGTTCGCCGAC |
| | CCAACCGTAGATGGGACACCACTGGAACCAGGGCCGGTAAG |
| | TCCAAGCAGCCGCCGCCGTTAGCCCAAGAGCAACAACAGCGC |
| | CAAGGCTACCGCTCATGGCGCGGGCACAAGAACGCCATAGTT |
| | GCTTGCTTGCAAGACTGTGGGGGCAACATCTCCTTCGCCCGC |
| | CGCTTTCTTCTCTACCATCACGGCGTGGCCTTCCCCCGTAACA |
| | TCCTGCATTACTACCGTCATCTCTACAGCCCATACTGCACCGG |
| | CGGCAGCGGCAGCAACAGCAGCGGCCACACAGAAGCAAAGG |
| | CGACCGGATAGCAAGACTCTGACAAAGCCCAAGAAATCCAC |
| | AGCGGCGGCAGCAGCAGGAGGAGGAGCGCTGCGTCTGGCGC |
| | CCAACGAACCCGTATCGACCCGCGAGCTTAGAAACAGGATTT |
| | TTCCCACTCTGTATGCTATATTTCAACAGAGCAGGGGCCAAG |
| | AACAAGAGCTGAAAATAAAAAACAGGTCTCTGCGATCCCTCA |
| | CCCGCAGCTGCCTGTATCACAAAAGCGAAGATCAGCTTCGGC |
| | GCACGCTGGAAGACGCGGAGGCTCTCTTCAGTAAATACTGCG |
| | CGCTGACTCTTAAGGACTAGTTTCGCGCCCTTTCTCAAATTTA |
| | AGCGCGAAAACTACGTCATCTCCAGCGGCCACACCCGGCGCC |
| | AGCACCTGTTGTCAGCGCCATTATGAGCAAGGAAATTCCCAC |
| | GCCCTACATGTGGAGTTACCAGCCACAAATGGGACTTGCGGC |
| | TGGAGCTGCCCAAGACTACTCAACCCGAATAAACTACATGAG |
| | CGCGGGACCCCACATGATATCCCGGGTCAACGGAATACGCGC |
| | CCACCGAAACCGAATTCTCCTGGAACAGGCGGCTATTACCAC |
| | CACACCTCGTAATAACCTTAATCCCCGTAGTTGGCCCGCTGCC |
| | CTGGTGTACCAGGAAAGTCCCGCTCCCACCACTGTGGTACTT |
| | CCCAGAGACGCCCAGGCCGAAGTTCAGATGACTAACTCAGGG |
| | GCGCAGCTTGCGGGCGGCTTTCGTCACAGGGTGCGGTCGCCC |
| | GGGCAGGGTATAACTCACCTGACAATCAGAGGGCGAGGTATT |
| | CAGCTCAACGACGAGTCGGTGAGCTCCTCGCTTGGTCTCCGT |
| | CCGGACGGGACATTTCAGATCGGCGGCGCCGGCCGCTCTTCA |
| | TTCACGCCTCGTCAGGCAATCCTAACTCTGCAGACCTCGTCCT |
| | CTGAGCCGCGCTCTGGAGGCATTGGAACTCTGCAATTTATTG |
| | AGGAGTTTGTGCCATCGGTCTACTTTAACCCCTTCTCGGGACC |
| | TCCCGGCCACTATCCGGATCAATTTATTCCTAACTTTGACGCG |
| | GTAAAGGACTCGGCGGACGGCTACGACTGAATGTTAAGTGGA |
| | GAGGCAGAGCAACTGCGCCTGAAACACCTGGTCCACTGTCGC |
| | CGCCACAAGTGCTTTGCCCGCGACTCCGGTGAGTTTTGCTACT |
| | TTGAATTGCCCGAGGATCATATCGAGGGCCCGGCGCACGGCG |
| | TCCGGCTTACCGCCCAGGGAGAGCTTGCCCGTAGCCTGATTC |
| | GGGAGTTTACCCAGCGCCCCCTGCTAGTTGAGCGGGACAGGG |
| | GACCCTGTGTTCTCACTGTGATTTGCAACTGTCCTAACCCTGG |
| | ATTACATCAAGATCCTCTAGTTAATGTCAGGTCGCCTAAGTCG |
| | ATTAACTAGAGTACCCGGGGATCTTATTCCCTTTAACTAATAA |
| | AAAAAAATAATAAAGCATCACTTACTTAAAATCAGTTAGCAA |
| | ATTTCTGTCCAGTTTATTCAGCAGCACCTCCTTGCCCTCCTCC |
| | CAGCTCTGGTATTGCAGCTTCCTCCTGGCTGCAAACTTTCTCC |
| | ACAATCTAAATGGAATGTCAGTTTCCTCCTGTTCCTGTCCATC |
| | CGCACCCACTATCTTCATGTTGTTGCAGATGAAGCGCGCAAG |
| | ACCGTCTGAAGATACCTTCAACCCCGTGTATCCATATGACAC |
| | GGAAACCGGTCCTCCAACTGTGCCTTTTCTTACTCCTCCCTTT |
| | GTATCCCCCAATGGGTTTCAAGAGAGTCCCCCTGGGGTACTC |
| | TCTTTGCGCCTATCCGAACCTCTAGTTACCTCCAATGGCATGC |
| | TTGCGCTCAAAATGGGCAACGGCCTCTCTCTGGACGAGGCCG |
| | GCAACCTTACCTCCCAAAATGTAACCACTGTGAGCCCACCTC |
| | TCAAAAAAACCAAGTCAAACATAAACCTGGAAATATCTGCAC |
| | CCCTCACAGTTACCTCAGAAGCCCTAACTGTGGCTGCCGCCG |
| | CACCTCTAATGGTCGCGGGCAACACACTCACCATGCAATCAC |
| | AGGCCCCGCTAACCGTGCACGACTCCAAACTTAGCATTGCCA |
| | CCCAAGGACCCCTCACAGTGTCAGAAGGAAAGCTAGCCCTGC |
| | AAACATCAGGCCCCCTCACCACCACCGATAGCAGTACCCTTA |
| | CTATCACTGCCTCACCCCCTCTAACTACTGCCACTGGTAGCTT |
| | GGGCATTGACTTGAAAGAGCCCATTTATACACAAAATGGAAA |
| | ACTAGGACTAAAGTACGGGGCTCCTTTGCATGTAACAGACGA |
| | CCTAAACACTTTGACCGTAGCAACTGGTCCAGGTGTGACTAT |
| | TAATAATACTTCCTTGCAAACTAAAGTTACTGGAGCCTTGGGT |
| | TTTGATTCACAAGGCAATATGCAACTTAATGTAGCAGGAGGA |
| | CTAAGGATTGATTCTCAAAACAGACGCCTTATACTTGATGTTA |
| | GTTATCCGTTTGATGCTCAAAACCAACTAAATCTAAGACTAG |
| | GACAGGGCCCTCTTTTTATAAACTCAGCCCACAACTTGGATAT |
| | TAACTACAACAAAGGCCTTTACTTGTTTACAGCTTCAAACAAT |
| | TCCAAAAAGCTTGAGGTTAACCTAAGCACTGCCAAGGGGTTG |
| | ATGTTTGACGCTACAGCCATAGCCATTAATGCAGGAGATGGG |
| | CTTGAATTTGGTTCACCTAATGCACCAAACACAAATCCCCTCA |
| | AAACAAAAATTGGCCATGGCCTAGAATTTGATTCAAACAAGG |
| | CTATGGTTCCTAAACTAGGAACTGGCCTTAGTTTTGACAGCAC |
| | AGGTGCCATTACAGTAGGAAACAAAAATAATGATAAGCTAA |
| | CTTTGTGGACCACACCAGCTCCATCTCCTAACTGTAGACTAAA |
| | TGCAGAGAAAGATGCTAAACTCACTTTGGTCTTAACAAAATG |
| | TGGCAGTCAAATACTTGCTACAGTTTCAGTTTTGGCTGTTAAA |
| | GGCAGTTTGGCTCCAATATCTGGAACAGTTCAAAGTGCTCAT |
| | CTTATTATAAGATTTGACGAAAATGGAGTGCTACTAAACAAT |
| | TCCTTCCTGGACCCAGAATATTGGAACTTTAGAAATGGAGAT |
| | CTTACTGAAGGCACAGCCTATACAAACGCTGTTGGATTTATG |
| | CCTAACCTATCAGCTTATCCAAAATCTCACGGTAAAACTGCC |
| | AAAAGTAACATTGTCAGTCAAGTTTACTTAAACGGAGACAAA |
| | ACTAAACCTGTAACACTAACCATTACACTAAACGGTACACAG |
| | GAAACAGGAGACACAACTCCAAGTGCATACTCTATGTCATTT |
| | TCATGGGACTGGTCTGGCCACAACTACATTAATGAAATATTT |
| | GCCACATCCTCTTACACTTTTTCATACATTGCCCAAGAATAAA |
| | GAATCGTTTGTGTTATGTTTCAACGTGTTTATTTTTCAATTGCA |
| | GAAAATTTCAAGTCATTTTTCATTCAGTAGTATAGCCCCACCA |
| | CCACATAGCTTATACAGATCACCGTACCTTAATCAAACTCAC |
| | AGAACCCTAGTATTCAACCTGCCACCTCCCTCCCAACACACA |
| | GAGTACACAGTCCTTTCTCCCCGGCTGGCCTTAAAAAGCATC |
| | ATATCATGGGTAACAGACATATTCTTAGGTGTTATATTCCACA |
| | CGGTTTCCTGTCGAGCCAAACGCTCATCAGTGATATTAATAA |
| | ACTCCCCGGGCAGCTCACTTAAGTTCATGTCGCTGTCCAGCTG |
| | CTGAGCCACAGGCTGCTGTCCAACTTGCGGTTGCTTAACGGG |
| | CGGCGAAGGAGAAGTCCACGCCTACATGGGGGTAGAGTCAT |
| | AATCGTGCATCAGGATAGGGCGGTGGTGCTGCAGCAGCGCGC |
| | GAATAAACTGCTGCCGCCGCCGCTCCGTCCTGCAGGAATACA |
| | ACATGGCAGTGGTCTCCTCAGCGATGATTCGCACCGCCCGCA |
| | GCATAAGGCGCCTTGTCCTCCGGGCACAGCAGCGCACCCTGA |
| | TCTCACTTAAATCAGCACAGTAACTGCAGCACAGCACCACAA |
| | TATTGTTCAAAATCCCACAGTGCAAGGCGCTGTATCCAAAGC |
| | TCATGGCGGGGACCACAGAACCCACGTGGCCATCATACCACA |
| | AGCGCAGGTAGATTAAGTGGCGACCCCTCATAAACACGCTGG |
| | ACATAAACATTACCTCTTTTGGCATGTTGTAATTCACCACCTC |
| | CCGGTACCATATAAACCTCTGATTAAACATGGCGCCATCCAC |
| | CACCATCCTAAACCAGCTGGCCAAAACCTGCCCGCCGGCTAT |
| | ACACTGCAGGGAACCGGGACTGGAACAATGACAGTGGAGAG |
| | CCCAGGACTCGTAACCATGGATCATCATGCTCGTCATGATAT |
| | CAATGTTGGCACAACACAGGCACACGTGCATACACTTCCTCA |
| | GGATTACAAGCTCCTCCCGCGTTAGAACCATATCCCAGGGAA |
| | CAACCCATTCCTGAATCAGCGTAAATCCCACACTGCAGGGAA |
| | GACCTCGCACGTAACTCACGTTGTGCATTGTCAAAGTGTTAC |
| | ATTCGGGCAGCAGCGGATGATCCTCCAGTATGGTAGCGCGGG |
| | TTTCTGTCTCAAAAGGAGGTAGACGATCCCTACTGTACGGAG |
| | TGCGCCGAGACAACCGAGATCGTGTTGGTCGTAGTGTCATGC |
| | CAAATGGAACGCCGGACGTAGTCATATTTCCTGAAGCAAAAC |
| | CAGGTGCGGGCGTGACAAACAGATCTGCGTCTCCGGTCTCGC |
| | CGCTTAGATCGCTCTGTGTAGTAGTTGTAGTATATCCACTCTC |
| | TCAAAGCATCCAGGCGCCCCCTGGCTTCGGGTTCTATGTAAA |
| | CTCCTTCATGCGCCGCTGCCCTGATAACATCCACCACCGCAG |
| | AATAAGCCACACCCAGCCAACCTACACATTCGTTCTGCGAGT |
| | CACACACGGGAGGAGCGGGAAGAGCTGGAAGAACCATGTTT |
| | TTTTTTTTATTCCAAAAGATTATCCAAAACCTCAAAATGAAGA |
| | TCTATTAAGTGAACGCGCTCCCCTCCGGTGGCGTGGTCAAAC |
| | TCTACAGCCAAAGAACAGATAATGGCATTTGTAAGATGTTGC |
| | ACAATGGCTTCCAAAAGGCAAACGGCCCTCACGTCCAAGTGG |
| | ACGTAAAGGCTAAACCCTTCAGGGTGAATCTCCTCTATAAAC |
| | ATTCCAGCACCTTCAACCATGCCCAAATAATTCTCATCTCGCC |
| | ACCTTCTCAATATATCTCTAAGCAAATCCCGAATATTAAGTCC |
| | GGCCATTGTAAAAATCTGCTCCAGAGCGCCCTCCACCTTCAG |
| | CCTCAAGCAGCGAATCATGATTGCAAAAATTCAGGTTCCTCA |
| | CAGACCTGTATAAGATTCAAAAGCGGAACATTAACAAAAATA |
| | CCGCGATCCCGTAGGTCCCTTCGCAGGGCCAGCTGAACATAA |
| | TCGTGCAGGTCTGCACGGACCAGCGCGGCCACTTCCCCGCCA |
| | GGAACCATGACAAAAGAACCCACACTGATTATGACACGCATA |
| | CTCGGAGCTATGCTAACCAGCGTAGCCCCGATGTAAGCTTGT |
| | TGCATGGGCGGCGATATAAAATGCAAGGTGCTGCTCAAAAAA |
| | TCAGGCAAAGCCTCGCGCAAAAAAGAAAGCACATCGTAGTC |
| | ATGCTCATGCAGATAAAGGCAGGTAAGCTCCGGAACCACCAC |
| | AGAAAAAGACACCATTTTTCTCTCAAACATGTCTGCGGGTTTC |
| | TGCATAAACACAAAATAAAATAACAAAAAAACATTTAAACA |
| | TTAGAAGCCTGTCTTACAACAGGAAAAACAACCCTTATAAGC |
| | ATAAGACGGACTACGGCCATGCCGGCGTGACCGTAAAAAAA |
| | CTGGTCACCGTGATTAAAAAGCACCACCGACAGCTCCTCGGT |
| | CATGTCCGGAGTCATAATGTAAGACTCGGTAAACACATCAGG |
| | TTGATTCACATCGGTCAGTGCTAAAAAGCGACCGAAATAGCC |
| | CGGGGGAATACATACCCGCAGGCGTAGAGACAACATTACAG |
| | CCCCCATAGGAGGTATAACAAAATTAATAGGAGAGAAAAAC |
| | ACATAAACACCTGAAAAACCCTCCTGCCTAGGCAAAATAGCA |
| | CCCTCCCGCTCCAGAACAACATACAGCGCTTCCACAGCGGCA |
| | GCCATAACAGTCAGCCTTACCAGTAAAAAAGAAAACCTATTA |
| | AAAAAACACCACTCGACACGGCACCAGCTCAATCAGTCACAG |
| | TGTAAAAAAGGGCCAAGTGCAGAGCGAGTATATATAGGACT |
| | AAAAAATGACGTAACGGTTAAAGTCCACAAAAAACACCCAG |
| | AAAACCGCACGCGAACCTACGCCCAGAAACGAAAGCCAAAA |
| | AACCCACAACTTCCTCAAATCGTCACTTCCGTTTTCCCACGTT |
| | ACGTCACTTCCCATTTTAAGAAAACTACAATTCCCAACACAT |
| | ACAAGTTACTCCGCCCTAAAACCTACGTCACCCGCCCCGTTCC |
| | CACGCCCCGCGCCACGTCACAAACTCCACCCCCTCATTATCAT |
| | ATTGGCTTCAATCCAAAATAAGGTATATTATTGATGAT |
| SEQ | ATGGAGTCTCCCTCGGCCCCTCCCCACAGATGGTGCATCCCCT |
| ID | GGCAGAGGCTCCTGCTCACAGCCTCACTTCTAACCTTCTGGA |
| NO: | ACCCGCCCACCACTGCCAAGCTCACTATTGAATCCACGCCGT |
| 30 | TCAATGTCGCAGAGGGGAAGGAGGTGCTTCTACTTGTCCACA |
| | ATCTGCCCCAGCATCTTTTTGGCTACAGCTGGTACAAAGGTG |
| | AAAGAGTGGATGGCAACCGTCAAATTATAGGATATGTAATAG |
| | GAACTCAACAAGCTACCCCAGGGCCCGCATACAGTGGTCGAG |
| | AGATAATATACCCCAATGCATCCCTGCTGATCCAGAACATCA |
| | TCCAGAATGACACAGGATTCTACACCCTACACGTCATAAAGT |
| | CAGATCTTGTGAATGAAGAAGCAACTGGCCAGTTCCGGGTAT |
| | ACCCGGAGCTGCCCAAGCCCTCCATCTCCAGCAACAACTCCA |
| | AACCCGTGGAGGACAAGGATGCTGTGGCCTTCACCTGTGAAC |
| | CTGAGACTCAGGACGCAACCTACCTGTGGTGGGTAAACAATC |
| | AGAGCCTCCCGGTCAGTCCCAGGCTGCAGCTGTCCAATGGCA |
| | ACAGGACCCTCACTCTATTCAATGTCACAAGAAATGACACAG |
| | CAAGCTACAAATGTGAAACCCAGAACCCAGTGAGTGCCAGG |
| | CGCAGTGATTCAGTCATCCTGAATGTCCTCTATGGCCCGGATG |
| | CCCCCACCATTTCCCCTCTAAACACATCTTACAGATCAGGGG |
| | AAAATCTGAACCTCTCCTGCCACGCAGCCTCTAACCCACCTG |
| | CACAGTACTCTTGGTTTGTCAATGGGACTTTCCAGCAATCCAC |
| | CCAAGAGCTCTTTATCCCCAACATCACTGTGAATAATAGTGG |
| | ATCCTATACGTGCCAAGCCCATAACTCAGACACTGGCCTCAA |
| | TAGGACCACAGTCACGACGATCACAGTCTATGCAGAGCCACC |
| | CAAACCCTTCATCACCAGCAACAACTCCAACCCCGTGGAGGA |
| | TGAGGATGCTGTAGCCTTAACCTGTGAACCTGAGATTCAGAA |
| | CACAACCTACCTGTGGTGGGTAAATAATCAGAGCCTCCCGGT |
| | CAGTCCCAGGCTGCAGCTGTCCAATGACAACAGGACCCTCAC |
| | TCTACTCAGTGTCACAAGGAATGATGTAGGACCCTATGAGTG |
| | TGGAATCCAGAACGAATTAAGTGTTGACCACAGCGACCCAGT |
| | CATCCTGAATGTCCTCTATGGCCCAGACGACCCCACCATTTCC |
| | CCCTCATACACCTATTACCGTCCAGGGGTGAACCTCAGCCTCT |
| | CCTGCCATGCAGCCTCTAACCCACCTGCACAGTATTCTTGGCT |
| | GATTGATGGGAACATCCAGCAACACACACAAGAGCTCTTTAT |
| | CTCCAACATCACTGAGAAGAACAGCGGACTCTATACCTGCCA |
| | GGCCAATAACTCAGCCAGTGGCCACAGCAGGACTACAGTCAA |
| | GACAATCACAGTCTCTGCGGAGCTGCCCAAGCCCTCCATCTC |
| | CAGCAACAACTCCAAACCCGTGGAGGACAAGGATGCTGTGG |
| | CCTTCACCTGTGAACCTGAGGCTCAGAACACAACCTACCTGT |
| | GGTGGGTAAATGGTCAGAGCCTCCCAGTCAGTCCCAGGCTGC |
| | AGCTGTCCAATGGCAACAGGACCCTCACTCTATTCAATGTCA |
| | CAAGAAATGACGCAAGAGCCTATGTATGTGGAATCCAGAACT |
| | CAGTGAGTGCAAACCGCAGTGACCCAGTCACCCTGGATGTCC |
| | TCTATGGGCCGGACACCCCCATCATTTCCCCCCCAGACTCGTC |
| | TTACCTTTCGGGAGCGGACCTCAACCTCTCCTGCCACTCGGCC |
| | TCTAACCCATCCCCGCAGTATTCTTGGCGTATCAATGGGATAC |
| | CGCAGCAACACACACAAGTTCTCTTTATCGCCAAAATCACGC |
| | CAAATAATAACGGGACCTATGCCTGTTTTGTCTCTAACTTGGC |
| | TACTGGCCGCAATAATTCCATAGTCAAGAGCATCACAGTCTC |
| | TGCATCTGGAACTTCTCCTGGTCTCTCAGCTGGGGCCACTGTC |
| | GGCATCATGATTGGAGTGCTGGTTGGGGTTGCTCTGATATAG |
| SEQ | YLSGADLNL |
| ID | |
| NO: | |
| 31 | |
| SEQ | ATGACACCGGGCACCCAGTCTCCTTTCTTCCTGCTGCTGCTCC |
| ID | TCACAGTGCTTACAGTTGTTACGGGTTCTGGTCATGCAAGCTC |
| NO: | TACCCCAGGTGGAGAAAAGGAGACTTCGGCTACCCAGAGAA |
| 32 | GTTCAGTGCCCAGCTCTACTGAGAAGAATGCTGTGAGTATGA |
| | CCAGCAGCGTACTCTCCAGCCACAGCCCCGGTTCAGGCTCCT |
| | CCACCACTCAGGGACAGGATGTCACTCTGGCCCCGGCCACGG |
| | AACCAGCTTCAGGTTCAGCTGCCCTTTGGGGACAGGATGTCA |
| | CCTCGGTCCCAGTCACCAGGCCAGCCCTGGGCTCCACCACCC |
| | CGCCAGCCCACGATGTCACCTCAGCCCCGGACAACAAGCCAG |
| | CCCCGGGCTCCACCGCCCCCCCAGCCCACGGTGTCACCTCGT |
| | ATCTTGACACCAGGCCGGCCCCGGTTTATCTTGCCCCCCCAGC |
| | CCATGGTGTCACCTCGGCCCCGGACAACAGGCCCGCCTTGGG |
| | CTCCACCGCCCCTCCAGTCCACAATGTCACCTCGGCCTCAGGC |
| | TCTGCATCAGGCTCAGCTTCTACTCTGGTGCACAACGGCACCT |
| | CTGCCAGGGCTACCACAACCCCAGCCAGCAAGAGCACTCCAT |
| | TCTCAATTCCCAGCCACCACTCTGATACTCCTACCACCCTTGC |
| | CAGCCATAGCACCAAGACTGATGCCAGTAGCACTCACCATAG |
| | CACGGTACCTCCTCTCACCTCCTCCAATCACAGCACTTCTCCC |
| | CAGTTGTCTACTGGGGTCTCTTTCTTTTTCCTGTCTTTTCACAT |
| | TTCAAACCTCCAGTTTAATTCCTCTCTGGAAGATCCCAGCACC |
| | GACTACTACCAAGAGCTGCAGAGAGACATTTCTGAAATGTTT |
| | TTGCAGATTTATAAACAAGGGGGTTTTCTGGGCCTCTCCAATA |
| | TTAAGTTCAGGCCAGGATCTGTGGTGGTACAATTGACTCTGG |
| | CCTTCCGAGAAGGTACCATCAATGTCCACGACGTGGAGACAC |
| | AGTTCAATCAGTATAAAACGGAAGCAGCCTCTCGATATAACC |
| | TGACGATCTCAGACGTCAGCGTGAGTGATGTGCCATTTCCTTT |
| | CTCTGCCCAGTCTGGGGCTGGGGTGCCAGGCTGGGGCATCGC |
| | GCTGCTGGTGCTGGTCTGTGTTCTGGTTTATCTGGCCATTGTC |
| | TATCTCATTGCCTTGGCTGTCGCTCAGGTTCGCCGAAAGAACT |
| | ACGGGCAGCTGGACATCTTTCCAGCCCGGGATAAATACCATC |
| | CTATGAGCGAGTACGCTCTTTACCACACCCATGGGCGCTATG |
| | TGCCCCCTAGCAGTCTTTTCCGTAGCCCCTATGAGAAGGTTTC |
| | TGCAGGTAATGGTGGCAGCTATCTCTCTTACACAAACCCAGC |
| | AGTGGCAGCCGCTTCTGCCAACTTGTAG |
| SEQ | MTPGTQSPFFLLLLLTVLTVVTGSGHASSTPGGEKETSATQRSSV |
| ID | PSSTEKNAVSMTSSVLSSHSPGSGSSTTQGQDVTLAPATEPASGS |
| NO: | AALWGQDVTSVPVTRPALGSTTPPAHDVTSAPDNKPAPGSTAPP |
| 33 | AHGVTSYLDTRPAPVYLAPPAHGVTSAPDNRPALGSTAPPVHNV |
| | TSASGSASGSASTLVHNGTSARATTTPASKSTPFSIPSHHSDTPTT |
| | LASHSTKTDASSTHHSTVPPLTSSNHSTSPQLSTGVSFFFLSFHIS |
| | NLQFNSSLEDPSTDYYQELQRDISEMFLQIYKQGGFLGLSNIKFRP |
| | GSVVVQLTLAFREGTINVHDVETQFNQYKTEAASRYNLTISDVS |
| | VSDVPFPFSAQSGAGVPGWGIALLVLVCVLVYLAIVYLIALAVA |
| | QVRRKNYGQLDIFPARDKYHPMSEYALYHTHGRYVPPSSLFRSP |
| | YEKVSAGNGGSYLSYTNPAVAAASANL |
| SEQ | ATGAGCTCCCCTGGCACCGAGAGCGCGGGAAAGAGCCTGCA |
| ID | GTACCGAGTGGACCACCTGCTGAGCGCCGTGGAGAATGAGCT |
| NO: | GCAGGCGGGCAGCGAGAAGGGCGACCCCACAGAGCGCGAAC |
| 34 | TGCGCGTGGGCCTGGAGGAGAGCGAGCTGTGGCTGCGCTTCA |
| | AGGAGCTCACCAATGAGATGATCGTGACCAAGAACGGCAGG |
| | AGGATGTTTCCGGTGCTGAAGGTGAACGTGTCTGGCCTGGAC |
| | CCCAACGCCATGTACTCCTTCCTGCTGGACTTCGTGGCGGCGG |
| | ACAACCACCGCTGGAAGTACGTGAACGGGGAATGGGTGCCG |
| | GGGGGCAAGCCGGAGCCGCAGGCGCCCAGCTGCGTCTACATC |
| | CACCCCGACTCGCCCAACTTCGGGGCCCACTGGATGAAGGCT |
| | CCCGTCTCCTTCAGCAAAGTCAAGCTCACCAACAAGCTCAAC |
| | GGAGGGGGCCAGATCATGCTGAACTCCTTGCATAAGTATGAG |
| | CCTCGAATCCACATAGTGAGAGTTGGGGGTCCACAGCGCATG |
| | ATCACCAGCCACTGCTTCCCTGAGACCCAGTTCATAGCGGTG |
| | ACTGCTAGAAGTGATCACAAAGAGATGATGGAGGAACCCGG |
| | AGACAGCCAGCAACCTGGGTACTCCCAATGGGGGTGGCTTCT |
| | TCCTGGAACCAGCACCGTGTGTCCACCTGCAAATCCTCATCCT |
| | CAGTTTGGAGGTGCCCTCTCCCTCCCCTCCACGCACAGCTGTG |
| | ACAGGTACCCAACCCTGAGGAGCCACCGGTCCTCACCCTACC |
| | CCAGCCCCTATGCTCATCGGAACAATTCTCCAACCTATTCTGA |
| | CAACTCACCTGCATGTTTATCCATGCTGCAATCCCATGACAAT |
| | TGGTCCAGCCTTGGAATGCCTGCCCATCCCAGCATGCTCCCCG |
| | TGAGCCACAATGCCAGCCCACCTACCAGCTCCAGTCAGTACC |
| | CCAGCCTGTGGTCTGTGAGCAACGGCGCCGTCACCCCGGGCT |
| | CCCAGGCAGCAGCCGTGTCCAACGGGCTGGGGGCCCAGTTCT |
| | TCCGGGGCTCCCCCGCGCACTACACACCCCTCACCCATCCGG |
| | TCTCGGCGCCCTCTTCCTCGGGATCCCCACTGTACGAAGGGG |
| | CGGCCGCGGCCACAGACATCGTGGACAGCCAGTACGACGCC |
| | GCAGCCCAAGGCCGCCTCATAGCCTCATGGACACCTGTGTCG |
| | CCACCTTCCATGTGA |

1. A composition comprising a replication-defective virus vector comprising a nucleic acid sequence encoding a HER2/neu antigen that is a fragment of a native HER2/neu protein.

2. The composition of claim 1, wherein the HER2/neu antigen does not have an intracellular domain of a native HER2/neu protein.

3. The composition of claim 1, wherein the HER2/neu antigen has a transmembrane domain and an extracellular domain of a native HER2/neu protein.

4. The composition of claim 1, wherein the HER2/neu antigen has a sequence at least 80%, at least 85%, at least 90%, at least 92%, at least 95%, at least 97%, or at least 99% identical to SEQ ID NO: 1 or SEQ ID NO: 2, the nucleic acid sequence has a sequence at least 80%, at least 85%, at least 90%, at least 92%, at least 95%, at least 97%, or at least 99% identical to SEQ ID NO: 1 or positions 1033-3107 of SEQ ID NO: 3, and/or the replication-defective virus vector has a sequence at least 80%, at least 85%, at least 90%, at least 92%, at least 95%, at least 97%, or at least 99% identical to SEQ ID NO: 3.

5. The composition of claim 1, wherein the replication-defective virus vector is an adenovirus vector.

6. The composition of claim 5, wherein the adenovirus vector comprises a deletion in an E1 region, an E2b region, an E3 region, an E4 region, or a combination thereof.

7. (canceled)

8. (canceled)

9. The composition of claim 1, wherein the composition comprises from at least 1×10⁹ to at least 5×10¹² virus particles.

10.-13. (canceled)

14. The composition of claim 1, wherein the replication-defective virus vector further comprises a nucleic acid sequences encoding a costimulatory molecule.

15. The composition of claim 1, wherein the replication-defective virus vector further comprises a nucleic acid sequence encoding an immunological fusion partner.

16. The composition of claim 15, wherein the costimulatory molecule comprises B7, ICAM-1, LFA-3, or a combination thereof.

17. (canceled)

18. The composition of claim 1, wherein the composition further comprises a plurality of nucleic acid sequences encoding a plurality of costimulatory molecules positioned in the same replication-defective virus vector.

19. The composition of claim 1, wherein the composition further comprises a plurality of nucleic acid sequences encoding a plurality of costimulatory molecules positioned in separate replication-defective virus vectors.

20. The composition of claim 1, wherein the composition further comprises a nucleic acid sequence encoding one or more target antigens or immunological epitopes thereof.

21. The composition of claim 1, wherein the replication-defective virus vector further comprises a nucleic acid sequence encoding one or more target antigens or immunological epitopes thereof.

22. (canceled)

23. The composition of claim 20, wherein the one or more target antigens is folate receptor alpha, WT1, p53, MAGE-A1, MAGE-A2, MAGE-A3, MAGE-A4, MAGE-A6, MAGE-A10, MAGE-A12, BAGE, DAM-6, -10, GAGE-1, -2, -8, GAGE-3, -4, -5, -6, -7B, NA88-A, NY-ESO-1, MART-1, MC1R, Gp100, Tyrosinase, TRP-1, TRP-2, ART-4, CAMEL, CEA, Cyp-B, HER2/neu, BRCA1, BRACHYURY, BRACHYURY(TIVS7-2, polymorphism), BRACHYURY (IVS7 T/C polymorphism), T BRACHYURY, T, hTERT, hTRT, iCE, MUC1, MUC1 (VNTR polymorphism), MUC1-c, MUC1n, MUC2, PRAME, P15, RU1, RU2, SART-1, SART-3, WT1, AFP, β-catenin/m, Caspase-8/m, CEA, CDK-4/m, HER3, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, ETV6/AML, LDLR/FUT, Pml/RARα, or TEL/AML1, or a modified variant, a splice variant, a functional epitope, an epitope agonist, or a combination thereof.

24.-32. (canceled)

33. The composition of claim 1, wherein the replication-defective virus vector further comprises a selectable marker.

34. (canceled)

35. A pharmaceutical composition comprising the composition according to claim 1 and a pharmaceutically acceptable carrier.

36. A host cell comprising the composition according to claim 1.

37. A method of preparing a tumor vaccine, the method comprising preparing a pharmaceutical composition according to claim 35.

38. A method of enhancing an immune response in a subject in need thereof, the method comprising administering a therapeutically effective amount of the composition of claim 1 to the subject.

39. A method of treating a cancer in a subject in need thereof, the method comprising administering a therapeutically effective amount of the composition of claim 1 to the subject.

40.-83. (canceled)


SEQUENCE LISTING (105 sequences)

SEQ ID NO: 1 - DNA, 2075 bp, Artificial Sequence
  Description of Artificial Sequence Synthetic polynucleotide
ctagaatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcgtctctgcggtggttggcattctgctggtcgtggtcttgggggtggtctttgggatcctcatcaagcgacggcagcagaagatccggaagtacacgtaatctagataa

SEQ ID NO: 2 - protein, 688 aa, Artificial Sequence
  Description of Artificial Sequence Synthetic polypeptide
MRMELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIVSAVVGILLVVVLGVVFGILIKRRQQKIRKYT

SEQ ID NO: 3 - DNA, 32295 bp, Artificial Sequence
  Description of Artificial Sequence Synthetic polynucleotide
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactgtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctggtaccgtcgacgcggccgctcgagcctaagcttctagatgcatgctcgagcggccgccagtgtgatggatatctgcagaattcgcccttgctctagaatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcgtctctgcggtggttggcattctgctggtcgtggtcttgggggtggtctttgggatcctcatcaagcgacggcagcagaagatccggaagtacacgtaatctagataagatatccgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcggatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgataattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaatgtcaggtcgcctaagtcgattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgat

SEQ ID NO: 4 - DNA, 576 bp, Artificial Sequence
  Description of Artificial Sequence Synthetic polynucleotide
tagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcag

SEQ ID NO: 5 - DNA, 240 bp, Artificial Sequence
  Description of Artificial Sequence Synthetic polynucleotide
atctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatctta

SEQ ID NO: 6 - protein, 9 aa, Artificial Sequence
  Description of Artificial Sequence Synthetic peptide
EQVWGMAVR

SEQ ID NO: 7 - protein, 15 aa, Artificial Sequence
  Description of Artificial Sequence Synthetic peptide
CQGPEQVWGMAVREL

SEQ ID NO: 8 - protein, 9 aa, Artificial Sequence
  Description of Artificial Sequence Synthetic peptide
GETVTMPCP

SEQ ID NO: 9 - protein, 15 aa, Artificial Sequence
  Description of Artificial Sequence Synthetic peptide
NVGETVTMPCPKVFS

SEQ ID NO: 10 - protein, 9 aa, Artificial Sequence
  Description of Artificial Sequence Synthetic peptide
GLGAQCSEA

SEQ ID NO: 11 - protein, 15 aa, Artificial Sequence
  Description of Artificial Sequence Synthetic peptide
NNGLGAQCSEAVTLN

SEQ ID NO: 12 - protein, 9 aa, Artificial Sequence
  Description of Artificial Sequence Synthetic peptide
RKLTTELTI

SEQ ID NO: 13 - protein, 15 aa, Artificial Sequence
  Description of Artificial Sequence Synthetic peptide
LGPERRKLTTELTII

SEQ ID NO: 14 - protein, 9 aa, Artificial Sequence
  Description of Artificial Sequence Synthetic peptide
PERRKLTTE

SEQ ID NO: 15 - protein, 9 aa, Artificial Sequence
  Description of Artificial Sequence Synthetic peptide
MDWVWMDTT

SEQ ID NO: 16 - protein, 15 aa, Artificial Sequence
  Description of Artificial Sequence Synthetic peptide
AVMDWVWMDTTLSLS

SEQ ID NO: 17 - protein, 9 aa, Artificial Sequence
  Description of Artificial Sequence Synthetic peptide
VWMDTTLSL

SEQ ID NO: 18 - protein, 9 aa, Artificial Sequence
  Description of Artificial Sequence Synthetic peptide
GKTLNPSQT

SEQ ID NO: 19 - protein, 15 aa, Artificial Sequence
  Description of Artificial Sequence Synthetic peptide
SWFREGKTLNPSQTS

SEQ ID NO: 20 - protein, 9 aa, Artificial Sequence
  Description of Artificial Sequence Synthetic peptide
REGKTLNPS

SEQ ID NO: 21 - protein, 9 aa, Artificial Sequence
  Description of Artificial Sequence Synthetic peptide
VRNATSYRC

SEQ ID NO: 22 - protein, 15 aa, Artificial Sequence
  Description of Artificial Sequence Synthetic peptide
LPNVTVRNATSYRCG

SEQ ID NO: 23 - protein, 9 aa, Artificial Sequence
  Description of Artificial Sequence Synthetic peptide
NVTVRNATS

SEQ ID NO: 24 - protein, 9 aa, Artificial Sequence
  Description of Artificial Sequence Synthetic peptide
FAMAQIPSL

SEQ ID NO: 25 - protein, 15 aa, Artificial Sequence
  Description of Artificial Sequence Synthetic peptide
PFAMAQIPSLSLRAV

SEQ ID NO: 26 - protein, 9 aa, Artificial Sequence
  Description of Artificial Sequence Synthetic peptide
AQIPSLSLR

SEQ ID NO: 27 - DNA, 1992 bp, Homo sapiens
atgagggcgaacgacgctctgcaggtgctgggcttgcttttcagcctggcccggggctccgaggtgggcaactctcaggcagtgtgtcctgggactctgaatggcctgagtgtgaccggcgatgctgagaaccaataccagacactgtacaagctctacgagaggtgtgaggtggtgatggggaaccttgagattgtgctcacgggacacaatgccgacctctccttcctgcagtggattcgagaagtgacaggctatgtcctcgtggccatgaatgaattctctactctaccattgcccaacctccgcgtggtgcgagggacccaggtctacgatgggaagtttgccatcttcgtcatgttgaactataacaccaactccagccacgctctgcgccagctccgcttgactcagctcaccgagattctgtcagggggtgtttatattgagaagaacgataagctttgtcacatggacacaattgactggagggacatcgtgagggaccgagatgctgagatagtggtgaaggacaatggcagaagctgtcccccctgtcatgaggtttgcaaggggcgatgctggggtcctggatcagaagactgccagacattgaccaagaccatctgtgctcctcagtgtaatggtcactgctttgggcccaaccccaaccagtgctgccatgatgagtgtgccgggggctgctcaggccctcaggacacagactgctttgcctgccggcacttcaatgacagtggagcctgtgtacctcgctgtccacagcctcttgtctacaacaagctaactttccagctggaacccaatccccacaccaagtatcagtatggaggagtttgtgtagccagctgtccccataactttgtggtggatcaaacatcctgtgtcagggcctgtcctcctgacaagatggaagtagataaaaatgggctcaagatgtgtgagccttgtgggggactatgtcccaaagcctgtgagggaacaggctctgggagccgcttccagactgtggactcgagcaacattgatggatttgtgaactgcaccaagatcctgggcaacctggactttctgatcaccggcctcaatggagacccctggcacaagatccctgccctggacccagagaagctcaatgtcttccggacagtacgggagatcacaggttacctgaacatccagtcctggccgccccacatgcacaacttcagtgttttttccaatttgacaaccattggaggcagaagcctctacaaccggggcttctcattgttgatcatgaagaacttgaatgtcacatctctgggcttccgatccctgaaggaaattagtgctgggcgtatctatataagtgccaataggcagctctgctaccaccactctttgaactggaccaaggtgcttcgggggcctacggaagagcgactagacatcaagcataatcggccgcgcagagactgcgtggcagagggcaaagtgtgtgacccactgtgctcctctgggggatgctggggcccaggccctggtcagtgcttgtcctgtcgaaattatagccgaggaggtgtctgtgtgacccactgcaactttctgaatggggagcctcgagaatttgcccatgaggccgaatgcttctcctgccacccggaatgccaacccatggagggcactgccacatgcaatggctcgggctctgatacttgtgctcaatgtgcccattttcgagatgggccccactgtgtgagcagctgcccccatggagtcctaggtgccaagggcccaatctacaagtacccagatgttcagaatgaatgtcggccctgccatgagaactgcacccaggggtgtaaaggaccagagcttcaagactgtttaggacaaacactggtgctgatcggcaaaacccatctgacaatggctttgacagtgatagcaggattggtagtgattttcatgatgctgggcggcactttttaa

SEQ ID NO: 28 - protein, 9 aa, Homo sapiens
YLSGANLNL

SEQ ID NO: 29 - DNA, 32315 bp, Artificial Sequence
  Description of Artificial Sequence Synthetic polynucleotide
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactgtaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagagatctggtaccgtcgacgcggccgctcgagcctaagcttggtaccgagctcggatccactagtaacggccgccagtgtgctggaattcggcttaaaggtacccagagcagacagccgccaccatggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcggacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatagcagccctggtgtagtttcttcatttcaggaagactgacagttgttttgcttcttccttaaagcatttgcaacagctacagtctaaaattgcttctttaccaaggatatttacagaaaagactctgaccagagatcgagaccatcctctagataagatatccgatccaccggatctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcggatctgggcgtggttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgataattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagcgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatcctctagttaatgtcaggtcgcctaagtcgattaactagagtacccggggatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgat

SEQ ID NO: 30 - DNA, 2109 bp, Homo sapiens
atggagtctccctcggcccctccccacagatggtgcatcccctggcagaggctcctgctcacagcctcacttctaaccttctggaacccgcccaccactgccaagctcactattgaatccacgccgttcaatgtcgcagaggggaaggaggtgcttctacttgtccacaatctgccccagcatctttttggctacagctggtacaaaggtgaaagagtggatggcaaccgtcaaattataggatatgtaataggaactcaacaagctaccccagggcccgcatacagtggtcgagagataatataccccaatgcatccctgctgatccagaacatcatccagaatgacacaggattctacaccctacacgtcataaagtcagatcttgtgaatgaagaagcaactggccagttccgggtatacccggagctgcccaagccctccatctccagcaacaactccaaacccgtggaggacaaggatgctgtggccttcacctgtgaacctgagactcaggacgcaacctacctgtggtgggtaaacaatcagagcctcccggtcagtcccaggctgcagctgtccaatggcaacaggaccctcactctattcaatgtcacaagaaatgacacagcaagctacaaatgtgaaacccagaacccagtgagtgccaggcgcagtgattcagtcatcctgaatgtcctctatggcccggatgcccccaccatttcccctctaaacacatcttacagatcaggggaaaatctgaacctctcctgccacgcagcctctaacccacctgcacagtactcttggtttgtcaatgggactttccagcaatccacccaagagctctttatccccaacatcactgtgaataatagtggatcctatacgtgccaagcccataactcagacactggcctcaataggaccacagtcacgacgatcacagtctatgcagagccacccaaacccttcatcaccagcaacaactccaaccccgtggaggatgaggatgctgtagccttaacctgtgaacctgagattcagaacacaacctacctgtggtgggtaaataatcagagcctcccggtcagtcccaggctgcagctgtccaatgacaacaggaccctcactctactcagtgtcacaaggaatgatgtaggaccctatgagtgtggaatccagaacgaattaagtgttgaccacagcgacccagtcatcctgaatgtcctctatggcccagacgaccccaccatttccccctcatacacctattaccgtccaggggtgaacctcagcctctcctgccatgcagcctctaacccacctgcacagtattcttggctgattgatgggaacatccagcaacacacacaagagctctttatctccaacatcactgagaagaacagcggactctatacctgccaggccaataactcagccagtggccacagcaggactacagtcaagacaatcacagtctctgcggagctgcccaagccctccatctccagcaacaactccaaacccgtggaggacaaggatgctgtggccttcacctgtgaacctgaggctcagaacacaacctacctgtggtgggtaaatggtcagagcctcccagtcagtcccaggctgcagctgtccaatggcaacaggaccctcactctattcaatgtcacaagaaatgacgcaagagcctatgtatgtggaatccagaactcagtgagtgcaaaccgcagtgacccagtcaccctggatgtcctctatgggccggacacccccatcatttcccccccagactcgtcttacctttcgggagcggacctcaacctctcctgccactcggcctctaacccatccccgcagtattcttggcgtatcaatgggataccgcagcaacacacacaagttctctttatcgccaaaatcacgccaaataataacgggacctatgcctgttttgtctctaacttggctactggccgcaataattccatagtcaagagcatcacagtctctgcatctggaacttctcctggtctctcagctggggccactgtcggcatcatgattggagtgctggttggggttgctctgatatag

SEQ ID NO: 31 - protein, 9 aa, Homo sapiens
YLSGADLNL

SEQ ID NO: 32 - DNA, 1428 bp, Homo sapiens
atgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccctttggggacaggatgtcacctcggtcccagtcaccaggccagccctgggctccaccaccccgccagcccacgatgtcacctcagccccggacaacaagccagccccgggctccaccgcccccccagcccacggtgtcacctcgtatcttgacaccaggccggccccggtttatcttgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggtttatctggccattgtctatctcattgccttggctgtcgctcaggttcgccgaaagaactacgggcagctggacatctttccagcccgggataaataccatcctatgagcgagtacgctctttaccacacccatgggcgctatgtgccccctagcagtcttttccgtagcccctatgagaaggtttctgcaggtaatggtggcagctatctctcttacacaaacccagcagtggcagccgcttctgccaacttgtag

SEQ ID NO: 33 - protein, 475 aa, Artificial Sequence
  Description of Artificial Sequence Synthetic polypeptide
MTPGTQSPFFLLLLLTVLTVVTGSGHASSTPGGEKETSATQRSSVPSSTEKNAVSMTSSVLSSHSPGSGSSTTQGQDVTLAPATEPASGSAALWGQDVTSVPVTRPALGSTTPPAHDVTSAPDNKPAPGSTAPPAHGVTSYLDTRPAPVYLAPPAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVYLAIVYLIALAVAQVRRKNYGQLDIFPARDKYHPMSEYALYHTHGRYVPPSSLFRSPYEKVSAGNGGSYLSYTNPAVAAASANL

SEQ ID NO: 34 - DNA, 1233 bp, Homo sapiens
atgagctcccctggcaccgagagcgcgggaaagagcctgcagtaccgagtggaccacctgctgagcgccgtggagaatgagctgcaggcgggcagcgagaagggcgaccccacagagcgcgaactgcgcgtgggcctggaggagagcgagctgtggctgcgcttcaaggagctcaccaatgagatgatcgtgaccaagaacggcaggaggatgtttccggtgctgaaggtgaacgtgtctggcctggaccccaacgccatgtactccttcctgctggacttcgtggcggcggacaaccaccgctggaagtacgtgaacggggaatgggtgccggggggcaagccggagccgcaggcgcccagctgcgtctacatccaccccgactcgcccaacttcggggcccactggatgaaggctcccgtctccttcagcaaagtcaagctcaccaacaagctcaacggagggggccagatcatgctgaactccttgcataagtatgagcctcgaatccacatagtgagagttgggggtccacagcgcatgatcaccagccactgcttccctgagacccagttcatagcggtgactgctagaagtgatcacaaagagatgatggaggaacccggagacagccagcaacctgggtactcccaatgggggtggcttcttcctggaaccagcaccgtgtgtccacctgcaaatcctcatcctcagtttggaggtgccctctccctcccctccacgcacagctgtgacaggtacccaaccctgaggagccaccggtcctcaccctaccccagcccctatgctcatcggaacaattctccaacctattctgacaactcacctgcatgtttatccatgctgcaatcccatgacaattggtccagccttggaatgcctgcccatcccagcatgctccccgtgagccacaatgccagcccacctaccagctccagtcagtaccccagcctgtggtctgtgagcaacggcgccgtcaccccgggctcccaggcagcagccgtgtccaacgggctgggggcccagttcttccggggctcccccgcgcactacacacccctcacccatccggtctcggcgccctcttcctcgggatccccactgtacgaaggggcggccgcggccacagacatcgtggacagccagtacgacgccgcagcccaaggccgcctcatagcctcatggacacctgtgtcgccaccttccatgtga

SEQ ID NO: 35 - protein, 132 aa, Mycobacterium tuberculosis
TAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPA

SEQ ID NO: 36 - protein, 230 aa, Artificial Sequence
  Description of Artificial Sequence Synthetic polypeptide
MHHHHHHTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFDDDDKDPPDPHQPDMTKGYCPGGRWGFGDLAVCDGEKYPDGSFWHQWMQTWFTGPQFYFDCVSGGEPLPGPPPPGGCGGAIPSEQPNAP

SEQ ID NO: 37 - protein, 578 aa, Artificial Sequence
  Description of Artificial Sequence Synthetic polypeptide
MHHHHHHTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFPLVPRGSPMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQDVRRVPGVAPTLVRSASETSEKRPFMCAYSGCNKRYFKLSHLQMHSRKHTGEKPYQCDFKDCERRFFRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 38 - protein, 220 aa, Artificial Sequence [sequence omitted]

SEQ ID NO: 39 - protein, 729 aa, Artificial Sequence
  Description of Artificial Sequence Synthetic polypeptide
MHHHHHHTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFMVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAGDIAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNTAAS

SEQ ID NO: 40 - protein, 30 aa, Artificial Sequence
  Description of Artificial Sequence Synthetic polypeptide
TAASDNFQLSQGGQGFAIPIGQAMAIAGQI

SEQ ID NO: 41 - protein, 128 aa, Artificial Sequence
  Description of Artificial Sequence Synthetic polypeptide
TAASDNFQLSQGGQGFAIPIGQAMAIAGQIKLPTVHIGPTAFLGLGVVDNNGNGARVQRVVGSAPAASLGISTGDVITAVDGAPINSATAMADALNGHHPGDVISVTWQTKSGGTRTGNVTLAEGPPA

SEQ ID NO: 42 - protein, 128 aa, Mycobacterium tuberculosis
TAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAE

SEQ ID NO: 43 - protein, 355 aa, Mycobacterium tuberculosis
MSNSRRRSLRWSWLLSVLAAVGLGLATAPAQAAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPA

SEQ ID NO: 44 - protein, 364 aa, Haemophilus influenzae
MKLKTLALSLLAAGVLAGCSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETKDGKQAQVYPNRFPLWKSHFRIHTFEDEIEFIQGLEKSTGKKVGIYPEIKAPWFHHQNGKDIAAETLKVLKKYGYDKKTDMVYLQTFDFNELKRIKTELLPQMGMDLKLVQLIAYTDWKETQEKDPKGYWVNYNYDWMFKPGAMAEVVKYADGVGPGWYMLVNKEESKPDNIVYTPLVKELAQYNVEVHPYTVRKDALPAFFTDVNQMYDVLLNKSGATGVFTDFPDTGVEFLKGIK

SEQ ID NO: 45 - protein, 313 aa, Streptococcus pneumonae
MEINVSKLRTDLPQVGVQPYRQVHAHSTGNPHSTVQNEADYHWRKDPELGFFSHIVGNGCIMQVGPVDNGAWDVGGGWNAETYAAVELIESHSTKEEFMTDYRLYIELLRNLADEAGLPKTLDTGSLAGIKTHEYCTNNQPNNHSDHVDPYPYLAKWGISREQFKHDIENGLTIETGWQKNDTGYWYVHSDGSYPKDKFEKINGTWYYFDSSGYMLADRWRKHTDGNWYWFDNSGEMATGWKKIADKWYYFNEEGAMKTGWVKYKDTWYYLDAKEGAMVSNAFIQSADGTGWYYLKPDGTLADRPEFRMSQMA

SEQ ID NO: 46 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 47 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 48 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 49 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 50 - protein, 662 aa, Homo sapiens
MEPLVTWVVPLLFLFLLSRQGAACRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 51 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 52 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 53 - protein, 152 aa, Homo sapiens
MSRLPVLLLLQLLVRPGLQAPMTQTTSLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 54 - protein, 153 aa, Homo sapiens [sequence omitted]

SEQ ID NO: 55 - protein, 134 aa, Homo sapiens
MRMLLHLSLLALGAAYVYAIPTEIPTSALVKETLALLSTHRTLLIANETLRIPVPVHKNHQLCTEEIFQGIGTLESQTVQGGTVERLFKNLSLIKKYIDGQKKKCGEERRRVNQFLDYLQEFLGVMNTEWIIES

SEQ ID NO: 56 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 57 - protein, 140 aa, Homo sapiens
MVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 58 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 59 - protein, 145 aa, Homo sapiens [sequence omitted]

SEQ ID NO: 60 - protein, 136 aa, Homo sapiens [sequence omitted]

SEQ ID NO: 61 - protein, 656 aa, Homo sapiens
MEGDGSDPEPPDAGEDSKSENGENAPIYCICRKPDINCFMIGCDNCNEWFHGDCIRITEKMAKAIREWYCRECREKDPKLEIRYRHKKSRERDGNERDSSEPRDEGGGRKRPVPDPNLQRRAGSGTGVGAMLARGSASPHKSSPQPLVATPSQHHQQQQQQIKRSARMCGECEACRRTEDCGHCDFCRDMKKFGGPNKIRQKCRLRQCQLRARESYKYFPSSLSPVTPSESLPRPRRPLPTQQQPQPSQKLGRIREDEGAVASSTVKEPPEATATPEPLSDEDLPLDPDLYQDFCAGAFDDNGLPWMSDTEESPFLDPALRKRAVKVKHVKRREKKSEKKKEERYKRHRQKQKHKDKWKHPERADAKDPASLPQCLGPGCVRPAQPSSKYCSDDCGMKLAANRIYEILPQRIQQWQQSPCIAEEHGKKLLERIRREQQSARTRLQEMERRFHELEAIILRAKQQAVREDEESNEGDSDDTDLQIFCVSCGHPINPRVALRHMERCYAKYESQTSFGSMYPTRIEGATRLFCDVYNPQSKTYCKRLQVLCPEHSRDPKVPADEVCGCPLVRDVFELTGDFCRLPKRQCNRHYCWEKLRRAEVDLERVRVWYKLDELFEQERNVRTAMTNRAGLLALMLHQTIQHDPLTTDLRSSADR

SEQ ID NO: 62 - protein, 124 aa, Vibrio cholerae
MIKLKFGVFFTVLLSSAYAHGTPQNITDLCAEYHNTQIYTLNDKIFSYTESLAGKREMAIITFKNGAIFQVEVPGSQHIDSQKKAIERMKDTLRIAYLTEAKVEKLCVWNNKTPHAIAAISMAN

SEQ ID NO: 63 - protein, 258 aa, Vibrio cholerae
MVKIIFVFFIFLSSFSYANDDKLYRADSRPPDEIKQSGGLMPRGQNEYFDRGTQMNINLYDHARGTQTGFVRHDDGYVSTSISLRSAHLVGQTILSGHSTYYIYVIATAPNMFNVNDVLGAYSPHPDEQEVSALGGIPYSQIYGWYRVHFGVLDEQLHRNRGYRDRYYSNLDIAPAADGYGLAGFPPEHRAWREEPWIHHAPPGCGNAPRSSMSNTCDEKTQSLGVKFLDEYQSKVKRQIFSGYQSDIDTHNRIKDEL

SEQ ID NO: 64 - protein, 124 aa, Vibrio cholerae
MIKLKFGVFFTVLLSSAYAHGTPQNITDLCAEYHNTQIHTLNDKILSYTESLAGNREMAIITFKNGATFQVEVPGSQHIDSQKKAIERMKDTLRIAYLTEAKVEKLCVWNNKTPHAIAAISMAN

SEQ ID NO: 65 - protein, 18 aa, Homo sapiens
DPNAPKRPPSAFFLFCSE

SEQ ID NO: 66 - protein, 96 aa, Homo sapiens
MCCTKSLLLAALMSVLLLHLCGESEAASNFDCCLGYTDRILHPKFIVGFTRQLANEGCDINAIIFHTKKKLSVCANPKQTWVKYIVRLLSKKVKNM

SEQ ID NO: 67 - protein, 92 aa, Homo sapiens
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 68 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 69 - protein, 204 aa, Homo sapiens
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 70 - protein, 7 aa, Artificial Sequence
  Description of Artificial Sequence Synthetic peptide
QEINSSY

SEQ ID NO: 71 - protein, 7 aa, Artificial Sequence
  Description of Artificial Sequence Synthetic peptide
SHPRLSA

SEQ ID NO: 72 - protein, 7 aa, Artificial Sequence
  Description of Artificial Sequence Synthetic peptide
SMPNPMV

SEQ ID NO: 73 - protein, 7 aa, Artificial Sequence
  Description of Artificial Sequence Synthetic peptide
GLQQVLL

SEQ ID NO: 74 - protein, 7 aa, Artificial Sequence
  Description of Artificial Sequence Synthetic peptide
HELSVLL

SEQ ID NO: 75 - protein, 7 aa, Artificial Sequence
  Description of Artificial Sequence Synthetic peptide
YAPQRLP

SEQ ID NO: 76 - protein, 7 aa, Artificial Sequence
  Description of Artificial Sequence Synthetic peptide
TPRTLPT

SEQ ID NO: 77 - protein, 7 aa, Artificial Sequence
  Description of Artificial Sequence Synthetic peptide
APVHSSI

SEQ ID NO: 78 - protein, 7 aa, Artificial Sequence
  Description of Artificial Sequence Synthetic peptide
APPHALS

SEQ ID NO: 79 - protein, 7 aa, Artificial Sequence
  Description of Artificial Sequence Synthetic peptide
TFSNRFI

SEQ ID NO: 80 - protein, 7 aa, Artificial Sequence
  Description of Artificial Sequence Synthetic peptide
VVPTPPY

SEQ ID NO: 81 - protein, 7 aa, Artificial Sequence
  Description of Artificial Sequence Synthetic peptide
ELAPDSP

SEQ ID NO: 82 - protein, 69 aa, Shigella dysenteria 1
TPDCVTGKVEYTKYNDDDTFTVKVGDKELFTNRWNLQSLLLSAQITGMTVTIKQNACHNGGGFSEVIFR

SEQ ID NO: 83 - protein, 560 aa, Corynephage omega
MSRKLFASILIGALLGIGAPPSAHAGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 84 - protein, 114 aa, Artificial Sequence
  Description of Artificial Sequence Synthetic polypeptide
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 85 - protein, 297 aa, Artificial Sequence
  Description of Artificial Sequence Synthetic polypeptide
ITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 535 aa, Corynebacterium diphtheriae
GADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 87 - protein, 1331 aa, Homo sapiens [sequence omitted]

SEQ ID NO: 88 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 89 - protein, 476 aa, Homo sapiens
RAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSPIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 90 - protein, 234 aa, Homo sapiens
MCFPKVLSDDMKKLKARMVMLLPTSAQGLGAWVSACDTEDTVGHLGPWRDKDPALWCQLCLSSQHQAIERFYDKMQNAESGRGQVMSSLAELEDDFKEGYLETVAAYYEEQHPELTPLLEKERDGLRCRGNRSPVPDVEDPATEEPGESFCDKVMRWFQAMLQRLQTWWHGVLAWVKEKVVALVHAVQALWKQFQSFCCSLSELFMSSFQSYGAPRGDKEELTPQKCSEPQSSK

SEQ ID NO: 91 - protein, 11 aa, Artificial Sequence
  Description of Artificial Sequence Synthetic peptide
MAVPMQLSCSR

SEQ ID NO: 92 - protein, 4 aa, Artificial Sequence
  Description of Artificial Sequence Synthetic peptide
RSTG

SEQ ID NO: 93 - protein, 2 aa, Artificial Sequence
  Description of Artificial Sequence Synthetic peptide
TR

SEQ ID NO: 94 - protein, 3 aa, Artificial Sequence
  Description of Artificial Sequence Synthetic peptide
RSQ

SEQ ID NO: 95 - protein, 5 aa, Artificial Sequence
  Description of Artificial Sequence Synthetic peptide
RSAGE

SEQ ID NO: 96 - protein, 2 aa, Artificial Sequence
  Description of Artificial Sequence Synthetic peptide
RS

SEQ ID NO: 97 - protein, 2 aa, Artificial Sequence
  Description of Artificial Sequence Synthetic peptide
GG

SEQ ID NO: 98 - protein, 9 aa, Artificial Sequence
  Description of Artificial Sequence Synthetic peptide
GSGGSGGSG

SEQ ID NO: 99 - protein, 11 aa, Artificial Sequence
  Description of Artificial Sequence Synthetic peptide
GGSGGSGGSGG

SEQ ID NO: 100 - protein, 14 aa, Artificial Sequence
  Description of Artificial Sequence Synthetic peptide
GGSGGSGGSGGSGG

SEQ ID NO: 101 - protein, 17 aa, Artificial Sequence
  Description of Artificial Sequence Synthetic peptide
GGSGGSGGSGGSGGSGG

SEQ ID NO: 102 - protein, 20 aa, Artificial Sequence
  Description of Artificial Sequence Synthetic peptide
GGSGGSGGSGGSGGSGGSGG

SEQ ID NO: 103 - protein, 23 aa, Artificial Sequence
  Description of Artificial Sequence Synthetic peptide
GGSGGSGGSGGSGGSGGSGGSGG

SEQ ID NO: 104 - protein, 16 aa, Artificial Sequence
  Description of Artificial Sequence Synthetic peptide
GGSGGSGGSGGSGGSG

SEQ ID NO: 105 - protein, 16 aa, Artificial Sequence
  Description of Artificial Sequence Synthetic peptide
GSGGSGGSGGSGGSGG